SEQUENCE LISTING (4 sequences)

SEQ ID NO: 1 - DNA, 24 bp, synthetic construct
  misc_feature 1..24: note = Description of sequence: Component of compound NSC-617145
  source 1..24: mol_type = other DNA
gcactggccgtcgttttacggtcg

SEQ ID NO: 2 - DNA, 23 bp, synthetic construct
  misc_feature 1..23: note = Description of sequence: Component of compound NSC-617145
  source 1..23: mol_type = other DNA
tccaagtaaaacgacggccagtg

SEQ ID NO: 3 - DNA, 2208 bp, Homo sapiens
  misc_feature 1..2208: note = Description of sequence: Gene sequence of WRN-BV08
  source 1..2208: mol_type = genomic DNA
atgaacgagggcgaagaagacgacgacaaggacttcctgtggcctgcccctaacgaagaacaagtgacatgcctgaagatgtacttcggacacagtagcttcaagcctgtgcaatggaaggtcatccactccgtgctggaagaaagaagggacaacgtggctgtgatggctaccggatacggtaagtccctgtgcttccagtaccctcccgtgtacgtgggcaagatcggtctggtgatctcccctctgatctctctgatggaggaccaggtgctgcaattgaagatgtccaacatccccgcttgcttcctgggttccgctcaaagtgagaacgtgctgacagacatcaagctgggcaagtaccgcatcgtgtacgtgacccctgagtactgctccggtaacatgggtctgctgcaacagctggaggctgacatcggaatcaccctgatcgctgtggacgaggctcactgcatctccgagtggggacacgacttccgcgactccttccgtaagctgggatccttgaagaccgctctccctatggtgcctatcgtggccctgaccgccactgcttcctcctccatccgcgaggacatcgtgcgttgcctgaacctgcgcaaccctcagatcacttgcaccggtttcgaccgccctaacttgtacctcgaggtgcgtcgcaagaccggtaacatcctccaggacctgcagcctttcctggtcaagacctcctcccactgggaatttgagggccctaccatcatctactgcccttcccgcaagatgacccagcaagtcaccggcgagctgcgcaagctcaacctctcctgcggtacctaccacgctggtatgtccttctccacccgcaaggacatccaccaccgcttcgtccgtgacgaaatccaatgcgtcatcgctaccatcgctttcggaatgggcatcaacaaggctgacatccgccaggtgatccactacggcgcccccaaggacatggaatcctactaccaggaaatcggtcgcgccggtcgcgacggtctgcagtcttcctgtcacgtgctgtgggcccccgctgacatcaacctgaaccgccacctgctgaccgaaatccgcaacgagaagttccgcctgtacaagctcaagatgatggctaagatggagaagtacctgcactcctcccgctgtcgccgtcagatcatcctctcccacttcgaggacaagcaagtgcaaaaggctagcctgggtatcatgggcaccgaaaagtgttgtgacaactgccgctcccgcctcgaccactgctactccatggacgacagcgaggacacctcctgggacttcggtcctcaagctttcaagctcttgtccgctgtggacatcctgggcgagaagttcggtatcggtctccccatcctcttcctgcgtggtagcaactcccaacgcctggctgaccagtaccgccgccactccctcttcggtaccggtaaggaccagaccgagtcctggtggaaggctttctctcgccaactgatcaccgaaggtttcctggtggaggtgtcccgctacaacaagttcatgaagatctgcgctctcactaagaagggaaggaactggctgcacaaggctaacactgagtcccaatccctcatcctgcaggctaacgaggagctgtgccctaagaagttcctgctgccttcctccaagaccgtgtcctccggaacaaaggaacactgctacaaccaagtccctgtggagctctccaccgagaagaagtccaacctggagaagctgtacagctacaagccttgcgacaagatcagctccggttccaacatcagcaagaagtccatcatggtgcaatcccctgaaaaggcctactccagctcccaacctgtcatctccgctcaagagcaagagacccagatcgtgctgtacggtaagctggtcgaagcccgccaaaagcacgctaacaagatggacgtccctcccgctatcctcgccaccaacaagatcctcgtggatatggctaagatgcgccccaccaccgtcgagaacgtgaagcgcatcgacggtgtctccgagggtaaggccgctatgctggctcctctgctggaagtgatcaagcacttctgccagaccaactccgtgcagaccgacctgttcagtagtgagaacctgtacttccaaggccaccatcatcatcatcatcaccactaa

SEQ ID NO: 4 - protein, 735 aa, Homo sapiens
  REGION 1..735: note = Description of sequence: Protein sequence of WRN-BV08
MNEGEEDDDKDFLWPAPNEEQVTCLKMYFGHSSFKPVQWKVIHSVLEERRDNVAVMATGYGKSLCFQYPPVYVGKIGLVISPLISLMEDQVLQLKMSNIPACFLGSAQSENVLTDIKLGKYRIVYVTPEYCSGNMGLLQQLEADIGITLIAVDEAHCISEWGHDFRDSFRKLGSLKTALPMVPIVALTATASSSIREDIVRCLNLRNPQITCTGFDRPNLYLEVRRKTGNILQDLQPFLVKTSSHWEFEGPTIIYCPSRKMTQQVTGELRKLNLSCGTYHAGMSFSTRKDIHHRFVRDEIQCVIATIAFGMGINKADIRQVIHYGAPKDMESYYQEIGRAGRDGLQSSCHVLWAPADINLNRHLLTEIRNEKFRLYKLKMMAKMEKYLHSSRCRRQIILSHFEDKQVQKASLGIMGTEKCCDNCRSRLDHCYSMDDSEDTSWDFGPQAFKLLSAVDILGEKFGIGLPILFLRGSNSQRLADQYRRHSLFGTGKDQTESWWKAFSRQLITEGFLVEVSRYNKFMKICALTKKGRNWLHKANTESQSLILQANEELCPKKFLLPSSKTVSSGTKEHCYNQVPVELSTEKKSNLEKLYSYKPCDKISSGSNISKKSIMVQSPEKAYSSSQPVISAQEQETQIVLYGKLVEARQKHANKMDVPPAILATNKILVDMAKMRPTTVENVKRIDGVSEGKAAMLAPLLEVIKHFCQTNSVQTDLFSSENLYFQGHHHHHHHH

(19) United States
(12) Patent Application Publication
(10) Pub. No.: US 2026/0356840 A1
(43) Pub. Date: Nov. 12, 2026

(54) [1,2,4]TRIAZOLO[1,5-A]PYRIMIDINONE WRN INHIBITORS

(71) Applicant: Nimbus Wadjet, Inc., Boston, MA (US)

(72) Inventors: Florian Bartels, Berlin (DE); Justin Caravella, Cambridge, MA (US); Nathan E. Genung, Boston, MA (US); Silvana Marcel Leit de Moradei, Burlington, MA (US); Derun Li, Boston, MA (US); Angela V. West, Franklin, MA (US)

(21) Appl. No.: 19/476,514

(22) PCT Filed: Apr. 19, 2024
(86) PCT No.: PCT/US2024/025525

§ 371 Date: Oct. 17, 2025

Related U.S. Application Data
(60) Provisional application No. 63/496,323, filed on Apr. 21, 2023.

(51) Int. Cl. C07D 487/04; A61K 31/519; C07D 519/00

(52) U.S. Cl. CPC C07D 487/04; A61K 31/519; C07D 519/00

(57) ABSTRACT

The present disclosure is directed to [1,2,4]triazolo[1,5-a]pyrimidin-7(4H)-one based compounds, including compounds of Formula (I) and pharmaceutically acceptable salts thereof, and compositions thereof, as well as methods of treatment of cancers such as those involving WRN protein.

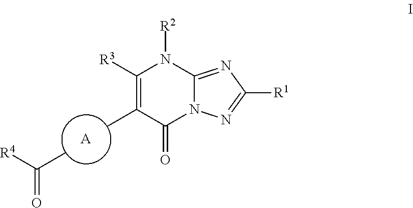

### CROSS-REFERENCE TO RELATED APPLICATIONS

This application claims the benefit of U.S. Provisional Application No. U.S. 63/496,323, filed Apr. 21, 2023; the entire contents of which is incorporated herein by reference.

### FIELD OF INVENTION

The invention provides bicyclic compounds and compositions, the use thereof and methods using the compounds, for inhibiting Werner Syndrome RecQ DNA helicase (WRN) and methods of treating disease using said compounds, in particular the use in treating cancer, and in particular the treatment of cancer characterized as microsatellite instability-high (MSI-H) or mismatch repair deficient (dMMR), including colorectal, gastric and endometrial cancer. The invention also provides the use of said compounds as research chemicals, intermediate compounds, combinations, processes and formulations.

### SEQUENCE LISTING

This application contains a Sequence Listing which has been submitted in .xml format via EFS and is hereby incorporated by reference. The ST.26 copy, created on Mar. 30, 2023, is named 407274-77WRP1_ST26.xml and is 8,751 bytes in size.

### BACKGROUND

Loss of DNA mismatch repair is a common initiating event in cancer development occurring in 10-30% of colorectal, endometrial, ovarian and gastric cancers (Aaltonen, L. A. et al. Clues to the pathogenesis of familial colorectal cancer, Science 260, 812-816 (1993), Bonneville R et al., Landscape of Microsatellite Instability Across 39 Cancer Types. JCO Precis Oncol. 1: PO.17.00073 (2017)). Cancers that are deficient in mismatch repair (dMMR) have a high mutational burden, and frequent deletion and insertion events in repetitive DNA tracts, a phenotype known as microsatellite instability (MSI). While progress has been made in the treatment of microsatellite instability high (MSI-H) cancers, and the demonstration that pembrolizumab (anti-PD1) treatment led to significantly longer progression-free survival than chemotherapy when received as first-line therapy for MSI-H-dMMR metastatic colorectal cancer (CRC) which resulted in the recent approval of pembrolizumab as first-line treatment of these cancers, there is still a significant unmet medical need in CRC and other MSI-H indications (Andre T., et al. Pembrolizumab in Microsatellite-Instability-High Advanced Colorectal Cancer. N Engl J Med 383(23):22072218 (2020)). Several large-scale functional genomics screens across large panels of cell lines, including Novartis with 398 cell lines from the Cancer Cell Line Encyclopedia (CCLE) (McDonald E. R. et al., Project DRIVE: A Compendium of Cancer Dependencies and Synthetic Lethal Relationships Uncovered by Large-Scale, Deep RNAi Screening. Cell 170 (3): 577-592 (2017)), have identified the Werner Syndrome RecQ helicase (WRN) as being selectively required for the survival of cell lines with defective mismatch repair that have become MSI-H (Behan, F. M. et al. Prioritization of cancer therapeutic targets using CRISPR-Cas9 screens. Nature 568, 511-516 (2019), Chan, E. M. et al. WRN helicase is a synthetic lethal target in microsatellite unstable cancers. Nature 568, 551-556 (2019). Kategaya, L., Perumal, S. K., Hager, J. H. & Belmont, L. D. Werner syndrome helicase is required for the survival of cancer cells with microsatellite instability. iScience 13, 488-497 (2019), Lieb, S. et al. Werner syndrome helicase is a selective vulnerability of microsatellite instability-high tumor cells. eLife 8, e43333 (2019)). WRNis synthetically lethal with MSI cancers. Depletion of WRN leads to anti-proliferative effects and results in activation of multiple DNA damage signaling markers, induction of cell cycle arrest and apoptosis in MSI-H cancer models but not cancer cells with an intact MMR pathway (otherwise known as microsatellite stable or MSS). The anti-proliferative effects of WRN depletion could not be rescued with a helicase deficient WRN construct, demonstrating that helicase activity of WRN is required for MSI-H viability. These findings indicate that WRN helicase provides a DNA repair and maintenance function that is essential for cell survival in MSI cancers. Recently, the mechanism of WRN dependence has been elucidated. It has been shown that dinucleotide TA repeats are selectively unstable in MSI cells and undergo large scale expansions. These expanded TA repeats form secondary DNA structures that require the WRN helicase for unwinding (van Wietmarschen, N. et al. Repeat expansions confer WRN dependence in microsatellite-unstable cancers. Nature 586, 292-298, 2020). In the absence of WRN (or upon WRN helicase inhibition), expanded TA repeats in MSI cells are subject to nuclease cleavage and chromosome breakage. Thus, inhibiting the WRN helicase is an attractive strategy for the treatment of MSI-H cancers.

### SUMMARY

There remains a need for new treatments and therapies for the treatment of cancer, and in particular cancers characterized as microsatellite instability-high (MSI-H) or mismatch repair deficient (dMMR), including colorectal, gastric or endometrial cancer. The invention provides compounds, pharmaceutically acceptable salts thereof, pharmaceutical compositions thereof and combinations thereof, said compounds being inhibitors of Werner Syndrome RecQ DNA Helicase (WRN). The invention further provides methods of treating, preventing, or ameliorating a disease or condition, comprising administering to a subject in need thereof an effective amount of a WRN inhibitor. The invention also provides compounds, pharmaceutically acceptable salts thereof, pharmaceutical compositions thereof and combinations thereof, said compounds being useful for the treatment of cancer, in particular cancers characterized as microsatellite instability-high (MSI-H) or mismatch repair deficient (dMMR). Also provided are compounds that bind to, and/or inhibit WRN, and are therefore useful as research chemicals, e.g., as a chemical probe, and as tool compounds. Various embodiments of the invention are described herein.

In one aspect, the disclosure provides a compound of Formula I, or a pharmaceutically acceptable salt thereof:

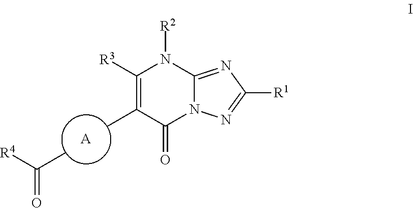

wherein R¹, R², R³, R₄, and Ring A are as described and defined herein.

In another aspect, the invention provides a pharmaceutical composition comprising a compound of Formula I of the present invention and one or more pharmaceutically acceptable carriers.

In another aspect, the invention provides a combination, in particular a pharmaceutical combination, comprising a compound of Formula I of the present invention and one or more therapeutically active agents.

In another aspect, the invention provides a compound of Formula I of the present invention for use as a medicament, in particular for the treatment of a disorder or disease which can be treated by WRN inhibition.

In another aspect, the invention provides a compound of Formula I of the present invention for use in the treatment of cancer, particularly wherein the cancer is characterized as microsatellite instability-high (MSI-H) or mismatch repair deficient (dMMR).

In another aspect, the invention provides a method of treating a disorder or disease which can be treated by WRN inhibition in a subject, comprising administering to the subject a therapeutically effective amount of a compound of Formula I of the present invention.

In another aspect, the invention provides a method of treating cancer in a subject, more particularly wherein the cancer is characterized as microsatellite instability-high (MSI-H) or mismatch repair deficient (dMMR), comprising administering to the subject a therapeutically effective amount of a compound of Formula I of the present invention.

In another aspect, the invention provides the use of a compound of Formula I of the present invention in the manufacture of a medicament for the treatment of a disorder or disease which can be treated by WRN inhibition.

In another aspect, the invention provides a compound of Formula I of the present invention for use as a research chemical, for example as a chemical probe or as a tool compound.

In another aspect, the invention provides a solid form, process or intermediate as described herein.

### DETAILED DESCRIPTION

### 1. General Description of Certain Embodiments of the Invention

In one aspect, the disclosure provides a compound of Formula I, or a pharmaceutically acceptable salt thereof:

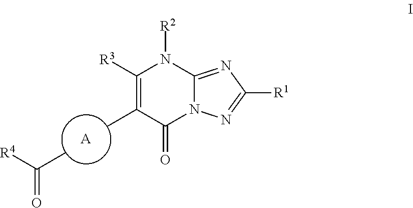

wherein Ring A represents:
   a) a 4-7 membered saturated or partially unsaturated bivalent monocyclic ring system selected from carbocyclylene or heterocyclylene (having 1-4 heteroatoms independently selected from nitrogen, oxygen, and sulfur, and 0 or 1 nitrogen atoms in addition to the 1-4 heteroatoms); or
   b) a 4-12 membered saturated or partially unsaturated bivalent bicyclic ring system that is fused, bridged, or spirocyclic selected from carbocyclylene or heterocyclylene (having 1-4 heteroatoms independently selected from nitrogen, oxygen, and sulfur);
wherein Ring A is substituted with 0-4 independently selected R^{A} substituents;
R¹ is:
   a) a 5- or 6-membered heteroaryl (having 1-4 heteroatoms independently selected from nitrogen, oxygen, and sulfur) substituted with 1 or 2 groups independently selected from C₁-C₆alkoxy, C₃-C₆cycloalkyl, and C₃-C₆cycloalkoxy, wherein said 5- or 6-membered heteroaryl is further substituted with 0-3 independently selected R^{A};
   b) a 5- or 6-membered saturated or partially unsaturated heterocyclyl (having 1-2 heteroatoms independently selected from nitrogen, oxygen, and sulfur), said heterocyclyl substituted with 0-2 groups independently selected from halogen, oxo, —NR₂, optionally substituted C₁₋₄ aliphatic, —OR, azetidinyl optionally substituted with 1 or 2 independently selected halogen, and pyrrolidinyl optionally substituted with 1 or 2 independently selected halogen;
   c) a 6-8 membered saturated or partially unsaturated bridged bicyclic heterocyclyl (having 1-4 heteroatoms independently selected from nitrogen, oxygen, and sulfur), said heterocyclyl substituted with 0-2 groups independently selected from halogen, oxo, —NR₂, optionally substituted C₁₋₄ aliphatic, —OR, azetidinyl optionally substituted with 1 or 2 independently selected halogen, and pyrrolidinyl optionally substituted with 1 or 2 independently selected halogen; or
   d) a 3-7 membered optionally substituted carbocyclyl or an optionally substituted C₂-C₄alkenyl;
R^{A} is independently selected at each occurrence from the group consisting of halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, and an optionally substituted C₁₋₆ aliphatic group;
R² is C(R^{B})₂C(O)N(R)R^{2A},
R^{B} is independently selected at each occurrence from hydrogen, —CH₃, or —CH₂CH₃, or two R^{B} taken together with the carbon to which they are attached form a cyclopropyl ring;
R^{2A} is phenyl or pyridyl, each of which is optionally substituted with 1, 2, or 3 substituents independently selected from halogen, C₁-C₄alkyl, haloC₁-C₄alkyl, C₃-C₆cycloalkyl, haloC₃-C₆-cycloalkyl, —OH, —CN, C₁-C₄alkoxy, haloC₁-C₄alkoxy, —SF₅, two substituents on adjacent atoms of the phenyl or pyridyl together with their intervening atoms form a 4- to 7-membered carbocyclyl fused to the phenyl or pyridyl, and two substituents on adjacent atoms of the phenyl or pyridyl together with their intervening atoms form a 4- to 7-membered heterocyclyl (having 1-4 heteroatoms independently selected from nitrogen, oxygen, and sulfur) fused to the phenyl or pyridyl; or
R^{2A} is 2-benzimidazolyl or 3-quinolinyl, each of which is optionally substituted with 1, 2 or 3 substituents independently selected from halogen, C₁₋₄alkyl and —OH;
R³ is hydrogen, C₁-C₄alkyl, C₃-C₅cycloalkyl, C₁-C₄alkoxy, —NHR^{3A}, —N(R^{3A})₂ or C₁-C₄alkylthio each of which, besides hydrogen, is optionally substituted with —OH, 1-5 independently selected halogen, or C₁-C₄alkoxy;
each R^{3A} is independently selected at each occurrence from C₁-C₄alkyl;
R⁴ is selected from one of a), b), and c):
   a) R⁴ is a Ring B that is selected from the group consisting of:

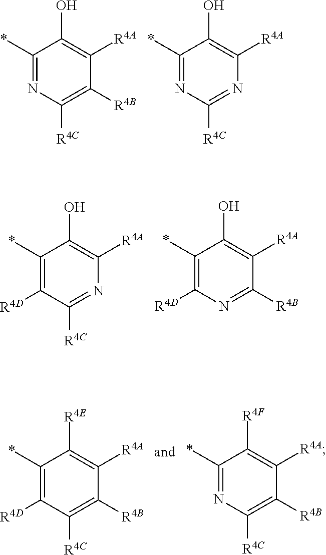

wherein * is a point of attachment to the carbonyl group that is bonded to Ring A in Formula I;
and wherein:
   any substituents that are present on Ring B selected from R^{4A}, R^{4B}, R^{4C}, R^{4D}, R^{4E}, and R^{F} are each independently selected from hydrogen; halogen; —CN; C₁-C₄alkyl; C₂-C₄alkenyl; C₂-C₄alkynyl; C₁-C₄alkoxy; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; or NR^{C}₂; or
   R^{4A} and R^{4B}, along with their intervening atoms, join to form 4- to 7-membered carbocyclyl or heterocyclyl (having 1-4 heteroatoms independently selected from nitrogen, oxygen, and sulfur) that is fused to Ring B; and any substituents that are present on Ring B selected from R^{4C}, R^{4D}, R^{4E}, and R^{4F} are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-Cocycloalkyl; C₃-C₆cycloalkoxy; or NR^{C}₂; or
   R^{4B} and R^{4C}, along with their intervening atoms, join to form 4- to 7-membered carbocyclyl or heterocyclyl (having 1-4 heteroatoms independently selected from nitrogen, oxygen, and sulfur) that is fused to Ring B; and any substituents that are present on Ring B selected from R^{4A}, R^{4D}, R^{4E}, and R^{4F} are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; or NR^{C}₂; or
   R^{4C} and R^{4D}, along with their intervening atoms, join to form 4- to 7-membered carbocyclyl or heterocyclyl (having 1-4 heteroatoms independently selected from nitrogen, oxygen, and sulfur) that is fused to Ring B; and any substituents that are present on Ring B selected from R^{4A}, R^{4B}, and R^{4E} are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃, haloC₁-C₄alkoxy; C₃-C₆cycloalkyl; C₃-C₆cycloalkoxy; or NR^{C}₂; or
   R^{4E} is halogen or —OH, and R^{4A}, R^{4B}, R^{4C}, and R^{4D} are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; or NR^{C}₂; or
   R^{4E} and R^{4A}, along with their intervening atoms, join to form 5- to 6-membered heteroaryl (having 1-4 heteroatoms independently selected from nitrogen, oxygen, and sulfur) that is fused to Ring B; and R^{4B}, R^{4C}, and R^{4D} are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; or NR^{C}₂ or
   R^{4F} and R^{4A}, along with their intervening atoms, join to form 5- to 6-membered heteroaryl (having 1-4 heteroatoms independently selected from nitrogen, oxygen, and sulfur) that is fused to Ring B; and R^{4B} and R^{4C} are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; or NR^{C}₂; and
   R^{C} is independently selected at each occurrence from hydrogen or C₁-C₄alkyl optionally substituted with —OH, —OCH₃, or —OCH₂CH₃; or NR^{C}₂, taken in combination form a heterocyclic ring selected from azetidinyl, pyrrolidinyl, or piperidinyl, said heterocyclic ring optionally substituted with —CH₃; or
   b) R⁴ is a 5-membered heteroaryl (having 1 heteroatom independently selected from nitrogen, oxygen, and sulfur and 0, 1, 2, or 3 additional ring nitrogen atoms), wherein said heteroaryl is optionally substituted with 0-4 groups independently selected from halogen, —OH, —CN, C₁-C₄alkyl, haloC₁-C₄alkyl, cycloC₃-C₆alkyl, and C₁-C₄alkoxy; or
   c) R⁴ is a C₁-C₄alkyl, C₁-C₄alkoxy, or cycloC₃-C₆alkyl, each of which is substituted with 0-3 groups independently selected from halogen, —CN, —OH, C₁-C₄alkyl, C₁-C₄alkoxy, optionally substituted 5- to 6-membered heterocyclyl, and optionally substituted 5- to 6-membered heterocyclyloxy;
each R is independently hydrogen, or an optionally substituted C₁₋₆ aliphatic group, an optionally substituted phenyl, an optionally substituted 3-7 membered saturated or partially unsaturated carbocyclic ring, an optionally substituted 3-7 membered saturated or partially unsaturated heterocyclic ring (having 1-2 heteroatoms independently selected from nitrogen, oxygen, and sulfur), or an optionally substituted 5-6 membered heteroaryl ring (having 1-4 heteroatoms independently selected from nitrogen, oxygen, and sulfur); or
two R groups on the same atom are taken together with the same atom to form an optionally substituted 4-7 membered saturated ring, partially unsaturated ring, or heteroaryl ring (wherein said 4-7 membered saturated ring, partially unsaturated ring, or heteroaryl ring, has 0-3 heteroatoms independently selected from nitrogen, oxygen, and sulfur);
with the proviso that one or two of the following provisions 1)-7) is satisfied, further provided that two of 1), 2), and 3) are not concurrently satisfied, and further provided that two of 4), 5), 6) and 7) are not concurrently satisfied:
   1) R¹ is a) a 5- or 6-membered heteroaryl (having 1-4 heteroatoms independently selected from nitrogen, oxygen, and sulfur) substituted with 1 or 2 groups independently selected from C₁-C₆alkoxy, C₃-C₆cycloalkyl, or C₃-C₆cycloalkoxy, wherein said 5- or 6-membered heteroaryl is further substituted with 0-3 independently selected R^{A};
   2) R¹ is c) 6-8 membered saturated or partially unsaturated bridged bicyclic heterocyclyl (having 1-4 heteroatoms independently selected from nitrogen, oxygen, and sulfur), said heterocyclyl substituted with 0-2 groups independently selected from halogen, oxo, —NR₂, optionally substituted C₁₋₄ aliphatic, —OR, azetidinyl optionally substituted with 1 or 2 independently selected halogen, and pyrrolidinyl optionally substituted with 1 or 2 independently selected halogen;
   3) R¹ is d) a 3-7 membered optionally substituted carbocyclyl or an optionally substituted C₂-C₄alkenyl;
   4) R⁴ is a 5-membered heteroaryl (having 1 heteroatom independently selected from nitrogen, oxygen, and sulfur and 0, 1, 2, or 3 additional ring nitrogen atoms), wherein said heteroaryl is optionally substituted with 0-4 groups independently selected from halogen, —OH, —CN, C₁-C₄alkyl, haloC₁-C₄alkyl, cycloC₃-C₆alkyl, and C₁-C₄alkoxy;
   5) R⁴ is selected from:

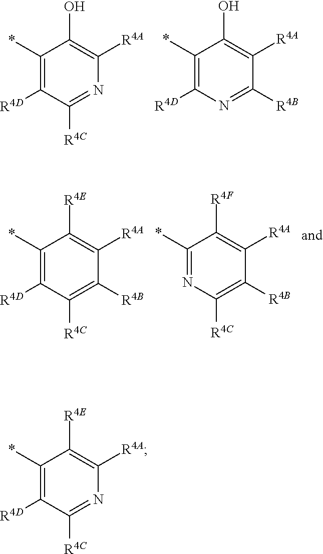

6) R⁴ is:

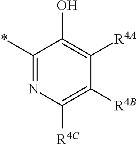

wherein
   R^{4A} and R^{4B}, along with their intervening atoms, join to form 4- to 7-membered optionally substituted carbocyclyl or optionally substituted heterocyclyl (having 1-4 heteroatoms independently selected from nitrogen, oxygen, and sulfur), or wherein R^{4B} and R^{4C} along with their intervening atoms, join to form 4- to 7-membered optionally substituted carbocyclyl or optionally substituted heterocyclyl (having 1-4 heteroatoms independently selected from nitrogen, oxygen, and sulfur);
7) R⁴ is a C₁-C₄alkyl, C₁-C₄alkoxy, or cycloC₃-C₆alkyl, each of which is substituted with 0-3 groups independently selected from halogen, —CN, —OH, C₁-C₄alkyl, optionally substituted 5- to 6-membered heterocyclyl, and C₁-C₄alkoxy.

In another aspect, the invention provides a method of treating a disorder or disease which can be treated by WRN inhibition in a subject, comprising administering to the subject a therapeutically effective amount of a compound of Formula I of the present invention.

### 2. Compounds and Definitions

Compounds of the present invention include those described generally herein, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry,” Thomas Sorrell, University Science Books, Sausalito: 1999, and “March's Advanced Organic Chemistry,” 5^{th} Ed., Ed.: Smith, M. B. and March, J., John Wiley & Sons, New York: 2001.

The term “aliphatic” or “aliphatic group,” as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bridged bicyclic” refers to any bicyclic ring system, i.e., carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 5-12 ring members and 0-4 heteroatoms independently selected from nitrogen, oxygen, or sulfur unless otherwise specified, a bridged bicyclic group is optionally substituted with one or more substituents as set forth for aliphatic groups. Additionally or alternatively, any substitutable nitrogen of a bridged bicyclic group is optionally substituted. The term “alkyl” refers to a C₁₋₁₂ straight or branched saturated aliphatic group. In certain instances, alkyl refers to a C₁₋₈ straight or branched saturated aliphatic group or a C₁₋₆ straight or branched saturated aliphatic group. The term “lower alkyl” refers to a C₁₋₄ straight or branched alkyl group.

Exemplary lower alkyl groups are methyl (—CH₃), ethyl (—CH₂CH₃), propyl, isopropyl (also referred to interchangeably herein as 2-propyl, iPr, iPr and i-Pr), butyl, isobutyl (also referred to interchangeably herein as 2-butyl, iBu, ⁱBu and i-Bu) and tert-butyl (also referred to interchangeably herein as 2-methyl-2-butyl, tBu, ^{t}Bu and t-Bu).

The term “alkenyl” refers to a C₂₋₁₂ straight or branched partially unsaturated aliphatic group comprising at least one unsaturated carbon carbon double bond. In certain instances, alkenyl refers to a C₂₋₈ or a C₂₋₆ straight or branched partially unsaturated aliphatic group comprising at least one unsaturated carbon carbon double bond. The term “lower alkenyl” refers to a C₂₋₄ straight or branched partially unsaturated aliphatic group comprising at least one unsaturated carbon carbon double bond. Alkenyl groups include both cis (Z) and trans (E) regioisomers. Exemplary lower alkenyl groups are vinyl, allyl, 2-propenyl, and butenyl isomers (—CH₂CH₂CH—CH₂, —CH₂CH═CHCH₃ and —CH—CHCH₂CH₃).

The term “alkynyl” refers to a C₂₋₁₂ straight or branched partially unsaturated aliphatic group comprising at least one unsaturated carbon carbon triple bond. In certain instances, alkynyl refers to a C₂₋₈ or a C₂₋₆ straight or branched partially unsaturated aliphatic group comprising at least one unsaturated carbon carbon triple bond. The term “lower alkynyl” refers to a C₂₋₄ straight or branched partially unsaturated aliphatic group comprising at least one unsaturated carbon carbon triple bond. Exemplary lower alkynyl groups are ethynyl, 1-propynyl, 2-propynyl, 1-butynyl, 2-butynyl, and 3-butynyl.

The term “haloalkyl” refers to a straight or branched alkyl group that is substituted with one or more halogen atoms. The term “lower haloalkyl” refers to a C₁-4 straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁻ (as in N-substituted pyrrolidinyl).

The term “unsaturated,” as used herein, means that a moiety has one or more units of unsaturation.

As used herein, the term “bivalent C₁₋₈ (or C₁₋₆ i.e., C₁-C₆) saturated or unsaturated, straight or branched, hydrocarbon chain,” refers to bivalent alkylene, alkenylene, and alkynylene chains that are straight or branched as defined herein.

As used herein, the term “bivalent,” to describe a cyclic (and noncyclic) group refers to, for example, bivalent carbocyclylene, phenylene, heterocyclylene, and heteroarylene that are bivalent moieties of carbocycles, phenyls, heterocycles, and heteroaryls described herein. Non-limiting examples include

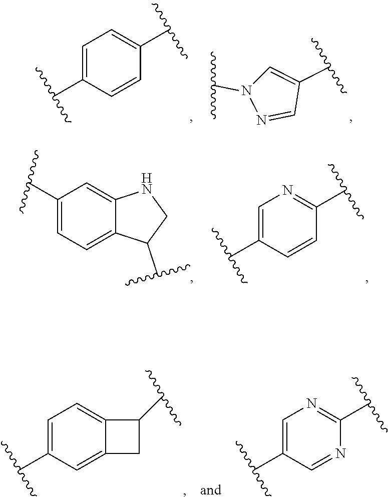 [SMILES: *c1ccc(*)cc1 ; *c1cnn(*)c1 ; *c1ccc2c(c1)NCC2* ; *c1ccc(*)nc1 ; *c1ccc2c(c1)CC2* ; *c1cnc(*)nc1]

“Carbocyclylene” as used herein refers to a carbocyclic or cycloalkyl moiety that is bivalent as described above (i.e., attached at two different points to the rest of the compound). Non-limiting examples include cyclopropylene, cyclobutylene, cyclopentylene, or cyclohexylene as shown below.

| | | Different examples of | Different examples of |
|---|---|---|---|
| cyclopropylene | cyclobutylene | cyclopentylene | cyclohexylene |
| 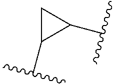 [SMILES: *C1CC1*] | 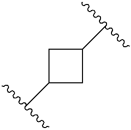 [SMILES: *C1CC(*)C1] | 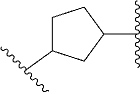 [SMILES: *C1CCC(*)C1] | 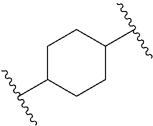 [SMILES: *C1CCC(*)CC1] |
| | | 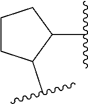 [SMILES: *C1CCCC1*] | 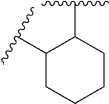 [SMILES: *C1CCCCC1*] |
| | | | 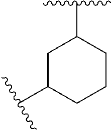 [SMILES: *C1CCCC(*)C1] |

A carbocyclylene may be saturated as in the examples shown above or partially unsaturated as in the examples shown below.

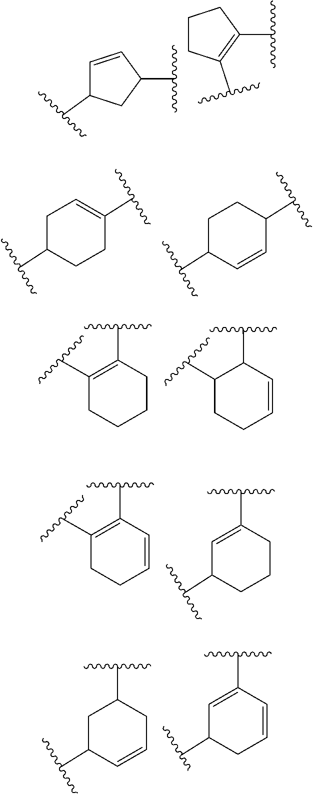

A carbocyclylene may be multi-cyclic, for example, bicyclic or tricyclic. Such multi-cyclic carbocyclylene systems may be saturated or partially unsaturated (while one ring of the bicyclic system may be aromatic it is to be understood that multi-cyclic ring systems that are not in their entirety aromatic may also fall under the definition of carbocyclylene). The rings may form bridged, fused, or spiro systems. Non-limiting examples are shown below.

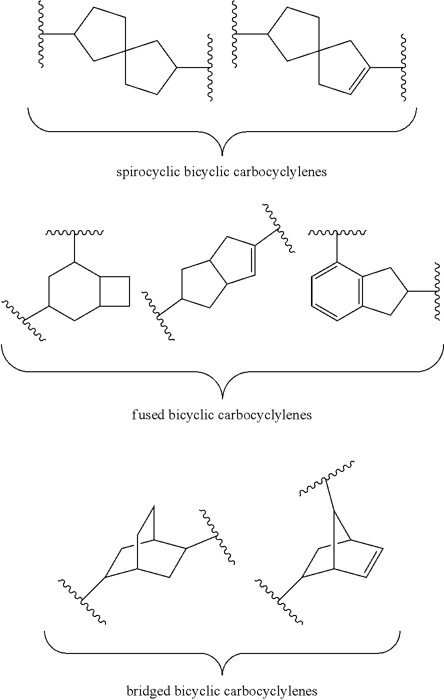

“Heterocyclylene” as used herein refers to a heterocyclic or heterocyclyl moiety that is bivalent as described above (i.e., attached at two different points to the rest of the compound) and may also be saturated or partially unsaturated. Non-limiting examples include those shown below. Heterocyclylene is understood to include bicyclic heterocyclylene systems. Non-limiting examples of bicyclic heterocyclylene moieties are also shown below and said bicyclic systems may be spirocyclic, fused, or bridged and may be saturated or partially unsaturated.

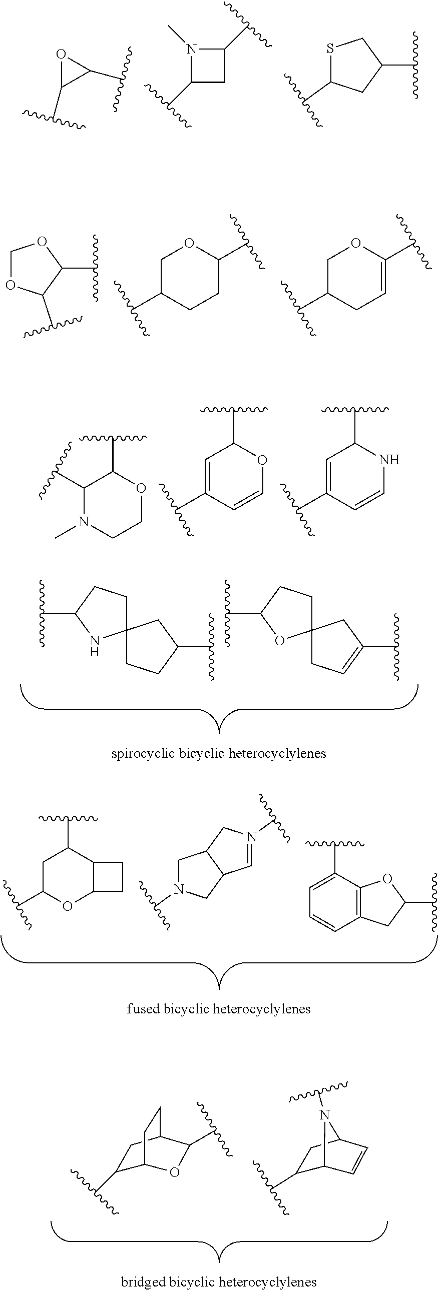

“Phenylene” as used herein refers to a phenyl moiety that is bivalent as described above (i.e., attached at two different points to the rest of the compound). Examples are shown below.

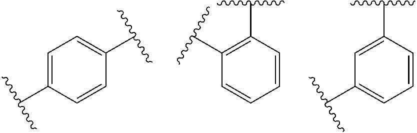 [SMILES: *c1ccc(*)cc1 ; *c1ccccc1* ; *c1cccc(*)c1]

“Arylene” as used herein refers to an mono or multi-cyclic aryl (i.e., phenyl or a multi-cyclic aryl) moiety that is bivalent as described above (i.e., attached at two different points to the rest of the compound), wherein the arylene group contains no heteroatoms. Examples are shown below.

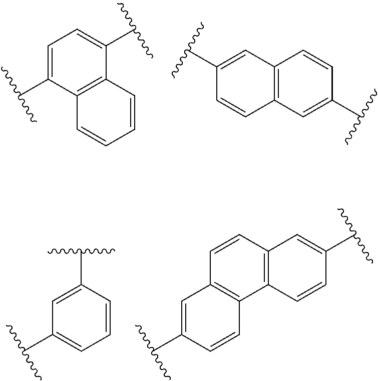

“Heteroarylene,” as used herein refers to a mono or multi-cyclic aryl ring system that contains at least one heteroatom wherein the ring system is bivalent as described above (i.e., attached at two different points to the rest of the compound). Examples are shown below.

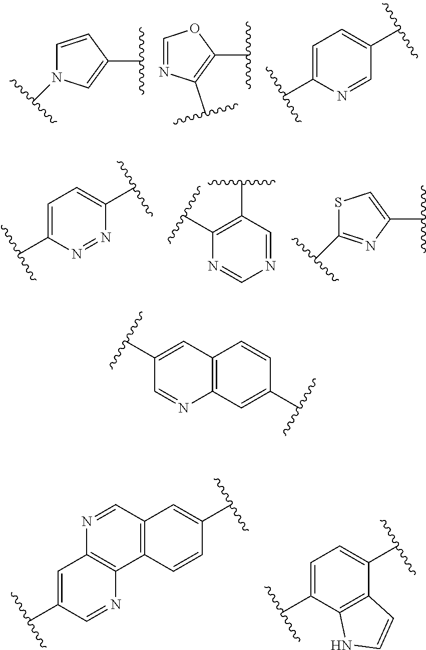

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

“Carbocyclyl (or heterocyclyl, aryl, phenyl, or heteroaryl) fused to” another phenyl, aryl, heteroaryl, carbocyclyl, or heterocyclyl, for example, a “phenyl or pyridyl” as used herein, may be referred to as “partially unsaturated” without said “carbocyclyl (or heterocyclyl, aryl, phenyl, or heteroaryl) fused to” the other ring requiring further unsaturation besides the carbon carbon bond which it shares with the ring to which it is fused (i.e., the “phenyl or pyridyl”). This is illustrated below.

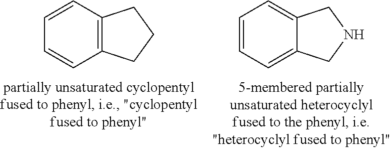 [SMILES: c1ccc2c(c1)CCC2 ; c1ccc2c(c1)CNC2]

A further example below shows a carbocyclyl moiety fused to a Ring B as defined in the embodiments herein. Said carbocyclyl does not explicitly require a descriptor of “partially unsaturated” to describe said carbocyclyl because it shares two carbons with the aromatic pyridine to which it is fused. Such language is used herein to describe such systems, for example, “R^{4A} and R^{4B}, along with their intervening atoms, join to form 4- to 7-membered carbocyclyl that is fused to Ring B” as shown in the image below. As such, “Ring B” may refer to a monocyclic ring (i.e., the pyridine shown below and its substituents which do not form a fused ring), without any further fused rings created by its substituents (i.e., R^{4A} and R^{4B}). Any further fused ring created by the substituents of Ring B is described as being “fused to Ring B.” Likewise, R^{4A} and R^{4B}, along with their intervening atoms, join to form 4- to 7-membered carbocyclyl or heterocyclyl (having 1-4 heteroatoms independently selected from nitrogen, oxygen, and sulfur) that is fused to Ring B (not pictured), is subject to the same interpretation.

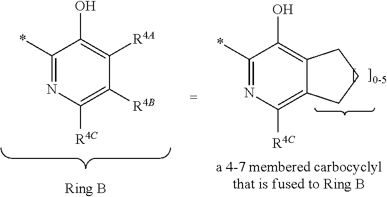

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, 9 or 10 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, pyrazinyl, pyrazolyl, pyridazinyl, pyridyl, pyrimidinyl, triazinyl, thienyl, furanyl, pyrrolyl, imidazolyl, pyrazolyl, triazolyl (i.e., 1,2,3-triazolyl), 1,2,4-triazolyl, 1,2,5-triazolyl, 1,3,4-triazolyl, tetrazolyl, oxazolyl, isoxazolyl, oxadiazolyl, 1,2,3-oxadiazolyl, 1,2,4-oxadiazolyl, 1,2,5-oxadiazolyl, 1,3,4-oxadiazolyl, thiazolyl, isothiazolyl, thiadiazolyl, indolizinyl, purinyl, naphthyridinyl, and pteridinyl. The terms “heteroaryl” and “heteroar-,” as used herein, also include groups in which a heteroaromatic ring is fused to one or more aryl, cycloaliphatic, or heterocyclyl rings, where unless otherwise specified, the radical or point of attachment is on the heteroaromatic ring or on one of the rings to which the heteroaromatic ring is fused. Nonlimiting examples include indolyl, isoindolyl, benzothienyl, benzofuranyl, dibenzofuranyl, indazolyl, indolizinyl, isoindolin-1-only, 1,2-dihydro-3H-pyrrolo[3,4-c]pyridin-3-onyl, 2,3-dihydro-1H-pyrrolo[3,4-c]pyridin-1-onyl, imidazo[1,2-a]pyridyl, imidazo[1,5-a]pyridyl, pyrazolo[1,5-a]pyridyl, pyrrolo[1,2-b]pyridazinyl, pyrrolo[1,2-a]pyrimidinyl, imidazo[1,2-b]pyridazinyl, imidazo[1,2-a]pyrimidinyl, benzimidazolyl, benzthiazolyl, quinolyl, isoquinolyl, cinnolinyl, phthalazinyl, quinazolinyl, quinoxalinyl, 4H/-quinolizinyl, carbazolyl, acridinyl, phenazinyl, phenothiazinyl, phenoxazinyl, tetrahydroquinolinyl, and tetrahydroisoquinolinyl. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or +NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, oxetanyl, azetidinyl, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, 2-oxa-6-azaspiro[3.3]heptane, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be mono- or bicyclic. The term “heterocyclylalkyl” refers to an alkyl group substituted by a heterocyclyl, wherein the alkyl and heterocyclyl portions independently are optionally substituted.

“Arylene” or “heteroarylene,” as used herein (i.e., phenylene), refers to any bivalent aryl or heterocyclyl described herein, that is a bisradical substituted at each of two substitutable positions of the ring system as described in detail supra.

“Heterocyclyloxy,” as used herein, refers to an —OR group wherein the R is a heterocyclyl. Nonlimiting examples are shown below.

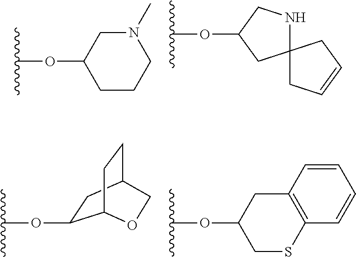

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄B(OR^{º})₂; —(CH₂)₀₋₄R^{º}; —(CH₂)₀₋₄OR^{º}; —O(CH₂)₀₋₄R^{º}; —O—(CH₂)₀₋₄C(O)OR^{º}; —(CH₂)₀₋₄CH(OR^{º})₂; —(CH₂)₀₋₄SR^{º}; —(CH₂)₀₋₄Ph, which may be substituted with R^{º}; —(CH₂)₀₋₄O(CH₂)₀₋₁Ph which may be substituted with R^{º}; —CH═CHPh, which may be substituted with R^{º}; —(CH₂)₀₋₄O(CH₂)₀₋₁-pyridyl which may be substituted with R^{º}; —NO₂; —CN; —N₃; —(CH₂)₀₋₄N(R^{º})₂; —(CH₂)₀₋₄N(R^{º})C(O)R^{º}; —N(R^{º}C(S)R^{º}; —(CH₂)₀₋₄N(R^{º}C(O)NR^{º}₂; —N(R^{º}C(S)NR^{º}₂; —(CH₂)₀₋₄N(R^{º}C(O)OR^{º}; —N(R^{º}N(R^{º})C(O)R^{º}; —N(R^{º}N(R^{º})C(O)NR^{º}₂; —N(R^{º}N(R^{º}C(O)OR^{º}; —N(R^{º}C(NR^{º})N(R^{º}₂; —(CH₂)₀₋₄C(O)R^{º}; —C(S)R^{º}; —(CH₂)₀₋₄C(O)OR^{º}; —(CH₂)₀₋₄C(O)SR^{º}; —(CH₂)₀₋₄C(O)OSiR^{º}₃; —(CH₂)₀₋₄OC(O)R^{º}; —OC(O)(CH₂)₀₋₄SR^{º}; —(CH₂)₀₋₄SC(O)R^{º}; —(CH₂)₀₋₄C(O)NR^{º}₂; C(S)NR^{º}₂; —C(S)SR^{º}; —SC(S)SR^{º}; —(CH₂)₀₋₄OC(O)NR^{º}₂; —C(O)N(OR^{º})R^{º}; —C(O)C(O)R^{º}; —C(O)CH₂C(O)R^{º}; —C(NOR^{º})R^{º}; —(CH₂)₀₋₄SSR^{º}; —(CH₂)₀₋₄S(O)₂R^{º}; —(CH₂)₀₋₄S(O)₂OR^{º}; —(CH₂)₀₋₄OS(O)₂R^{º}; —S(O)₂NR^{º}₂; —(CH₂)₀₋₄S(O)R^{º}; —N(R^{º}S(O)₂NR^{º}₂; —N(R^{º}S(O)₂R^{º}; —N(OR^{º})R^{º}; —C(NH)NR^{º}₂; —(CH₂)₀₋₄P(O)₂R^{º}; —(CH₂)₀₋₄P(O)R^{º}₂; —(CH₂)₀₋₄OP(O)R^{º}₂; —(CH₂)₀₋₄OP(O)(OR^{º})₂; —SiR^{º}₃; —(C₁₋₄ straight or branched alkylene)O—N(R^{º})₂; or —(C₁-4 straight or branched) alkylene) C(O)O—N(R^{º})₂, wherein each R^{º} may be substituted as defined below and is independently hydrogen, C₁₋₆ aliphatic, —SO₂-C₁₋₄ aliphatic (i.e., —SO₂CH₃)—CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{º}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{º} (or the ring formed by taking two independent occurrences of R^{º} together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR_{®}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₆ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{º} include ═O and ═S.

Suitable divalent substituents on a saturated carbon atom of an “optionally substituted” group, which includes instances of R^{º} (or the ring formed by taking two independent occurrences of R^{º} together with their intervening atoms), include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{•}, -(haloR^{•}), —OH, —OR^{•}, —O(haloR^{•}), —CN, —C(O)OH, —C(O)OR•, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of Rt are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺ (C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, Z and E conformational isomers and Rₐ (or M) and Sₐ (or P) atropisomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention. In certain embodiments, Ring A of a provided compound may be substituted with one or more deuterium atoms.

The structures as drawn represent relative configurations, unless labeled as absolute configurations. The invention contemplates individual enantiomers and racemic mixtures.

### 3. Description of Exemplary Embodiments

In one aspect, the disclosure provides a compound of Formula I, or a pharmaceutically acceptable salt thereof:

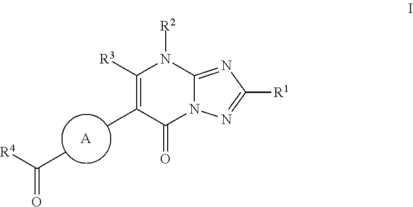

wherein Ring A represents:
   a) a 4-7 membered saturated or partially unsaturated bivalent monocyclic ring system selected from carbocyclylene or heterocyclylene (having 1-4 heteroatoms independently selected from nitrogen, oxygen, and sulfur, and 0 or 1 nitrogen atoms in addition to the 1-4 heteroatoms); or
   b) a 4-12 membered saturated or partially unsaturated bivalent bicyclic ring system that is fused, bridged, or spirocyclic selected from carbocyclylene or heterocyclylene (having 1-4 heteroatoms independently selected from nitrogen, oxygen, and sulfur);
wherein Ring A is substituted with 0-4 independently selected R^{A} substituents;
R¹ is:
   a) a 5- or 6-membered heteroaryl (having 1-4 heteroatoms independently selected from nitrogen, oxygen, and sulfur) substituted with 1 or 2 groups independently selected from C₁-C₆alkoxy, C₃-C₆cycloalkyl, and C₃-C₆cycloalkoxy, wherein said 5- or 6-membered heteroaryl is further substituted with 0-3 independently selected R^{A};
   b) a 5- or 6-membered saturated or partially unsaturated heterocyclyl (having 1-2 heteroatoms independently selected from nitrogen, oxygen, and sulfur), said heterocyclyl substituted with 0-2 groups independently selected from halogen, oxo, —NR₂, optionally substituted C₁₋₄ aliphatic, —OR, azetidinyl optionally substituted with 1 or 2 independently selected halogen, and pyrrolidinyl optionally substituted with 1 or 2 independently selected halogen;
   c) a 6-8 membered saturated or partially unsaturated bridged bicyclic heterocyclyl (having 1-4 heteroatoms independently selected from nitrogen, oxygen, and sulfur), said heterocyclyl substituted with 0-2 groups independently selected from halogen, oxo, —NR₂, optionally substituted C₁₋₄ aliphatic, —OR, azetidinyl optionally substituted with 1 or 2 independently selected halogen, and pyrrolidinyl optionally substituted with 1 or 2 independently selected halogen; or
   d) a 3-7 membered optionally substituted carbocyclyl or an optionally substituted C₂-C₄alkenyl;
R^{A} is independently selected at each occurrence from the group consisting of halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR,
—OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, and an optionally substituted C₁₋₆ aliphatic group;
R² is C(R^{B})₂C(O)N(R)R^{2A};
R^{B} is independently selected at each occurrence from hydrogen, —CH₃, or —CH₂CH₃, or two R^{B} taken together with the carbon to which they are attached form a cyclopropyl ring;
R^{2A} is phenyl or pyridyl, each of which is optionally substituted with 1, 2, or 3 substituents independently selected from halogen, C₁-C₄alkyl, haloC₁-C₄alkyl, C₃-C₆cycloalkyl, haloC₃-C₆-cycloalkyl, —OH, —CN, C₁-C₄alkoxy, haloC₁-C₄alkoxy, —SF₅, two substituents on adjacent atoms of the phenyl or pyridyl together with their intervening atoms form a 4- to 7-membered carbocyclyl fused to the phenyl or pyridyl, and two substituents on adjacent atoms of the phenyl or pyridyl together with their intervening atoms form a 4- to 7-membered heterocyclyl (having 1-4 heteroatoms independently selected from nitrogen, oxygen, and sulfur) fused to the phenyl or pyridyl; or
R^{2A} is 2-benzimidazolyl or 3-quinolinyl, each of which is optionally substituted with 1, 2 or 3 substituents independently selected from halogen, C₁₋₄alkyl and —OH;
R³ is hydrogen, C₁-C₄alkyl, C₃-C₅cycloalkyl, C₁-C₄alkoxy, —NHR^{3A}, —N(R^{3A})₂ or C₁-C₄alkylthio each of which, besides hydrogen, is optionally substituted with —OH, 1-5 independently selected halogen, or C₁-C₄alkoxy;
each R^{3A} is independently selected at each occurrence from C₁-C₄alkyl;
R⁴ is selected from one of a), b), and c):
   a) R⁴ is a Ring B that is selected from the group consisting of:

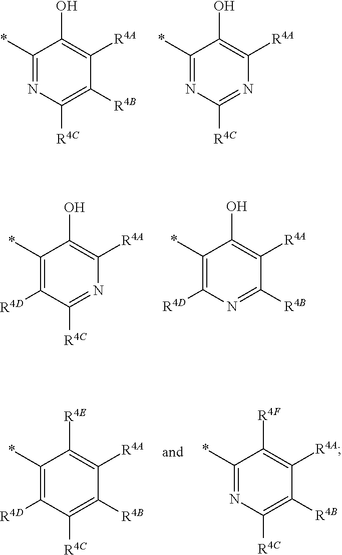

wherein * is a point of attachment to the carbonyl group that is bonded to Ring A in Formula I;
and wherein:
   any substituents that are present on Ring B selected from R^{4A}, R^{4B}, R^{4C}, R^{4D}, R^{4E}, and R^{F} are each independently selected from hydrogen; halogen; —CN; C₁-C₄alkyl; C₂-C₄alkenyl; C₂-C₄alkynyl; C₁-C₄alkoxy; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; or NR^{C}₂; or
   R^{4A} and R^{4B}, along with their intervening atoms, join to form 4- to 7-membered carbocyclyl or heterocyclyl (having 1-4 heteroatoms independently selected from nitrogen, oxygen, and sulfur) that is fused to Ring B; and any substituents that are present on Ring B selected from R^{4C}, R^{4D}, R^{4E}, and R^{4F} are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; or NR^{C}₂; or
   R^{4B} and R^{4C}, along with their intervening atoms, join to form 4- to 7-membered carbocyclyl or heterocyclyl (having 1-4 heteroatoms independently selected from nitrogen, oxygen, and sulfur) that is fused to Ring B; and any substituents that are present on Ring B selected from R^{4A}, R^{4D}, R^{4E}, and R^{4F} are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; or NR^{C}₂; or
   R^{4C} and R^{4D}, along with their intervening atoms, join to form 4- to 7-membered carbocyclyl or heterocyclyl (having 1-4 heteroatoms independently selected from nitrogen, oxygen, and sulfur) that is fused to Ring B; and any substituents that are present on Ring B selected from R^{4A}, R^{4B}, and R^{4E} are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; or NR^{C}₂; or
   R^{4E} is halogen or —OH, and R^{4A}, R^{4B}, R^{4C}, and R_{4D} are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; or NR^{C}₂; or
   R^{4E} and R^{4A}, along with their intervening atoms, join to form 5- to 6-membered heteroaryl (having 1-4 heteroatoms independently selected from nitrogen, oxygen, and sulfur) that is fused to Ring B; and R^{4B}, R^{4C}, and R^{4D} are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; or NR^{C}₂; or
   R^{4F} and R^{4A}, along with their intervening atoms, join to form 5- to 6-membered heteroaryl (having 1-4 heteroatoms independently selected from nitrogen, oxygen, and sulfur) that is fused to Ring B; and R^{4B} and R^{4C} are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; or NR^{C}₂; and
   R^{C} is independently selected at each occurrence from hydrogen or C₁-C₄alkyl optionally substituted with —OH, —OCH₃, or —OCH₂CH₃; or NR^{C}₂, taken in combination form a heterocyclic ring selected from azetidinyl, pyrrolidinyl, or piperidinyl, said heterocyclic ring optionally substituted with —CH₃; or
   b) R⁴ is a 5-membered heteroaryl (having 1 heteroatom independently selected from nitrogen, oxygen, and sulfur and 0, 1, 2, or 3 additional ring nitrogen atoms), wherein said heteroaryl is optionally substituted with 0-4 groups independently selected from halogen, —OH, —CN, C₁-C₄alkyl, haloC₁-C₄alkyl, cycloC₃-C₆alkyl, and C₁-C₄alkoxy; or
   c) R⁴ is a C₁-C₄alkyl, C₁-C₄alkoxy, or cycloC₃-C₆alkyl, each of which is substituted with 0-3 groups independently selected from halogen, —CN, —OH, C₁-C₄alkyl, C₁-C₄alkoxy, optionally substituted 5- to 6-membered heterocyclyl, and optionally substituted 5- to 6-membered heterocyclyloxy;
each R is independently hydrogen, or an optionally substituted C₁₋₆ aliphatic group, an optionally substituted phenyl, an optionally substituted 3-7 membered saturated or partially unsaturated carbocyclic ring, an optionally substituted 3-7 membered saturated or partially unsaturated heterocyclic ring (having 1-2 heteroatoms independently selected from nitrogen, oxygen, and sulfur), or an optionally substituted 5-6 membered heteroaryl ring (having 1-4 heteroatoms independently selected from nitrogen, oxygen, and sulfur); or
two R groups on the same atom are taken together with the same atom to form an optionally substituted 4-7 membered saturated ring, partially unsaturated ring, or heteroaryl ring (wherein said 4-7 membered saturated ring, partially unsaturated ring, or heteroaryl ring, has 0-3 heteroatoms independently selected from nitrogen, oxygen, and sulfur);
with the proviso that one or two of the following provisions 1)-7) is satisfied, further provided that two of 1), 2), and 3) are not concurrently satisfied, and further provided that two of 4), 5), 6) and 7) are not concurrently satisfied:
   1) R¹ is a) a 5- or 6-membered heteroaryl (having 1-4 heteroatoms independently selected from nitrogen, oxygen, and sulfur) substituted with 1 or 2 groups independently selected from C₁-C₆alkoxy, C₃-C₆cycloalkyl, or C₃-C₆cycloalkoxy, wherein said 5- or 6-membered heteroaryl is further substituted with 0-3 independently selected R^{A};
   2) R¹ is c) 6-8 membered saturated or partially unsaturated bridged bicyclic heterocyclyl (having 1-4 heteroatoms independently selected from nitrogen, oxygen, and sulfur), said heterocyclyl substituted with 0-2 groups independently selected from halogen, oxo, —NR₂, optionally substituted C₁₋₄ aliphatic, —OR, azetidinyl optionally substituted with 1 or 2 independently selected halogen, and pyrrolidinyl optionally substituted with 1 or 2 independently selected halogen;
   3) R¹ is d) a 3-7 membered optionally substituted carbocyclyl or an optionally substituted C₂-C₄alkenyl;
   4) R⁴ is a 5-membered heteroaryl (having 1 heteroatom independently selected from nitrogen, oxygen, and sulfur and 0, 1, 2, or 3 additional ring nitrogen atoms), wherein said heteroaryl is optionally substituted with 0-4 groups independently selected from halogen, —OH, —CN, C₁-C₄alkyl, haloC₁-C₄alkyl, cycloC₃-C₆alkyl, and C₁-C₄alkoxy;
   5) R⁴ is selected from:

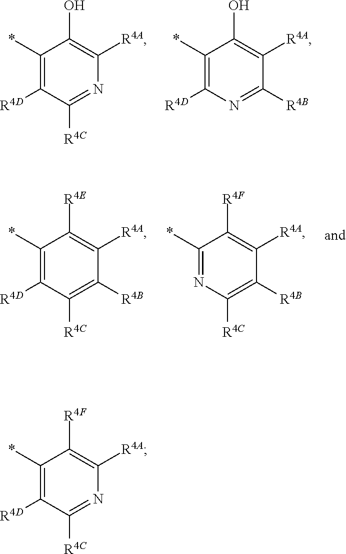

6) R⁴ is:

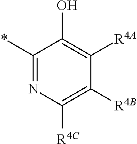

wherein
   R^{4A} and R^{4B}, along with their intervening atoms, join to form 4- to 7-membered optionally substituted carbocyclyl or optionally substituted heterocyclyl (having 1-4 heteroatoms independently selected from nitrogen, oxygen, and sulfur), or wherein R^{4B} and R^{4C} along with their intervening atoms, join to form 4- to 7-membered optionally substituted carbocyclyl or optionally substituted heterocyclyl (having 1-4 heteroatoms independently selected from nitrogen, oxygen, and sulfur);
7) R⁴ is a C₁-C₄alkyl, C₁-C₄alkoxy, or cycloC₃-C₆alkyl, each of which is substituted with 0-3 groups independently selected from halogen, —CN, —OH, C₁-C₄alkyl, optionally substituted 5- to 6-membered heterocyclyl, and C₁-C₄alkoxy.

As described generally above, Ring A is
a) a 4-7 membered saturated or partially unsaturated bivalent monocyclic ring system selected from carbocyclylene or heterocyclylene (having 1-4 heteroatoms independently selected from nitrogen, oxygen, and sulfur, and 0 or 1 nitrogen atoms in addition to the 1-4 heteroatoms); or
b) a 4-12 membered saturated or partially unsaturated bivalent bicyclic ring system that is fused, bridged, or spirocyclic selected from carbocyclylene or heterocyclylene (having 1-4 heteroatoms independently selected from nitrogen, oxygen, and sulfur),
wherein Ring A is substituted with 0-4 independently selected R^{A} substituents.

In some embodiments, Ring A is a 4-7 membered saturated or partially unsaturated bivalent monocyclic ring system selected from carbocyclylene or heterocyclylene (having 1-4 heteroatoms independently selected from nitrogen, oxygen, and sulfur, and 0 or 1 nitrogen atoms in addition to the 1-4 heteroatoms), wherein Ring A is substituted with 0-4 independently selected R^{A} substituents. In some embodiments, Ring A is a 4-7 membered saturated or partially unsaturated bivalent monocyclic carbocyclylene, wherein Ring A is substituted with 0-4 independently selected R^{A} substituents. In some embodiments, Ring A is a 4-7 membered saturated or partially unsaturated bivalent monocyclic heterocyclylene (having 1-4 heteroatoms independently selected from nitrogen, oxygen, and sulfur, and 0 or 1 nitrogen atoms in addition to the 1-4 heteroatoms), wherein Ring A is substituted with 0-4 independently selected R^{A} substituents.

In some embodiments, Ring A is a 4-12 membered saturated or partially unsaturated bivalent bicyclic ring system that is fused, bridged, or spirocyclic selected from carbocyclylene or heterocyclylene (having 1-4 heteroatoms independently selected from nitrogen, oxygen, and sulfur), wherein Ring A is substituted with 0-4 independently selected R^{A} substituents. In some embodiments, Ring A is a 4-12 membered saturated or partially unsaturated bivalent bicyclic ring system that is fused, bridged, or spirocyclic and is a carbocyclylene, wherein Ring A is substituted with 0-4 independently selected R^{A} substituents. In some embodiments, Ring A is a 4-12 membered saturated or partially unsaturated bivalent bicyclic ring system that is fused, bridged, or spirocyclic and is a heterocyclylene (having 1-4 heteroatoms independently selected from nitrogen, oxygen, and sulfur), wherein Ring A is substituted with 0-4 independently selected R^{A} substituents.

In some embodiments, Ring A is a 4-12 membered saturated or partially unsaturated bivalent bicyclic ring system comprising 2 fused rings. In some embodiments, Ring A is a 4-12 membered saturated or partially unsaturated bivalent bicyclic ring system comprising a spirocyclic ring system. In some embodiments, Ring A is a 4-12 membered saturated or partially unsaturated bivalent bicyclic ring system comprising a bridged ring system.

In some embodiments, Ring A is

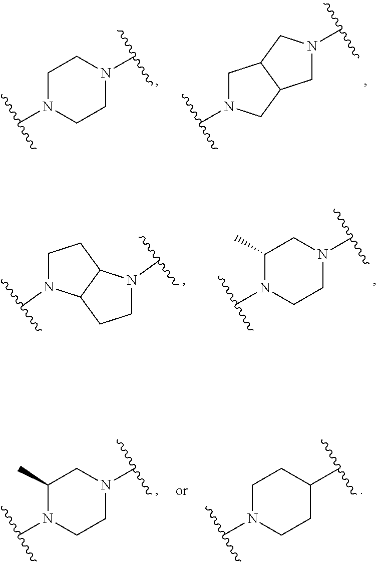

In some embodiments, Ring A is

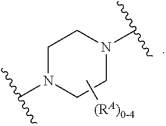

In some embodiments, Ring A is as selected from one of the substituents of Table 1.

As described generally above, R¹ is:
a) a 5- or 6-membered heteroaryl (having 1-4 heteroatoms independently selected from nitrogen, oxygen, and sulfur) substituted with 1 or 2 groups independently selected from C₁-C₆alkoxy, C₃-C₆cycloalkyl, and C₃-C₆cycloalkoxy, wherein said 5- or 6-membered heteroaryl is further substituted with 0-3 independently selected R^{A};
b) a 5- or 6-membered saturated or partially unsaturated heterocyclyl (having 1-2 heteroatoms independently selected from nitrogen, oxygen, and sulfur), said heterocyclyl substituted with 0-2 groups independently selected from halogen, oxo, —NR₂, optionally substituted C₁₋₄ aliphatic, —OR, azetidinyl optionally substituted with 1 or 2 independently selected halogen, and pyrrolidinyl optionally substituted with 1 or 2 independently selected halogen;
c) a 6-8 membered saturated or partially unsaturated bridged bicyclic heterocyclyl (having 1-4 heteroatoms independently selected from nitrogen, oxygen, and sulfur), said heterocyclyl substituted with 0-2 groups independently selected from halogen, oxo, —NR₂, optionally substituted C₁₋₄ aliphatic, —OR, azetidinyl optionally substituted with 1 or 2 independently selected halogen, and pyrrolidinyl optionally substituted with 1 or 2 independently selected halogen; or
d) a 3-7 membered optionally substituted carbocyclyl or an optionally substituted C₂-C₄alkenyl.

In some embodiments, R¹ is a 5- or 6-membered heteroaryl (having 1-4 heteroatoms independently selected from nitrogen, oxygen, and sulfur) substituted with 1 or 2 groups independently selected from C₁-C₆alkoxy, C₃-C₆cycloalkyl, and C₃-C₆cycloalkoxy, wherein said 5- or 6-membered heteroaryl is further substituted with 0-3 independently selected R^{A}. In some embodiments, R¹ is a 5- or 6-membered saturated or partially unsaturated heterocyclyl (having 1-2 heteroatoms independently selected from nitrogen, oxygen, and sulfur), said heterocyclyl substituted with 0-2 groups independently selected from halogen, oxo, —NR₂, optionally substituted C₁₋₄ aliphatic, —OR, azetidinyl optionally substituted with 1 or 2 independently selected halogen, and pyrrolidinyl optionally substituted with 1 or 2 independently selected halogen. In some embodiments, R¹ is a 6-8 membered saturated or partially unsaturated bridged bicyclic heterocyclyl (having 1-4 heteroatoms independently selected from nitrogen, oxygen, and sulfur), said heterocyclyl substituted with 0-2 groups independently selected from halogen, oxo, —NR₂, optionally substituted C₁₋₄ aliphatic, —OR, azetidinyl optionally substituted with 1 or 2 independently selected halogen, and pyrrolidinyl optionally substituted with 1 or 2 independently selected halogen. In some embodiments, R¹ is a 3-7 membered optionally substituted carbocyclyl. In some embodiments, R¹ is an optionally substituted C₂-C₄alkenyl. In some embodiments, R¹ is cylopropyl substituted C₂-C₄alkenyl. In some embodiments, R¹ is methyl substituted C₂alkenyl.

In some embodiments, R¹ is a 6-membered partially unsaturated heterocyclyl (having 1 oxygen atom). In some embodiments, R¹ is a 6-membered heteroaryl (having 1 nitrogen atom), said heteroaryl substituted with 1 or 2 groups independently selected from C₁-C₆alkoxy, C₃-C₆cycloalkyl, and C₃-C₆cycloalkoxy, wherein said heteroaryl is further substituted with 0-1 R^{A}, wherein R^{A} is an optionally substituted C₁₋₆ aliphatic group. In some embodiments, R¹ is a 6-membered heteroaryl (having 2 nitrogen atoms), said heteroaryl substituted with 1 or 2 groups independently selected from C₁-C₆alkoxy, C₃-C₆cycloalkyl, and C₃-C₆cycloalkoxy, wherein said heteroaryl is further substituted with 0-1 R^{A}, wherein R^{A} is an optionally substituted C₁₋₆ aliphatic group.

In some embodiments, R¹ is a 5-membered heteroaryl (having 1-3 heteroatoms independently selected from nitrogen, oxygen, and sulfur) substituted with 1 or 2 groups independently selected from C₁-C₆alkoxy, C₃-C₆cycloalkyl, and C₃-C₆cycloalkoxy, wherein said 5-membered heteroaryl is optionally further substituted with 0-3 independently selected R^{A}. In some embodiments, R¹ is a 5-membered heteroaryl (having 1-3 heteroatoms independently selected from nitrogen, oxygen, and sulfur) substituted with 1 or 2 groups independently selected from C₁-C₆alkoxy, C₃-C₆cycloalkyl, and C₃-C₆cycloalkoxy. In some embodiments, R¹ is a 5-membered heteroaryl (having 2 nitrogen atoms) substituted with 1 or 2 groups independently selected from C₁-C₆alkoxy, C₃-C₆cycloalkyl, and C₃-C₆cycloalkoxy, wherein said 5-membered heteroaryl is optionally further substituted with 0-1 R^{A}, wherein R^{A} is hydroxyl substituted C₁-C₄alkyl.

In some embodiments, R¹ is a 5- or 6-membered heteroaryl (having 1-4 heteroatoms is independently selected from nitrogen, oxygen, and sulfur) substituted with one group of C₁-C₆alkoxy or C₃-C₆cycloalkyl, wherein said 5- or 6-membered heteroaryl is optionally further substituted with 0-3 independently selected R^{A}.

In some embodiments, R¹ is pyridyl substituted with C₁-C₄alkoxy and further substituted with 0-2 R^{A} substituents.

In some embodiments, R¹ is 5-membered heteroaryl (having 1 heteroatom independently selected from nitrogen, oxygen, and sulfur, and 0 or 1 additional ring nitrogen atoms), wherein said 5-membered heteroaryl is substituted with a C₃-C₅cycloalkyl and further substituted with 0 to 2 R^{A} substituents.

In some embodiments, R¹ is
b) a 5- or 6-membered saturated or partially unsaturated heterocyclyl (having 1-2 heteroatoms independently selected from nitrogen, oxygen, and sulfur), said heterocyclyl substituted with 0-2 groups independently selected from halogen, oxo, —NR₂, optionally substituted C₁₋₄ aliphatic, —OR, azetidinyl optionally substituted with 1 or 2 independently selected halogen, and pyrrolidinyl optionally substituted with 1 or 2 independently selected halogen; or
c) a 6-8 membered saturated or partially unsaturated bridged bicyclic heterocyclyl (having 1-4 heteroatoms independently selected from nitrogen, oxygen, and sulfur), said heterocyclyl substituted with 0-2 groups independently selected from halogen, oxo, —NR₂, optionally substituted C₁₋₄ aliphatic, —OR, azetidinyl optionally substituted with 1 or 2 independently selected halogen, and pyrrolidinyl optionally substituted with 1 or 2 independently selected halogen;
provided that each optionally substituted C₁₋₄ aliphatic or —OR substituent of b) or c), is independently unsubstituted or has substitutions limited to —OH; C₁-C₂alkoxy; or 1, 2 or 3 independently selected halogens.

In some embodiments, R¹ is b) a 5- or 6-membered saturated or partially unsaturated heterocyclyl (having 1-2 heteroatoms independently selected from nitrogen, oxygen, and sulfur), said heterocyclyl substituted with 0-2 groups independently selected from halogen, oxo, —NR₂, optionally substituted C₁₋₄ aliphatic, —OR, azetidinyl optionally substituted with 1 or 2 independently selected halogen, and pyrrolidinyl optionally substituted with 1 or 2 independently selected halogen.

In some embodiments, R¹ is a 5- or 6-membered heteroaryl (having 1-4 heteroatoms independently selected from nitrogen, oxygen, and sulfur) substituted with 1 group selected from C₁-C₆alkoxy and C₃-C₆cycloalkyl, wherein said 5- or 6-membered heteroaryl is further substituted with 0-3 independently selected R^{A}.

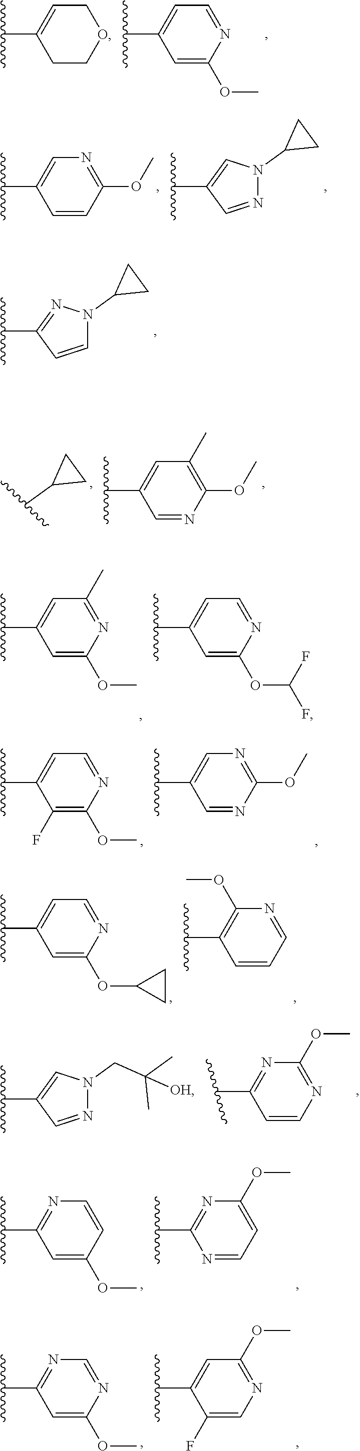

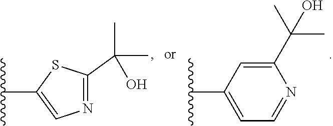

In some embodiments, R¹ is

In some embodiments, R¹ is as selected from one of the substituents of Table 1.

As described generally above, R^{A} is independently selected at each occurrence from the group-consisting of halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, an optionally substituted C₁₋₆ aliphatic group.

In some embodiments, R^{A} is independently selected at each occurrence from the group consisting of halogen, —OR, or an optionally substituted C₁₋₆ aliphatic group. In some embodiments, R^{A} is independently selected at each occurrence from a halogen. In some embodiments, R^{A} is independently selected at each occurrence from an —OR. In some embodiments, R^{A} is independently selected at each occurrence from an optionally substituted C₁₋₆ aliphatic group.

In some embodiments, R^{A} is as selected from one of the substituents of Table 1.

As described generally above, R² is C(R^{B})₂C(O)N(R)R^{2A}. In some embodiments, R² is CH₂C(O)N(H)R^{2A}. In some embodiments, R² is C(R^{B})₂C(O)N(H)R^{2A}, wherein R^{2A} is phenyl optionally substituted with 1, 2, or 3 substituents independently selected from halogen, C₁-C₄alkyl, or haloC₁-C₄alkyl. In some embodiments, R² is

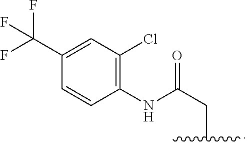 [SMILES: *CC(=O)Nc1ccc(C(F)(F)F)cc1Cl]

In some embodiments, R² is as selected from one of the substituents of Table 1.

As described generally above, R^{B} is independently selected at each occurrence from hydrogen, —CH₃, or —CH₂CH₃, or two R^{B} taken together with the carbon to which they are attached form a cyclopropyl ring. In some embodiments, R^{B} is independently selected at each occurrence from hydrogen, —CH₃, or —CH₂CH₃. In some embodiments, R^{B} is hydrogen. In some embodiments, two R^{B} taken together with the carbon to which they are attached form a cyclopropyl ring.

In some embodiments, R^{B} is as selected from one of the substituents of Table 1.

As described generally above, R^{2A} is phenyl or pyridyl, each of which is optionally substituted with 1, 2, or 3 substituents independently selected from halogen, C₁-C₄alkyl, haloC₁-C₄alkyl, C₃-C₆cycloalkyl, haloC₃-C₆cycloalkyl, —OH, —CN, C₁-C₄alkoxy, haloC₁-C₄alkoxy, —SF₃, two substituents on adjacent atoms of the phenyl or pyridyl together with their intervening atoms form a 4- to 7-membered carbocyclyl fused to the phenyl or pyridyl, and two substituents on adjacent atoms of the phenyl or pyridyl together with their intervening atoms form a 4- to 7-membered heterocyclyl (having 1-4 heteroatoms independently selected from nitrogen, oxygen, and sulfur) fused to the phenyl or pyridyl; or R^{2A} is 2-benzimidazolyl or 3-quinolinyl, each of which is optionally substituted with 1, 2 or 3 substituents independently selected from halogen, C₁₋₄alkyl and —OH.

In some embodiments, R^{2A} is phenyl optionally substituted with 1, 2, or 3 substituents independently selected from halogen, C₁-C₄alkyl, haloC₁-C₄alkyl, C₃-C₆cycloalkyl, haloC₃-C₆-cycloalkyl, —OH, —CN, C₁-C₄alkoxy, haloC₁-C₄alkoxy, —SF₅, two substituents on adjacent atoms of the phenyl together with their intervening atoms form a 4- to 7-membered carbocyclyl fused to the phenyl, and two substituents on adjacent atoms of the phenyl together with their intervening atoms form a 4- to 7-membered heterocyclyl (having 1-4 heteroatoms independently selected from nitrogen, oxygen, and sulfur) fused to the phenyl.

In some embodiments, R^{2A} is phenyl optionally substituted with 1, 2, or 3 substituents independently selected from halogen, C₁-C₄alkyl, haloC₁-C₄alkyl, C₃-C₆cycloalkyl, haloC₃-C₆-cycloalkyl, —OH, —CN, C₁-C₄alkoxy, haloC₁-C₄alkoxy, and —SF₅. In some embodiments, R^{2A} is phenyl optionally substituted with 1, 2, or 3 substituents independently selected from halogen, C₁-C₄alkyl, and haloC₁-C₄alkyl. In some embodiments, R^{2A} is phenyl optionally substituted with a halogen, C₁-C₄alkyl, and haloC₁-C₄alkyl. In some embodiments, R^{2A} is phenyl optionally substituted with 2 substituents independently selected from halogen, C₁-C₄alkyl, and haloC₁-C₄alkyl. In some embodiments, R^{2A} is phenyl optionally substituted with 3 substituents independently selected from halogen, C₁-C₄alkyl, and haloC₁-C₄alkyl.

In some embodiments, R^{2A} is pyridyl optionally substituted with 1, 2, or 3 substituents independently selected from halogen, C₁-C₄alkyl, haloC₁-C₄alkyl, C₃-C₆cycloalkyl, haloC₃-C₆-cycloalkyl, —OH, —CN, C₁-C₄alkoxy, haloC₁-C₄alkoxy, —SF₅, two substituents on adjacent atoms of the pyridyl together with their intervening atoms form a 4- to 7-membered carbocyclyl fused to the pyridyl, and two substituents on adjacent atoms of the pyridyl together with their intervening atoms form a 4- to 7-membered heterocyclyl (having 1-4 heteroatoms independently selected from nitrogen, oxygen, and sulfur) fused to the pyridyl.

In some embodiments, R^{2A} is pyridyl optionally substituted with 1, 2, or 3 substituents independently selected from halogen, C₁-C₄alkyl, haloC₁-C₄alkyl, C₃-C₆cycloalkyl, haloC₃-C₆-cycloalkyl, —OH, —CN, C₁-C₄alkoxy, haloC₁-C₄alkoxy, and —SF₅. In some embodiments, R^{2A} is pyridyl optionally substituted with 1, 2, or 3 substituents independently selected from halogen, C₁-C₄alkyl, and haloC₁-C₄alkyl. In some embodiments, R^{2A} is pyridyl optionally substituted with a halogen, C₁-C₄alkyl, or haloC₁-C₄alkyl. In some embodiments, R^{2A} is pyridyl optionally substituted with 2 substituents independently selected from halogen, C₁-C₄alkyl, and haloC₁-C₄alkyl. In some embodiments, R^{2A} is pyridyl optionally substituted with 3 substituents independently selected from halogen, C₁-C₄alkyl, and haloC₁-C₄alkyl.

In some embodiments, R^{2A} is 2-benzimidazolyl or 3-quinolinyl, each of which is optionally substituted with 1, 2 or 3 substituents independently selected from halogen, C₁₋₄alkyl and —OH. In some embodiments, R^{2A} is 2-benzimidazolyl optionally substituted with 1, 2 or 3 substituents independently selected from halogen, C₁₋₄alkyl and —OH. In some embodiments, R^{2A} is 3-quinolinyl optionally substituted with 1, 2 or 3 substituents independently selected from halogen, C₁₋₄alkyl and —OH.

In some embodiments, R^{2A} is phenyl comprising a —CF₃ substituent or pyridyl comprising a —CF₃ substituent.

In some embodiments, R^{2A} is as selected from one of the substituents of Table 1.

As described generally above, R³ is hydrogen, C₁-C₄alkyl, C₃-C₅cycloalkyl, C₁-C₄alkoxy, —NHR^{3A}, —N(R^{3A})₂ or C₁-C₄alkylthio each of which, besides hydrogen, is optionally substituted with —OH, 1-5 independently selected halogen, or C₁-C₄alkoxy.

In some embodiments, R³ is hydrogen. In some embodiments, R³ is C₁-C₄alkyl optionally substituted with —OH, 1-5 independently selected halogen, or C₁-C₄alkoxy. In some embodiments, R³ is C₁-C₄alkyl. In some embodiments, R³ is —CH₂CH₃. In some embodiments, R³ is —CH₃. In some embodiments, R³ is C₃-C₅cycloalkyl, C₁-C₄alkoxy, —NHR^{3A}, —N(R^{3A})₂ or C₁-C₄alkylthio optionally substituted with —OH, 1-5 independently selected halogen, or C₁-C₄alkoxy. In some embodiments, R³ is C₃-C₅cycloalkyl optionally substituted with —OH, 1-5 independently selected halogen, or C₁-C₄alkoxy. In some embodiments, R³ is C₁-C₄alkoxy optionally substituted with —OH, 1-5 independently selected halogen, or C₁-C₄alkoxy. In some embodiments, R³ is —NHR^{3A} optionally substituted with —OH, 1-5 independently selected halogen, or C₁-C₄alkoxy. In some embodiments, R³ is —N(R^{3A})₂ optionally substituted with —OH, 1-5 independently selected halogen, or C₁-C₄alkoxy. In some embodiments, R³ is C₁-C₄alkylthio optionally substituted with —OH, 1-5 independently selected halogen, or C₁-C₄alkoxy. In some embodiments, R³ is selected from the group consisting of C₁-C₄alkyl and C₃-C₅cycloalkyl.

In some embodiments, R³ is as selected from one of the substituents of Table 1.

As described generally above, each R^{3A} is independently selected at each occurrence from C₁-C₄alkyl. In some embodiments, R^{3A} is —CH₃. In some embodiments, R^{3A} is —CH₂CH₃. In some embodiments, R^{3A} is propyl. In some embodiments, R^{3A} is butyl.

In some embodiments, R^{3A} is as selected from one of the substituents of Table 1.

As described generally above, R⁴ is selected from one of a), b), and c):
a) R⁴ is a Ring B that is selected from the group consisting of

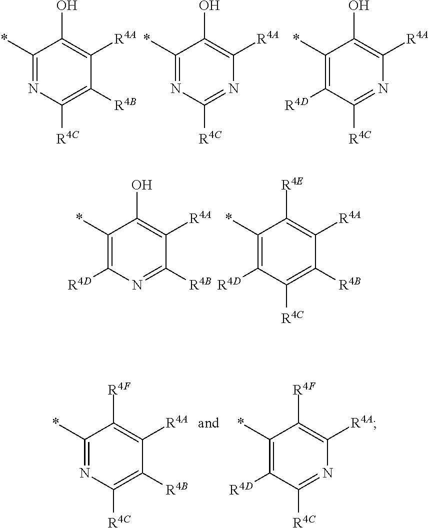

wherein * is a point of attachment to the carbonyl group that is bonded to Ring A in Formula I;
and wherein:
   any substituents that are present on Ring B selected from R^{4A}, R^{4B}, R^{4C}, R^{4D}, R^{4E}, and R^{F} are each independently selected from hydrogen; halogen; —CN; C₁-C₄alkyl; C₂-C₄alkenyl; C₂-C₄alkynyl; C₁-C₄alkoxy; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; or NR^{C}₂; or
   R^{4A} and R^{4B}, along with their intervening atoms, join to form 4- to 7-membered optionally substituted carbocyclyl or optionally substituted heterocyclyl (having 1-4 heteroatoms independently selected from nitrogen, oxygen, and sulfur) that is fused to Ring B; and any substituents that are present on Ring B selected from R^{4C}, R^{4D}, R^{4E}, and R^{4F} are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; or NR^{C}₂; or
   R^{4B} and R^{4C}, along with their intervening atoms, join to form 4- to 7-membered optionally substituted carbocyclyl or optionally substituted heterocyclyl (having 1-4 heteroatoms independently selected from nitrogen, oxygen, and sulfur) that is fused to Ring B; and any substituents that are present on Ring B selected from R^{4A}, R^{4D}, R^{4E}, and R^{4F} are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; or NR^{C}₂; or
   R^{4C} and R^{4D}, along with their intervening atoms, join to form 4- to 7-membered optionally substituted carbocyclyl or optionally substituted heterocyclyl (having 1-4 heteroatoms independently selected from nitrogen, oxygen, and sulfur) that is fused to Ring B; and any substituents that are present on Ring B selected from R^{4A}, R^{4B}, and R^{4E} are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; or NR^{C}₂; or
   R^{4E} is halogen or —OH, and R^{4A}, R^{4B}, R^{4C}, and R^{4D} are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; or NR^{C}₂; or
   R^{4E} and R^{4A}, along with their intervening atoms, join to form 5- to 6-membered optionally substituted heteroaryl (having 1-4 heteroatoms independently selected from nitrogen, oxygen, and sulfur) that is fused to Ring B; and R^{4B}, R^{4C}, and R^{4D} are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; or NR^{C}₂; or
   R^{4F} and R^{4A}, along with their intervening atoms, join to form 5- to 6-membered optionally substituted heteroaryl (having 1-4 heteroatoms independently selected from nitrogen, oxygen, and sulfur) that is fused to Ring B; and any substituents that are present on Ring B selected from R^{4B}, R^{4C}, and R^{4D} are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; or NR^{C}₂; and
   R^{C} is independently selected at each occurrence from hydrogen or C₁-C₄alkyl optionally substituted with —OH, —OCH₃, or —OCH₂CH₃; or NR^{C}₂, taken in combination form a heterocyclic ring selected from azetidinyl, pyrrolidinyl, or piperidinyl, said heterocyclic ring optionally substituted with —CH₃; or
b) R⁴ is a 5-membered heteroaryl (having 1 heteroatom independently selected from nitrogen, oxygen, and sulfur and 0, 1, 2, or 3 additional ring nitrogen atoms), wherein said heteroaryl is optionally substituted with 0-4 groups independently selected from halogen, —OH, —CN, C₁-C₄alkyl, haloC₁-C₄alkyl, cycloC₃-C₆alkyl, and C₁-C₄alkoxy; or
c) R⁴ is a C₁-C₄alkyl, C₁-C₄alkoxy, or cycloC₃-C₆alkyl, each of which is substituted with 0-3 groups independently selected from halogen, —CN, —OH, C₁-C₄alkyl, C₁-C₄alkoxy, optionally substituted 5- to 6-membered heterocyclyl, and optionally substituted 5- to 6-membered heterocyclyloxy;

In some embodiments, R⁴ is Ring B of the following structure:

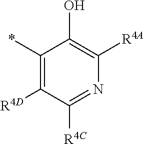

wherein * is a point of attachment to the carbonyl group that is bonded to Ring A in Formula I;
and wherein:
   R^{4A}, R^{4C}, and R^{4D} are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; or NR^{C}₂; or
   R^{4C} and R^{4D}, along with their intervening atoms, join to form 4- to 7-membered carbocyclyl or heterocyclyl (having 1-4 heteroatoms independently selected from nitrogen, oxygen, and sulfur) that is fused to Ring B; and R^{4A} is hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; or NR^{C}₂; and
   R^{C} is independently selected at each occurrence from hydrogen or C₁-C₄alkyl optionally substituted with —OH, —OCH₃, or —OCH₂CH₃; or NR^{C}₂, taken in combination form a heterocyclic ring selected from azetidinyl, pyrrolidinyl, or piperidinyl, said heterocyclic ring optionally substituted with —CH₃.

In some embodiments, R⁴ is Ring B of the following structure:

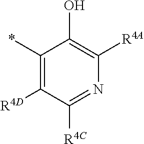

wherein * is a point of attachment to the carbonyl group that is bonded to Ring A in Formula I;
and wherein:
   R^{4A} is —OCH₃, —OCH₂CH₃, or —OCHF₂;
   R^{4C} and R^{4D} are each independently selected from hydrogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; or NR^{C}₂; and
   R^{C} is independently selected at each occurrence from hydrogen or C₁-C₄alkyl optionally substituted with —OH, —OCH₃, or —OCH₂CH₃; or NR^{C}₂, taken in combination form a heterocyclic ring selected from azetidinyl, pyrrolidinyl, or piperidinyl, said heterocyclic ring optionally substituted with —CH₃; or
R⁴ is a 5-membered heteroaryl (having 1 heteroatom independently selected from nitrogen, oxygen, and sulfur and 0, 1, 2, or 3 additional ring nitrogen atoms), wherein said heteroaryl is optionally substituted with 0-4 substituents independently selected from halogen, —OH, —CN, C₁-C₄alkyl, haloC₁-C₄alkyl, cycloC₃-C₆alkyl, and C₁-C₄alkoxy.

In some embodiments, R⁴ is Ring B of the following structure:

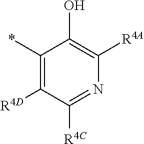

wherein * is a point of attachment to the carbonyl group that is bonded to Ring A in Formula I;
and wherein:
   R^{4A} is —OCH₃, —OCH₂CH₃, or —OCHF₂;
   R^{4C} and R^{4D} are each independently selected from hydrogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; or NR^{C}₂; and
   R^{C} is independently selected at each occurrence from hydrogen or C₁-C₄alkyl optionally substituted with —OH, —OCH₃, or —OCH₂CH₃; or NR^{C}₂, taken in combination form a heterocyclic ring selected from azetidinyl, pyrrolidinyl, or piperidinyl, said heterocyclic ring optionally substituted with —CH₃.

In some embodiments, R⁴ is Ring B of the following structure:

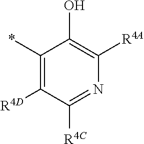

wherein * is a point of attachment to the carbonyl group that is bonded to Ring A in Formula I;
and wherein:
   R^{4A}, R^{4C}, and R^{4D} are each independently selected from hydrogen; halogen; and C₁-C₄alkyl.

In some embodiments, R⁴ is Ring B of the following structure:

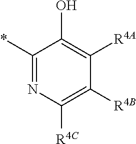

wherein * is a point of attachment to the carbonyl group that is bonded to Ring A in Formula I;
and wherein:
   R^{4A}, R^{4B}, and R^{4C} are each independently selected from hydrogen; halogen; —CN; C₁-C₄alkyl; C₂-C₄alkenyl; C₂-C₄alkynyl; C₁-C₄alkoxy; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; or NR^{C}₂; or
   R^{4A} and R^{4B}, along with their intervening atoms, join to form 4- to 7-membered carbocyclyl or heterocyclyl (having 1-4 heteroatoms independently selected from nitrogen, oxygen, and sulfur) that is fused to Ring B; and R^{4C} is hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; or NR^{C}₂; or
   R^{4B} and R^{4C}, along with their intervening atoms, join to form 4- to 7-membered carbocyclyl or heterocyclyl (having 1-4 heteroatoms independently selected from nitrogen, oxygen, and sulfur) that is fused to Ring B; and R^{4A} is hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; or NR^{C}₂; and
   R^{C} is independently selected at each occurrence from hydrogen or C₁-C₄alkyl optionally substituted with —OH, —OCH₃, or —OCH₂CH₃; or NR^{C}₂, taken in combination form a heterocyclic ring selected from azetidinyl, pyrrolidinyl, or piperidinyl, said heterocyclic ring optionally substituted with —CH₃.

In some embodiments, R⁴ is Ring B of the following structure:

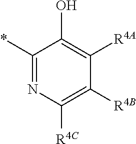

wherein * is a point of attachment to the carbonyl group that is bonded to Ring A in Formula I;
and wherein:
   R^{4A} and R^{4B}, along with their intervening atoms, join to form 4- to 7-membered carbocyclyl or heterocyclyl (having 1-4 heteroatoms independently selected from nitrogen, oxygen, and sulfur) that is fused to Ring B; and
   R^{4C} is hydrogen.

In some embodiments, R⁴ is Ring B of the following structure:

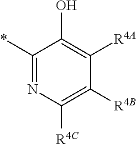

wherein * is a point of attachment to the carbonyl group that is bonded to Ring A in Formula I;
and wherein:
   R^{4A} and R^{4B}, along with their intervening atoms, join to form 5-membered heterocyclyl (having 1 oxygen atom) that is fused to Ring B; and
   R^{4C} is hydrogen.

In some embodiments, R⁴ is Ring B of the following structure:

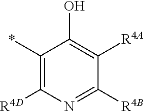

wherein * is a point of attachment to the carbonyl group that is bonded to Ring A in Formula I;
and wherein:
   R^{4A}, R^{4B}, and R^{4D} are each independently selected from hydrogen; halogen; —CN; C₁-C₄alkyl; C₂-C₄alkenyl; C₂-C₄alkynyl; C₁-C₄alkoxy; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; or NR^{C}₂; or
   R^{4A} and R^{4B}, along with their intervening atoms, join to form 4- to 7-membered carbocyclyl or heterocyclyl (having 1-4 heteroatoms independently selected from nitrogen, oxygen, and sulfur) that is fused to Ring B; and R^{4D} is hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; or NR^{C}₂; and
   R^{C} is independently selected at each occurrence from hydrogen or C₁-C₄alkyl optionally substituted with —OH, —OCH₃, or —OCH₂CH₃; or NR^{C}₂, taken in combination form a heterocyclic ring selected from azetidinyl, pyrrolidinyl, or piperidinyl, said heterocyclic ring optionally substituted with —CH₃.

In some embodiments, R⁴ is Ring B of the following structure:

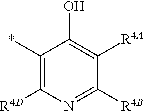

wherein * is a point of attachment to the carbonyl group that is bonded to Ring A in Formula I;
and wherein:
   R^{4A} and R^{4D} are each hydrogen; and
   R^{4B} is C₁-C₄alkyl.

In some embodiments, R⁴ is Ring B of the following structure:

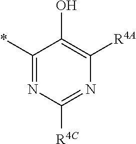

wherein * is a point of attachment to the carbonyl group that is bonded to Ring A in Formula I;
and wherein:
   R^{4A} and R^{4C} are each independently selected from hydrogen; halogen; —CN; C₁-C₄alkyl; C₂-C₄alkenyl; C₂-C₄alkynyl; C₁-C₄alkoxy; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; or NR^{C}₂; and
R^{C} is independently selected at each occurrence from hydrogen or C₁-C₄alkyl optionally substituted with —OH, —OCH₃, or —OCH₂CH₃; or NR^{C}₂, taken in combination form a heterocyclic ring selected from azetidinyl, pyrrolidinyl, or piperidinyl, said heterocyclic ring optionally substituted with —CH₃.

In some embodiments, R⁴ is Ring B of the following structure:

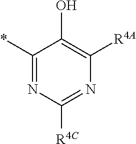

wherein * is a point of attachment to the carbonyl group that is bonded to Ring A in Formula I;
and wherein:
   R^{4A} and R^{4C} are each independently selected from hydrogen and C₁-C₄alkyl.

In some embodiments, R⁴ is Ring B of the following structure:

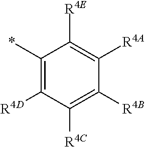

wherein * is a point of attachment to the carbonyl group that is bonded to Ring A in Formula I;
and wherein:
   R^{4A}, R^{4B}, R^{4C}, R^{4D}, and R^{4E} are each independently selected from hydrogen; halogen; —CN; C₁-C₄alkyl; C₂-C₄alkenyl; C₂-C₄alkynyl; C₁-C₄alkoxy; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; or NR^{C}₂; or
   R^{4A} and R^{4B}, along with their intervening atoms, join to form 4- to 7-membered carbocyclyl or heterocyclyl (having 1-4 heteroatoms independently selected from nitrogen, oxygen, and sulfur) that is fused to Ring B; and R^{4C}, R^{4D}, and R^{4E} are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; or NR^{C}₂; or
   R^{4C} and R^{4D}, along with their intervening atoms, join to form 4- to 7-membered carbocyclyl or heterocyclyl (having 1-4 heteroatoms independently selected from nitrogen, oxygen, and sulfur) that is fused to Ring B; and R^{4A}, R^{4B}, and R^{4E} are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; or NR^{C}₂; or
   R^{4E} is halogen or —OH, and R^{4A}, R^{4B}, R^{4C}, and R^{4D} are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; or NR^{C}₂; or
   R^{4E} and R^{4A}, along with their intervening atoms, join to form 5- to 6-membered heteroaryl (having 1-4 heteroatoms independently selected from nitrogen, oxygen, and sulfur) fused to Ring B; and R^{4B}, R^{4C}, and R^{4D} are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; or NR^{C}₂; and
   R^{C} is independently selected at each occurrence from hydrogen or C₁-C₄alkyl optionally substituted with —OH, —OCH₃, or —OCH₂CH₃; or NR^{C}₂, taken in combination form a heterocyclic ring selected from azetidinyl, pyrrolidinyl, or piperidinyl, said heterocyclic ring optionally substituted with —CH₃.

In some embodiments, R⁴ is Ring B of the following structure:

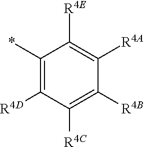

wherein * is a point of attachment to the carbonyl group that is bonded to Ring A in Formula I;
and wherein:
   R^{4A}, R^{4B}, R^{4C}, R^{4D}, and R^{4E} are each independently selected from hydrogen; halogen; C₁-C₄alkyl; and C₁-C₄alkoxy; or
   R^{4C} and R^{4D}, along with their intervening atoms, join to form a 4- to 7-membered heterocyclyl (having 1-3 nitrogen atoms) fused to Ring B; and R^{4A}, R^{4B}, and R^{4E} are each hydrogen.

In some embodiments, R⁴ is Ring B of the following structure:

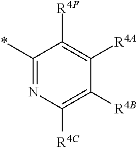

wherein * is a point of attachment to the carbonyl group that is bonded to Ring A in Formula I;
and wherein:
   R^{4F} and R^{4A}, along with their intervening atoms, join to form 5- to 6-membered heteroaryl (having 1-4 heteroatoms independently selected from nitrogen, oxygen, and sulfur) fused to Ring B; and R^{4B} and R^{4C} are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; or NR^{C}₂;
   R^{C} is independently selected at each occurrence from hydrogen or C₁-C₄alkyl optionally substituted with —OH, —OCH₃, or —OCH₂CH₃; or NR^{C}₂, taken in combination form a heterocyclic ring selected from azetidinyl, pyrrolidinyl, or piperidinyl, said heterocyclic ring optionally substituted with —CH₃.

In some embodiments, R⁴ is Ring B of the following structure:

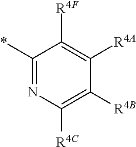

wherein * is a point of attachment to the carbonyl group that is bonded to Ring A in Formula I;
and wherein:
   R^{4F} and R^{4A}, along with their intervening atoms, join to form 5- to 6-membered heteroaryl (having 1-2 nitrogen atoms) fused to Ring B; and R^{4B} and R^{4C} are each hydrogen.

In some embodiments, R⁴ is Ring B of the following structure:

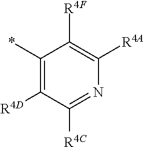

wherein * is a point of attachment to the carbonyl group that is bonded to Ring A in Formula I;
and wherein:
   R^{4A}, R^{4C}, R^{4D}, and R^{F} are each independently selected from hydrogen; halogen; —CN; C₁-Caalkyl; C₂-C₄alkenyl; C₂-C₄alkynyl; C₁-C₄alkoxy; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; or NR^{C}₂; or
   R^{4C} and R^{4D}, along with their intervening atoms, join to form 4- to 7-membered optionally substituted carbocyclyl or optionally substituted heterocyclyl (having 1-4 heteroatoms independently selected from nitrogen, oxygen, and sulfur) that is fused to Ring B; and R^{4A} and R^{4F} are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; or NR^{C}₂; or
   R^{4F} and R^{4A}, along with their intervening atoms, join to form 5- to 6-membered optionally substituted heteroaryl (having 1-4 heteroatoms independently selected from nitrogen, oxygen, and sulfur) that is fused to Ring B; and R^{4C} and R^{4D} are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; or NR^{C}₂; and
   R^{C} is independently selected at each occurrence from hydrogen or C₁-C₄alkyl optionally substituted with —OH, —OCH₃, or —OCH₂CH₃; or NR^{C}₂, taken in combination form a heterocyclic ring selected from azetidinyl, pyrrolidinyl, or piperidinyl, said heterocyclic ring optionally substituted with —CH₃.

In some embodiments, R⁴ is a 5-membered heteroaryl (having 1 heteroatom independently selected from nitrogen, oxygen, and sulfur and 0, 1, 2, or 3 additional ring nitrogen atoms), wherein said heteroaryl is optionally substituted with 0-4 groups independently selected from halogen, —OH, —CN, C₁-C₄alkyl, haloC₁-C₄alkyl, cycloC₃-C₆alkyl, and C₁-C₄alkoxy.

In some embodiments, R⁴ is a 5-membered heteroaryl (having 1 heteroatom independently selected from nitrogen, oxygen, and sulfur and 0, 1, 2, or 3 additional ring nitrogen atoms), wherein said heteroaryl is optionally substituted with 0-4 groups independently selected from OH, —CH₃, —CHF₂, cyclopropyl, and —OCH₃.

In some embodiments, R⁴ is a C₁-C₄alkyl, C₁-C₄alkoxy, or cycloC₃-C₆alkyl, each of which is substituted with 0-3 groups independently selected from halogen, —CN, —OH, C₁-C₄alkyl, C₁-C₄alkoxy, optionally substituted 5- to 6-membered heterocyclyl, and optionally substituted 5- to 6-membered heterocyclyloxy. In some embodiments, R⁴ is a C₁-C₄alkyl, substituted with 0-3 independently selected halogen, —CN, —OH, C₁-C₄alkyl, and C₁-C₄alkoxy. In some embodiments, R⁴ is a C₁-C₄alkoxy, substituted with 0-3 independently selected halogen, —CN, —OH, C₁-C₄alkyl, and C₁-C₄alkoxy. In some embodiments, R⁴ is a cycloC₃-C₆alkyl, substituted with 0-3 independently selected halogen, —CN, —OH, C₁-C₄alkyl, and C₁-C₄alkoxy.

In some embodiments, R⁴ is an isoxazolyl substituted with —OH or C₁-C₄alkoxy.

In some embodiments, R⁴ is a 5-membered heteroaryl (having 1 heteroatom independently selected from nitrogen, oxygen, and sulfur and 0, 1, 2, or 3 additional ring nitrogen atoms) selected from the group consisting of thiophenyl, imidazolyl, pyrazolyl, tetrazolyl, thiazolyl, isothiazolyl, 1,2,5-thiadiazolyl, 1,3,4-thiadiazolyl, oxazolyl, isoxazolyl, 1,2,4-oxadiazolyl, 1,2,3-triazolyl, 1,2,4-triazolyl, wherein said heteroaryl is optionally substituted with 0-4 groups independently selected from halogen, —OH, —CN, C₁-C₄alkyl, haloC₁-C₄alkyl, cycloC₃-C₆alkyl, and C₁-C₄alkoxy.

In some embodiments, R⁴ is

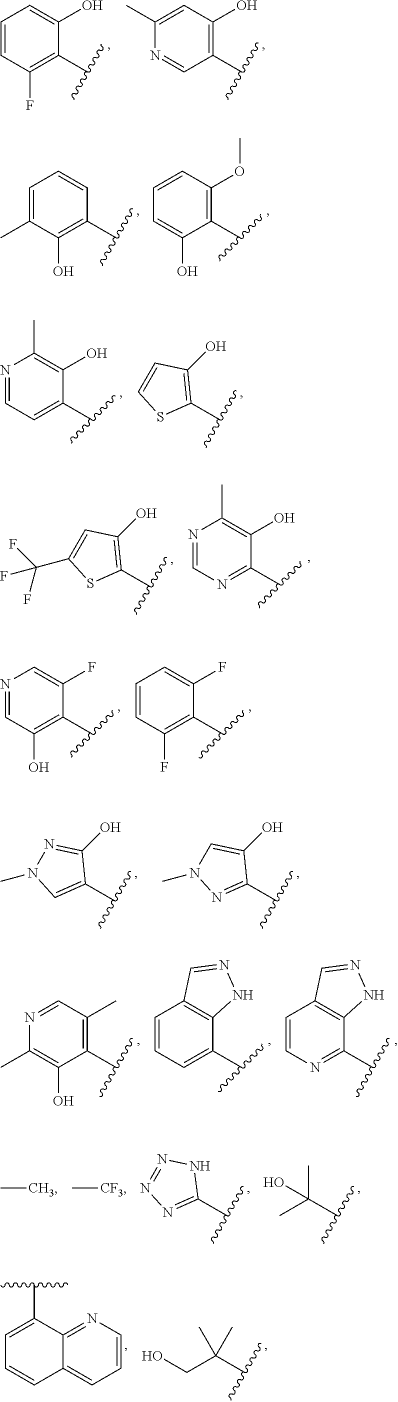

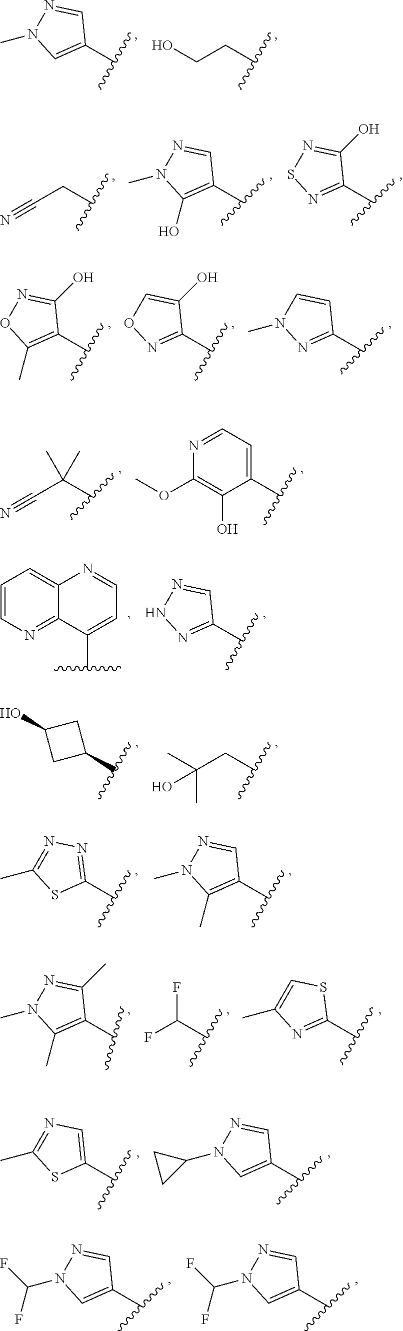

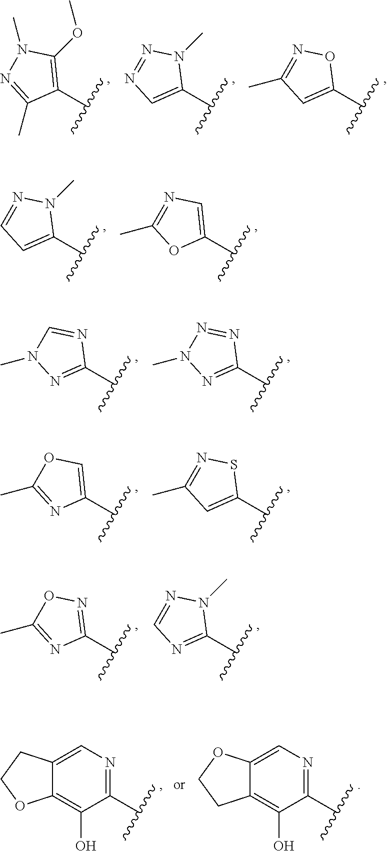

In some embodiments, R⁴ is as shown in a substituent of Table 1.

As described generally above, each R is independently hydrogen, or an optionally substituted C₁₋₆ aliphatic group, an optionally substituted phenyl, an optionally substituted 3-7 membered saturated or partially unsaturated carbocyclic ring, an optionally substituted 3-7 membered saturated or partially unsaturated heterocyclic ring (having 1-2 heteroatoms independently selected from nitrogen, oxygen, and sulfur), or an optionally substituted 5-6 membered heteroaryl ring (having 1-4 heteroatoms independently selected from nitrogen, oxygen, and sulfur); or two R groups on the same atom are taken together with the same atom to form an optionally substituted 4-7 membered saturated ring, partially unsaturated ring, or heteroaryl ring (wherein said 4-7 membered saturated ring, partially unsaturated ring, or heteroaryl ring, has 0-3 heteroatoms independently selected from nitrogen, oxygen, and sulfur).

In some embodiments, each R is independently hydrogen, or an optionally substituted C₁₋₆ aliphatic group, an optionally substituted phenyl, an optionally substituted 3-7 membered saturated or partially unsaturated carbocyclic ring, an optionally substituted 3-7 membered saturated or partially unsaturated heterocyclic ring (having 1-2 heteroatoms independently selected from nitrogen, oxygen, and sulfur), or an optionally substituted 5-6 membered heteroaryl ring (having 1-4 heteroatoms independently selected from nitrogen, oxygen, and sulfur).

In some embodiments, two R groups on the same atom are taken together with the same atom to form an optionally substituted 4-7 membered saturated ring, partially unsaturated ring, or heteroaryl ring (wherein said 4-7 membered saturated ring, partially unsaturated ring, or heteroaryl ring, has 0-3 heteroatoms independently selected from nitrogen, oxygen, and sulfur).

In some embodiments, each R is independently hydrogen or a C₁₋₆ alkyl. In some embodiments, R is hydrogen.

In some embodiments, each R is as selected from one or more of the substituents of Table 1.

As described generally above, Formula I has the proviso that one or two of the following provisions 1)-7) is satisfied, further provided that two of 1), 2), and 3) are not concurrently satisfied, and further provided that two of 4), 5), 6) and 7) are not concurrently satisfied:
1) R¹ is a) a 5- or 6-membered heteroaryl (having 1-4 heteroatoms independently selected from nitrogen, oxygen, and sulfur) substituted with 1 or 2 groups independently selected from C₁-C₆alkoxy, C₃-C₆cycloalkyl, or C₃-C₆cycloalkoxy, wherein said 5- or 6-membered heteroaryl is further substituted with 0-3 independently selected R^{A};
2) R¹ is c) 6-8 membered saturated or partially unsaturated bridged bicyclic heterocyclyl (having 1-4 heteroatoms independently selected from nitrogen, oxygen, and sulfur), said heterocyclyl substituted with 0-2 groups independently selected from halogen, oxo, —NR₂, optionally substituted C₁₋₄ aliphatic, —OR, azetidinyl optionally substituted with 1 or 2 independently selected halogen, and pyrrolidinyl optionally substituted with 1 or 2 independently selected halogen;
3) R¹ is d) a 3-7 membered optionally substituted carbocyclyl or an optionally substituted C₂-C₄alkenyl;
4) R⁴ is a 5-membered heteroaryl (having 1 heteroatom independently selected from nitrogen, oxygen, and sulfur and 0, 1, 2, or 3 additional ring nitrogen atoms), wherein said heteroaryl is optionally substituted with 0-4 groups independently selected from halogen, —OH, —CN, C₁-C₄alkyl, haloC₁-C₄alkyl, cycloC₃-C₆alkyl, and C₁-C₄alkoxy;
5) R⁴ is selected from:

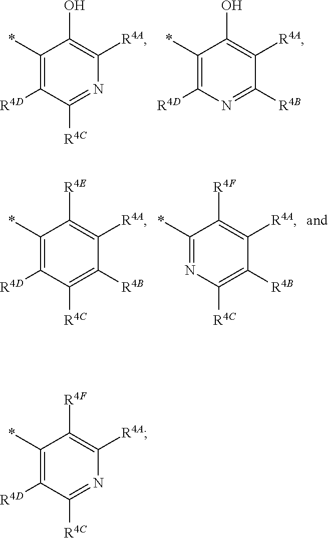

6) R⁴ is:

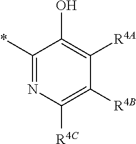

wherein
   R^{4A} and R^{4B}, along with their intervening atoms, join to form 4- to 7-membered optionally substituted carbocyclyl or optionally substituted heterocyclyl (having 1-4 heteroatoms independently selected from nitrogen, oxygen, and sulfur), or wherein R^{4B} and R^{4C} along with their intervening atoms, join to form 4- to 7-membered optionally substituted carbocyclyl or optionally substituted heterocyclyl (having 1-4 heteroatoms independently selected from nitrogen, oxygen, and sulfur);
7) R⁴ is a C₁-C₄alkyl, C₁-C₄alkoxy, or cycloC₃-Coalkyl, each of which is substituted with 0-3 groups independently selected from halogen, —CN, —OH, C₁-C₄alkyl, optionally substituted 5- to 6-membered heterocyclyl, and C₁-C₄alkoxy.

In some embodiments, only provision 1) is satisfied. In some embodiments, only provision 2) is satisfied. In some embodiments, only provision 3) is satisfied. In some embodiments, only provision 4) is satisfied. In some embodiments, only provision 5) is satisfied. In some embodiments, only provision 6) is satisfied. In some embodiments, only provision 7) is satisfied.

In some embodiments, provisions 1) and 4) are satisfied. In some embodiments, provisions 1) and 5) are satisfied. In some embodiments, provisions 1) and 6) are satisfied. In some embodiments, provisions 1) and 7) are satisfied.

In some embodiments, provisions 2) and 4) are satisfied. In some embodiments, provisions 2) and 5) are satisfied. In some embodiments, provisions 2) and 6) are satisfied. In some embodiments, provisions 2) and 7) are satisfied.

In some embodiments, provisions 3) and 4) are satisfied. In some embodiments, provisions 3) and 5) are satisfied. In some embodiments, provisions 3) and 6) are satisfied. In some embodiments, provisions 3) and 7) are satisfied.

In some embodiments, the compound of Formula I is a compound of Formula IIa, IIb, or IIc:

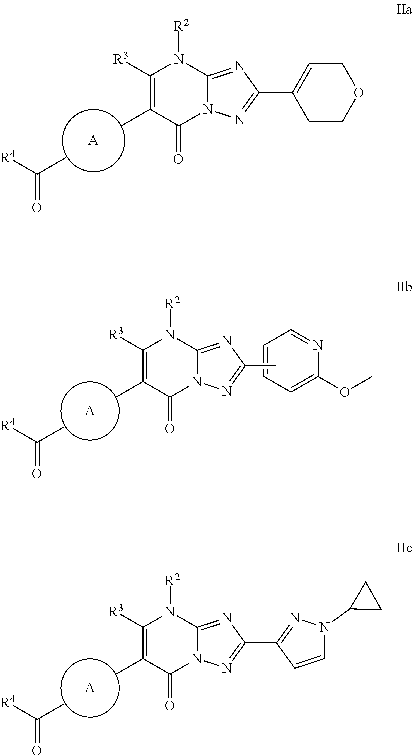

wherein Ring A, R², R³, and R⁴, are as defined herein, both singly and in combination.

In some embodiments, the compound of Formula I is a compound of Formula IIIa, IIIb, or IIIc:

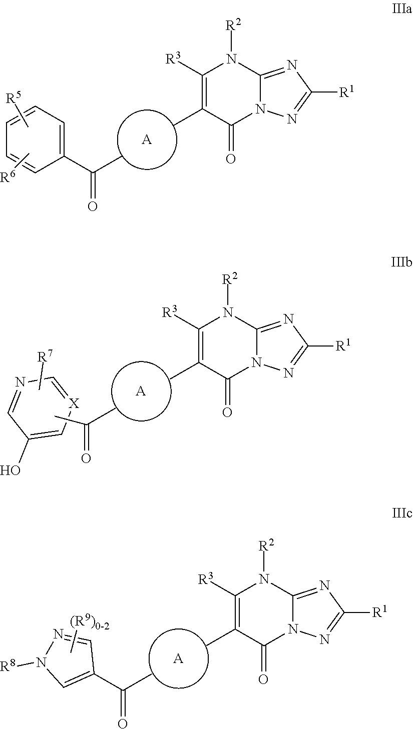

wherein Ring A, R¹, R², and R³, are as defined herein, both singly and in combination, and wherein:
X is CH, CR⁷, or N;
R⁵ is-OH or halogen;
R⁶ is halogen, C₁₋₄alkyl, or C₁₋₄alkoxy;
each R⁷ is independently hydrogen, halogen, C₁₋₄alkyl, or C₁₋₄alkoxy;
R⁸ is C₁₋₄alkyl;
each of the 0-2 instances of R⁹ is independently a hydrogen or C₁₋₄alkyl. In some embodiments:
X is CH or N;
R⁵ is —OH or fluoro;
R⁶ is fluoro, —CH₃, or —OCH₃;
each R⁷ is independently hydrogen, fluoro, —CH₃, or —OCH₃;
R⁸ is —CH₃;
each instance of R⁹ is independently a hydrogen or —CH₃.

In some embodiments, the compound of Formula I is a compound of Formula IVa, IVb, or IVc:

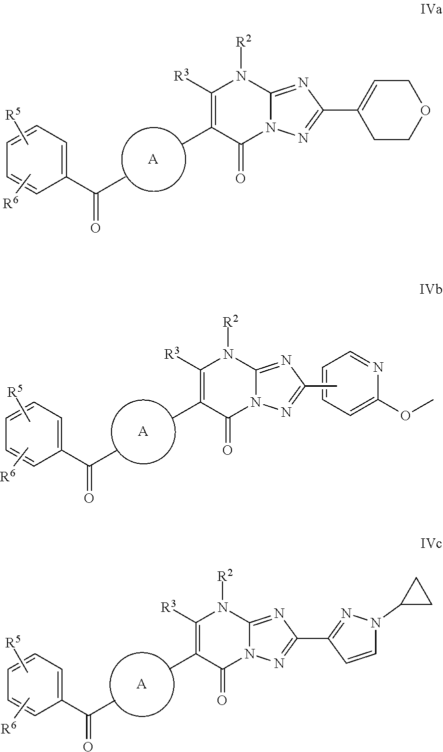

wherein Ring A, R², and R³, are as defined herein, both singly and in combination and wherein: R⁵ is —OH or halogen; and
R⁶ is halogen, C₁₋₄alkyl, or C₁₋₄alkoxy. In some embodiments: R⁵ is —OH or fluoro; and R⁶ is fluoro, —CH₃, or —OCH₃.

In some embodiments, the compound of Formula I is a compound of Formula Va, Vb, or Vc:

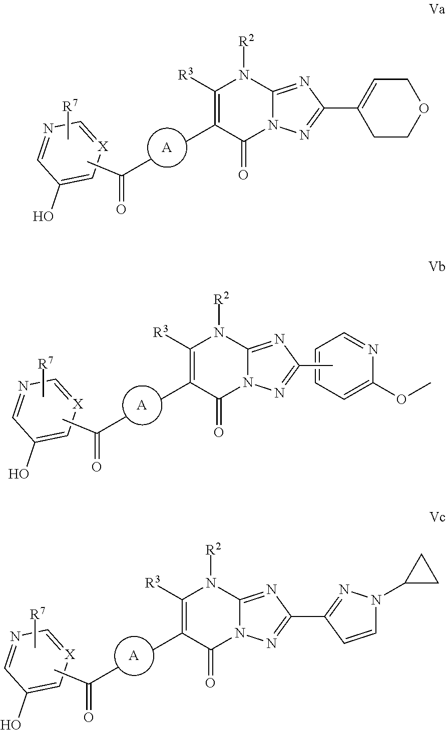

wherein Ring A, R², and R³, are as defined herein, both singly and in combination, and wherein: X is CH, CR⁷, or N; and each R⁷ is independently hydrogen, halogen, C₁₋₄alkyl, or C₁₋₄alkoxy. In some embodiments: X is CH or N; and R⁷ is hydrogen, fluoro, —CH₃, or —OCH₃.

In some embodiments, the compound of Formula I is a compound of Formula VIa, VIb, or VIc:

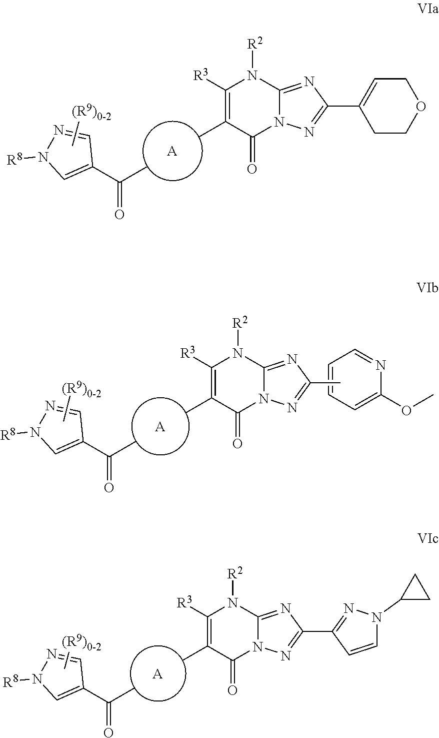

wherein Ring A, R², and R³, are as defined herein, both singly and in combination, and wherein: R⁸ is C₁₋₄alkyl; and each of the 0-2 instances of R⁹ are independently a hydrogen or C₁₋₄alkyl. In some embodiments: R⁸ is —CH₃; and each instance of R⁹ is independently a hydrogen or —CH₃.

In some embodiments, the compound of Formula I is a compound of Formula VIIa, VIIb, or VIIc:

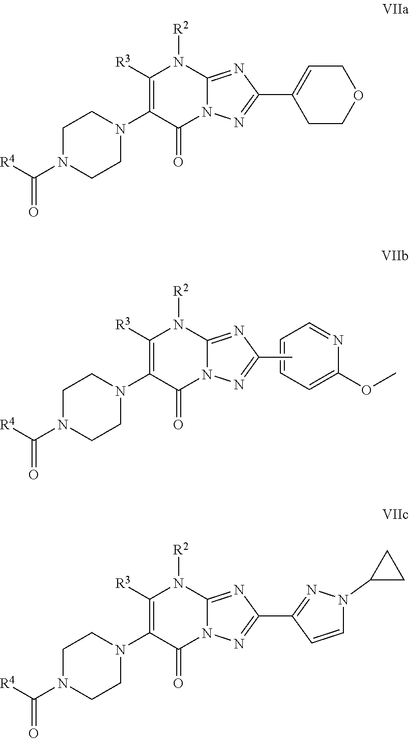

wherein R², R³, and R⁴, are as defined herein, both singly and in combination.

In some embodiments, the compound of Formula I is a compound of Formula VIIIa, VIIIb, or VIIIc:

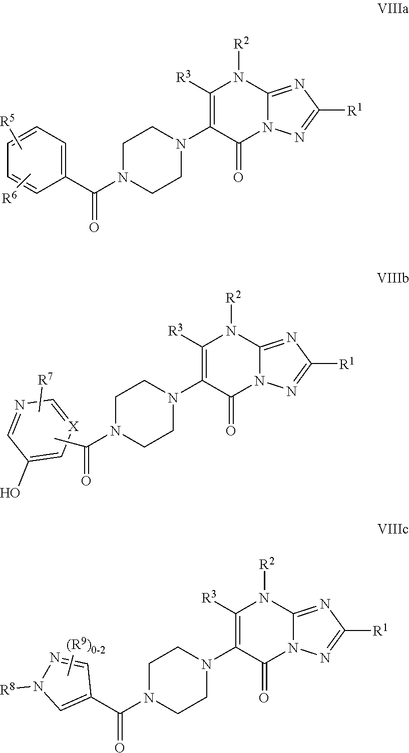

wherein R¹, R², and R³, are as defined herein, both singly and in combination, and wherein:
X is CH, CR⁷, or N;
R⁵ is —OH or halogen;
R⁶ is halogen, C₁₋₄alkyl, or C₁₋₄alkoxy;
each R⁷ is independently hydrogen, halogen, C₁₋₄alkyl, or C₁₋₄alkoxy;
R⁸ is C₁₋₄alkyl; and
each of the 0-2 instances of R⁹ are independently a hydrogen or C₁₋₄alkyl. In some embodiments:
X is CH or N;
R⁵ is —OH or fluoro;
R⁶ is fluoro, —CH₃, or —OCH₃;
each R⁷ is independently hydrogen, fluoro, —CH₃, or —OCH₃;
R⁸ is —CH₃; and
each instance of R⁹ is independently a hydrogen or —CH₃.

In some embodiments, the compound of Formula I is a compound of Formula IXa, IXb, or IXc:

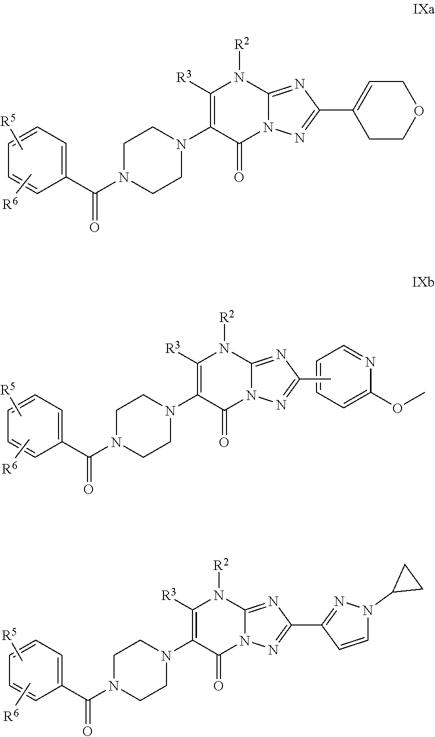

wherein R² and R³, are as defined herein, both singly and in combination and wherein:
R⁵ is —OH or halogen; and
R⁶ is halogen, C₁₋₄alkyl, or C₁₋₄alkoxy. In some embodiments: R⁵ is —OH or fluoro; and R⁶ is fluoro, —CH₃, or —OCH₃.

In some embodiments, the compound of Formula I is a compound of Formula Xa, Xb, or Xc:

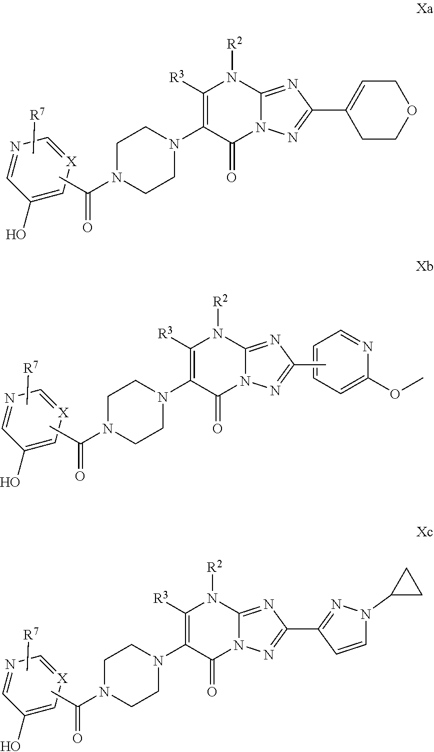

wherein R² and R³, are as defined herein, both singly and in combination, and wherein:
X is CH, CR⁷, or N; and
each R⁷ is independently hydrogen, halogen, C₁₋₄alkyl, or C₁₋₄alkoxy.

In some embodiments: X is CH or N; and each R⁷ is independently hydrogen, fluoro, —CH₃, or —OCH₃.

In some embodiments, the compound of Formula I is a compound of Formula XIa, XIb, or XIc:

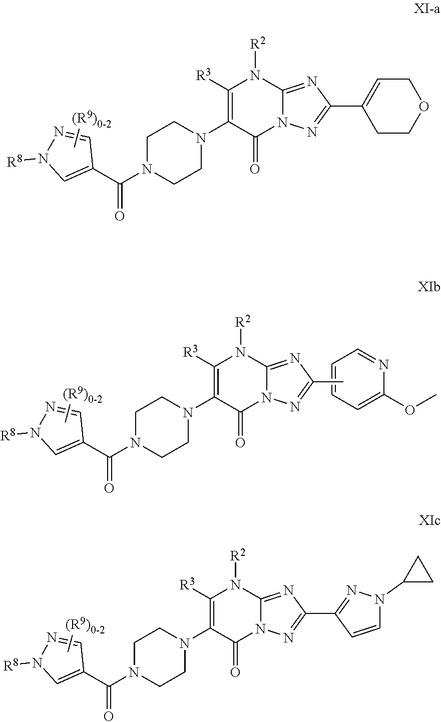

wherein R² and R³, are as defined herein, both singly and in combination, and wherein:
R⁸ is C₁₋₄alkyl; and

each of the 0-2 instances of R⁹ are independently a hydrogen or C₁₋₄alkyl. In some embodiments:
R⁸ is —CH₃; and each instance of R⁹ is independently a hydrogen or —CH₃.

In some embodiments, the compound of Formula I is selected from one of those depicted in Table 1, or a pharmaceutically acceptable salt thereof. Table 1 identifies compounds by their IUPAC name and Table 2 lists the same compounds and shows their chemical structure. In the event of any discrepancy between Table 1's name for a compound and Table 2's structure for that same compound, Table 2's compound structures will dominate and identify the compound corresponding to each respective compound number (I-#) in Table 1.

TABLE 1

| No. | IUPAC Name |
|---|---|
| I-1 | N-[2-chloro-4-(trifluoromethyl)phenyl]-2-[2-(3,6-dihydro-2H-pyran-4-yl)-5-ethyl-6- |
| | [4-(2-fluoro-6-hydroxybenzoyl)piperazin-1-yl]-7-oxo-4H,7H-[1,2,4]triazolo[1,5- |
| | a]pyrimidin-4-yl]acetamide |
| I-2 | N-[2-chloro-4-(trifluoromethyl)phenyl]-2-[2-(3,6-dihydro-2H-pyran-4-yl)-5-ethyl-6- |
| | [4-(4-hydroxy-6-methylpyridine-3-carbonyl)piperazin-1-yl]-7-oxo-4H,7H- |
| | [1,2,4]triazolo[1,5-a]pyrimidin-4-yl]acetamide |
| I-3 | N-[2-chloro-4-(trifluoromethyl)phenyl]-2-[2-(3,6-dihydro-2H-pyran-4-yl)-5-ethyl-6- |
| | [4-(2-hydroxy-3-methylbenzoyl)piperazin-1-yl]-7-oxo-4H,7H-[1,2,4]triazolo[1,5- |
| | a]pyrimidin-4-yl]acetamide |
| I-4 | N-[2-chloro-4-(trifluoromethyl)phenyl]-2-[2-(3,6-dihydro-2H-pyran-4-yl)-5-ethyl-6- |
| | [4-(2-hydroxy-6-methoxybenzoyl)piperazin-1-yl]-7-oxo-4H,7H-[1,2,4]triazolo[1,5- |
| | a]pyrimidin-4-yl]acetamide |
| I-5 | N-[2-chloro-4-(trifluoromethyl)phenyl]-2-[2-(3,6-dihydro-2H-pyran-4-yl)-5-ethyl-6- |
| | [4-(3-hydroxy-2-methylpyridine-4-carbonyl)piperazin-1-yl]-7-oxo-4H,7H- |
| | [1,2,4]triazolo[1,5-a]pyrimidin-4-yl]acetamide |
| I-6 | N-[2-chloro-4-(trifluoromethyl)phenyl]-2-[2-(3,6-dihydro-2H-pyran-4-yl)-5-ethyl-6- |
| | [4-(3-hydroxythiophene-2-carbonyl)piperazin-1-yl]-7-oxo-4H,7H-[1,2,4]triazolo[1,5- |
| | a]pyrimidin-4-yl]acetamide |
| I-7 | N-[2-chloro-4-(trifluoromethyl)phenyl]-2-[2-(3,6-dihydro-2H-pyran-4-yl)-5-ethyl-6- |
| | {4-[3-hydroxy-5-(trifluoromethyl)thiophene-2-carbonyl]piperazin-1-yl}-7-oxo- |
| | 4H,7H-[1,2,4]triazolo[1,5-a]pyrimidin-4-yl]acetamide |
| I-8 | N-[2-chloro-4-(trifluoromethyl)phenyl]-2-[2-(3,6-dihydro-2H-pyran-4-yl)-5-ethyl-6- |
| | [5-(5-hydroxy-6-methylpyrimidine-4-carbonyl)-octahydropyrrolo[3,4-c]pyrrol-2-yl]- |
| | 7-oxo-4H,7H-[1,2,4]triazolo[1,5-a]pyrimidin-4-yl]acetamide |
| I-9 | N-[2-chloro-4-(trifluoromethyl)phenyl]-2-[2-(3,6-dihydro-2H-pyran-4-yl)-5-ethyl-6- |
| | [4-(3-fluoro-5-hydroxypyridine-4-carbonyl)piperazin-1-yl]-7-oxo-4H,7H- |
| | [1,2,4]triazolo[1,5-a]pyrimidin-4-yl]acetamide |
| I-10 | N-[2-chloro-4-(trifluoromethyl)phenyl]-2-{6-[4-(2,6-difluorobenzoyl)piperazin-1-yl]- |
| | 2-(3,6-dihydro-2H-pyran-4-yl)-5-ethyl-7-oxo-4H,7H-[1,2,4]triazolo[1,5-a]pyrimidin- |
| | 4-yl}acetamide |
| I-11 | N-[2-chloro-4-(trifluoromethyl)phenyl]-2-[2-(3,6-dihydro-2H-pyran-4-yl)-5-ethyl-6- |
| | [4-(3-hydroxy-1-methyl-1H-pyrazole-4-carbonyl)piperazin-1-yl]-7-oxo-4H,7H- |
| | [1,2,4]triazolo[1,5-a]pyrimidin-4-yl]acetamide |
| I-12 | N-[2-chloro-4-(trifluoromethyl)phenyl]-2-[2-(3,6-dihydro-2H-pyran-4-yl)-5-ethyl-6- |
| | [4-(4-hydroxy-1-methyl-1H-pyrazole-3-carbonyl)piperazin-1-yl]-7-oxo-4H,7H- |
| | [1,2,4]triazolo[1,5-a]pyrimidin-4-yl]acetamide |
| I-13 | N-[2-chloro-4-(trifluoromethyl)phenyl]-2-[2-(3,6-dihydro-2H-pyran-4-yl)-5-ethyl-6- |
| | [4-(3-hydroxy-2,5-dimethylpyridine-4-carbonyl)piperazin-1-yl]-7-oxo-4H,7H- |
| | [1,2,4]triazolo[1,5-a]pyrimidin-4-yl]acetamide |
| I-14 | N-[2-chloro-4-(trifluoromethyl)phenyl]-2-[2-(3,6-dihydro-2H-pyran-4-yl)-5-ethyl-6- |
| | [4-(1H-indazole-7-carbonyl)piperazin-1-yl]-7-oxo-4H,7H-[1,2,4]triazolo[1,5- |
| | a]pyrimidin-4-yl]acetamide |
| I-15 | N-[2-chloro-4-(trifluoromethyl)phenyl]-2-[2-(3,6-dihydro-2H-pyran-4-yl)-5-ethyl-7- |
| | oxo-6-(4-{1H-pyrazolo[3,4-c]pyridine-7-carbonyl} piperazin-1-yl)-4H,7H- |
| | [1,2,4]triazolo[1,5-a]pyrimidin-4-yl]acetamide |
| I-16 | 2-[6-(4-acetylpiperazin-1-yl)-2-(3,6-dihydro-2H-pyran-4-yl)-5-ethyl-7-oxo-4H,7H- |
| | [1,2,4]triazolo[1,5-a]pyrimidin-4-yl]-N-[2-chloro-4- |
| | (trifluoromethyl)phenyl]acetamide |
| I-17 | N-[2-chloro-4-(trifluoromethyl)phenyl]-2-[2-(3,6-dihydro-2H-pyran-4-yl)-5-ethyl-7- |
| | oxo-6-[4-(1H-1,2,3,4-tetrazole-5-carbonyl)piperazin-1-yl]-4H,7H-[1,2,4]triazolo[1,5- |
| | a]pyrimidin-4-yl]acetamide |
| I-18 | N-[2-chloro-4-(trifluoromethyl)phenyl]-2-[2-(3,6-dihydro-2H-pyran-4-yl)-5-ethyl-6- |
| | [4-(2-hydroxy-2-methylpropanoyl)piperazin-1-yl]-7-oxo-4H,7H-[1,2,4]triazolo[1,5- |
| | a]pyrimidin-4-yl]acetamide |
| I-19 | N-[2-chloro-4-(trifluoromethyl)phenyl]-2-[2-(3,6-dihydro-2H-pyran-4-yl)-5-ethyl-7- |
| | oxo-6-[4-(quinoline-8-carbonyl)piperazin-1-yl]-4H,7H-[1,2,4]triazolo[1,5- |
| | a]pyrimidin-4-yl]acetamide |
| I-20 | N-[2-chloro-4-(trifluoromethyl)phenyl]-2-[2-(3,6-dihydro-2H-pyran-4-yl)-5-ethyl-6- |
| | [4-(3-hydroxy-2,2-dimethylpropanoyl)piperazin-1-yl]-7-oxo-4H,7H- |
| | [1,2,4]triazolo[1,5-a]pyrimidin-4-yl]acetamide |
| I-21 | N-[2-chloro-4-(trifluoromethyl)phenyl]-2-{5-ethyl-6-[4-(5-hydroxy-6- |
| | methylpyrimidine-4-carbonyl)piperazin-1-yl]-2-(2-methoxypyridin-4-yl)-7-oxo- |
| | 4H,7H-[1,2,4]triazolo[1,5-a]pyrimidin-4-yl}acetamide |
| I-22 | N-[2-chloro-4-(trifluoromethyl)phenyl]-2-{5-ethyl-6-[4-(5-hydroxy-6- |
| | methylpyrimidine-4-carbonyl)piperazin-1-yl]-2-(6-methoxypyridin-3-yl)-7-oxo- |
| | 4H,7H-[1,2,4]triazolo[1,5-a]pyrimidin-4-yl}acetamide |
| I-23 | N-[2-chloro-4-(trifluoromethyl)phenyl]-2-[2-(3,6-dihydro-2H-pyran-4-yl)-5-ethyl-6- |
| | [4-(1-methyl-1H-pyrazole-4-carbonyl)piperazin-1-yl]-7-oxo-4H,7H- |
| | [1,2,4]triazolo[1,5-a]pyrimidin-4-yl]acetamide |
| I-24 | N-[2-chloro-4-(trifluoromethyl)phenyl]-2-[2-(3,6-dihydro-2H-pyran-4-yl)-5-ethyl-6- |
| | [4-(3-hydroxypropanoyl)piperazin-1-yl]-7-oxo-4H,7H-[1,2,4]triazolo[1,5- |
| | a]pyrimidin-4-yl]acetamide |
| I-25 | N-[2-chloro-4-(trifluoromethyl)phenyl]-2-{6-[4-(2-cyanoacetyl)piperazin-1-yl]-2- |
| | (3,6-dihydro-2H-pyran-4-yl)-5-ethyl-7-oxo-4H,7H-[1,2,4]triazolo[1,5-a]pyrimidin-4- |
| | yl}acetamide |
| I-26 | N-[2-chloro-4-(trifluoromethyl)phenyl]-2-[2-(3,6-dihydro-2H-pyran-4-yl)-5-ethyl-6- |
| | [4-(5-hydroxy-1-methyl-1H-pyrazole-4-carbonyl)piperazin-1-yl]-7-oxo-4H,7H- |
| | [1,2,4]triazolo[1,5-a]pyrimidin-4-yl]acetamide |
| I-27 | N-[2-chloro-4-(trifluoromethyl)phenyl]-2-[2-(3,6-dihydro-2H-pyran-4-yl)-5-ethyl-6- |
| | [4-(4-hydroxy-1,2,5-thiadiazole-3-carbonyl)piperazin-1-yl]-7-oxo-4H,7H- |
| | [1,2,4]triazolo[1,5-a]pyrimidin-4-yl]acetamide |
| I-28 | N-[2-chloro-4-(trifluoromethyl)phenyl]-2-[2-(3,6-dihydro-2H-pyran-4-yl)-5-ethyl-6- |
| | [4-(3-hydroxy-5-methyl-1,2-oxazole-4-carbonyl)piperazin-1-yl]-7-oxo-4H,7H- |
| | [1,2,4]triazolo[1,5-a]pyrimidin-4-yl]acetamide |
| I-29 | N-[2-chloro-4-(trifluoromethyl)phenyl]-2-[2-(3,6-dihydro-2H-pyran-4-yl)-5-ethyl-6- |
| | [4-(4-hydroxy-1,2-oxazole-3-carbonyl)piperazin-1-yl]-7-oxo-4H,7H- |
| | [1,2,4]triazolo[1,5-a]pyrimidin-4-yl]acetamide |
| I-30 | N-[2-chloro-4-(trifluoromethyl)phenyl]-2-[2-(3,6-dihydro-2H-pyran-4-yl)-5-ethyl-6- |
| | [4-(1-methyl-1H-pyrazole-3-carbonyl)piperazin-1-yl]-7-oxo-4H,7H- |
| | [1,2,4]triazolo[1,5-a]pyrimidin-4-yl]acetamide |
| I-31 | N-[2-chloro-4-(trifluoromethyl)phenyl]-2-{6-[4-(2-cyano-2,2- |
| | dimethylacetyl)piperazin-1-yl]-2-(3,6-dihydro-2H-pyran-4-yl)-5-ethyl-7-oxo-4H,7H- |
| | [1,2,4]triazolo[1,5-a]pyrimidin-4-yl}acetamide |
| I-32 | N-[2-chloro-4-(trifluoromethyl)phenyl]-2-[2-(1-cyclopropyl-1H-pyrazol-4-yl)-5-ethyl- |
| | 6-[4-(5-hydroxy-6-methylpyrimidine-4-carbonyl)piperazin-1-yl]-7-oxo-4H,7H- |
| | [1,2,4]triazolo[1,5-a]pyrimidin-4-yl]acetamide |
| I-33 | N-[2-chloro-4-(trifluoromethyl)phenyl]-2-[2-(1-cyclopropyl-1H-pyrazol-3-yl)-5-ethyl- |
| | 6-[4-(5-hydroxy-6-methylpyrimidine-4-carbonyl)piperazin-1-yl]-7-oxo-4H,7H- |
| | [1,2,4]triazolo[1,5-a]pyrimidin-4-yl]acetamide |
| I-34 | N-[2-chloro-4-(trifluoromethyl)phenyl]-2-[2-(3,6-dihydro-2H-pyran-4-yl)-5-ethyl-6- |
| | [4-(3-hydroxy-2-methoxypyridine-4-carbonyl)piperazin-1-yl]-7-oxo-4H,7H- |
| | [1,2,4]triazolo[1,5-a]pyrimidin-4-yl]acetamide |
| I-35 | N-[2-chloro-4-(trifluoromethyl)phenyl]-2-{5-ethyl-6-[4-(4-hydroxy-1-methyl-1H- |
| | pyrazole-3-carbonyl)piperazin-1-yl]-2-(2-methoxypyridin-4-yl)-7-oxo-4H,7H- |
| | [1,2,4]triazolo[1,5-a]pyrimidin-4-yl}acetamide |
| I-36 | N-[2-chloro-4-(trifluoromethyl)phenyl]-2-{2-cyclopropyl-5-ethyl-6-[4-(4-hydroxy-1- |
| | methyl-1H-pyrazole-3-carbonyl)piperazin-1-yl]-7-oxo-4H,7H-[1,2,4]triazolo[1,5- |
| | a]pyrimidin-4-yl}acetamide |
| I-37 | N-[2-chloro-4-(trifluoromethyl)phenyl]-2-[2-(3,6-dihydro-2H-pyran-4-yl)-5-ethyl-6- |
| | [4-(1,5-naphthyridine-4-carbonyl)piperazin-1-yl]-7-oxo-4H,7H-[1,2,4]triazolo[1,5- |
| | a]pyrimidin-4-yl]acetamide |
| I-38 | N-[2-chloro-4-(trifluoromethyl)phenyl]-2-[2-(3,6-dihydro-2H-pyran-4-yl)-5-ethyl-7- |
| | oxo-6-[4-(2H-1,2,3-triazole-4-carbonyl)piperazin-1-yl]-4H,7H-[1,2,4]triazolo[1,5- |
| | a]pyrimidin-4-yl]acetamide |
| I-39 | N-[2-chloro-4-(trifluoromethyl)phenyl]-2-[2-(3,6-dihydro-2H-pyran-4-yl)-5-ethyl-7- |
| | oxo-6-{4-[(1s,3s)-3-hydroxycyclobutanecarbonyl]piperazin-1-yl}-4H,7H- |
| | [1,2,4]triazolo[1,5-a]pyrimidin-4-yl]acetamide |
| I-40 | N-[2-chloro-4-(trifluoromethyl)phenyl]-2-[2-(3,6-dihydro-2H-pyran-4-yl)-5-ethyl-6- |
| | [4-(3-hydroxy-3-methylbutanoyl)piperazin-1-yl]-7-oxo-4H,7H-[1,2,4]triazolo[1,5- |
| | a]pyrimidin-4-yl]acetamide |
| I-41 | N-[2-chloro-4-(trifluoromethyl)phenyl]-2-[2-(3,6-dihydro-2H-pyran-4-yl)-5-ethyl-6- |
| | [4-(5-methyl-1,3,4-thiadiazole-2-carbonyl)piperazin-1-yl]-7-oxo-4H,7H- |
| | [1,2,4]triazolo[1,5-a]pyrimidin-4-yl]acetamide |
| I-42 | N-[2-chloro-4-(trifluoromethyl)phenyl]-2-{5-ethyl-6-[4-(5-hydroxy-6- |
| | methylpyrimidine-4-carbonyl)piperazin-1-yl]-2-(6-methoxy-5-methylpyridin-3-yl)-7- |
| | oxo-4H,7H-[1,2,4]triazolo[1,5-a]pyrimidin-4-yl}acetamide |
| I-43 | N-[2-chloro-4-(trifluoromethyl)phenyl]-2-{5-ethyl-6-[4-(5-hydroxy-6- |
| | methylpyrimidine-4-carbonyl)piperazin-1-yl]-2-(2-methoxy-6-methylpyridin-4-yl)-7- |
| | oxo-4H,7H-[1,2,4]triazolo[1,5-a]pyrimidin-4-yl}acetamide |
| I-44 | N-[2-chloro-4-(trifluoromethyl)phenyl]-2-{2-[2-(difluoromethoxy)pyridin-4-yl]-5- |
| | ethyl-6-[4-(5-hydroxy-6-methylpyrimidine-4-carbonyl)piperazin-1-yl]-7-oxo-4H,7H- |
| | [1,2,4]triazolo[1,5-a]pyrimidin-4-yl}acetamide |
| I-45 | N-[2-chloro-4-(trifluoromethyl)phenyl]-2-[5-ethyl-2-(3-fluoro-2-methoxypyridin-4- |
| | yl)-6-[4-(5-hydroxy-6-methylpyrimidine-4-carbonyl)piperazin-1-yl]-7-oxo-4H,7H- |
| | [1,2,4]triazolo[1,5-a]pyrimidin-4-yl]acetamide |
| I-46 | 2-[6-(4-acetylpiperazin-1-yl)-5-ethyl-2-(2-methoxypyridin-4-yl)-7-oxo-4H,7H- |
| | [1,2,4]triazolo[1,5-a]pyrimidin-4-yl]-N-[2-chloro-4- |
| | (trifluoromethyl)phenyl]acetamide |
| I-47 | N-[2-chloro-4-(trifluoromethyl)phenyl]-2-[5-ethyl-2-(2-methoxypyridin-4-yl)-7-oxo- |
| | 6-[4-(2,2,2-trifluoroacetyl)piperazin-1-yl]-4H,7H-[1,2,4]triazolo[1,5-a]pyrimidin-4- |
| | yl]acetamide |
| I-48 | N-[2-chloro-4-(trifluoromethyl)phenyl]-2-[5-ethyl-2-(2-methoxypyridin-4-yl)-6-[4-(1- |
| | methyl-1H-pyrazole-4-carbonyl)piperazin-1-yl]-7-oxo-4H,7H-[1,2,4]triazolo[1,5- |
| | a]pyrimidin-4-yl]acetamide |
| I-49 | N-[2-chloro-4-(trifluoromethyl)phenyl]-2-{6-[4-(1,5-dimethyl-1H-pyrazole-4- |
| | carbonyl)piperazin-1-yl]-5-ethyl-2-(2-methoxypyridin-4-yl)-7-oxo-4H,7H- |
| | [1,2,4]triazolo[1,5-a]pyrimidin-4-yl}acetamide |
| I-50 | N-[2-chloro-4-(trifluoromethyl)phenyl]-2-[5-ethyl-2-(2-methoxypyridin-4-yl)-7-oxo- |
| | 6-[4-(1,3,5-trimethyl-1H-pyrazole-4-carbonyl)piperazin-1-yl]-4H,7H- |
| | [1,2,4]triazolo[1,5-a]pyrimidin-4-yl]acetamide |
| I-51 | N-[2-chloro-4-(trifluoromethyl)phenyl]-2-[5-ethyl-2-(2-methoxypyrimidin-5-yl)-6-[4- |
| | (1-methyl-1H-pyrazole-4-carbonyl)piperazin-1-yl]-7-oxo-4H,7H-[1,2,4]triazolo[1,5- |
| | a]pyrimidin-4-yl]acetamide |
| I-52 | N-[2-chloro-4-(trifluoromethyl)phenyl]-2-[2-(2-cyclopropoxypyridin-4-yl)-5-ethyl-6- |
| | [4-(1-methyl-1H-pyrazole-4-carbonyl)piperazin-1-yl]-7-oxo-4H,7H- |
| | [1,2,4]triazolo[1,5-a]pyrimidin-4-yl]acetamide |
| I-53 | N-[2-chloro-4-(trifluoromethyl)phenyl]-2-{6-[4-(2,2-difluoroacetyl)piperazin-1-yl]-5- |
| | ethyl-2-(2-methoxypyridin-4-yl)-7-oxo-4H,7H-[1,2,4]triazolo[1,5-a]pyrimidin-4- |
| | yl}acetamide |
| I-54 | N-[2-chloro-4-(trifluoromethyl)phenyl]-2-[5-ethyl-2-(2-methoxypyridin-3-yl)-6-[4-(1- |
| | methyl-1H-pyrazole-4-carbonyl)piperazin-1-yl]-7-oxo-4H,7H-[1,2,4]triazolo[1,5- |
| | a]pyrimidin-4-yl]acetamide |
| I-55 | 2-[6-(4-acetylpiperazin-1-yl)-5-ethyl-2-[1-(2-hydroxy-2-methylpropyl)-1H-pyrazol-4- |
| | yl]-7-oxo-4H,7H-[1,2,4]triazolo[1,5-a]pyrimidin-4-yl]-N-[2-chloro-4- |
| | (trifluoromethyl)phenyl]acetamide |
| I-56 | N-[2-chloro-4-(trifluoromethyl)phenyl]-2-{5-ethyl-2-[1-(2-hydroxy-2-methylpropyl)- |
| | 1H-pyrazol-4-yl]-6-[4-(1-methyl-1H-pyrazole-4-carbonyl)piperazin-1-yl]-7-oxo- |
| | 4H,7H-[1,2,4]triazolo[1,5-a]pyrimidin-4-yl}acetamide |
| I-57 | N-[2-chloro-4-(trifluoromethyl)phenyl]-2-[5-ethyl-2-(2-methoxypyridin-4-yl)-6-[4-(4- |
| | methyl-1,3-thiazole-2-carbonyl)piperazin-1-yl]-7-oxo-4H,7H-[1,2,4]triazolo[1,5- |
| | a]pyrimidin-4-yl]acetamide |
| I-58 | N-[2-chloro-4-(trifluoromethyl)phenyl]-2-[5-ethyl-2-(2-methoxypyridin-4-yl)-6-[4-(5- |
| | methyl-1,3-thiazole-2-carbonyl)piperazin-1-yl]-7-oxo-4H,7H-[1,2,4]triazolo[1,5- |
| | a]pyrimidin-4-yl]acetamide |
| I-59 | N-[2-chloro-4-(trifluoromethyl)phenyl]-2-[5-ethyl-2-(2-methoxypyridin-4-yl)-6-[4-(2- |
| | methyl-1,3-thiazole-5-carbonyl)piperazin-1-yl]-7-oxo-4H,7H-[1,2,4]triazolo[1,5- |
| | a]pyrimidin-4-yl]acetamide |
| I-60 | N-[2-chloro-4-(trifluoromethyl)phenyl]-2-{6-[4-(1-cyclopropyl-1H-pyrazole-4- |
| | carbonyl)piperazin-1-yl]-5-ethyl-2-(2-methoxypyridin-4-yl)-7-oxo-4H,7H- |
| | [1,2,4]triazolo[1,5-a]pyrimidin-4-yl}acetamide |
| I-61 | N-[2-chloro-4-(trifluoromethyl)phenyl]-2-(6-{4-[1-(difluoromethyl)-1H-pyrazole-4- |
| | carbonyl]piperazin-1-yl}-5-ethyl-2-(2-methoxypyridin-4-yl)-7-oxo-4H,7H- |
| | [1,2,4]triazolo[1,5-a]pyrimidin-4-yl)acetamide |
| I-62 | N-[2-chloro-4-(trifluoromethyl)phenyl]-2-{5-ethyl-6-[4-(5-methoxy-1,3-dimethyl-1H- |
| | pyrazole-4-carbonyl)piperazin-1-yl]-2-(2-methoxypyridin-4-yl)-7-oxo-4H,7H- |
| | [1,2,4]triazolo[1,5-a]pyrimidin-4-yl}acetamide |
| I-63 | N-[2-chloro-4-(trifluoromethyl)phenyl]-2-[5-ethyl-2-(2-methoxypyridin-4-yl)-6-[4-(1- |
| | methyl-1H-1,2,3-triazole-5-carbonyl)piperazin-1-yl]-7-oxo-4H,7H-[1,2,4]triazolo[1,5- |
| | a]pyrimidin-4-yl]acetamide |
| I-64 | N-[2-chloro-4-(trifluoromethyl)phenyl]-2-[5-ethyl-2-(2-methoxypyrimidin-4-yl)-6-[4- |
| | (1-methyl-1H-pyrazole-4-carbonyl)piperazin-1-yl]-7-oxo-4H,7H-[1,2,4]triazolo[1,5- |
| | a]pyrimidin-4-yl]acetamide |
| I-65 | N-[2-chloro-4-(trifluoromethyl)phenyl]-2-[5-ethyl-2-(2-methoxypyridin-4-yl)-6-[4-(3- |
| | methyl-1,2-oxazole-5-carbonyl)piperazin-1-yl]-7-oxo-4H,7H-[1,2,4]triazolo[1,5- |
| | a]pyrimidin-4-yl]acetamide |
| I-66 | N-[2-chloro-4-(trifluoromethyl)phenyl]-2-[5-ethyl-2-(2-methoxypyridin-4-yl)-6-[4-(1- |
| | methyl-1H-pyrazole-5-carbonyl)piperazin-1-yl]-7-oxo-4H,7H-[1,2,4]triazolo[1,5- |
| | a]pyrimidin-4-yl]acetamide |
| I-67 | N-[2-chloro-4-(trifluoromethyl)phenyl]-2-[5-ethyl-2-(2-methoxypyridin-4-yl)-6-[4-(2- |
| | methyl-1,3-oxazole-5-carbonyl)piperazin-1-yl]-7-oxo-4H,7H-[1,2,4]triazolo[1,5- |
| | a]pyrimidin-4-yl]acetamide |
| I-68 | N-[2-chloro-4-(trifluoromethyl)phenyl]-2-[2-(3,6-dihydro-2H-pyran-4-yl)-5-ethyl-6- |
| | [4-(1-methyl-1H-pyrazole-4-carbonyl)-octahydropyrrolo[3,2-b]pyrrol-1-yl]-7-oxo- |
| | 4H,7H-[1,2,4]triazolo[1,5-a]pyrimidin-4-yl]acetamide |
| I-69 | N-[2-chloro-4-(trifluoromethyl)phenyl]-2-[5-ethyl-2-(4-methoxypyridin-2-yl)-6-[4-(1- |
| | methyl-1H-pyrazole-4-carbonyl)piperazin-1-yl]-7-oxo-4H,7H-[1,2,4]triazolo[1,5- |
| | a]pyrimidin-4-yl]acetamide |
| I-70 | N-[2-chloro-4-(trifluoromethyl)phenyl]-2-[5-ethyl-2-(4-methoxypyrimidin-2-yl)-6-[4- |
| | (1-methyl-1H-pyrazole-4-carbonyl)piperazin-1-yl]-7-oxo-4H,7H-[1,2,4]triazolo[1,5- |
| | a]pyrimidin-4-yl]acetamide |
| I-71 | N-[2-chloro-4-(trifluoromethyl)phenyl]-2-[5-ethyl-2-(6-methoxypyrimidin-4-yl)-6-[4- |
| | (1-methyl-1H-pyrazole-4-carbonyl)piperazin-1-yl]-7-oxo-4H,7H-[1,2,4]triazolo[1,5- |
| | a]pyrimidin-4-yl]acetamide |
| I-72 | N-[2-chloro-4-(trifluoromethyl)phenyl]-2-[5-ethyl-2-(5-fluoro-2-methoxypyridin-4- |
| | yl)-6-[4-(1-methyl-1H-pyrazole-4-carbonyl)piperazin-1-yl]-7-oxo-4H,7H- |
| | [1,2,4]triazolo[1,5-a]pyrimidin-4-yl]acetamide |
| I-73 | N-[2-chloro-4-(trifluoromethyl)phenyl]-2-[5-ethyl-2-(2-methoxypyridin-4-yl)-6-[4-(1- |
| | methyl-1H-1,2,4-triazole-3-carbonyl)piperazin-1-yl]-7-oxo-4H,7H-[1,2,4]triazolo[1,5- |
| | a]pyrimidin-4-yl]acetamide |
| I-74 | N-[2-chloro-4-(trifluoromethyl)phenyl]-2-[5-ethyl-2-(2-methoxypyridin-4-yl)-6-[4-(2- |
| | methyl-2H-1,2,3,4-tetrazole-5-carbonyl)piperazin-1-yl]-7-oxo-4H,7H- |
| | [1,2,4]triazolo[1,5-a]pyrimidin-4-yl]acetamide |
| I-75 | N-[2-chloro-4-(trifluoromethyl)phenyl]-2-[5-ethyl-2-(2-methoxypyridin-4-yl)-6-[4-(2- |
| | methyl-1,3-oxazole-4-carbonyl)piperazin-1-yl]-7-oxo-4H,7H-[1,2,4]triazolo[1,5- |
| | a]pyrimidin-4-yl]acetamide |
| I-76 | N-[2-chloro-4-(trifluoromethyl)phenyl]-2-[5-ethyl-2-(2-methoxypyridin-4-yl)-6-[4-(3- |
| | methyl-1,2-thiazole-5-carbonyl)piperazin-1-yl]-7-oxo-4H,7H-[1,2,4]triazolo[1,5- |
| | a]pyrimidin-4-yl]acetamide |
| I-77 | N-[2-chloro-4-(trifluoromethyl)phenyl]-2-[5-ethyl-2-(2-methoxypyridin-4-yl)-6- |
| | [(3R)-3-methyl-4-(1-methyl-1H-pyrazole-4-carbonyl)piperazin-1-yl]-7-oxo-4H,7H- |
| | [1,2,4]triazolo[1,5-a]pyrimidin-4-yl]acetamide |
| I-78 | N-[2-chloro-4-(trifluoromethyl)phenyl]-2-[5-ethyl-2-(2-methoxypyridin-4-yl)-6-[4-(5- |
| | methyl-1,2,4-oxadiazole-3-carbonyl)piperazin-1-yl]-7-oxo-4H,7H-[1,2,4]triazolo[1,5- |
| | a]pyrimidin-4-yl]acetamide |
| I-79 | N-[2-chloro-4-(trifluoromethyl)phenyl]-2-[5-ethyl-2-(2-methoxypyridin-4-yl)-6-[4-(1- |
| | methyl-1H-1,2,4-triazole-5-carbonyl)piperazin-1-yl]-7-oxo-4H,7H-[1,2,4]triazolo[1,5- |
| | a]pyrimidin-4-yl]acetamide |
| I-80 | N-[2-chloro-4-(trifluoromethyl)phenyl]-2-{5-ethyl-6-[4-(3-hydroxy-2- |
| | methoxypyridine-4-carbonyl)piperazin-1-yl]-2-(2-methoxypyridin-4-yl)-7-oxo- |
| | 4H,7H-[1,2,4]triazolo[1,5-a]pyrimidin-4-yl}acetamide |
| I-81 | N-[2-chloro-4-(trifluoromethyl)phenyl]-2-{6-[1-(5-hydroxy-6-methylpyrimidine-4- |
| | carbonyl)piperidin-4-yl]-2-(2-methoxypyridin-4-yl)-7-oxo-4H,7H-[1,2,4]triazolo[1,5- |
| | a]pyrimidin-4-yl}acetamide |
| I-82 | N-[2-chloro-4-(pentafluoro-lambda6-sulfanyl)phenyl]-2-[5-ethyl-2-(2- |
| | methoxypyridin-4-yl)-6-[4-(1-methyl-1H-pyrazole-4-carbonyl)piperazin-1-yl]-7-oxo- |
| | 4H,7H-[1,2,4]triazolo[1,5-a]pyrimidin-4-yl]acetamide |
| I-83 | N-[2-chloro-4-(trifluoromethyl)phenyl]-2-[2-(3,6-dihydro-2H-pyran-4-yl)-5-ethyl-6- |
| | (4-{7-hydroxy-2H,3H-furo[3,2-c]pyridine-6-carbonyl}piperazin-1-yl)-7-oxo-4H,7H- |
| | [1,2,4]triazolo[1,5-a]pyrimidin-4-yl]acetamide |
| I-84 | N-[2-chloro-4-(trifluoromethyl)phenyl]-2-[2-(3,6-dihydro-2H-pyran-4-yl)-5-ethyl-6- |
| | (4-{4-hydroxy-2H,3H-furo[2,3-c]pyridine-5-carbonyl}piperazin-1-yl)-7-oxo-4H,7H- |
| | [1,2,4]triazolo[1,5-a]pyrimidin-4-yl]acetamide |
| I-85 | N-[2-chloro-4-(trifluoromethyl)phenyl]-2-[5-ethyl-6-(4-{4-hydroxy-2H,3H-furo[2,3- |
| | c]pyridine-5-carbonyl}piperazin-1-yl)-2-(2-methoxypyridin-4-yl)-7-oxo-4H,7H- |
| | [1,2,4]triazolo[1,5-a]pyrimidin-4-yl]acetamide |
| I-86 | N-[2-chloro-4-(trifluoromethyl)phenyl]-2-[5-ethyl-6-(4-{7-hydroxy-2H,3H-furo[3,2- |
| | c]pyridine-6-carbonyl}piperazin-1-yl)-2-(2-methoxypyridin-4-yl)-7-oxo-4H,7H- |
| | [1,2,4]triazolo[1,5-a]pyrimidin-4-yl]acetamide |
| I-87 | 2-[6-(4-acetylpiperazin-1-yl)-5-ethyl-2-[2-(2-hydroxypropan-2-yl)-1,3-thiazol-5-yl]-7- |
| | oxo-4H,7H-[1,2,4]triazolo[1,5-a]pyrimidin-4-yl]-N-[2-chloro-4- |
| | (trifluoromethyl)phenyl]acetamide |
| I-88 | N-[2-chloro-4-(trifluoromethyl)phenyl]-2-{5-ethyl-6-[4-(4-hydroxy-1,2-oxazole-3- |
| | carbonyl)piperazin-1-yl]-2-(2-methoxypyridin-4-yl)-7-oxo-4H,7H-[1,2,4]triazolo[1,5- |
| | a]pyrimidin-4-yl}acetamide |

### 4. Pharmaceutical Compositions, Methods of Treatment and Uses of Compounds

In another aspect, the present invention provides a pharmaceutical composition comprising a compound of the present invention, or a pharmaceutically acceptable salt thereof, and a pharmaceutically acceptable carrier. In a further embodiment, the composition comprises at least two pharmaceutically acceptable carriers, such as those described herein. The pharmaceutical composition can be formulated for particular routes of administration such as oral administration, parenteral administration (e.g. by injection, infusion, transdermal or topical administration), and rectal administration, in particular oral administration. Topical administration may also pertain to inhalation or intranasal application. The pharmaceutical compositions of the present invention can be made up in a solid form (including, without limitation, capsules, tablets, pills, granules, powders or suppositories), or in a liquid form (including, without limitation, solutions, suspensions or emulsions). Tablets may be either film coated or enteric coated according to methods known in the art. Typically, the pharmaceutical compositions are tablets or gelatin capsules comprising the active ingredient together with one or more of:
a) diluents, e.g., lactose, dextrose, sucrose, mannitol, sorbitol, cellulose and/or glycine;
b) lubricants, e.g., silica, talcum, stearic acid, its magnesium or calcium salt and/or polyethylene glycol; for tablets also
c) binders, e.g., magnesium aluminum silicate, starch paste, gelatin, tragacanth, methylcellulose, sodium carboxymethylcellulose and/or polyvinylpyrrolidone; if desired
d) disintegrants, e.g., starches, agar, alginic acid or its sodium salt, or effervescent mixtures; and
e) absorbents, colorants, flavors and sweeteners.

Typical approaches to solubilize compounds for parenteral administration are the optimization of the pH or the use of co-solvents (e.g. PEG300, PEG400, propylene glycol, or ethanol). If these approaches are, for any reason, not feasible, the use of surfactants may be considered (e.g. Tween® 80 or Cremophor EL®). Cyclodextrins are established as safe solubilizing agents. Compounds with a high solubility in natural oils may be solubilized in parenteral fat emulsions.

There is also provided a pharmaceutical composition comprising a compound of Formula I as described herein, or a pharmaceutically acceptable salt thereof, and one or more pharmaceutically acceptable carriers.

### Uses

The compounds of Formula I of the present invention in free form or in pharmaceutically acceptable salt form, exhibit valuable pharmacological properties, e.g. WRN inhibiting properties, e.g. as indicated in vitro tests as provided in the next sections, and are therefore indicated for therapy, or for use as research chemicals, e.g. as a chemical probe, and as tool compounds.

Also provided is a compound of Formula I, as described herein. Said compound can be used as a research chemical, a compound herein comprising an added biotin moiety, for example a tool compound or chemical probe, in particular for research on WRN. In another embodiment there is provided the use of a compound of Formula I, as described herein, as a research chemical, for example tool compound or chemical probe, in particular for research on WRN.

There is also provided a compound of Formula I as described herein, or a pharmaceutically acceptable salt thereof, for use in the treatment of cancer. Cancers that may be treated by WRN inhibition include cancers that are characterized as microsatellite instability-high (MSI-H) or mismatch repair deficient (dMMR). In particular, a compound of Formula I as described herein, or a pharmaceutically acceptable salt thereof, may be useful in the treatment of a cancer that is characterized as microsatellite instability-high (MSI-H) or mismatch repair deficient (dMMR).

There is also provided a compound of Formula I as described herein, or a pharmaceutically acceptable salt thereof, for use as a medicament. In particular, said use is:
for the treatment of a disease that is treated by WRN inhibition,
for the treatment of cancer,
for the treatment of cancer that is characterized as microsatellite instability-high (MSI-H) or mismatch repair deficient (dMMR),
for the treatment of cancer that is characterized as microsatellite instability-high (MSI-H) or mismatch repair deficient (dMMR), such as colorectal, gastric, prostate, endometrial, adrenocortical, uterine, cervical, esophageal, breast, kidney and ovarian cancer,
for the treatment of cancer that is characterized as microsatellite instability-high (MSI-H) or mismatch repair deficient (dMMR) is selected from colorectal, gastric, prostate and endometrial cancer, or
for the treatment of cancer wherein the cancer characterized as microsatellite instability-high (MSI-H) or mismatch repair deficient (dMMR) is selected from uterine corpus endometrial carcinoma, colon adenocarcinoma, stomach adenocarcinoma, rectal adenocarcinoma, adrenocortical carcinoma, uterine carcinosarcoma, cervical squamous cell carcinoma, endocervical adenocarcinoma, esophageal carcinoma, breast carcinoma, kidney renal clear cell carcinoma, prostate cancer and ovarian serous cystadenocarcinoma.

There is also provided a method of:
modulating WRN activity in a subject, wherein the method comprises administering to the subject a therapeutically effective amount of the compound of Formula I as described herein, or a pharmaceutically acceptable salt thereof,
inhibiting WRN in a subject, wherein the method comprises administering to the subject a therapeutically effective amount of the compound of Formula I as described herein, or a pharmaceutically acceptable salt thereof,
treating a disorder or disease which can be treated by WRN inhibition in a subject, comprising administering to the subject a therapeutically effective amount of the compound of Formula I as described herein, or a pharmaceutically acceptable salt thereof,
treating cancer in a subject, comprising administering to the subject a therapeutically effective amount of the compound of Formula I as described herein, or a pharmaceutically acceptable salt thereof,
treating cancer in a subject, comprising administering a compound of Formula I as described herein, wherein the cancer is characterized as microsatellite instability-high (MSI-H) or mismatch repair deficient (dMMR). In particular, the cancer characterized as microsatellite instability-high (MSI-H) or mismatch repair deficient (dMMR) is selected from colorectal, gastric, prostate, endometrial, adrenocortical, uterine, cervical, esophageal, breast, kidney and ovarian cancer. More particularly, the cancer characterized as microsatellite instability-high (MSI-H) or mismatch repair deficient (dMMR) is selected from colorectal, gastric, prostate and endometrial cancer. Examples include uterine corpus endometrial carcinoma, colon adenocarcinoma, stomach adenocarcinoma, rectal adenocarcinoma, adrenocortical carcinoma, uterine carcinosarcoma, cervical squamous cell carcinoma, endocervical adenocarcinoma, esophageal carcinoma, breast carcinoma, kidney renal clear cell carcinoma, prostate cancer and ovarian serous cystadenocarcinoma.

There is also provided the use of a compound of Formula I as described herein, or a pharmaceutically acceptable salt thereof:
in therapy,
in the manufacture of a medicament,
in the manufacture of a medicament for the treatment of cancer. In particular, said cancer is characterized as microsatellite instability-high (MSI-H) or mismatch repair deficient (dMMR),
in the manufacture of a medicament for treatment of a disease which may be treated by WRN inhibition,
wherein in particular, the cancer is characterized by microsatellite instability-high (MSI-H) or mismatch repair deficient (dMMR), for example colorectal, gastric, prostate, endometrial, adrenocortical, uterine, cervical, esophageal, breast, kidney and ovarian cancer, in particular, colorectal, gastric, prostate or endometrial cancer, or uterine corpus endometrial carcinoma, colon adenocarcinoma, stomach adenocarcinoma, rectal adenocarcinoma, adrenocortical carcinoma, uterine carcinosarcoma, cervical squamous cell carcinoma, endocervical adenocarcinoma, esophageal carcinoma, breast carcinoma, kidney renal clear cell carcinoma and ovarian serous cystadenocarcinoma.

In some embodiments, the subject has or is identified as having a microsatellite instable (MSI-H) cancer, e.g., in reference to a control, e.g., a normal, subject. In one embodiment, the subject has MSI-H advanced solid tumors, a colorectal cancer (CRC), endometrial, uterine, stomach or other MSI-H cancer. In some embodiments, the subject has a colorectal (CRC), endometrial or stomach cancer, which cancer has or is identified as having a microsatellite instability (MSI-H), e.g., in reference to a control, e.g., a normal, subject. Such identification techniques are known in the art.

### Forms

Depending on the choice of the starting materials and procedures, the compounds can be present in the form of one of the possible stereoisomers or as mixtures thereof, for example as pure optical isomers, or as stereoisomer mixtures, such as racemates and diastereoisomer mixtures, depending on the number of asymmetric carbon atoms. The present invention is meant to include all such possible stereoisomers, including racemic mixtures, diasteriomeric mixtures and optically pure forms. Optically active (R)- and (S)-stereoisomers may be prepared using chiral synthons or chiral reagents, or resolved using conventional techniques. If the compound contains a double bond, the substituent may be E or Z configuration. If the compound contains a disubstituted cycloalkyl, the cycloalkyl substituent may have a cis- or trans-configuration. All tautomeric forms are also intended to be included.

Any formula given herein is intended to represent unlabeled forms as well as isotopically labeled forms of the compounds, in addition to the deuteration specifically claimed in Formula I. Isotopically labeled compounds have structures depicted by the formulae given herein except that one or more atoms are replaced by an atom having a selected atomic mass or mass number. Isotopes that can be incorporated into compounds of the invention include, for example, isotopes of hydrogen.

Further, incorporation of certain isotopes, particularly deuterium (i.e., 2H or D) may afford certain therapeutic advantages resulting from greater metabolic stability, for example increased in vivo half-life or reduced dosage requirements or an improvement in therapeutic index or tolerability. It is understood that deuterium in this context is regarded as a substituent of a compound of the present invention. The concentration of deuterium, may be defined by the isotopic enrichment factor. The term “isotopic enrichment factor” as used herein means the ratio between the isotopic abundance and the natural abundance of a specified isotope. If a substituent in a compound of this invention is denoted as being deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). It should be understood that the term “isotopic enrichment factor” can be applied to any isotope in the same manner as described for deuterium.

Other examples of isotopes that can be incorporated into compounds of the invention include isotopes of hydrogen, carbon, nitrogen, oxygen, phosphorous, fluorine, and chlorine, such as 3H, 11C, 13C, 14C, 15N, 18F 31P, 32P, 35S, 36Cl, 123I, 124I, and 125I, respectively. Accordingly it should be understood that the invention includes compounds that incorporate one or more of any of the aforementioned isotopes, including for example, radioactive isotopes, such as 3H and 14C, or those into which non-radioactive isotopes, such as 2H and 13C are present. Such isotopically labelled compounds are useful in metabolic studies (with 14C), reaction kinetic studies (with, for example 2H or 3H), detection or imaging techniques, such as positron emission tomography (PET) or single-photon emission computed tomography (SPECT) including drug or substrate tissue distribution assays, or in radioactive treatment of patients. In particular, an 18F or labeled compound may be particularly desirable for PET or SPECT studies. Isotopically-labeled compounds of the present invention can generally be prepared by conventional techniques known to those skilled in the art or by processes analogous to those described in the accompanying Examples and Preparations using an appropriate isotopically-labeled reagents in place of the non-labeled reagent previously employed.

A “compound of the present invention” or a “compound of Formula I” includes a zwitterion thereof, a non-zwitterion thereof (non-charged form), or a pharmaceutically acceptable salt of said zwitterionic or non-zwitterionic form thereof. “Zwitterion” or “zwitterionic form” means a compound containing both positive and negatively charged functional groups.

The term “cancer” refers to a disease characterized by the rapid and uncontrolled growth of aberrant cells. Cancer cells can spread locally or through the bloodstream and lymphatic system to other parts of the body. Examples of various cancers are described herein and include but are not limited to colorectal, gastric, endometrial, prostate, adrenocortical, uterine, cervical, esophageal, breast, kidney, ovarian cancer and the like.

The terms “tumor” and “cancer” are used interchangeably herein, e.g., both terms encompass solid and liquid, e.g., diffuse or circulating, tumors. As used herein, the term “cancer” or “tumor” includes premalignant, as well as malignant cancers and tumors.

“WRN inhibitor” or “WRN helicase inhibitor” as used herein means a compound that inhibits Werner Syndrome RecQ DNA helicase (WRN). The term “WRN” as used herein refers to the protein of Werner Syndrome RecQ DNA helicase. The term “WRN” includes mutants, fragments, variants, isoforms, and homologs of full-length wild-type WRN. In one embodiment, the protein is encoded by the WRN gene (Entrez gene ID 7486; Ensembl ID ENSG00000165392). Exemplary WRN sequences are available at the Uniprot database under accession number Q14191.

“Disease or condition mediated by WRN” includes a disease or condition, such as cancer, which is treated by WRN inhibition. In particular this can include cancers characterized as microsatellite instability-high (MSI-H) or mismatch repair deficient (dMMR).

“Microsatellite unstable cancer,” “microsatellite instability-high cancer,” “microsatellite high cancer” and “MSI-high cancer,” “MSIhi” and “MSI-H” when used herein, are used interchangeably, and describe cancers that have a high number of alterations in the length of simple repetitive genomic sequences within microsatellites.

The determination of MSI-H or dMMR tumor status for patients can be performed using, e.g., polymerase chain reaction (PCR) tests for MSI-H status or immunohistochemistry (IHC) tests for dMMR. Methods for identification of MSI-H or dMMR tumor status are described, e.g., in Ryan et al. Crit Rev Oncol Hematol. 2017; 116:38-57; Dietmaier and Hofstadter. Lab Invest 2001, 81:1453-1456; and Kawakami et al. Curr Treat Options Oncol. 2015; 16(7): 30).

Microsatellite instability can be found in colorectal cancer, gastric cancer and endometrial cancer in particular, but also in adrenocortical, uterine, cervical, esophageal, breast, kidney, prostate and ovarian cancers. Examples of microsatellite high cancers include uterine corpus endometrial carcinoma, colon adenocarcinoma, stomach adenocarcinoma, rectal adenocarcinoma, adrenocortical carcinoma, uterine carcinosarcoma, cervical squamous cell carcinoma, endocervical adenocarcinoma, esophageal carcinoma, breast carcinoma, kidney renal clear cell carcinoma and ovarian serous cystadenocarcinoma.

A cancer that has “defective mismatch repair” (dMMR) or “dMMR character” includes cancer types associated with documented MLH1, PMS2, MSH2, MSH3, MSH6, MLH3, and PMS1 mutations or epigenetic silencing, microsatellite fragile sites, or other gene inactivation mechanisms, including but not limited to cancers of the lung, breast, kidney, large intestine, ovary, prostate, upper aerodigestive tract, stomach, endometrium, liver, pancreas, haematopoietic and lymphoid tissue, skin, thyroid, pleura, autonomic ganglia, central nervous system, soft tissue, pediatric rhabdoid sarcomas, melanomas and other cancers. A cell or cancer with “defective” mismatch repair has a significantly reduced (e.g., at least about 25%, 30%, 40%, 50%, 60%, 70%, 80% or 90% decrease) amount of mismatch repair. In some cases, a cell or cancer which is defective in mismatch repair will perform no mismatch repair.

As used herein, the term “pharmaceutical composition” refers to a compound of the invention, or a pharmaceutically acceptable salt thereof, together with at least one pharmaceutically acceptable carrier, in a form suitable for oral or parenteral administration.

As used herein, the term “pharmaceutically acceptable carrier” refers to a substance useful in the preparation or use of a pharmaceutical composition and includes, for example, suitable diluents, solvents, dispersion media, surfactants, antioxidants, preservatives, isotonic agents, buffering agents, emulsifiers, absorption delaying agents, salts, drug stabilizers, binders, excipients, disintegration agents, lubricants, wetting agents, sweetening agents, flavoring agents, dyes, and combinations thereof, as would be known to those skilled in the art (see, for example, Remington The Science and Practice of Pharmacy, 22nd Ed. Pharmaceutical Press, 2013, pp. 1049-1070).

The terms “synthetic lethality,” and “synthetically lethal” are used to refer to reduced cell viability and/or a reduced rate of cell proliferation caused by a combination of mutations or approaches to cause loss of function (e.g., RNA interference or protein function inhibition) in two or more genes but not by the loss of function of only one of these genes.

The term “a therapeutically effective amount” of a compound of the present invention refers to an amount of the compound of the present invention that will elicit the biological or medical response of a subject, for example, reduction or inhibition of an enzyme or a protein activity, or ameliorate symptoms, alleviate conditions, slow or delay disease progression, or prevent a disease, etc. In some embodiments, the methods of the invention comprise administration of a therapeutically effective amount of a compound herein.

In one embodiment, the term “a therapeutically effective amount” refers to the amount of the compound of the present invention that, when administered to a subject, is effective to (1) at least partially alleviate, prevent and/or ameliorate a condition, or a disorder or a disease (i) mediated by WRN, or (ii) associated with WRN activity, or (iii) characterized by activity (normal or abnormal) of WRN; or (2) reduce or inhibit the activity of WRN.

In another embodiment, the term “a therapeutically effective amount” refers to the amount of the compound of the present invention that, when administered to a cell, or a tissue, or a non-cellular biological material, or a medium, is effective to at least partially reducing or inhibiting the activity of WRN, or reducing WRN protein levels.

As used herein, the term “subject” refers to primates (e.g., humans, male or female), dogs, rabbits, guinea pigs, pigs, rats and mice. In certain embodiments, the subject is a primate, a rat or a mouse. In yet other embodiments, the subject is a human.

As used herein, the term “inhibit,” “inhibition” or “inhibiting” refers to the reduction or suppression of a given condition, symptom, or disorder, or disease, or a significant decrease in the baseline activity of a biological activity or process.

As used herein, the term “treat,” “treating” or “treatment” of any disease or disorder refers to alleviating or ameliorating the disease or disorder (i.e., slowing or arresting the development of the disease or at least one of the clinical symptoms thereof); or alleviating or ameliorating at least one physical parameter or biomarker associated with the disease or disorder, including those which may not be discernible to the patient.

As used herein, the term “prevent,” “preventing” or “prevention” of any disease or disorder refers to the prophylactic treatment of the disease or disorder; or delaying the onset or progression of the disease or disorder.

As used herein, a subject is “in need of” a treatment if such subject would benefit biologically, medically or in quality of life from such treatment.

As used herein, the term “a,” “an,” “the” and similar terms used in the context of the present invention (especially in the context of the claims) are to be construed to cover both the singular and plural unless otherwise indicated herein or clearly contradicted by the context.

“May join” means joins or does not join.

“May be replaced by deuterium” means is replaced by deuterium, or is not replaced by deuterium.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed.

### Isomeric Forms

Any asymmetric atom (e.g., carbon or the like) of the compound(s) of the present invention can be present in racemic or enantiomerically enriched, for example the (R)-, (S)- or (R, S)-configuration. In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the (R)- or(S)-configuration. Substituents at atoms with unsaturated double bonds may, if possible, be present in cis-(Z)- or trans-(E)-form.

Accordingly, as used herein a compound of the present invention can be in the form of one of the possible stereoisomers, rotamers, atropisomers, tautomers or mixtures thereof, for example, as substantially pure geometric (cis or trans) stereoisomers, diastereomers, optical isomers (antipodes), racemates or mixtures thereof.

Any resulting mixtures of stereoisomers can be separated on the basis of the physicochemical differences of the constituents, into the pure or substantially pure geometric or optical isomers, diastereomers, racemates, for example, by chromatography and/or fractional crystallization.

Any resulting racemates of compounds of the present invention or of intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds of the present invention into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic compounds of the present invention or racemic intermediates can also be resolved by chiral chromatography, e.g., high pressure liquid chromatography (HPLC) using a chiral adsorbent.

Compounds of the invention, i.e. compounds of Formula I that contain groups capable of acting as donors and/or acceptors for hydrogen bonds may be capable of forming co-crystals with suitable co-crystal formers. These co-crystals may be prepared from compounds of Formula I by known co-crystal forming procedures. Such procedures include grinding, heating, co-subliming, co-melting, or contacting in solution compounds of Formula I with the co-crystal former under crystallization conditions and isolating co-crystals thereby formed. Suitable co-crystal formers include those described in WO 2004/078163. Hence the invention further provides co-crystals comprising a compound of Formula I.

Furthermore, the compounds of the present invention, including their salts, can also be obtained in the form of their hydrates, or include other solvents used for their crystallization.

The compounds of the present invention may inherently or by design form solvates with pharmaceutically acceptable solvents (including water); therefore, it is intended that the invention embrace both solvated and unsolvated forms. The term “solvate” refers to a molecular complex of a compound of the present invention (including pharmaceutically acceptable salts thereof) with one or more solvent molecules. Such solvent molecules are those commonly used in the pharmaceutical art, which are known to be innocuous to the recipient, e.g., water, ethanol, and the like. The term “hydrate” refers to the complex where the solvent molecule is water.

### Dosage Forms

The pharmaceutical composition or combination of the present invention may, for example, be in unit dosage of about 1-1000 mg of active ingredient(s) for a subject of about 50-70 kg.

### Combinations

“Combination” refers to either a fixed combination in one dosage unit form, or a combined administration where a compound of Formula I, or a pharmaceutically acceptable salt thereof, and a combination partner (e.g. another drug as explained below, also referred to as “therapeutic agent” or “co-agent”) may be administered independently at the same time or separately within time intervals, especially where these time intervals allow that the combination partners show a cooperative, e.g. synergistic effect. The single components may be packaged in a kit or separately. One or both of the components (e.g., powders or liquids) may be reconstituted or diluted to a desired dose prior to administration. The terms “co-administration” or “combined administration” or the like as utilized herein are meant to encompass administration of the selected combination partner to a single subject in need thereof (e.g. a patient), and are intended to include treatment regimens in which the agents are not necessarily administered by the same route of administration or at the same time. The term “pharmaceutical combination” as used herein means a product that results from the mixing or combining of more than one therapeutic agent and includes both fixed and non-fixed combinations of the therapeutic agents. The term “fixed combination” means that the therapeutic agents, e.g. a compound of the present invention and a combination partner, are both administered to a patient simultaneously in the form of a single entity or dosage.

The term “non-fixed combination” means that the therapeutic agents, e.g. a compound of the present invention and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g. the administration of three or more therapeutic agents.

The combinations described herein can include a compound of Formula I and one or more additional therapeutic agents, e.g., one or more anti-cancer agents, cytotoxic or cytostatic agents, hormone treatment, vaccines, and/or other immunotherapies. In other embodiments, the combination is further administered or used in combination with other therapeutic treatment modalities, including surgery, radiation, cryosurgery, and/or thermotherapy. Such combination therapies may advantageously utilize lower dosages of the administered therapeutic agents, thus avoiding possible toxicities or complications associated with the treatment.

There is also provided a combination comprising a compound of Formula I as described herein, or a pharmaceutically acceptable salt thereof, as described herein, and one or more additional therapeutically active agents. The additional therapeutic agent is, for example, a chemical compound, peptide, antibody, antibody fragment or nucleic acid, which is therapeutically active or enhances the therapeutic activity when administered to a patient in combination with a compound of the present disclosure. In particular, an additional therapeutically active agent is:
an anti-cancer agent,
a chemotherapy,
a chemotherapy selected from anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate,
liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX0), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®), in particular fluorouracil (5-FU) and irinotecan (Camptosar®).
a PD-1 inhibitor,
an anti-PD-1 antibody molecule,
a PD-1 inhibitor selected from spartalizumab (Novartis), nivolumab (Bristol-Myers Squibb), pembrolizumab (Merck & Co), pidilizumab (CureTech), MED10680 (Medimmune), cemiplimab (REGN2810, Regeneron), dostarlimab (TSR-042, Tesaro), PF-06801591 (Pfizer), tislelizumab (BGB-A317, Beigene), BGB-108 (Beigene), INCSHR1210 (Incyte), balstilimab (AGEN2035, Agenus), sintilimab (InnoVent), toripalimab (Shanghai Junshi Bioscience), camrelizumab (Jiangsu Hengrui Medicine Co.), AMP-224 (Amplimmune), penpulimab (Akeso Biopharma Inc), zimberelimab (Arcus Biosciences Inc), and prolgolimab (Biocad Ltd),
spartalizumab, or
tislelizumab (BGB-A317, Beigene).

In a further embodiment, the additional therapeutically active agent is the chemotherapy irinotecan (Camptosar®).

In another embodiment, the additional therapeutically active agent is an inhibitor of PD-1, e.g., human PD-1. In another embodiment, the immunomodulator is an inhibitor of PD-L1, e.g., human PD-L1. In one embodiment, the inhibitor of PD-1 or PD-L1 is an antibody molecule to PD-1 or PD-L1. In another embodiment, the additional therapeutically active agent is an anti-PD-1 antibody molecule.

In a further embodiment, the PD-1 inhibitor is an anti-PD-1 antibody molecule as described in US 2015/0210769, published on Jul. 30, 2015, entitled “Antibody Molecules to PD-1 and Uses Thereof.”

In another embodiment, there is provided a combination of a compound of Formula I or a pharmaceutically acceptable salt thereof, and a chemotherapy, and a PD-1 inhibitor. In particular, the chemotherapy and PD-1 inhibitor are selected from those described above. In some embodiments, the PD-1 inhibitor (e.g., tislelizumab) is administered at a flat dose of between about 100 mg to about 600 mg. In some embodiments, the PD-1 inhibitor is administered at a dose of between about 100 mg to about 500 mg. In some embodiments, the PD-1 inhibitor is administered at a dose of between about 100 mg to about 400 mg. In some embodiments, the PD-1 inhibitor is administered at a dose of between about 100 mg to about 300 mg. In some embodiments, the PD-1 inhibitor is administered at a dose of between about 100 mg to about 200 mg. In some embodiments, the PD-1 inhibitor is administered at a dose of between about 200 mg to about 600 mg. In some embodiments, the PD-1 inhibitor is administered at a dose of between about 200 mg to about 500 mg. In some embodiments, the PD-1 inhibitor is administered at a dose of between about 200 mg to about 400 mg. In some embodiments, the PD-1 inhibitor is administered at a dose of between about 200 mg to about 300 mg. In some embodiments, the PD-1 inhibitor is administered at a dose of between about 300 mg to about 600 mg. In some embodiments, the PD-1 inhibitor is administered at a dose of between about 300 mg to about 500 mg. In some embodiments, the PD-1 inhibitor is administered at a dose of between about 300 mg to about 400 mg. In some embodiments, the PD-1 inhibitor is administered at a dose of between about 400 mg to about 600 mg. In some embodiments, the PD-1 inhibitor is administered at a dose of between about 400 mg to about 500 mg. In some embodiments, the PD-1 inhibitor is administered at a dose of between about 500 mg to about 600 mg. In some embodiments, the PD-1 inhibitor is administered at a dose of between about 600 mg to about 700 mg. In some embodiments, the PD-1 inhibitor is administered at a dose of between about 700 mg to about 800 mg. In some embodiments, the PD-1 inhibitor is administered at a dose of between about 800 mg to about 900 mg. In some embodiments, the PD-1 inhibitor is administered at a dose of between about 900 mg to about 1000 mg.

In some embodiments, the PD-1 inhibitor (e.g., tislelizumab) is administered at a flat dose of about 100 mg. In some embodiments, the PD-1 inhibitor is administered at a dose of about 200 mg. In some embodiments, the PD-1 inhibitor is administered at a dose of about 300 mg. In some embodiments, the PD-1 inhibitor is administered at a dose of about 400 mg. In some embodiments, the PD-1 inhibitor is administered at a dose of about 500 mg. In some embodiments, the PD-1 inhibitor is administered at a dose of about 600 mg. In some embodiments, the PD-1 inhibitor is administered at a dose of about 700 mg. In some embodiments, the PD-1 inhibitor is administered at a dose of about 800 mg. In some embodiments, the PD-1 inhibitor is administered at a dose of about 900 mg. In some embodiments, the PD-1 inhibitor is administered at a dose of about 1000 mg.

In some embodiments, the PD-1 inhibitor (e.g., tislelizumab) is administered once every ten weeks. In some embodiments, the PD-1 inhibitor is administered once every nine weeks. In some embodiments, the PD-1 inhibitor is administered once every eight weeks. In some embodiments, the PD-1 inhibitor is administered once every seven weeks. In some embodiments, the PD-1 inhibitor is administered once every six weeks. In some embodiments, the PD-1 inhibitor is administered once every five weeks. In some embodiments, the PD-1 inhibitor is administered once every four weeks. In some embodiments, the PD-1 inhibitor is administered once every three weeks. In some embodiments, the PD-1 inhibitor is administered once every two weeks. In some embodiments, the PD-1 inhibitor is administered once every week.

In some embodiments, the PD-1 inhibitor (e.g., tislelizumab) is administered intravenously.

In some embodiments, the PD-1 inhibitor (e.g., tislelizumab) is administered over a period of about 20 minutes to 40 minutes (e.g., about 30 minutes). In some embodiments, the PD-1 inhibitor is administered over a period of about 30 minutes. In some embodiments, the PD-1 inhibitor is administered over a period of about an hour. In some embodiments, the PD-1 inhibitor is administered over a period of about two hours. In some embodiments, the PD-1 inhibitor is administered over a period of about three hours. In some embodiments, the PD-1 inhibitor is administered over a period of about four hours. In some embodiments, the PD-1 inhibitor is administered over a period of about five hours. In some embodiments, the PD-1 inhibitor is administered over a period of about six hours.

In some embodiments, the PD-1 inhibitor (e.g., tislelizumab) is administered at a dose between about 300 mg to about 500 mg (e.g., about 400 mg), intravenously, once every four weeks. In some embodiments, the PD-1 inhibitor is administered at a dose between about 200 mg to about 400 mg (e.g., about 300 mg), intravenously, once every three weeks. In some embodiments, tislelizumab is administered at a dose of 400 mg, once every four weeks. In some embodiments, tislelizumab is administered at a dose of 300 mg, once every three weeks.

In some embodiments, the PD-1 inhibitor (e.g., tislelizumab) is administered at a dose between about 300 mg to about 500 mg (e.g., about 400 mg), intravenously, over a period of about 20 minutes to about 40 minutes (e.g., about 30 minutes), once every two weeks. In some embodiments, the PD-1 inhibitor is administered at a dose between about 200 mg to about 400 mg (e.g., about 300 mg), intravenously, over a period of about 20 minutes to about 40 minutes (e.g., about 30 minutes), once every three weeks.

In some embodiments, the PD-1 inhibitor (e.g., tislelizumab) is administered at a dose of about 100 mg per week. For example, if a 10-week dose is given to a patient, then the PD-1 inhibitor (e.g., tislelizumab) can be given at 1000 mg. If a 9-week dose is given, then the PD-1 inhibitor (e.g., tislelizumab) can be given at 900 mg. If an 8-week dose is given, then the PD-1 inhibitor (e.g., tislelizumab) can be given at 800 mg. If a 7-week dose is given, then the PD-1 inhibitor (e.g., tislelizumab) can be given at 700 mg. If a 6-week dose is given, then the PD-1 inhibitor (e.g., tislelizumab) can be given at 600 mg. If a 5-week dose is given, then the PD-1 inhibitor (e.g., tislelizumab) can be given at 500 mg. If a 4-week dose is given, then the PD-1 inhibitor (e.g., tislelizumab) can be given at 400 mg. If a 3-week dose is given, then the PD-1 inhibitor (e.g., tislelizumab) can be given at 300 mg. If a 2-week dose is given, then the PD-1 inhibitor (e.g., tislelizumab) can be given at 200 mg. If a 1-week dose is given, then the PD-1 inhibitor (e.g., tislelizumab) can be given at 100 mg.

For example, if an anti-PD-1 antibody, such as tislelizumab is used, it can be administered at a dose of 200 mg as an intravenous infusion, once every three weeks. Alternatively, tislelizumab can be administered at a dose of 300 mg as an intravenous infusion, once every four weeks. If an anti-PD-1 antibody, such as tislelizumab is used, it can be administered at a dose of 300 mg as an intravenous infusion, once every three weeks. Alternatively, tislelizumab can be administered at a dose of 400 mg as an intravenous infusion, once every four weeks.

The above-mentioned compounds, which can be used in combination with a compound of the present invention, can be prepared and administered as described in the art, such as in the documents cited above.

In one embodiment, the invention provides a product comprising a compound of the present invention and at least one other therapeutic agent as a combined preparation for simultaneous, separate or sequential use in therapy. In one embodiment, the therapy is the treatment of a disease or condition mediated by WRN. Products provided as a combined preparation include a composition comprising the compound of Formula I and the other therapeutic agent(s) together in the same pharmaceutical composition, or the compound of the present invention and the other therapeutic agent(s) in separate form, e.g. in the form of a kit.

In one embodiment, the invention provides a kit comprising two or more separate pharmaceutical compositions, at least one of which contains a compound of the present invention. In one embodiment, the kit comprises means for separately retaining said compositions, such as a container, divided bottle, or divided foil packet. An example of such a kit is a blister pack, as typically used for the packaging of tablets, capsules and the like.

The kit of the invention may be used for administering different dosage forms, for example, oral and parenteral, for administering the separate compositions at different dosage intervals, or for titrating the separate compositions against one another. To assist compliance, the kit of the invention typically comprises directions for administration.

In the combination therapies of the invention, the compound of the present invention and the other therapeutic agent may be manufactured and/or formulated by the same or different manufacturers. Moreover, the compound of the present invention and the other therapeutic may be brought together into a combination therapy: (i) prior to release of the combination product to physicians (e.g. in the case of a kit comprising the compound of the present invention and the other therapeutic agent); (ii) by the physician themselves (or under the guidance of the physician) shortly before administration; (iii) in the patient themselves, e.g. during sequential administration of the compound of the present invention and the other therapeutic agent.

Accordingly, the invention provides the use of a compound of the present invention for treating a disease or condition mediated by WRN, wherein the medicament is prepared for administration with another therapeutic agent. The invention also provides the use of another therapeutic agent for treating a disease or condition mediated by WRN, wherein the medicament is administered with a compound of the present invention.

The invention also provides a compound of the present invention for use in treating a disease or condition mediated by WRN, wherein the compound of the present invention is prepared for administration with another therapeutic agent. The invention also provides another therapeutic agent for use in treating a disease or condition mediated by WRN, wherein the other therapeutic agent is prepared for administration with a compound of the present invention. The invention also provides a compound of the present invention for use in treating a disease or condition mediated by WRN, wherein the compound of the present invention is administered with another therapeutic agent. The invention also provides another therapeutic agent for use in a method of treating a disease or condition mediated by WRN, wherein the other therapeutic agent is administered with a compound of the present invention.

The invention also provides the use of a compound of the present invention for treating a disease or condition mediated by WRN, wherein the patient has previously (e.g. within 24 hours) been treated with another therapeutic agent. The invention also provides the use of another therapeutic agent for treating a disease or condition mediated by WRN, wherein the patient has previously (e.g. within 24 hours) been treated with compound of the present invention.

### 5. General Synthetic Methods of Producing Compounds of the Disclosure

Compounds of the present disclosure, in general, may be prepared by first making Intermediate-4 via bromination of A as shown in Scheme 1.

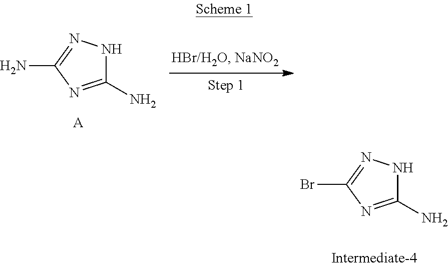

Intermediate-4 is then used in the synthetic sequence shown in Scheme 2 below to establish the bicyclic [1,2,4]triazolo[1,5-a]pyrimidin-7(4H)-one core seen in Intermediate-2 and in the final compounds of Formula I. Ethyl 3-oxopentanoate B is subjected to bromination with N-bromosuccinimide (NBS) to produce C. Nucleophilic displacement of bromide from C using nucleophile D, under basic conditions, produces E. E is reacted with Intermediate-4 to create an in-situ species (not shown) that intramolecularly cyclizes to establish the bicyclic core in Intermediate-2.

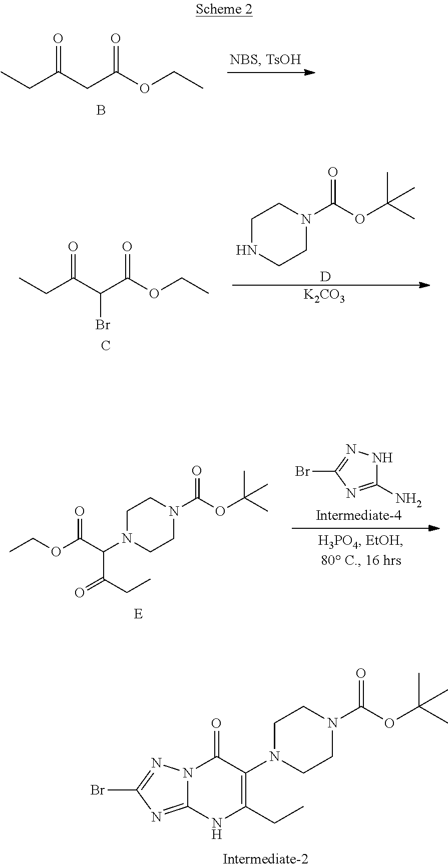

As shown in Scheme 3, Intermediate-2 may be cross-coupled with F to attach the R¹ moiety and form G. Intermediate-3 (prepared as shown below in Scheme 4) or another surrogate electrophile possessing the R² moiety of the genus, may undergo nucleophilic displacement of iodide by the secondary amine of G to form H. H may be deprotected and amidation with R⁴-containing carboxylic acid J may furnish compounds of the disclosure K. Those having ordinary skill in the art will be able to attach R¹, R² (e.g., Intermediate-3's substituent), and R⁴ in different orders of sequence, using similar reactions and deprotecting schemes known in the art. Examples of such alternative orders of attaching substituents R¹, R², and R⁴, to the intermediates in synthesis are illustrated in Example 1.

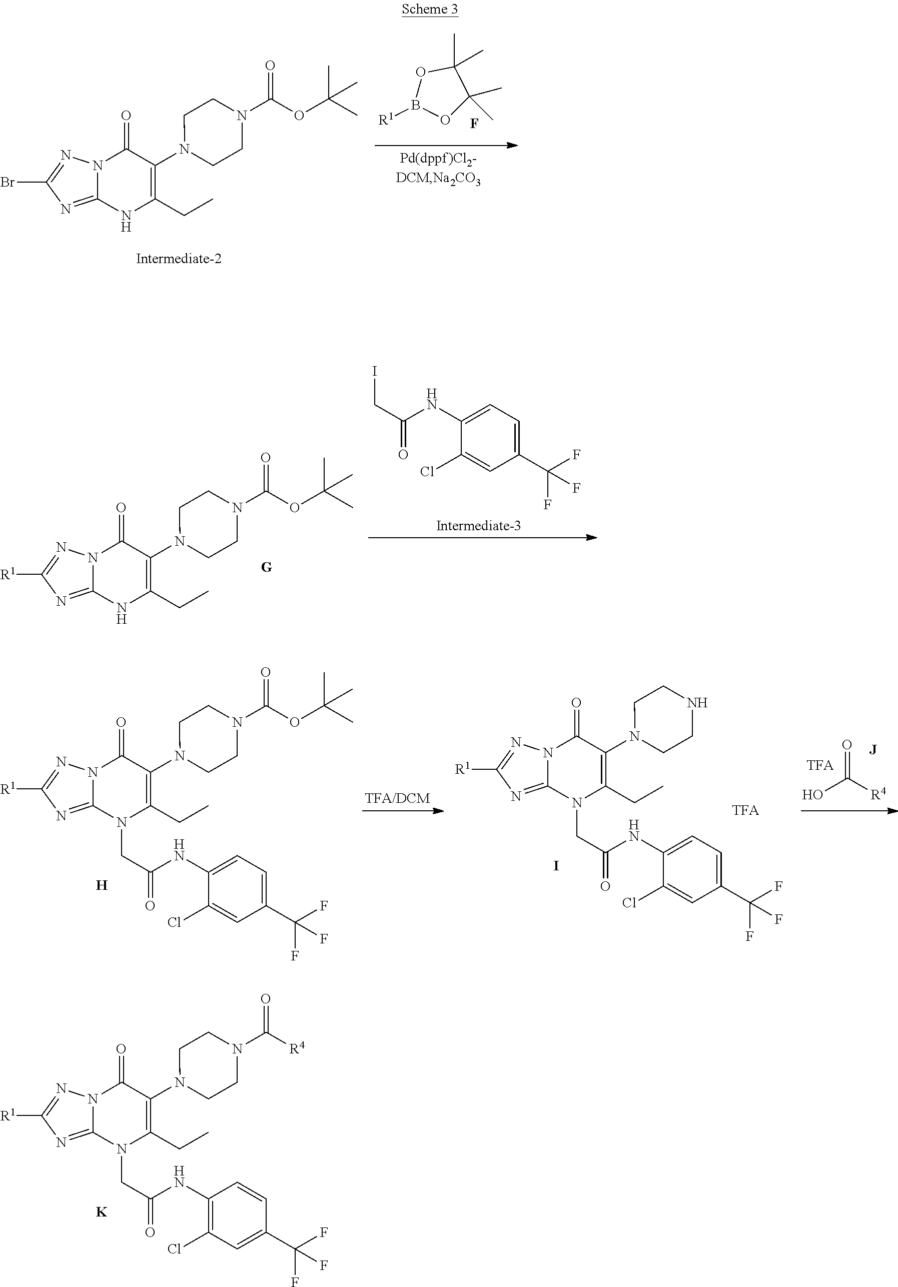

Synthesis of Intermediate-3 which is representative of R² moieties within the present genus may be prepared as shown below in Scheme 4. Substituted anilines such as L may nucleophilically attack a chlorinated acyl chloride M to furnish N which may then be subjected to iodination to furnish Intermediate-3 used in Scheme 3.

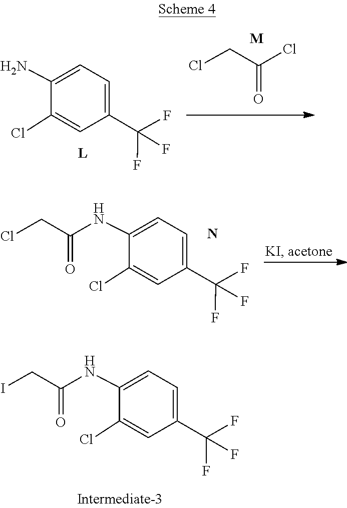

Those having ordinary skill in the art will be able to adapt such synthetic procedures to afford variably substituted compounds of Formula I for synthesis of the compounds of the disclosure.

### EXAMPLES

As depicted in the Examples below, in certain exemplary embodiments, compounds are prepared according to the procedures provided herein. It will be appreciated that, although the methods depict the synthesis of certain compounds of the present disclosure, the methods, and other methods known to one of ordinary skill in the art, can be applied to all compounds and subclasses and species of each of these compounds, as described herein.

### LIST OF ABBREVIATIONS

TEA: triethylamine
H₂O: water
ACN: acetonitrile
THF: tetrahydrofuran
FA: formic acid
AcOH: acetic acid
Na₂SO₄: sodium sulfate
K₂CO₃: potassium carbonate
EtOAc: ethyl acetate
NH₄HCO₃: ammonium bicarbonate
NaHCO₃: sodium bicarbonate
HCl: hydrochloric acid
DCM: dichloromethane
NaOH: sodium hydroxide
DMF: N,N-dimethyl formamide
EtOH: ethanol
MeOH: methanol
CDCl₃: deuterated chloroform
DIEA: N,N-diisopropylethylamine
H₂: hydrogen
N₂: nitrogen
DMSO: dimethylsulfoxide
DMSO-d₆: deuterated dimethyl sulfoxide
TFA: trifluoroacetic acid
Na₂CO₃: sodium carbonate
Pd(dppf)Cl₂: [1,1′-bis(diphenylphosphino)ferrocene]dichloropalladium(II), complex
Pd(dppf)₂Cl₂−CH₂Cl₂:1,1′-Bis(diphenylphosphino)ferrocene-palladium(II)dichloride dichloromethane complex
NBS: N-bromosuccinimide
LiOH: lithium hydroxide
EDCI: N-(3-dimethylaminopropyl)-N′-ethylcarbodiimide hydrochloride
LCMS: liquid chromatography-mass spectrometry
HPLC: high-performance liquid chromatography
Pd/C: palladium on carbon
PE: petroleum ether
aq.: aqueous
M: molar concentration
br: broad
TsOH: 4-methylbenzenesulfonic acid
K₃PO₄: tripotassium phosphate
s: singlet
m: multiplet
d: doublet
t: triplet
q: quartet
ppm: parts per million
KI: potassium iodide
H₃PO₄: phosphoric acid
HBr: hydrobromic acid
NaNO₂: sodium nitrite
eq: equivalent
NIS: N-iodosuccinimide
PCy₃ Pd G3: methanesulfonato(tricyclohexylphosphine)(2′-amino-1,1′-biphenyl-2-yl)palladium(II) dichloromethane adduct
cataCXium A Pd-G3: methanesulfonato(diadamantyl-n-butylphosphino)-2′-amino-1,1′-biphenyl-2-yl)palladium(II)
MeOD: deuterated methanol
NCS: N-chlorosuccinimide
LiHMDS: lithium hexamethyldisilazide
NH₄Cl: ammonium chloride
NaOH: sodium hydroxide
N: normality
AIBN: 2,2′-azobis(2-methylpropionitrile)
CCl₄: carbon tetrachloride
MeMgBr: methylmagnesium bromide
CO: carbon monoxide
HI: hydroiodic acid
Ts: (4-methylphenyl)sulfonyl group
Cs₂CO₃: cesium carbonate

### Example 1: Synthesis of Compounds of the Disclosure

### Synthesis of N-(2-chloro-4-(trifluoromethyl)phenyl)-2-(5-ethyl-6-(4-(5-hydroxy-6-methylpyrimidine-4-carbonyl)piperazin-1-yl)-2-(2-methoxypyridin-4-yl)-7-oxo-[1,2,4]triazolo[1,5-a]pyrimidin-4 (7H)-yl)acetamide I-21

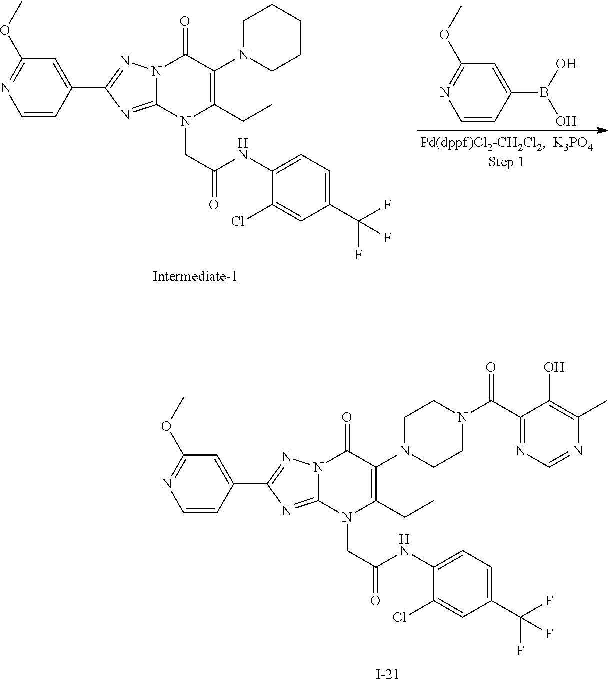

### Step 1. Synthesis of N-(2-chloro-4-(trifluoromethyl)phenyl)-2-(5-ethyl-6-(4-(5-hydroxy-6-methylpyrimidine-4-carbonyl)piperazin-1-yl)-2-(2-methoxypyridin-4-yl)-7-oxo-[1,2,4]triazolo[1,5-a]pyrimidin-4 (7H)-yl)acetamide

To a solution of 2-(2-bromo-5-ethyl-6-(4-(5-hydroxy-6-methylpyrimidine-4-carbonyl)piperazin-1-yl)-7-oxo-[1,2,4]triazolo[1,5-a]pyrimidin-4 (7H)-yl)-N-(2-chloro-4-(trifluoromethyl)phenyl)acetamide (Intermediate-1) (30 mg, 43 μmol, 1.0 eq) and (2-methoxypyridin-4-yl)boronic acid (7 mg, 47 μmol, 1.1 eq) in 1,4-dioxane (1.0 mL) and H₂O (0.2 mL) was added Pd(dppf)Cl₂—CH₂Cl₂ (4 mg, 4 μmol, 0.1 eq) and K₃PO₄ (27 mg, 0.13 mmol, 3.0 eq) at room temperature. The mixture was heated to 100°C and stirred at 100 °C for 16 h under N₂ atmosphere. The reaction mixture was diluted with H₂O (20 mL), then extracted with DCM (20 mL*2). The combined organic layers were dried over Na₂SO₄, filtered and the filtrate was concentrated under reduced pressure. The residue was purified by reverse phase HPLC (C18 column, H₂O (10 mmol/L NH₄HCO₃)-ACN) to afford the title compound.

¹H NMR (400 MHz, CDCl₃) δ ppm 11.81 (br s, 1H), 8.93 (br s, 1H), 8.60 (s, 1H), 8.49 (d, 1H), 8.29 (br d, 1H), 7.71 (br d, 1H), 7.60 (br d, 2H), 7.54 (br d, 1H), 5.81-5.60 (m, 1H), 5.20 (br s, 2H), 4.90-4.74 (m, 1H), 4.00 (s, 3H), 3.91-3.76 (m, 2H), 3.52 (br s, 1H), 3.23 (br d, 2H), 3.15-3.02 (m, 1H), 2.94-2.72 (m, 2H), 2.58 (s, 3H), 1.42-1.32 (m, 3H).

LCMS: 727.4 [M+H]⁺.

### Synthesis of N-(2-chloro-4-(trifluoromethyl)phenyl)-2-(2-(1-cyclopropyl-1H-pyrazol-4-yl)-5-ethyl-6-(4-(5-hydroxy-6-methylpyrimidine-4-carbonyl)piperazin-1-yl)-7-oxo-[1,2,4]triazolo[1,5-a]pyrimidin-4 (7H)-yl)acetamide I-32

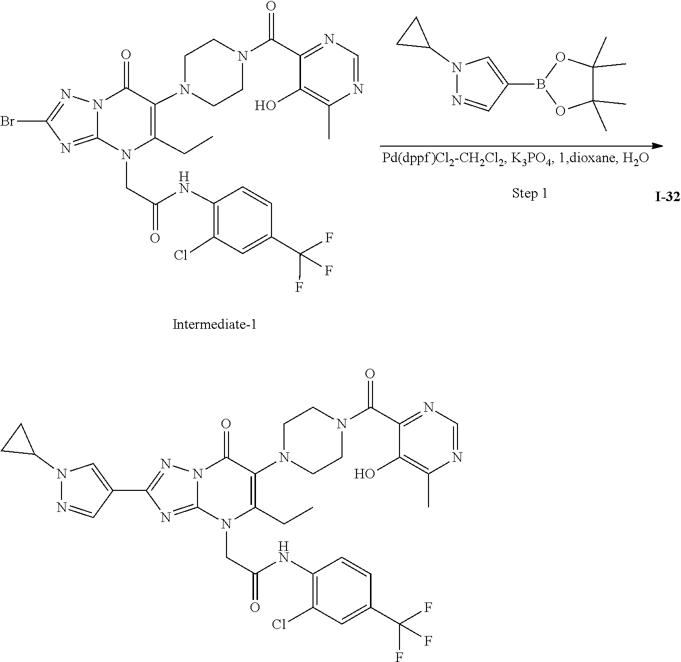

### Step 1. Synthesis of N-(2-chloro-4-(trifluoromethyl)phenyl)-2-(2-(1-cyclopropyl-1H-pyrazol-4-yl)-5-ethyl-6-(4-(5-hydroxy-6-methylpyrimidine-4-carbonyl)piperazin-1-yl)-7-oxo-[1,2,4]triazolo[1,5-a]pyrimidin-4 (7H)-yl)acetamide

To a solution of 2-(2-bromo-5-ethyl-6-(4-(5-hydroxy-6-methylpyrimidine-4-carbonyl)piperazin-1-yl)-7-oxo-[1,2,4]triazolo[1,5-a]pyrimidin-4 (7H)-yl)-N-(2-chloro-4-(trifluoromethyl)phenyl)acetamide (Intermediate-1) (50 mg, 72 μmol, 1.0 eq) and 1-cyclopropyl-4-(4,4,5,5-tetramethyl-1,3,2-dioxaborolan-2-yl)-1H-pyrazole (18 mg, 79 μmol, 1.1 eq) in 1,4-dioxane (1.0 mL) and H₂O (0.2 mL) was added Pd(dppf)Cl₂—CH₂Cl₂ (6 mg, 7 μmol, 0.1 eq) and K₃PO₄ (46 mg, 0.22 mmol, 3.0 eq) at room temperature. Then the mixture was heated to 100°C and stirred at 100 °C for 16 h under N₂ atmosphere. The reaction mixture was diluted with H₂O (10 mL) and extracted with DCM (50 mL*3). The combined organic phase was dried over Na₂SO₄, filtered and the filtrate was concentrated under reduced pressure. The residue was purified by reverse phase HPLC (C18 column, H₂O (FA)-ACN) to afford the title compound.

LCMS: 726.2 [M+H]⁺.

¹H NMR (400 MHz, CDCl₃) δ ppm 11.81 (br s, 1H), 9.15 (br s, 1H), 8.63-8.56 (m, 1H), 8.51 (d, 1H), 8.22-8.02 (m, 2H), 7.62 (s, 1H), 7.54 (d, 1H), 5.80-5.58 (m, 1H), 5.14 (br s, 2H), 4.91-4.69 (m, 1H), 3.92-3.75 (m, 2H), 3.69-3.66 (m, 1H), 3.51-3.49 (m, 1H), 3.26-3.15 (m, 2H), 3.14-3.01 (m, 1H), 2.93-2.71 (m, 2H), 2.57 (s, 3H), 1.36 (br s, 3H), 1.18 (d, 2H), 1.09 (d, 2H).

### Synthesis of N-(2-chloro-4-(trifluoromethyl)phenyl)-2-(2-(3,6-dihydro-2H-pyran-4-yl)-5-ethyl-6-(4-(3-hydroxy-2-methoxyisonicotinoyl)piperazin-1-yl)-7-oxo-[1,2,4]triazolo[1,5-a]pyrimidin-4 (7H)-yl)acetamide I-34

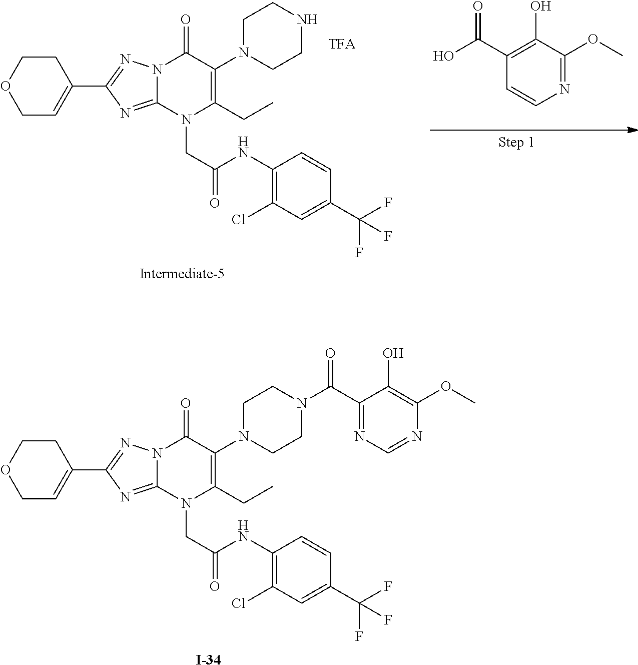 [SMILES: CCc1c(N2CCNCC2)c(=O)n2nc(C3=CCOCC3)nc2n1CC(=O)Nc1ccc(C(F)(F)F)cc1Cl ; COc1nccc(C(=O)O)c1O ; "Intermediate-5" ; CCc1c(N2CCN(C(=O)c3ncnc(OC)c3O)CC2)c(=O)n2nc(C3=CCOCC3)nc2n1CC(=O)Nc1ccc(C(F)(F)F)cc1Cl ; "I-34" ; "TFA"]

### Step 1: Synthesis of N-(2-chloro-4-(trifluoromethyl)phenyl)-2-(2-(3,6-dihydro-2H-pyran-4-yl)-5-ethyl-6-(4-(3-hydroxy-2-methoxyisonicotinoyl)piperazin-1-yl)-7-oxo-[1,2,4]triazolo[1,5-a]pyrimidin-4 (7H)-yl)acetamide

To a solution of N-(2-chloro-4-(trifluoromethyl)phenyl)-2-(2-(3,6-dihydro-2H-pyran-4-yl)-5-ethyl-7-oxo-6-(piperazin-1-yl)-[1,2,4]triazolo[1,5-a]pyrimidin-4 (7H)-yl)acetamide trifluoroacetate (Intermediate-5) (50 mg, 74 μmol, 1.0 eq) and 3-hydroxy-2-methoxyisonicotinic acid (18 mg, 106 μmol, 1.45 eq) in pyridine (1.0 mL) was added EDCI (25 mg, 0.13 mmol, 1.8 eq) at room temperature. Then the mixture was stirred at room temperature for 15 h. The reaction mixture was poured into ice-H₂O (10 mL) and extracted with EtOAc (10 mL*3). The combined organic layers were concentrated in vacuum. The residue was dissolved in THF (2 mL), H₂O (1 mL) and MeOH (1 mL), LiOH—H₂O (7 mg, 167 μmol, 3 eq) was added to the resulting solution at room temperature. The mixture was stirred at room temperature for 1 h. The mixture was diluted H₂O (10 mL) and extracted with EtOAc (10 mL*3). The combined organic phase was washed with brine (10 mL), dried over anhydrous Na₂SO₄, filtered and the filtrate was concentrated in vacuum. The residue was purified by reverse phase HPLC (C18 column, H₂O (FA)-ACN) to afford the title compound.

LCMS: 717.4 [M+H]⁺.

¹H NMR (400 MHz, CDCl₃) δ ppm 8.96 (br s, 1H), 8.39 (br d, 1H), 7.65 (br s, 1H), 7.56 (s, 1H), 7.46 (br d, 1H), 6.90-6.77 (m, 2H), 5.15-4.92 (m, 2H), 4.33-4.29 (m, 2H), 3.97 (s, 3H), 3.84-3.82 (m, 2H), 3.69-3.62 (m, 2H), 3.21-3.01 (m, 4H), 2.65-2.63 (m, 6H), 1.24-1.22 (m, 3H).

### Synthesis of N-(2-chloro-4-(trifluoromethyl)phenyl)-2-(5-ethyl-6-(4-(3-hydroxy-2-methoxyisonicotinoyl)piperazin-1-yl)-2-(2-methoxypyridin-4-yl)-7-oxo-[1,2,4]triazolo[1,5-a]pyrimidin-4 (7H)-yl)acetamide I-80

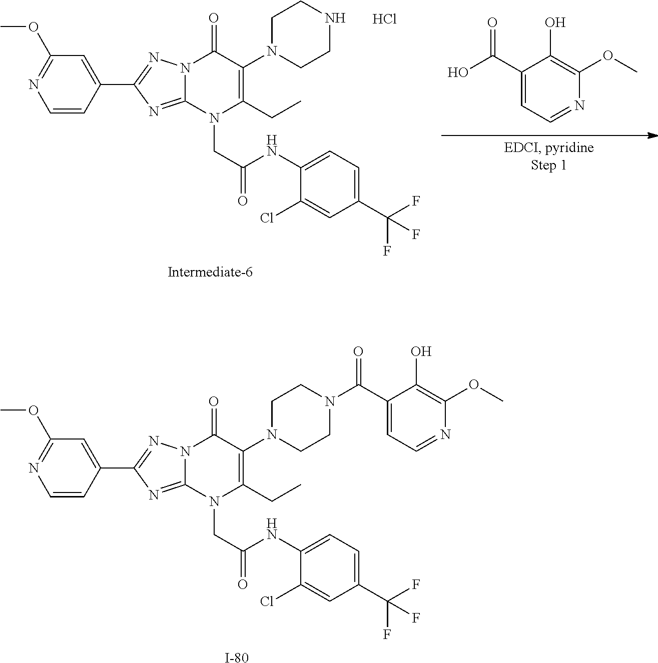

### Step 1: Synthesis of N-(2-chloro-4-(trifluoromethyl)phenyl)-2-(5-ethyl-6-(4-(3-hydroxy-2-methoxyisonicotinoyl)piperazin-1-yl)-2-(2-methoxypyridin-4-yl)-7-oxo-[1,2,4]triazolo[1,5-a]pyrimidin-4 (7H)-yl)acetamide

To a mixture of N-(2-chloro-4-(trifluoromethyl)phenyl)-2-(5-ethyl-2-(2-methoxypyridin-4-yl)-7-oxo-6-(piperazin-1-yl)-[1,2,4]triazolo[1,5-a]pyrimidin-4 (7H)-yl)acetamide hydrochloride (Intermediate-6) (40 mg, 64 μmol, 1.0 eq) and 3-hydroxy-2-methoxyisonicotinic acid (11 mg, 65 μmol, 1.0 eq) in pyridine (1 mL) was added EDCI (26 mg, 135 μmol, 2.13 eq), the resulting mixture was stirred at room temperature for 1 h and then concentrated in vacuo. The residue was purified by reverse phase HPLC (C18 column, H₂O (0.1% FA)-ACN) to afford the title compound.

¹H NMR (400 MHz, CDCl₃) δ ppm 8.90 (s, 1H), 8.48 (d, 1H), 8.31 (d, 1H), 7.74 (d, 2H), 7.65-7.59 (m, 2H), 7.54 (d, 1H), 7.01 (d, 1H), 6.86 (d, 1H), 5.19 (s, 2H), 4.66-3.60 (m, 10H), 3.34-3.11 (m, 4H), 2.83-2.71 (m, 2H), 1.35 (t, 3H).

LCMS: 742.4 [M+H]⁺.

### Synthesis of N-(2-chloro-4-(trifluoromethyl)phenyl)-2-(6-(1-(5-hydroxy-6-methylpyrimidine-4-carbonyl)piperidin-4-yl)-2-(2-methoxypyridin-4-yl)-7-oxo-[1,2,4]triazolo[1,5-a]pyrimidin-4 (7H)-yl)acetamide I-81

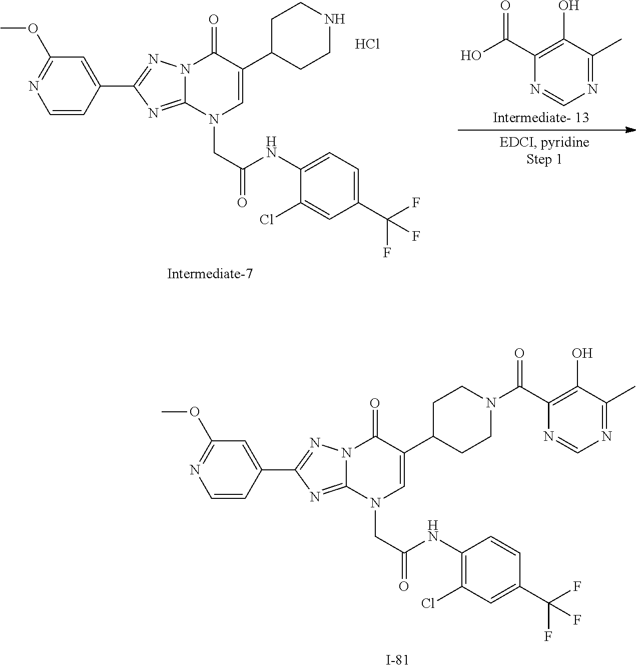

### Step 1. Synthesis of N-(2-chloro-4-(trifluoromethyl)phenyl)-2-(6-(1-(5-hydroxy-6-methylpyrimidine-4-carbonyl)piperidin-4-yl)-2-(2-methoxypyridin-4-yl)-7-oxo-[1,2,4]triazolo[1,5-a]pyrimidin-4 (7H)-yl)acetamide

To a solution of N-(2-chloro-4-(trifluoromethyl)phenyl)-2-(2-(2-methoxypyridin-4-yl)-7-oxo-6-(piperidin-4-yl)-[1,2,4]triazolo[1,5-a]pyrimidin-4 (7H)-yl)acetamide hydrochloride (Intermediate-7) (30 mg, 50 μmol, 1.00 eq) and 5-hydroxy-6-methylpyrimidine-4-carboxylic acid (Intermediate-13) (41 mg, 0.27 mmol, 5.4 eq) in pyridine (1 mL) was added EDCI (41 mg, 0.21 mmol, 4.2 eq). The resulting mixture was stirred at room temperature overnight and then concentrated in vacuo. The residue was purified by reverse phase HPLC (C18 column, H₂O (0.1% TFA)-ACN) to afford the title compound.

¹H NMR (400 MHz, MeOD) δ ppm 8.57 (s, 1H), 8.26 (d, 1H), 8.18 (d, 1H), 7.97 (s, 1H), 7.82 (s, 1H), 7.71 (d, 1H), 7.61 (br d, 1H), 7.56 (s, 1H), 5.34 (s, 2H), 4.93-4.90 (m, 1H), 4.09 (br d, 1H), 3.97 (s, 3H), 3.39-3.34 (m, 1H), 3.19 (br s, 1H), 3.08-2.87 (m, 1H), 2.52 (s, 3H), 2.13 (br d, 1H), 2.01 (br d, 1H), 1.81 (br s, 2H).

LCMS: 698.2 [M+H]⁺.

### Synthesis of N-(2-chloro-4-(pentafluoro-26-sulfaneyl)phenyl)-2-(5-ethyl-2-(2-methoxypyridin-4-yl)-6-(4-(1-methyl-1H-pyrazole-4-carbonyl)piperazin-1-yl)-7-oxo-[1,2,4]triazolo[1,5-a]pyrimidin-4 (7H)-yl)acetamide I-82

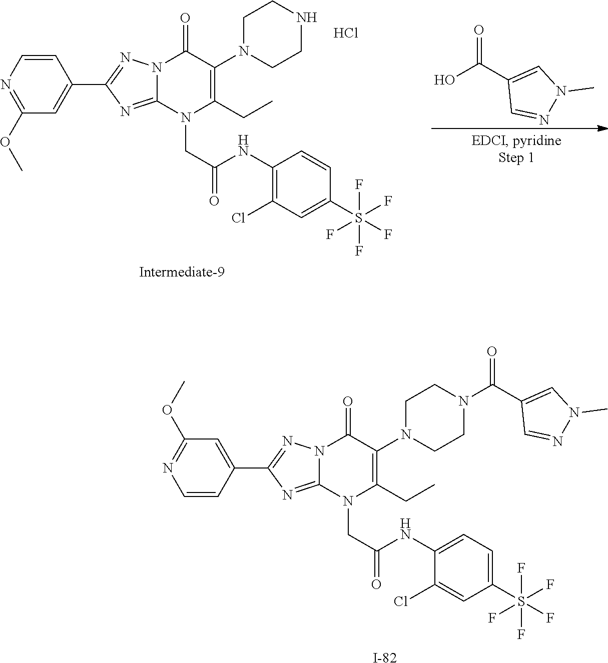

Step 1. Synthesis of N-(2-chloro-4-(pentafluoro-26-sulfaneyl)phenyl)-2-(5-ethyl-2-(2-methoxypyridin-4-yl)-6-(4-(1-methyl-1H-pyrazole-4-carbonyl)piperazin-1-yl)-7-oxo-[1,2,4]triazolo[1,5-a]pyrimidin-4 (7H)-yl)acetamide

To a solution of N-(2-chloro-4-(pentafluoro-26-sulfaneyl)phenyl)-2-(5-ethyl-2-(2-methoxypyridin-4-yl)-7-oxo-6-(piperazin-1-yl)-[1,2,4]triazolo[1,5-a]pyrimidin-4 (7H)-yl)acetamide hydrochloride (Intermediate-9) (50 mg, 73 μmol, 1.0 eq) in pyridine (3 mL) was added EDCI (59 mg, 0.31 mmol, 4.2 eq) and 1-methyl-1H-pyrazole-4-carboxylic acid (12 mg, 95 μmol, 1.3 eq). The resulting mixture was stirred at room temperature for 2 h. The mixture was diluted with H₂O (10 mL) and extracted with DCM (10 mL*3). The combined organic layer was dried over anhydrous Na₂SO₄, filtered, and the filtrate was concentrated in vacuo. The residue was purified by reverse phase HPLC (C18 column, H₂O (0.1% FA)-ACN) to afford the title compound.

¹H NMR (400 MHz, CDCl₃) δ ppm 8.99 (s, 1H), 8.49 (d, 1H), 8.29 (d, 1H), 7.83-7.61 (m, 5H), 7.58 (s, 1H), 5.18 (s, 2H), 4.81-4.20 (m, 2H), 4.00 (s, 3H), 3.94 (s, 3H), 3.83-3.67 (m, 2H), 3.41-2.99 (m, 4H), 2.77 (d, 2H), 1.36 (t, 3H).

LCMS: 757.3 [M+H]⁺.

### Synthesis of (R)—N-(2-chloro-4-(trifluoromethyl)phenyl)-2-(5-ethyl-2-(2-methoxypyridin-4-yl)-6-(3-methyl-4-(1-methyl-1H-pyrazole-4-carbonyl)piperazin-1-yl)-7-oxo-[1,2,4]triazolo[1,5-a]pyrimidin-4 (7H)-yl)acetamide I-77

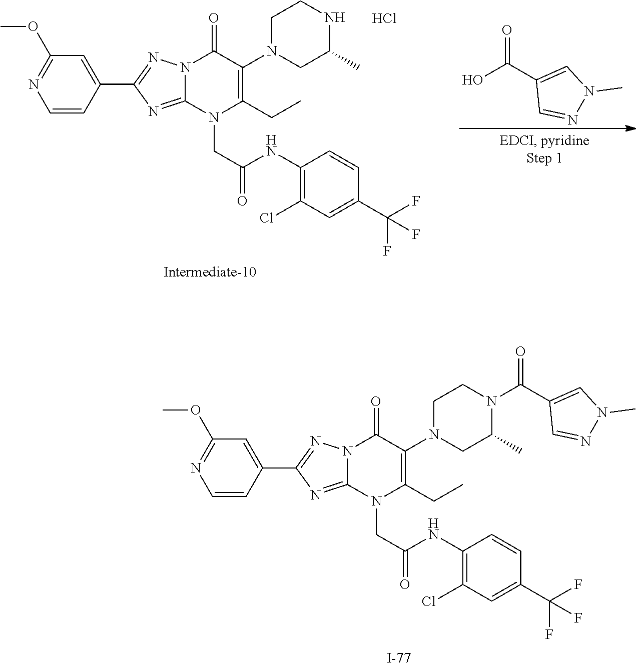

Step 1: Synthesis of (R)—N-(2-chloro-4-(trifluoromethyl)phenyl)-2-(5-ethyl-2-(2-methoxypyridin-4-yl)-6-(3-methyl-4-(1-methyl-1H-pyrazole-4-carbonyl)piperazin-1-yl)-7-oxo-[1,2,4]triazolo[1,5-a]pyrimidin-4 (7H)-yl)acetamide

To a solution of (R)—N-(2-chloro-4-(trifluoromethyl)phenyl)-2-(5-ethyl-2-(2-methoxypyridin-4-yl)-6-(3-methylpiperazin-1-yl)-7-oxo-[1,2,4]triazolo[1,5-a]pyrimidin-4 (7H)-yl)acetamide hydrochloride (Intermediate-10) (42 mg, 67 μmol, 1.0 eq) and 1-methyl-1H-pyrazole-4-carboxylic acid (11 mg, 87 μmol, 1.3 eq) in pyridine (1 mL) was added EDCI (20 mg, 0.10 mmol, 1.6 eq). The resulting mixture was stirred at room temperature for 1 h. The reaction mixture was diluted with H₂O (10 mL) and extracted with EtOAc (10 mL*3). The combined organic layer was dried over anhydrous Na₂SO₄, filtered, and the filtrate was concentrated in vacuo. The residue was purified by reverse phase HPLC (C18 column, H₂O (0.1% FA)-ACN) to afford the title compound.

¹H NMR (400 MHz, CDCl₃) δ ppm 9.00 (br s, 1H), 8.48 (d, 1H), 8.29 (d, 1H), 7.76 (br s, 1H), 7.71 (d, 1H), 7.67-7.62 (m, 2H), 7.59-7.53 (m, 2H), 5.22 (br s, 2H), 5.07-4.57 (m, 1H), 4.53-4.03 (m, 1H), 4.01-3.89 (m, 7H), 3.82-3.15 (m, 4H), 2.87-2.54 (m, 2H), 1.58-1.46 (m, 3H), 1.38 (t, 3H).

LCMS: 713.4 [M+H]⁺.

### Synthesis of N-(2-chloro-4-(trifluoromethyl)phenyl)-2-(5-ethyl-6-(4-(7-hydroxy-2,3-dihydrofuro[3,2-c]pyridine-6-carbonyl)piperazin-1-yl)-2-(2-methoxypyridin-4-yl)-7-oxo-[1,2,4]triazolo[1,5-a]pyrimidin-4 (7H)-yl)acetamide I-86

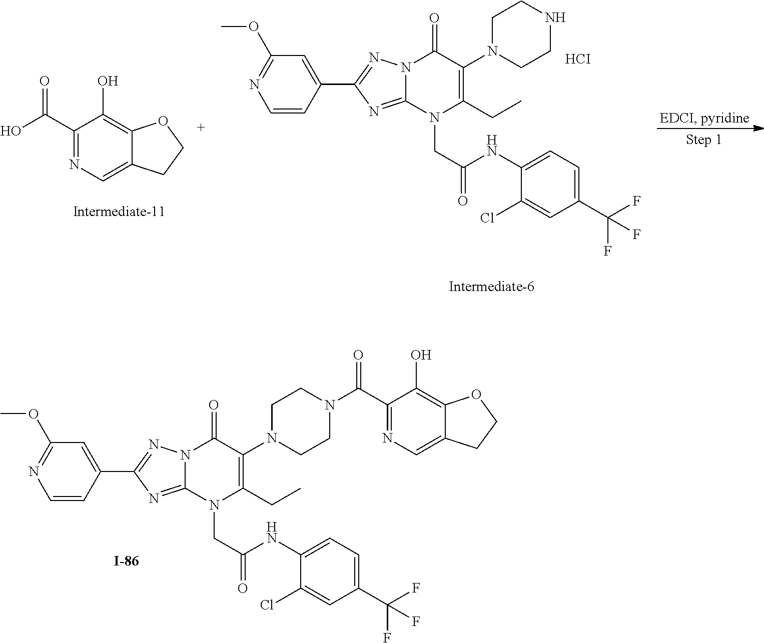

### Step 1. Synthesis of N-(2-chloro-4-(trifluoromethyl)phenyl)-2-(5-ethyl-6-(4-(7-hydroxy-2,3-dihydrofuro[3,2-c]pyridine-6-carbonyl)piperazin-1-yl)-2-(2-methoxypyridin-4-yl)-7-oxo-[1,2,4]triazolo[1,5-a]pyrimidin-4 (7H)-yl)acetamide

To a stirred solution of 7-hydroxy-2H,3H-furo[3,2-c]pyridine-6-carboxylic acid (Intermediate-11) (43 mg, 0.24 mmol, 3.00 eq) and N-[2-chloro-4-(trifluoromethyl)phenyl]-2-[5-ethyl-2-(2-methoxypyridin-4-yl)-7-oxo-6-(piperazin-1-yl)-[1,2,4]triazolo[1,5-a]pyrimidin-4-yl]acetamide hydrochloride (Intermediate-6) (50 mg, 80 μmol, 1.0 eq) in pyridine (2.00 mL) was added EDCI (92 mg, 0.48 mmol, 6.0 eq) at room temperature. The resulting mixture was stirred for 16 h at 50 °C. The reaction mixture was concentrated under reduced pressure. The residue was diluted with H₂O and extracted with EtOAc. The combined organic layers were concentrated under reduced pressure. LiOH (6 mg, 0.24 mmol, 3.0 eq) was added to a solution of the residue in THF (2.00 mL), H₂O (1.00 mL) and MeOH (1.00 mL) at room temperature. The mixture was stirred at room temperature for 1 h. The mixture was diluted H₂O and extracted with EtOAc. The combined organic phase was washed with brine, dried over anhydrous Na₂SO₄, filtered and the filtrate was concentrated in vacuum. The residue was purified by reverse Phase HPLC (C18 column, H₂O (10 mmol/L NH₄HCO₃)-ACN) to afford the title compound.

LCMS: 754 [M+H]⁺.

¹H NMR (400 MHz, DMSO-d₆) δ ppm 10.18 (br s, 2H), 8.34 (d, 1H), 8.04 (d, 1H), 7.97 (d, 1H), 7.91 (s, 1H), 7.72-7.70 (m, 1H), 7.63-7.61 (m, 1H), 7.38 (s, 1H), 5.39 (s, 2H), 4.69 (t, 2H), 4.53 (d, 1H), 3.91 (s, 3H), 3.57-3.43 (m, 3H), 3.29-3.19 (m, 3H), 3.05-2.93 (m, 3H), 2.83-2.81 (m, 1H), 2.67-2.63 (m, 1H), 1.21 (t, 3H).

### Synthesis of N-(2-chloro-4-(trifluoromethyl)phenyl)-2-(5-ethyl-6-(4-(4-hydroxy-2,3-dihydrofuro[2,3-c]pyridine-5-carbonyl)piperazin-1-yl)-2-(2-methoxypyridin-4-yl)-7-oxo-[1,2,4]triazolo[1,5-a]pyrimidin-4 (7H)-yl)acetamide I-85

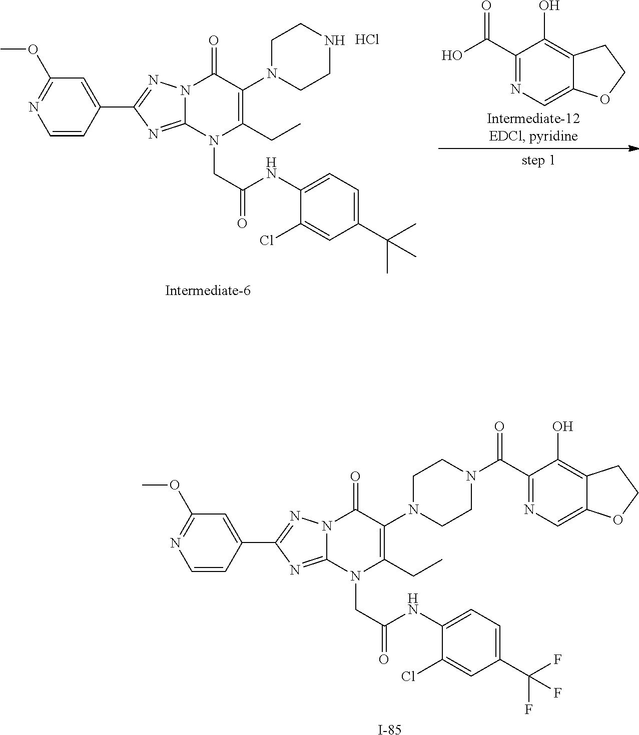

To a stirred solution of 4-hydroxy-2H,3H-furo[2,3-c]pyridine-5-carboxylic acid (Intermediate-12) (46 mg, 0.255 mmol, 3.19 eq) and N-[2-chloro-4-(trifluoromethyl)phenyl]-2-[5-ethyl-2-(2-methoxypyridin-4-yl)-7-oxo-6-(piperazin-1-yl)-[1,2,4]triazolo[1,5-a]pyrimidin-4-yl]acetamide hydrochloride (Intermediate-6) (50 mg, 0.080 mmol, 1.00 eq) in pyridine (2.00 mL) was add EDCI (97 mg, 0.51 mmol, 6.4 eq) at room temperature. The resulting mixture was stirred for overnight at 60°C under N₂. The resulting mixture was concentrated under reduced pressure. The residue was purified by reverse phase HPLC (C18 column, water (10 mmol/L NH₄HCO₃)-ACN) to afford the title compound.

¹H NMR (400 MHz, DMSO-d₆) δ ppm 10.61 (br s, 2H), 8.34 (d, 1H), 8.04 (d, 1H), 7.97 (s, 1H), 7.72-7.70 (m, 2H), 7.61 (d, 1H), 7.38 (s, 1H), 5.40 (s, 2H), 4.65 (t, 2H), 4.57 (br s, 1 H), 4.22 (br s, 1H), 3.91 (s, 3H), 3.54-3.52 (m, 2H), 3.51-3.50 (m, 1H), 3.19-3.17 (m, 2H), 3.10-2.97 (m, 3H), 2.90-2.71 (m, 2H), 1.23 (t, 3H).

LCMS: 754.2 [M+H]⁺.

### Intermediates

### Intermediate-1:2-(2-bromo-5-ethyl-6-(4-(5-hydroxy-6-methylpyrimidine-4-carbonyl)piperazin-1-yl)-7-oxo-[1,2,4]triazolo[1,5-a]pyrimidin-4(7H)-yl)-N-(2-chloro-4-(trifluoromethyl)phenyl)acetamide

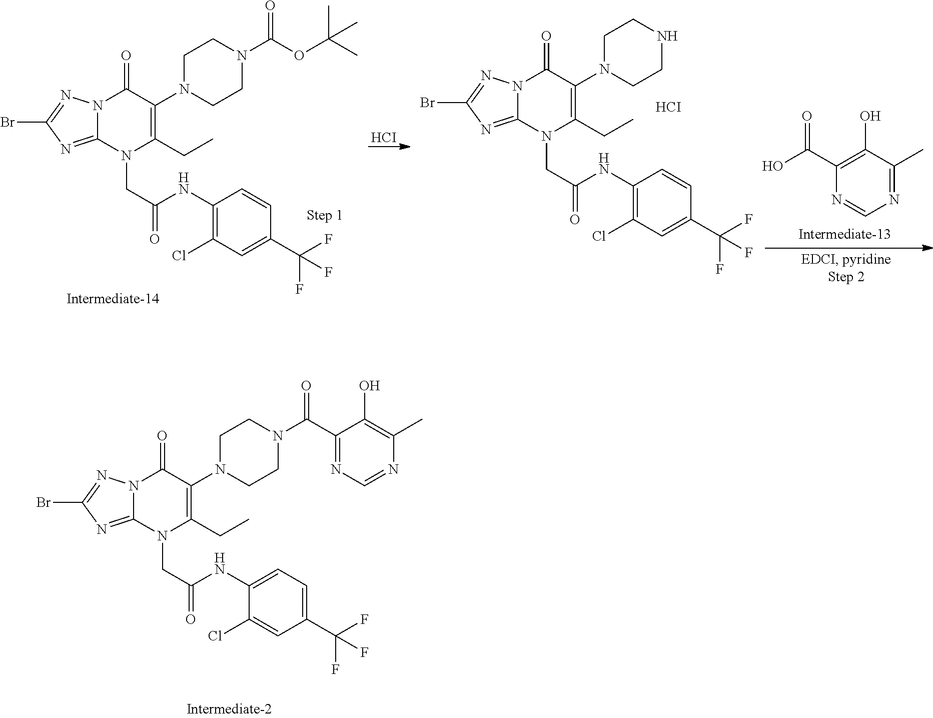

### Step 1. Synthesis of 2-(2-bromo-5-ethyl-7-oxo-6-(piperazin-1-yl)-[1,2,4]triazolo[1,5-a]pyrimidin-4 (7H)-yl)-N-(2-chloro-4-(trifluoromethyl)phenyl)acetamide hydrochloride

To a solution of 4 M HCl in 1,4-dioxane (20 mL) was added tert-butyl 4-(2-bromo-4-(2-((2-chloro-4-(trifluoromethyl)phenyl)amino)-2-oxoethyl)-5-ethyl-7-oxo-4,7-dihydro-[1,2,4]triazolo[1,5-a]pyrimidin-6-yl)piperazine-1-carboxylate (Intermediate-14) (5.00 g, 7.54 mmol, 1.0 eq) at room temperature and it was stirred at room temperature for 4 h. The reaction mixture was concentrated under reduced pressure to afford the title compound, which was used in the next step without further purification.

LCMS: 564.1 [M+H]⁺.

### Step 2. 2-(2-bromo-5-ethyl-6-(4-(5-hydroxy-6-methylpyrimidine-4-carbonyl)piperazin-1-yl)-7-oxo-[1,2,4]triazolo[1,5-a]pyrimidin-4(7H)-yl)-N-(2-chloro-4-(trifluoromethyl)phenyl)acetamide

To a solution of 5-hydroxy-6-methylpyrimidine-4-carboxylic acid (Intermediate-13) (1.65 g, 10.7 mmol, 1.13 eq) in pyridine (20 mL) was added EDCI (3.28 g, 17.1 mmol, 1.8 eq) and 2-(2-bromo-5-ethyl-7-oxo-6-(piperazin-1-yl)-[1,2,4]triazolo[1,5-a]pyrimidin-4(7H)-yl)-N-(2-chloro-4-(trifluoromethyl)phenyl)acetamide hydrochloride (5.70 g, 9.51 mmol, 1.0 eq) at room temperature. Then the mixture was stirred at room temperature for 16 h. The reaction mixture was diluted with H₂O (50 mL), then extracted with EtOAc (50 mL*2). The combined organic layers were dried over Na₂SO₄, filtered and the filtrate was concentrated under reduced pressure. The residue was purified by flash silica gel chromatography (eluent of EtOAc/PE) to afford the title compound.

LCMS: 698.1 [M+H]⁺.

### Intermediate-2: tert-butyl 4-(2-bromo-5-ethyl-7-oxo-4,7-dihydro-[1,2,4]triazolo[1,5-a]pyrimidin-6-yl)piperazine-1-carboxylate

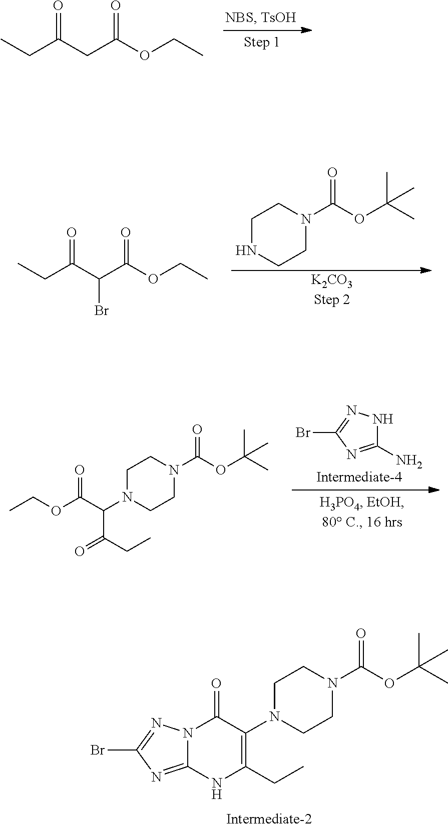

### Step 1. Synthesis of ethyl 2-bromo-3-oxopentanoate

To a solution of ethyl 3-oxopentanoate (50.00 g, 346.8 mmol, 1.0 eq) in DCM (500 mL) was added NBS (64.81 g, 364.2 mmol, 1.05 eq) and TsOH (11.94 g, 69.36 mmol, 0.2 eq) at room temperature. The resulting mixture was stirred at room temperature for 1 h. The reaction mixture was poured into H₂O (300 mL) and extracted with DCM (100 mL). The organic layer was washed with H₂O (200 mL*3) and brine (200 mL), dried over anhydrous Na₂SO₄, filtered, and concentrated in vacuum to afford the title compound, which was used in next step without further purification.

¹H NMR (400 MHz, CDCl₃) δ ppm 4.81 (s, 1H), 4.30 (q, 2H), 2.81 (q, 2H), 1.36-1.29 (m, 3H), 1.15 (t, 3H).

LCMS: 225.0 [M+H]⁺.

### Step 2. Synthesis of tert-butyl 4-(1-ethoxy-1,3-dioxopentan-2-yl)piperazine-1-carboxylate

To a solution of ethyl 2-bromo-3-oxopentanoate (40.00 g, 179.3 mmol, 1.0 eq) and K₂CO₃ (74.35 g, 538.0 mmol, 3.0 eq) in ACN (300 mL) was added tert-butyl piperazine-1-carboxylate (66.80 g, 358.6 mmol, 2.0 eq) and the mixture was stirred at room temperature for 1 h. The reaction mixture was filtered, and the filtrate was concentrated in vacuum. The residue was purified by column chromatography on silica (eluent of EtOAc/PE) to afford the title compound.

LCMS: 329.2 [M+H]⁺.

### Step 3. Synthesis of tert-butyl 4-(2-bromo-5-ethyl-7-oxo-4,7-dihydro-[1,2,4]triazolo[1,5-a]pyrimidin-6-yl)piperazine-1-carboxylate

A mixture of tert-butyl 4-(1-ethoxy-1,3-dioxopentan-2-yl)piperazine-1-carboxylate (17.73 g, 54.00 mmol, 1.1 eq), 3-bromo-1H-1,2,4-triazol-5-amine (Intermediate-4) (8.80 g, 54.0 mmol, 1.1 eq) and H₃PO₄ (4.81 g, 49.1 mmol, 1 eq) in EtOH (50 mL) was stirred at 80 °C for 16 h. The mixture was cooled to room temperature and poured into saturated NaHCO₃ (aq.) solution (200 mL) and extracted with EtOAc (100 mL*2). The combined organic phases were concentrated under reduced pressure. The residue was purified by flash silica gel chromatography (eluent of EtOAc/PE) to afford the title compound.

LCMS: 427.1 [M+H]⁺.

### Intermediate-3: N-(2-chloro-4-(trifluoromethyl)phenyl)-2-iodoacetamide

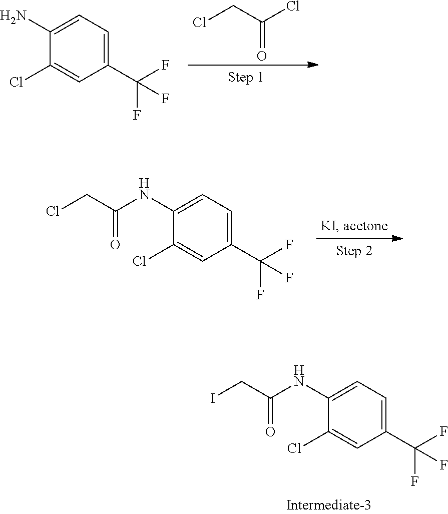 [SMILES: Nc1ccc(C(F)(F)F)cc1Cl ; O=C(Cl)CCl ; O=C(CCl)Nc1ccc(C(F)(F)F)cc1Cl ; O=C(CI)Nc1ccc(C(F)(F)F)cc1Cl]

Step 1. Synthesis of 2-chloro-N-(2-chloro-4-(trifluoromethyl)phenyl)acetamide

To a solution of 2-chloro-4-(trifluoromethyl) aniline (19.1 g, 97.7 mmol, 1.0 eq) and TEA (19.76 g, 195.3 mmol, 2.0 eq) in DCM (200 mL) was added dropwise a solution of 2-chloroacetyl chloride (11.03 g, 97.7 mmol, 1.0 eq) in DCM (50 mL) at 0 °C. After addition, the resulting mixture was warmed to room temperature and stirred at room temperature overnight. The reaction mixture was purified by column chromatography on silica gel (eluent of EtOAc/PE) to afford the title compound.

LCMS: 273.9 [M+H]⁺.

### Step 2. Synthesis of N-(2-chloro-4-(trifluoromethyl)phenyl)-2-iodoacetamide

To a solution of 2-chloro-N-(2-chloro-4-(trifluoromethyl)phenyl)acetamide (7.70 g, 28.3 mmol, 1.0 eq) in acetone (60 mL) was added KI (5.17 g, 31.1 mmol, 1.1 eq), the resulting mixture was stirred at 60 °C for 2 h. The mixture was cooled to room temperature, filtered and the filtrate was concentrated in vacuum to afford the title compound, which was used in the next step without further purification.

LCMS: 363.9 [M+H]⁺.

### Intermediate-4:3-bromo-1H-1,2,4-triazol-5-amine

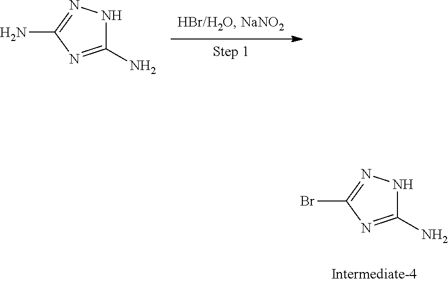 [SMILES: Nc1n[nH]c(N)n1 ; Nc1nc(Br)n[nH]1]

### Step 1. Synthesis of 3-bromo-1H-1,2,4-triazol-5-amine

To a solution of 1H-1,2,4-triazole-3,5-diamine (50.00 g, 504.6 mmol, 1.0 eq) in HBr (aq.) solution (40% in H₂O, 500 mL) was slowly added a NaNO₂ (41.78 g, 605.5 mmol, 1.2 eq) solution in H₂O (125 mL) at 0 °C. After addition, the mixture was warmed to room temperature and stirred at room temperature for 1 h. Then, the reaction temperature was raised to 40°C and stirred at 40 °C for 1 h. Then, the reaction temperature was raised to 60°C and stirred for another 1 h. The reaction mixture was cooled to 0°C and adjusted to pH=2 by 10% NaOH (aq.) solution. The resulting mixture was extracted with EtOAc (1000 mL) and the organic layer was discarded. The aqueous phase was adjusted to pH=7 by 10% NaOH (aq.) solution. The resulting mixture was extracted with EtOAc (1000 mL*3). The combined organic layers were dried over Na₂SO₄, filtered, and concentrated in vacuum to afford the title compound, which was used in next step without further purification.

¹H NMR (400 MHz, DMSO-d₆) δ ppm 12.27 (br s, 1H), 6.29 (br s, 2H).

### Intermediate-5: N-(2-chloro-4-(trifluoromethyl)phenyl)-2-(2-(3,6-dihydro-2H-pyran-4-yl)-5-ethyl-7-oxo-6-(piperazin-1-yl)-[1,2,4]triazolo[1,5-a]pyrimidin-4(7H)-yl)acetamide trifluoroacetate

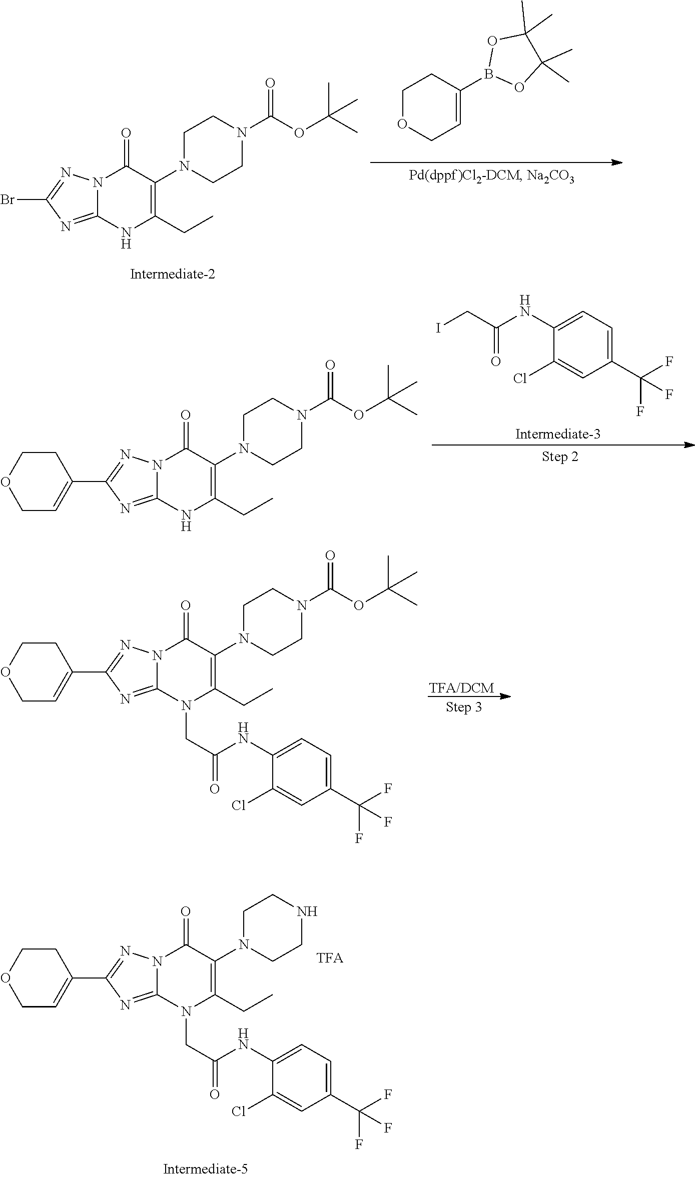

### Step 1. Synthesis of tert-butyl 4-(2-(3,6-dihydro-2H-pyran-4-yl)-5-ethyl-7-oxo-4,7-dihydro-[1,2,4]triazolo[1,5-a]pyrimidin-6-yl)piperazine-1-carboxylate

To a solution of tert-butyl 4-(2-bromo-5-ethyl-7-oxo-4,7-dihydro-[1,2,4]triazolo[1,5-a]pyrimidin-6-yl)piperazine-1-carboxylate (Intermediate-2) (2.00 g, 4.68 mmol, 1.0 eq), 2-(3,6-dihydro-2H-pyran-4-yl)-4,4,5,5-tetramethyl-1,3,2-dioxaborolane (1.47 g, 7.02 mmol, 1.5 eq) and Pd(dppf)Cl₂—CH₂Cl₂ (382 mg, 468 μmol, 0.1 eq) in 1,4-dioxane (20 mL) and H₂O (5 mL) was added Na₂CO₃ (992 mg, 9.36 mmol, 2.0 eq), the resulting mixture was stirred at 100°C overnight under N₂ atmosphere. The reaction mixture was cooled to room temperature, diluted with H₂O (30 mL) and extracted with EtOAc (30 mL*3). The combined organic layer was washed with brine (30 mL), dried over anhydrous Na₂SO₄, filtered, and concentrated in vacuo. The residue was purified by flash silica gel chromatography (eluent of EtOAc/PE) to afford the title compound.

LCMS: 431.2 [M+H]⁺.

### Step 2. Synthesis of tert-butyl 4-(4-(2-((2-chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazine-1-carboxylate

To a solution of tert-butyl 4-(2-(3,6-dihydro-2H-pyran-4-yl)-5-ethyl-7-oxo-4,7-dihydro-[1,2,4]triazolo[1,5-a]pyrimidin-6-yl)piperazine-1-carboxylate (1.00 g, 2.32 mmol, 1.0 eq) and N-(2-chloro-4-(trifluoromethyl)phenyl)-2-iodoacetamide (Intermediate-3) (929 mg, 2.56 mmol, 1.1 eq) in DMF (10 mL) was added DIEA (300 mg, 2.32 mmol, 404 μL, 1.0 eq), the resulting mixture was stirred at 45 °C for 3 h. The reaction mixture was diluted with H₂O (50 mL), extracted with DCM (50 mL*3), dried over Na₂SO₄, filtered and the filtrate was concentrated under reduced pressure. The residue was purified by flash silica gel chromatography (eluent of EtOAc/PE) to afford the title compound.

LCMS: 666.2 [M+H]⁺.

### Step 3. Synthesis of N-(2-chloro-4-(trifluoromethyl)phenyl)-2-(2-(3,6-dihydro-2H-pyran-4-yl)-5-ethyl-7-oxo-6-(piperazin-1-yl)-[1,2,4]triazolo[1,5-a]pyrimidin-4 (7H)-yl)acetamide trifluoroacetate

To a solution of tert-butyl 4-(4-(2-((2-chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazine-1-carboxylate (600 mg, 900 μmol, 1.0 eq) in DCM (6 mL) was added TFA (1.5 mL), the resulting mixture was stirred at room temperature for 0.5 h and then concentrated in vacuum to afford the title compound, which was used in the next step without further purification.

LCMS: 566.2 [M+H]⁺.

### Intermediate-6: N-(2-chloro-4-(trifluoromethyl)phenyl)-2-(5-ethyl-2-(2-methoxypyridin-4-yl)-7-oxo-6-(piperazin-1-yl)-[1,2,4]triazolo[1,5-a]pyrimidin-4(7H)-yl)acetamide hydrochloride

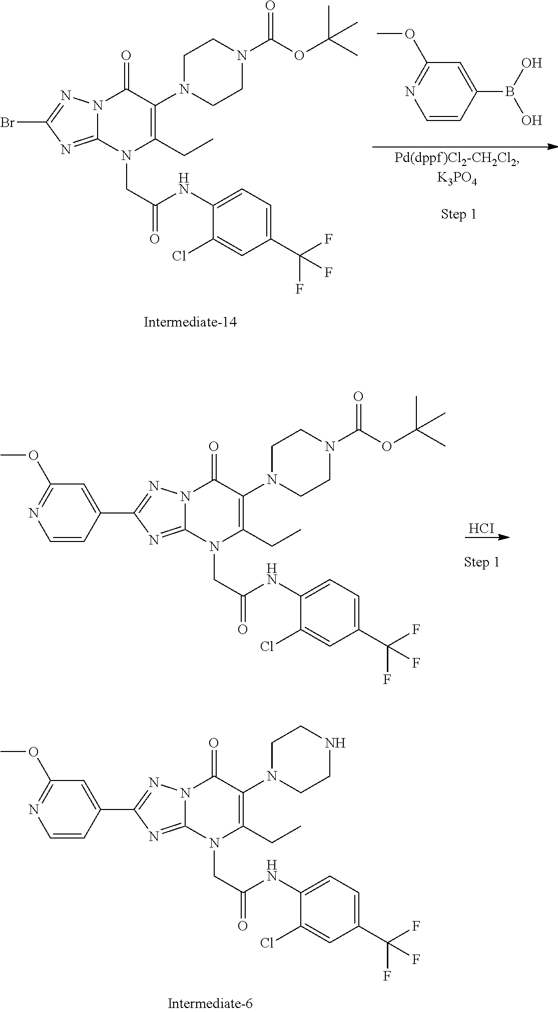

### Step 1: Synthesis of tert-butyl 4-(4-(2-((2-chloro-4-(trifluoromethyl)phenyl)amino)-2-oxoethyl)-5-ethyl-2-(2-methoxypyridin-4-yl)-7-oxo-4,7-dihydro-[1,2,4]triazolo[1,5-a]pyrimidin-6-yl)piperazine-1-carboxylate

To a solution of tert-butyl 4-(2-bromo-4-(2-((2-chloro-4-(trifluoromethyl)phenyl)amino)-2-oxoethyl)-5-ethyl-7-oxo-4,7-dihydro-[1,2,4]triazolo[1,5-a]pyrimidin-6-yl)piperazine-1-carboxylate (Intermediate-14) (1.00 g, 1.51 mmol, 1.0 eq) and (2-methoxypyridin-4-yl) boronic acid (254 mg, 1.66 mmol, 1.1 eq) in 1,4-dioxane (6 mL) and H₂O (2 mL) was added Pd(dppf)Cl₂—CH₂Cl₂ (123 mg, 151 μmol, 0.1 eq) and K₃PO₄ (961 mg, 4.53 mmol, 3.0 eq), the resulting mixture was heated to 100°C overnight under N₂ atmosphere. The reaction mixture was cooled to room temperature, diluted with H₂O (50 mL) and extracted with EtOAc (50 mL*3). The combined organic layers were washed with brine (30 mL), dried over anhydrous Na₂SO₄, filtered and the filtrate was concentrated in vacuo. The residue was purified by silica gel chromatography (EtOAc/PE) to afford the title compound.

LCMS: 691.4 [M+H]⁺.

### Step 2: Synthesis of N-(2-chloro-4-(trifluoromethyl)phenyl)-2-(5-ethyl-2-(2-methoxypyridin-4-yl)-7-oxo-6-(piperazin-1-yl)-[1,2,4]triazolo[1,5-a]pyrimidin-4 (7H)-yl)acetamide hydrochloride

To a solution of tert-butyl 4-(4-(2-((2-chloro-4-(trifluoromethyl)phenyl)amino)-2-oxoethyl)-5-ethyl-2-(2-methoxypyridin-4-yl)-7-oxo-4,7-dihydro-[1,2,4]triazolo[1,5-a]pyrimidin-6-yl)piperazine-1-carboxylate (500 mg, 723 μmol, 1.0 eq) in DCM (10 mL) was added a solution of 4 M HCl in 1,4-dioxane (10 mL), the resulting mixture was stirred at room temperature overnight and then concentrated in vacuum to afford the title compound, which was used in the next step without further purification.

LCMS: 591.2 [M+H]⁺.

### Intermediate-7: N-(2-chloro-4-(trifluoromethyl)phenyl)-2-(2-(2-methoxypyridin-4-yl)-7-oxo-6-(piperidin-4-yl)-[1,2,4]triazolo[1,5-a]pyrimidin-4 (7H)-yl)acetamide hydrochloride

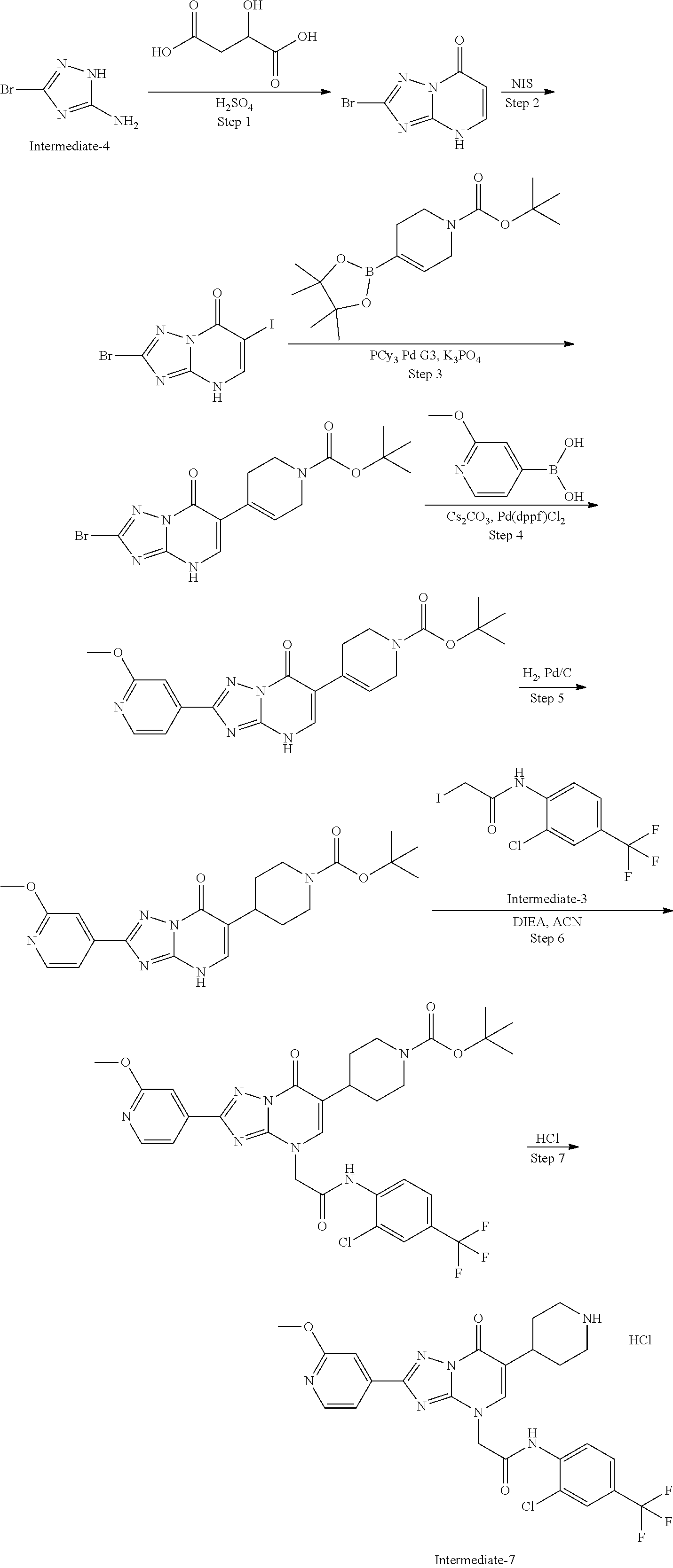

### Step 1. Synthesis of 2-bromo-[1,2,4]triazolo[1,5-a]pyrimidin-7 (4H)-one

To a three necked flask H₂SO₄ (46.00 g, 469.0 mmol, 25.00 mL, 15.3 eq) was cooled to 0 °C. Then 2-hydroxybutanedioic acid (4.32 g, 32.2 mmol, 1.05 eq) and 3-bromo-1H-1,2,4-triazol-5-amine (Intermediate-4) (5.00 g, 30.7 mmol, 1.0 eq) was added at 0°C carefully. The resulting mixture was stirred at room temperature overnight. Then warmed to 100 °C and stirred at 100 °C for 1 h. After cooled down to room temperature, the reaction mixture was filtered and the filter cake was washed with H₂O (50 mL), dried in vacuum to afford the title compound, which was used in the next step without further purification.

¹H NMR (400 MHz, DMSO-d₆) δ ppm 13.47 (d, 1H), 7.98 (d, 1H), 5.98 (d, 1H).

LCMS: 215.1 [M+H]⁺.

### Step 2. Synthesis of 2-bromo-6-iodo-[1,2,4]triazolo[1,5-a]pyrimidin-7 (4H)-one

To a solution of 2-bromo-4H-[1,2,4]triazolo[1,5-a]pyrimidin-7-one (1.00 g, 4.65 mmol, 1.0 eq) in THF (10 mL) was added NIS (1.26 g, 5.58 mmol, 1.20 eq). The resulting mixture was stirred at room temperature for 2 h under N₂ atmosphere. The reaction mixture was poured into H₂O (20 mL) and filtered, the filter cake was dried in vacuum to afford the title compound, which was used in the next step without purification.

¹H NMR (400 MHz, DMSO-d₆) δ ppm 8.51 (s, 1H).

LCMS: 340.9 [M+H]⁺.

### Step 3. Synthesis of tert-butyl 4-(2-bromo-7-oxo-4,7-dihydro-[1,2,4]triazolo[1,5-a]pyrimidin-6-yl)-3,6-dihydropyridine-1 (2H)-carboxylate

To a mixture of 2-bromo-6-iodo-4H-[1,2,4]triazolo[1,5-a]pyrimidin-7-one (800 mg, 2.35 mmol, 1.0 eq), tert-butyl 4-(4,4,5,5-tetramethyl-1,3,2-dioxaborolan-2-yl)-3,6-dihydro-2H-pyridine-1-carboxylate (871 mg, 2.82 mmol, 1.2 eq), K₃PO₄ (1.99 g, 9.39 mmol, 4.0 eq) in butanol (8.0 mL) and H₂O (1.6 mL) was added PCy₃ Pd G3 (172 mg, 235 μmol, 0.1 eq). The resulting mixture was stirred at 70 °C for 2 h under N₂ atmosphere. The reaction mixture was diluted with H₂O (30 mL), then extracted with DCM (30 mL*3). The combined organic layers were dried over anhydrous Na₂SO₄, filtered and the filtrate was concentrated under reduced pressure. The residue was purified by flash silica gel chromatography (eluent of EtOAc/PE) first, followed by reverse phase HPLC (C18 column, H₂O (0.1% TFA)-ACN) to afford the title compound.

¹H NMR (400 MHz, DMSO-d₆) δ ppm 7.91 (s, 1H), 6.39 (br s, 1H), 3.99-3.93 (m, 2H), 3.52-3.46 (m, 2H), 2.44-2.37 (m, 2H), 1.42 (s, 9H).

LCMS: 396.1 [M+H]⁺.

### Step 4. Synthesis of tert-butyl 4-(2-(2-methoxypyridin-4-yl)-7-oxo-4,7-dihydro-[1,2,4]triazolo[1,5-a]pyrimidin-6-yl)-3,6-dihydropyridine-1 (2H)-carboxylate

To a solution of tert-butyl 4-(2-bromo-7-oxo-4,7-dihydro-[1,2,4]triazolo[1,5-a]pyrimidin-6-yl)-3,6-dihydropyridine-1 (2H)-carboxylate (370 mg, 933 μmol, 1.0 eq), (2-methoxypyridin-4-yl)boronic acid (286 mg, 1.87 mmol, 2.0 eq) and Cs₂CO₃ (913 mg, 2.80 mmol, 3.0 eq) in 1,4-dioxane (10 mL) and H₂O (2 mL) was added Pd(dppf)Cl₂ (68 mg, 93 μmol, 0.1 eq). The resulting mixture was stirred at 100°C overnight under N₂ atmosphere. The reaction mixture was filtered through a pad of celite, the filtrate was poured into H₂O (10 mL) and extracted with DCM (30 mL*3). The organic phase was discarded, the aqueous phase was adjusted pH=5 by a 2 M solution of citric acid (aq.). The resulting suspension was filtered, the filter cake was dried in vacuum to afford the title compound, which was used in the next step without further purification.

LCMS: 425.2 [M+H]⁺.

### Step 5. Synthesis of tert-butyl 4-(2-(2-methoxypyridin-4-yl)-7-oxo-4,7-dihydro-[1,2,4]triazolo[1,5-a]pyrimidin-6-yl)piperidine-1-carboxylate

To a solution of tert-butyl 4-(2-(2-methoxypyridin-4-yl)-7-oxo-4,7-dihydro-[1,2,4]triazolo[1,5-a]pyrimidin-6-yl)-3,6-dihydropyridine-1 (2H)-carboxylate (100 mg, 236 μmol, 1.0 eq) in MeOH (3 mL) was added dry Pd/C (500 mg), the suspension was degassed and then purged with H₂ three times. The reaction mixture was stirred under H₂ (50 psi) atmosphere at 60°C overnight, then filtered and concentrated under reduced pressure to afford the title compound, which was used in the next step without further purification.

LCMS: 427.2 [M+H]⁺.

### Step 6. Synthesis of tert-butyl 4-(4-(2-((2-chloro-4-(trifluoromethyl)phenyl)amino)-2-oxoethyl)-2-(2-methoxypyridin-4-yl)-7-oxo-4,7-dihydro-[1,2,4]triazolo[1,5-a]pyrimidin-6-yl)piperidine-1-carboxylate

To a solution of tert-butyl 4-(2-(2-methoxypyridin-4-yl)-7-oxo-4,7-dihydro-[1,2,4]triazolo[1,5-a]pyrimidin-6-yl)piperidine-1-carboxylate (60 mg, 0.14 mmol, 1.0 eq) and N-(2-chloro-4-(trifluoromethyl)phenyl)-2-iodoacetamide (Intermediate-3) (77 mg, 0.21 mmol, 1.5 eq) in ACN (2 mL) was added DIEA (55 mg, 0.42 mmol, 74 μL, 3.0 eq). The resulting mixture was stirred at 60 °C for 1 h and then purified by flash silica gel chromatography (eluent of EtOAc/PE) to afford the title compound.

LCMS: 662.2 [M+H]⁺.

### Step 7. Synthesis of N-(2-chloro-4-(trifluoromethyl)phenyl)-2-(2-(2-methoxypyridin-4-yl)-7-oxo-6-(piperidin-4-yl)-[1,2,4]triazolo[1,5-a]pyrimidin-4 (7H)-yl)acetamide hydrochloride

To a solution of tert-butyl 4-(4-(2-((2-chloro-4-(trifluoromethyl)phenyl)amino)-2-oxoethyl)-2-(2-methoxypyridin-4-yl)-7-oxo-4,7-dihydro-[1,2,4]triazolo[1,5-a]pyrimidin-6-yl) piperidine-1-carboxylate (40 mg, 60 μmol, 1.0 eq) in DCM (1.0 mL) was added a solution of 4 M HCl in 1,4-dioxane (1.0 mL). The resulting mixture was stirred at room temperature for 10 minutes and then concentrated in vacuum to afford the title compound, which was used in the next step without further purification.

LCMS: 562.2 [M+H]⁺.

### Intermediate-8: N-(2-chloro-4-(pentafluoro-26-sulfaneyl)phenyl)-2-iodoacetamide

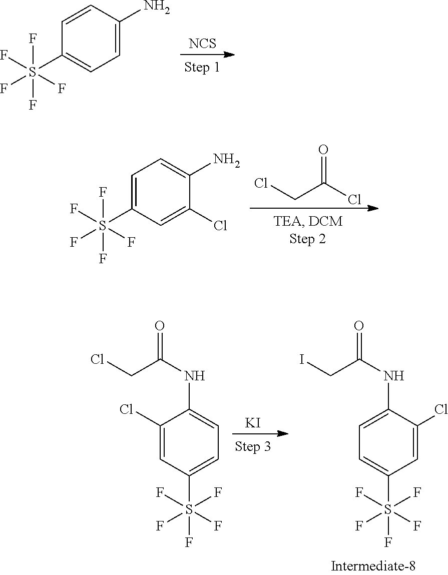

### Step 1. Synthesis of 2-chloro-4-(pentafluoro-λ⁶-sulfaneyl)aniline

To a solution of 4-(pentafluoro-λ⁶-sulfaneyl) aniline (5.00 g, 22.8 mmol, 1.0 eq) in ACN (50 mL) was added NCS (3.35 g, 25.1 mmol, 1.1 eq). The resulting mixture was stirred at 60 °C for 0.5 h and then purified by silica gel chromatography (EtOAc/PE) to afford the tittle compound.

LCMS: 254.2 [M+H]⁺.

### Step 2. Synthesis of 2-chloro-N-(2-chloro-4-(pentafluoro-26-sulfaneyl)phenyl)acetamide

To a solution of 2-chloro-4-(pentafluoro-26-sulfaneyl) aniline (1.00 g, 3.94 mmol, 1.0 eq) in DCM (10 mL) was added 2-chloroacetyl chloride (534 mg, 4.73 mmol, 1.2 eq) and TEA (798 mg, 7.89 mmol, 2.0 eq). The resulting mixture was stirred at room temperature for 2 h. The reaction mixture was diluted with H₂O (20 mL) and extracted with DCM (20 mL*2). The combined organic layers were dried over anhydrous Na₂SO₄, filtered and the filtrate was concentrated under reduced pressure. The residue was purified by silica gel chromatography (EtOAc/PE) to afford the tittle compound.

LCMS: 330.0 [M+H]⁺.

### Step 3. Synthesis of N-(2-chloro-4-(pentafluoro-26-sulfaneyl)phenyl)-2-iodoacetamide

To a solution of 2-chloro-N-(2-chloro-4-(pentafluoro-26-sulfaneyl)phenyl)acetamide (1.00 g, 3.03 mmol, 1.0 eq) in acetone (10 mL) was added KI (835 mg, 5.03 mmol, 1.66 eq). The resulting mixture was stirred at 80 °C for 2 h. The reaction diluted with H₂O (30 mL) and extracted with EtOAc (20 mL*3). The combined organic layers were dried over Na₂SO₄, filtered and the filtrate was concentrated under reduced pressure to afford the title compound, which was used in the next step without further purification.

LCMS: 421.9 [M+H]⁺.

### Intermediate-9: N-(2-chloro-4-(pentafluoro-26-sulfaneyl)phenyl)-2-(5-ethyl-2-(2-methoxypyridin-4-yl)-7-oxo-6-(piperazin-1-yl)-[1,2,4]triazolo[1,5-a]pyrimidin-4 (7H)-yl)acetamide hydrochloride

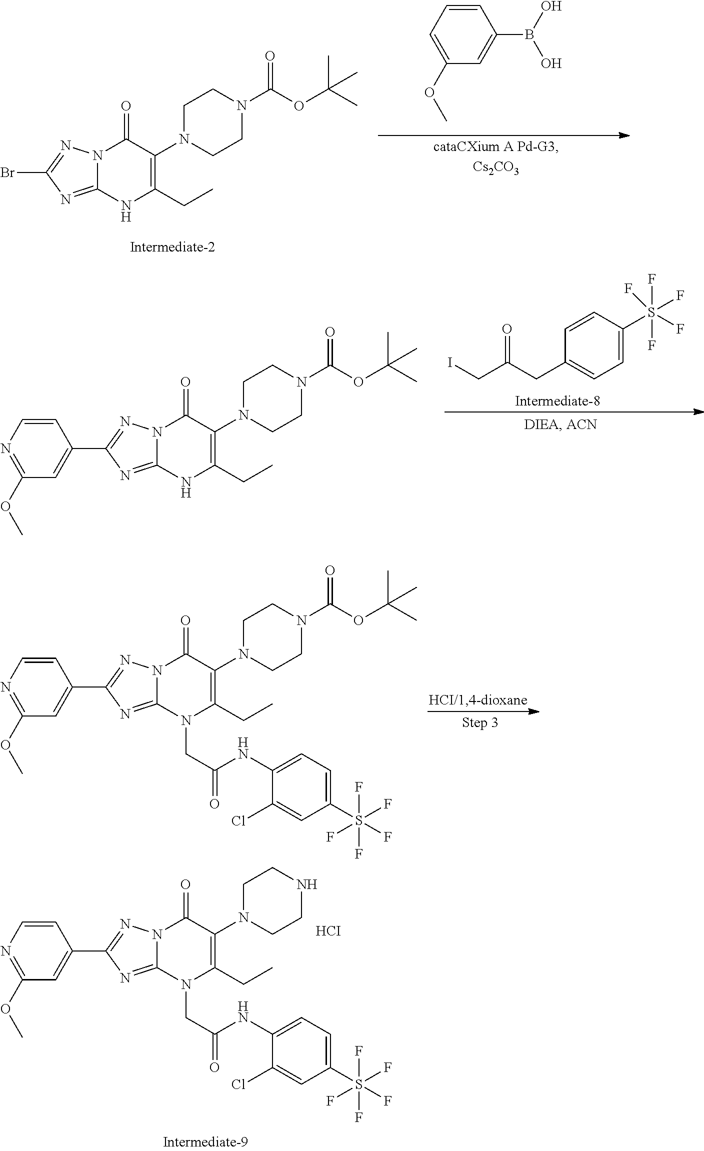

### Step 1. Synthesis of tert-butyl 4-(5-ethyl-2-(2-methoxypyridin-4-yl)-7-oxo-4,7-dihydro-[1,2,4]triazolo[1,5-a]pyrimidin-6-yl)piperazine-1-carboxylate

To a solution of (2-methoxypyridin-4-yl) boronic acid (2.86 g, 18.72 mmol, 4 eq) and tert-butyl 4-(2-bromo-5-ethyl-7-oxo-4,7-dihydro-[1,2,4]triazolo[1,5-a]pyrimidin-6-yl)piperazine-1-carboxylate (Intermediate-2) (2.00 g, 4.68 mmol, 1 eq) in 1,4-dioxane (30 mL) and H₂O (10 mL) was added Cs₂CO₃ (7.63 g, 23.40 mmol, 5 eq) and cataCXium A Pd-G3 (341 mg, 468 μmol, 0.1 eq). The resulting mixture was stirred at 90 °C for 2 h. The reaction mixture was diluted with H₂O (50 mL), then extracted with EtOAc (50 mL*3). The combined organic layers were dried over anhydrous Na₂SO₄, filtered and the filtrate was concentrated under reduced pressure. The residue was purified by silica gel chromatography (EtOAc/PE) to afford the tittle compound.

LCMS: 456.2 [M+H]⁺.

### Step 2. Synthesis of tert-butyl 4-(4-(2-((2-chloro-4-(pentafluoro-26-sulfaneyl)phenyl)amino)-2-oxoethyl)-5-ethyl-2-(2-methoxypyridin-4-yl)-7-oxo-4,7-dihydro-[1,2,4]triazolo[1,5-a]pyrimidin-6-yl)piperazine-1-carboxylate

To a solution of N-(2-chloro-4-(pentafluoro-26-sulfaneyl)phenyl)-2-iodoacetamide (Intermediate-8) (278 mg, 659 μmol, 1.5 eq) and tert-butyl 4-(5-ethyl-2-(2-methoxypyridin-4-yl)-7-oxo-4,7-dihydro-[1,2,4]triazolo[1,5-a]pyrimidin-6-yl)piperazine-1-carboxylate (200 mg, 439 μmol, 1.0 eq) in ACN (3 mL) was added DIEA (227 mg, 1.76 mmol, 4.0 eq). The resulting mixture was stirred at 80 °C for 2 h. The reaction mixture was diluted with H₂O (40 mL), then extracted with EtOAc (30 mL*3). The combined organic layers were dried over anhydrous Na₂SO₄, filtered and the filtrate was concentrated under reduced pressure. The residue was purified by silica gel chromatography (EtOAc/PE) to afford the tittle compound.

LCMS: 749.3 [M+H]⁺.

### Step 3. Synthesis of N-(2-chloro-4-(pentafluoro-26-sulfaneyl)phenyl)-2-(5-ethyl-2-(2-methoxypyridin-4-yl)-7-oxo-6-(piperazin-1-yl)-[1,2,4]triazolo[1,5-a]pyrimidin-4 (7H)-yl)acetamide hydrochloride

To a solution of tert-butyl 4-(4-(2-((2-chloro-4-(pentafluoro-26-sulfaneyl)phenyl)amino)-2-oxoethyl)-5-ethyl-2-(2-methoxypyridin-4-yl)-7-oxo-4,7-dihydro-[1,2,4]triazolo[1,5-a]pyrimidin-6-yl)piperazine-1-carboxylate (60 mg, 80 μmol, 1.0 eq) in DCM (3 mL) was added a solution of 4 M HCl in 1,4-dioxane (3 mL). The resulting mixture was stirred at 25 °C for 2 h and then concentrated under reduced pressure to afford the title compound, which was used in the next step without further purification.

LCMS: 649.3 [M+H]⁺.

### Intermediate-10: (R)—N-(2-chloro-4-(trifluoromethyl)phenyl)-2-(5-ethyl-2-(2-methoxypyridin-4-yl)-6-(3-methylpiperazin-1-yl)-7-oxo-[1,2,4]triazolo[1,5-a]pyrimidin-4(7H)-yl)acetamide hydrochloride

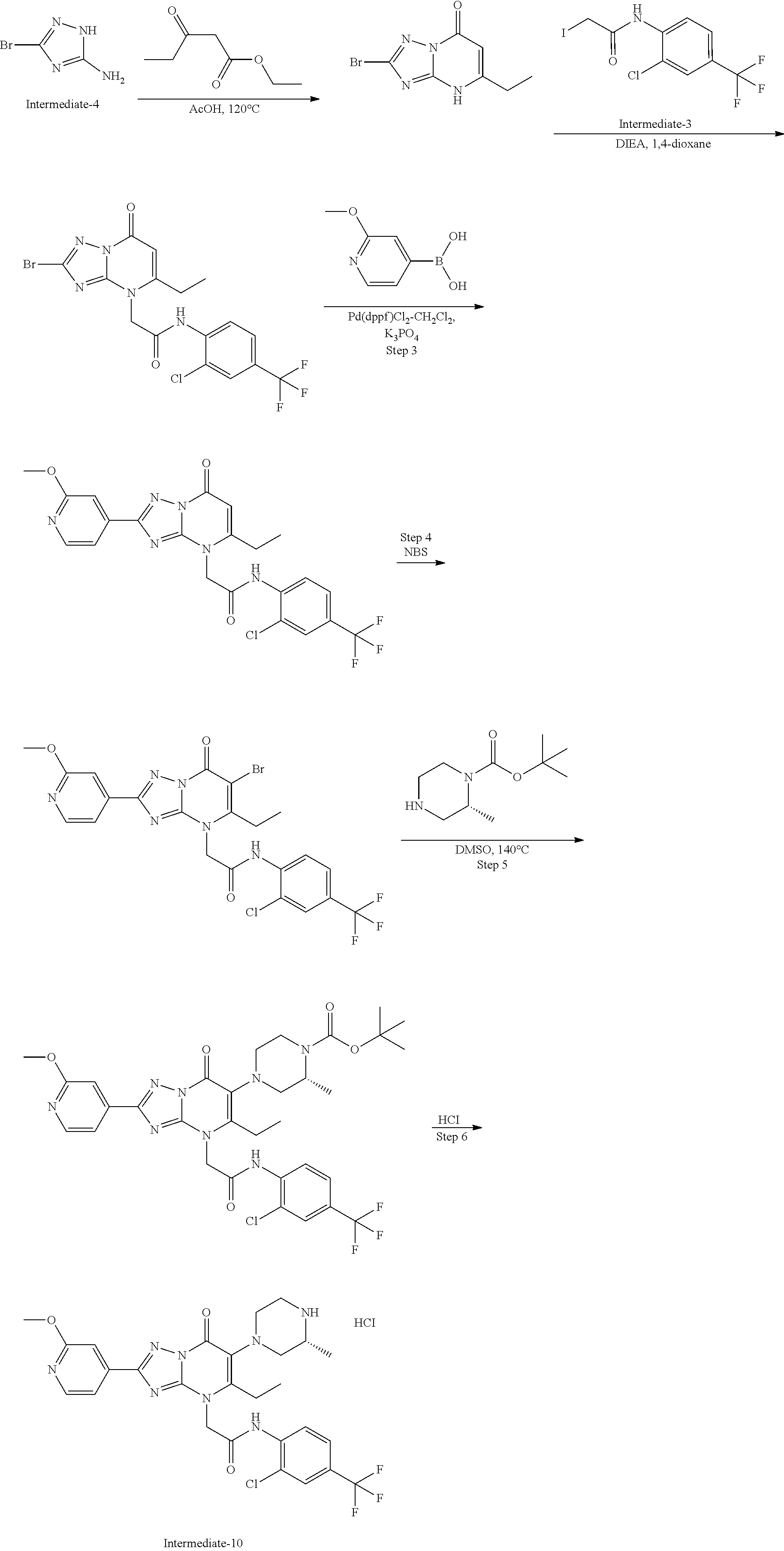

### Step 1: Synthesis of 2-bromo-5-ethyl-[1,2,4]triazolo[1,5-a]pyrimidin-7(4H)-one

To a solution of ethyl 3-oxopentanoate (3.32 g, 23.0 mmol, 1.25 eq) in AcOH (50 mL) was added 3-bromo-1H-1,2,4-triazol-5-amine (Intermediate-4) (3.00 g, 18.4 mmol, 1.0 eq). The resulting mixture was stirred at 120 °C overnight. The reaction mixture was cooled to 0°C and filtered, the filter cake was triturated in EtOH (30 mL) and then filtered. The filter cake was dried in vacuum to afford the title compound.

LCMS: 243.2 [M+H]⁺.

### Step 2: Synthesis of 2-(2-bromo-5-ethyl-7-oxo-[1,2,4]triazolo[1,5-a]pyrimidin-4(7H)-yl)-N-(2-chloro-4-(trifluoromethyl)phenyl)acetamide

To a solution of 2-bromo-5-ethyl-[1,2,4]triazolo[1,5-a]pyrimidin-7 (4H)-one (2.36 g, 9.71 mmol, 1.0 eq) and N-(2-chloro-4-(trifluoromethyl)phenyl)-2-iodoacetamide (Intermediate-3) (3.88 g, 10.7 mmol, 1.1 eq) in 1,4-dioxane (30 mL) was added DIEA (3.76 g, 29.1 mmol, 3.0 eq). The resulting mixture was stirred at 80 °C for 4 h. The reaction mixture was poured into ice H₂O (100 mL) and extracted with EtOAc (100 mL*3). The combined organic phase was washed with brine (20 mL), dried over Na₂SO₄ and concentrated in vacuum to afford the title compound, which was used in the next step without further purification.

LCMS: 478.2 [M+H]⁺.

### Step 3: N-(2-chloro-4-(trifluoromethyl)phenyl)-2-(5-ethyl-2-(2-methoxypyridin-4-yl)-7-oxo-[1,2,4]triazolo[1,5-a]pyrimidin-4(7H)-yl)acetamide

To a solution of 2-(2-bromo-5-ethyl-7-oxo-[1,2,4]triazolo[1,5-a]pyrimidin-4 (7H)-yl)-N-(2-chloro-4-(trifluoromethyl)phenyl)acetamide (2.20 g, 4.60 mmol, 1.0 eq) and (2-methoxypyridin-4-yl)boronic acid (844 mg, 5.52 mmol, 1.2 eq) in 1,4-dioxane (20 mL) and H₂O (10 mL) was added K₃PO₄ (2.93 g, 13.8 mmol, 3.0 eq) and Pd(dppf) Cl₂—CH₂Cl₂ (375 mg, 460 μmol, 0.1 eq). The resulting mixture was stirred at 80 °C under N₂ atmosphere for 1 h. The reaction mixture was cooled and poured into ice-H₂O (50 mL), and then extracted with EtOAc (50 mL*3). The combined organic phase was washed with brine (20 mL), dried over anhydrous Na₂SO₄, filtered, and concentrated in vacuum. The residue was purified by silica gel chromatography (EtOAc/PE) to afford the tittle compound.

LCMS: 507.2 [M+H]⁺.

### Step 4: Synthesis of 2-(6-bromo-5-ethyl-2-(2-methoxypyridin-4-yl)-7-oxo-[1,2,4]triazolo[1,5-a]pyrimidin-4 (7H)-yl)-N-(2-chloro-4-(trifluoromethyl)phenyl)acetamide

To a solution of N-(2-chloro-4-(trifluoromethyl)phenyl)-2-(5-ethyl-2-(2-methoxypyridin-4-yl)-7-oxo-[1,2,4]triazolo[1,5-a]pyrimidin-4 (7H)-yl)acetamide (700 mg, 1.38 mmol, 1.0 eq) in DMF (6 mL) was added NBS (246 mg, 1.38 mmol, 1.0 eq). The resulting mixture was stirred at room temperature for 1 h. The reaction was poured into ice-H₂O (100 mL) and filtered. The filter cake was dried in vacuum to afford the title compound, which was used in the next step without further purification.

LCMS: 585.2 [M+H]⁺.

### Step 5: Synthesis of tert-butyl (R)-4-(4-(2-((2-chloro-4-(trifluoromethyl)phenyl)amino)-2-oxoethyl)-5-ethyl-2-(2-methoxypyridin-4-yl)-7-oxo-4,7-dihydro-[1,2,4]triazolo[1,5-a]pyrimidin-6-yl)-2-methylpiperazine-1-carboxylate

To a solution of 2-(6-bromo-5-ethyl-2-(2-methoxypyridin-4-yl)-7-oxo-[1,2,4]triazolo[1,5-a]pyrimidin-4(7H)-yl)-N-(2-chloro-4-(trifluoromethyl)phenyl)acetamide (200 mg, 341 μmol, 1.0 eq) in DMSO (2 mL) was added tert-butyl (R)-2-methylpiperazine-1-carboxylate (410 mg, 2.05 mmol, 6.0 eq). The resulting mixture was stirred at 140 °C for 3 h.

The reaction mixture was concentrated in vacuum and then purified by reverse phase HPLC (C18 column, H₂O (0.1% FA)-ACN) to afford the title compound.

LCMS: 705.4 [M+H]⁺.

### Step 6: Synthesis of (R)—N-(2-chloro-4-(trifluoromethyl)phenyl)-2-(5-ethyl-2-(2-methoxypyridin-4-yl)-6-(3-methylpiperazin-1-yl)-7-oxo-[1,2,4]triazolo[1,5-a]pyrimidin-4 (7H)-yl)acetamide hydrochloride

To a solution of tert-butyl (R)-4-(4-(2-((2-chloro-4-(trifluoromethyl)phenyl)amino)-2-oxoethyl)-5-ethyl-2-(2-methoxypyridin-4-yl)-7-oxo-4,7-dihydro-[1,2,4]triazolo[1,5-a]pyrimidin-6-yl)-2-methylpiperazine-1-carboxylate (50 mg, 71 μmol, 1.0 eq) in DCM (2 mL) was added a solution of 4 M HCl in 1,4-dioxane (2 mL). The resulting mixture was stirred at room temperature for 1 h and then concentrated in vacuum to afford the title compound, which was used in the next step without further purification.

LCMS: 605.4 [M+H]⁺.

### Intermediate-11:7-hydroxy-2,3-dihydrofuro[3,2-c]pyridine-6-carboxylic acid

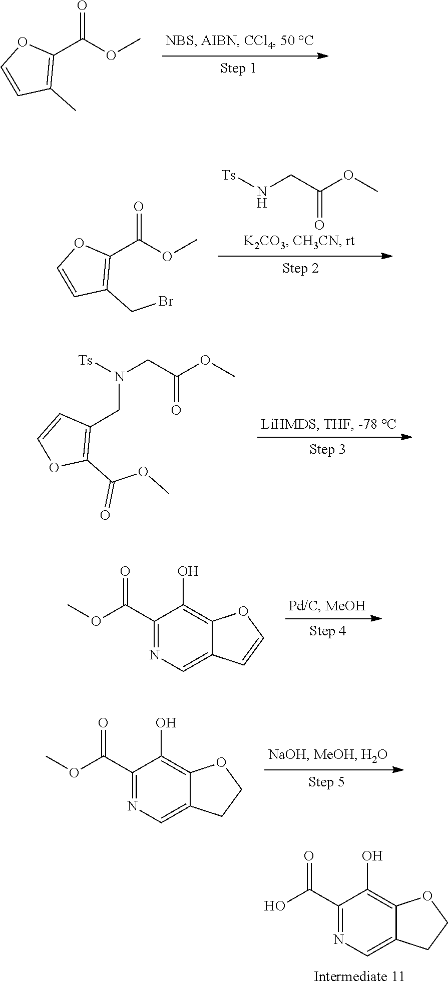

### Step 1. Synthesis of methyl 3-(bromomethyl)furan-2-carboxylate

To a solution of methyl 3-methylfuran-2-carboxylate (5.00 g, 35.7 mmol, 1.00 eq) in CCl₄ (50.0 mL) were added NBS (6.68 g, 37.5 mmol, 1.05 eq) and AIBN (2.35 g, 14.3 mmol, 0.40 eq) at room temperature. The mixture was degassed three times with N₂ and stirred at 50°C for 16 h. The resulting mixture was concentrated under reduced pressure. The residue was purified by flash silica gel chromatography (eluent of EtOAc/PE) to afford the title compound.

LCMS: 219/221 [M+H]⁺.

### Step 2. Synthesis of methyl 3-(((N-(2-methoxy-2-oxoethyl)-4-methylphenyl)sulfonamido)methyl)furan-2-carboxylate

To a solution of methyl 3-(bromomethyl) furan-2-carboxylate (4.70 g, 21.5 mmol, 1.00 eq) and K₂CO₃ (5.93 g, 43.0 mmol, 2.00 eq) in ACN (47.0 mL) was added methyl 2-(4-methylbenzenesulfonamido)acetate (5.23 g, 21.5 mmol, 1.00 eq) and the mixture was stirred at room temperature for 16 h. The reaction was filtered, and the filtrate was concentrated in vacuum. The residue was purified by column chromatography on silica (Eluent of EtOAc/PE) to afford the title compound.

LCMS: 382 [M+H]⁺.

### Step 3. Synthesis of methyl 7-hydroxyfuro[3,2-c]pyridine-6-carboxylate

To a solution of methyl 3-(((N-(2-methoxy-2-oxoethyl)-4-methylphenyl) sulfonamido)methyl) furan-2-carboxylate (1.80 g, 4.72 mmol, 1.00 eq) in THF (18.0 mL) was added a 1 M solution of LiHMDS (14.2 mL, 14.2 mmol, 3.00 eq) in THE dropwise at −78 °C under N₂ atmosphere. After addition, the reaction mixture was allowed to warm to 0 °C and stirred for 5 h under N₂ atmosphere. A saturated NH₄Cl (aq.) solution was added to the reaction mixture and the aq. phase was extracted with EtOAc. The organic layer was dried over anhydrous Na₂SO₄, filtered and concentrated under reduced pressure. The residue was purified by flash silica gel chromatography (Eluent of EtOAc/PE) to afford the title compound.

LCMS: 194 [M+H]⁺.

### Step 4. Synthesis of methyl 7-hydroxy-2,3-dihydrofuro[3,2-c]pyridine-6-carboxylate

To a mixture of methyl 7-hydroxyfuro[3,2-c]pyridine-6-carboxylate (760 mg, 3.93 mmol, 1.00 eq) in MeOH (10.0 mL) was added Pd/C (152 mg, 20%). The mixture was degassed and purged with H₂ gas (40 psi). Then it was stirred at 50 °C for 16 h. The mixture was filtered, and the filtrate was concentrated under reduced pressure. The residue was purified by flash silica gel chromatography (Eluent of DCM/MeOH) to afford the title compound.

LCMS: 196 [M+H]⁺.

### Step 5. Synthesis of 7-hydroxy-2,3-dihydrofuro[3,2-c]pyridine-6-carboxylic acid

To a mixture of methyl 7-hydroxy-2,3-dihydrofuro[3,2-c]pyridine-6-carboxylate (680 mg, 3.48 mmol, 1.00 eq) in H₂O (3.00 mL) and MeOH (3.00 mL) was added NaOH (557 mg, 13.9 mmol, 4.00 eq) at room temperature and the resulting mixture was stirred at 60 °C for 16 h. After completion, the reaction mixture was concentrated under reduced pressure. The residue was diluted with H₂O and acidized by 3 N HCl. The precipitated solids were collected by filtration and dried to afford the title compound, which was used in the next step directly without further purification.

LCMS: 182 [M+H]⁺.

### Intermediate-12:4-hydroxy-2,3-dihydrofuro[2,3-c]pyridine-5-carboxylic acid

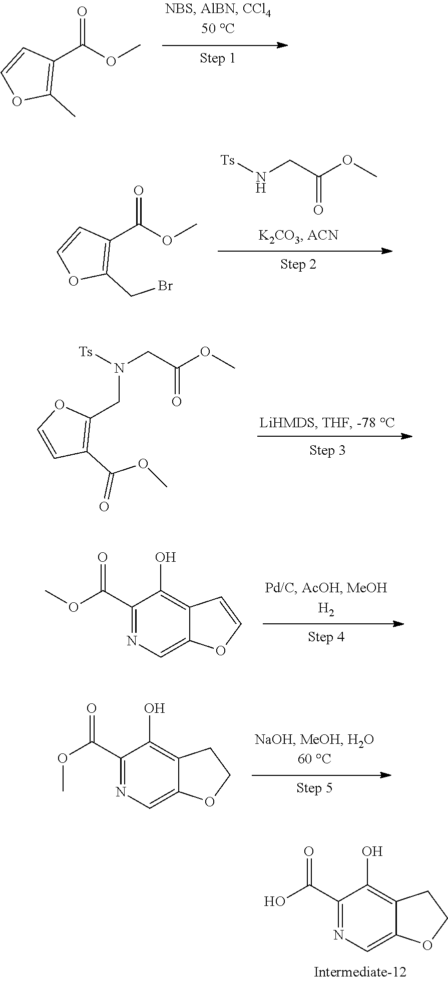

### Step 1. Synthesis of methyl 2-(bromomethyl) furan-3-carboxylate

To a stirred solution of methyl 2-methylfuran-3-carboxylate (10.0 g, 71.4 mmol, 1.00 eq) in CCl₄ (55.0 mL) were added NBS (15.2 g, 85.6 mmol, 1.20 eq) and AIBN (586 mg, 3.57 mmol, 0.05 eq) at room temperature. The resulting mixture was degassed three times with N₂ and stirred overnight at 50 °C under N₂. The reaction mixture was concentrated under reduced pressure. The residue was purified by flash silica gel chromatography (eluent of EtOAc/PE) to afford the title compound.

¹H NMR (400 MHz, DMSO-d₆) δ ppm 7.86 (d, 1H), 6.80 (d, 1H), 4.95 (s, 2H), 3.82 (s, 3H).

### Step 2. Synthesis of methyl 2-(((N-(2-methoxy-2-oxoethyl)-4-methylphenyl)sulfonamido)methyl)furan-3-carboxylate

To a stirred solution of methyl 2-(bromomethyl) furan-3-carboxylate (12.0 g, 54.8 mmol, 1.00 eq) and methyl 2-(4-methylbenzenesulfonamido)acetate (13.3 g, 54.8 mmol, 1.00 eq) in ACN (100 mL) was added K₂CO₃ (15.1 g, 110 mmol, 2.00 eq) at room temperature. The resulting mixture was degassed three times with N₂ and then stirred overnight at room temperature. The reaction mixture was filtered, and the filtrate was concentrated under reduced pressure. The residue was purified by flash silica gel chromatography (eluent of EtOAc/PE) to afford the title compound.

LCMS: 382.1 [M+H]⁺.

### Step 3. Synthesis of methyl 4-hydroxyfuro[2,3-c]pyridine-5-carboxylate

To a stirred solution of methyl 2-([N-(2-methoxy-2-oxoethyl) 4-methylbenzenesulfonamido]methylfuran-3-carboxylate (9.00 g, 23.6 mmol, 1.00 eq) in THF (50.0 mL) was added a 1 M solution of LiHMDS (70.0 mL, 70.0 mmol, 3.00 eq) in THF at −78 °C under N₂. The resulting mixture was stirred for 1 h at room temperature under N₂. The reaction mixture was quenched with saturated NH₄Cl solution at 0 °C and diluted with H₂O (200 mL). The resulting mixture was extracted with EtOAc (2×200 mL). The combined organic layers were washed with brine (3×100 mL), dried over anhydrous Na₂SO₄. After filtration, the filtrate was concentrated under reduced pressure. The residue was purified by flash silica gel chromatography (eluent of EtOAc/PE) to afford a crude product, which was purified by trituration with PE (250 mL) to afford the title compound.

LCMS: 194.0 [M+H]⁺.

### Step 4. Synthesis of methyl 4-hydroxy-2,3-dihydrofuro[2,3-c]pyridine-5-carboxylate

To a solution of methyl 4-hydroxyfuro[2,3-c]pyridine-5-carboxylate (1.00 g, 5.18 mmol, 1.00 eq) in AcOH (1.00 mL) and MeOH (10.0 mL) was added Pd/C (1.65 g, 10%). The mixture was degassed three times with H₂, and then stirred at room temperature for 1 h under H₂. The reaction mixture was filtered, and the filter cake was washed with MeOH. The filtrate was concentrated under reduced pressure and the residue was purified by reverse phase HPLC (C18 column, H₂O (10 mmol/L NH₄HCO₃)-ACN) to afford the title compound.

LCMS: 195.9 [M+H]⁺.

### Step 5. Synthesis of 4-hydroxy-2,3-dihydrofuro[2,3-c]pyridine-5-carboxylic acid

To a stirred solution of methyl 4-hydroxy-2H,3H-furo[2,3-c]pyridine-5-carboxylate (300 mg, 1.54 mmol, 1.00 eq) in MeOH (3.00 mL) were added NaOH (246 mg, 6.15 mmol, 4.00 eq) and H₂O (3.00 mL) at room temperature. The reaction mixture was stirred overnight at 60 °C. The mixture was acidified to pH=3 with 1 N HCl. The resulting mixture was extracted with EtOAc (3×15 mL). The combined organic layers were washed with brine (2×20 mL), dried over anhydrous Na₂SO₄. After filtration, the filtrate was concentrated under reduced pressure to afford the title compound, which was used in the next step directly without further purification.

LCMS: 181.9 [M+H]⁺.

Intermediate-13: Synthesis of 5-hydroxy-6-methylpyrimidine-4-carboxylic acid

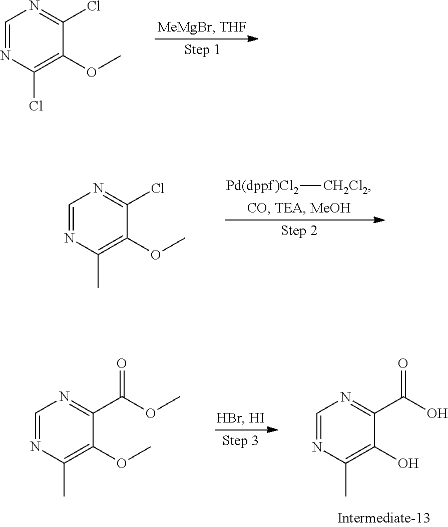

### Step 1: Synthesis of 4-chloro-5-methoxy-6-methylpyrimidine

To a mixture of 4,6-dichloro-5-methoxypyrimidine (30.00 g, 167.6 mmol, 1.0 eq) in THF (300 mL) was added a 3 M solution of MeMgBr (61.45 mL, 184.4 mmol, 1.1 eq) in diethyl ether dropwise at 0 °C and then the mixture was stirred at 5 °C for 1 h. The resulting mixture was poured into H₂O (200 mL) and extracted with EtOAc (100 mL*3). The combined organic phase was washed with brine (100 mL), dried over anhydrous Na₂SO₄, filtered and the filtrate was concentrated in vacuum. The residue was purified by flash silica gel chromatography (eluent of EtOAc/PE) to afford the title compound.

LCMS: 159.1 [M+H]⁺.

### Step 2: Synthesis of methyl 5-methoxy-6-methylpyrimidine-4-carboxylate

To a mixture of 4-chloro-5-methoxy-6-methylpyrimidine (22.00 g, 138.7 mmol, 1.0 eq) in MeOH (250 mL) was added Pd(dppf)Cl₂—CH₂Cl₂ (6.80 g, 8.32 mmol, 0.06 eq) and TEA (28.1 g, 278 mmol, 2.0 eq). The reaction was purged with CO (50 psi) and stirred at 50°C overnight. The resulting mixture was filtered, and the filtrate was concentrated under reduced pressure. The residue was purified by flash silica gel chromatography (eluent of EtOAc/PE) to afford the title compound.

LCMS: 183.1 [M+H]⁺.

### Step 3: Synthesis of 5-hydroxy-6-methylpyrimidine-4-carboxylic acid

A mixture of methyl 5-methoxy-6-methylpyrimidine-4-carboxylate (16.00 g, 87.83 mmol, 1.0 eq) in HBr solution (aq.) (68.5 mL, 68%) was stirred at 50 °C overnight. Then HI solution (aq.) (67.2 mL, 56%) was added and stirred at 50 °C for 6 h. The reaction mixture was cooled to room temperature and basified with 50% NaOH solution (aq.) to pH 9 at 0 °C, then adjusted to pH 7 with 2 M HCl solution (aq.) at 0 °C. The mixture was filtered, the filter cake was dried in vacuum to afford the title compound, which was used in the next step without further purification.

LCMS: 155.1 [M+H]⁺.

¹H NMR (400 MHz, DMSO-d₆) δ ppm 15.96-15.12 (m, 1H), 8.37 (s, 1H), 2.34 (s, 3H).

### Intermediate-14: tert-butyl 4-(2-bromo-4-(2-((2-chloro-4-(trifluoromethyl)phenyl)amino)-2-oxoethyl)-5-ethyl-7-oxo-4,7-dihydro-[1,2,4]triazolo[1,5-a]pyrimidin-6-yl)piperazine-1-carboxylate

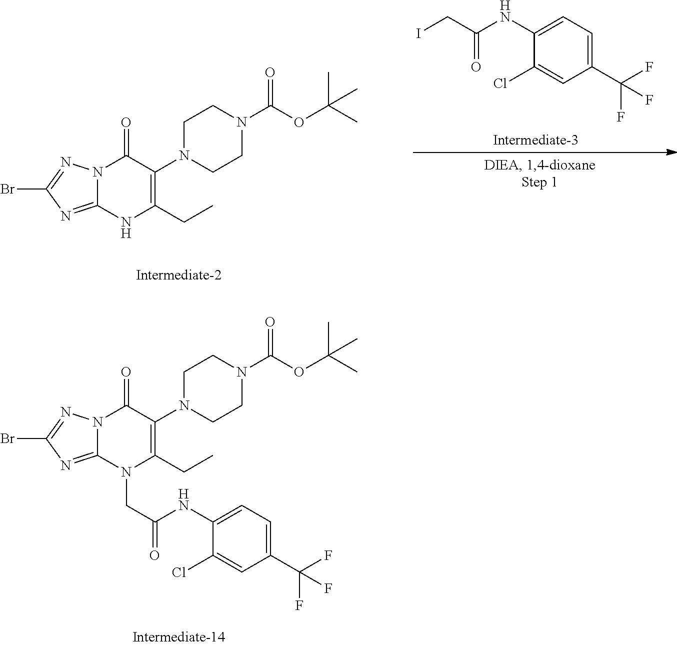 [SMILES: CCc1[nH]c2nc(Br)nn2c(=O)c1N1CCN(C(=O)OC(C)(C)C)CC1 ; O=C(CI)Nc1ccc(C(F)(F)F)cc1Cl ; CCc1c(N2CCN(C(=O)OC(C)(C)C)CC2)c(=O)n2nc(Br)nc2n1CC(=O)Nc1ccc(C(F)(F)F)cc1Cl]

### Step 1: Synthesis of tert-butyl 4-(2-bromo-4-(2-((2-chloro-4-(trifluoromethyl)phenyl)amino)-2-oxoethyl)-5-ethyl-7-oxo-4,7-dihydro-[1,2,4]triazolo[1,5-a]pyrimidin-6-yl)piperazine-1-carboxylate

To a solution of tert-butyl 4-(2-bromo-5-ethyl-7-oxo-4,7-dihydro-[1,2,4]triazolo[1,5-a]pyrimidin-6-yl)piperazine-1-carboxylate (Intermediate-2) (5.00 g, 11.7 mmol, 1.0 eq) and N-(2-chloro-4-(trifluoromethyl)phenyl)-2-iodoacetamide (Intermediate-3) (4.68 g, 12.9 mmol, 1.1 eq) in 1,4-dioxane (50 mL) was added DIEA (4.54 g, 35.1 mmol, 6.11 mL, 3.0 eq), the resulting mixture was stirred at 80 °C for 4 h. The mixture was cooled to room temperature, then diluted with water (50 mL) and extracted with EtOAc (40 mL*3). The combined organic phase was washed with brine (30 mL), dried over anhydrous Na₂SO₄, filtered, and concentrated in vacuo. The residue was purified by silica gel chromatography (EtOAc/PE) to afford the title compound.

LCMS: 684.3 [M+Na]⁺.

The compounds of the disclosure are shown below in Table 2 along with the LCMS method (see below table for method conditions), mass observed, and retention time of compound.

TABLE 2

| | | | (Mass_ | |
|---|---|---|---|---|
| | | | Observed) | (Retention_ |
| No. | | Method | [g/mol] | time) [min] |
| I-1 | 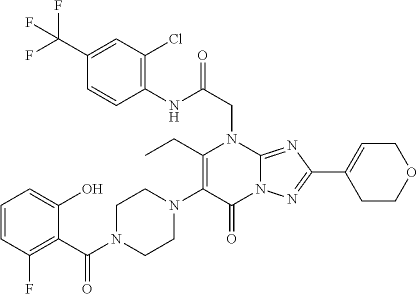 [SMILES: CCc1c(N2CCN(C(=O)c3c(O)cccc3F)CC2)c(=O)n2nc(C3=CCOCC3)nc2n1CC(=O)Nc1ccc(C(F)(F)F)cc1Cl] | LCMS 1 | 704.1 | 0.59 |
| I-2 | 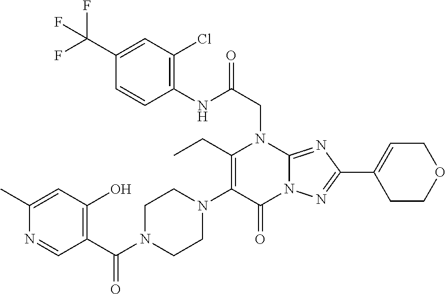 [SMILES: CCc1c(N2CCN(C(=O)c3cnc(C)cc3O)CC2)c(=O)n2nc(C3=CCOCC3)nc2n1CC(=O)Nc1ccc(C(F)(F)F)cc1Cl] | LCMS 1 | 701.1 | 0.53 |
| I-3 | 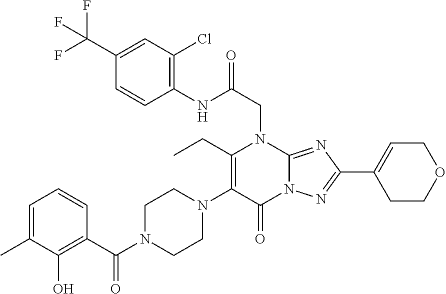 [SMILES: CCc1c(N2CCN(C(=O)c3cccc(C)c3O)CC2)c(=O)n2nc(C3=CCOCC3)nc2n1CC(=O)Nc1ccc(C(F)(F)F)cc1Cl] | LCMS 1 | 700.4 | 0.65 |
| I-4 | 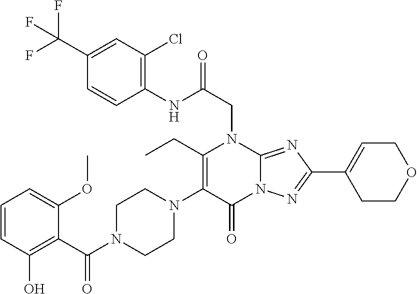 [SMILES: CCc1c(N2CCN(C(=O)c3c(O)cccc3OC)CC2)c(=O)n2nc(C3=CCOCC3)nc2n1CC(=O)Nc1ccc(C(F)(F)F)cc1Cl] | LCMS 1 | 716.4 | 0.61 |
| I-5 | 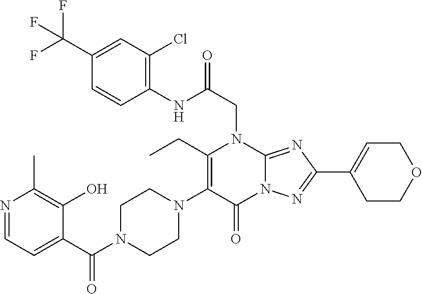 [SMILES: CCc1c(N2CCN(C(=O)c3ccnc(C)c3O)CC2)c(=O)n2nc(C3=CCOCC3)nc2n1CC(=O)Nc1ccc(C(F)(F)F)cc1Cl] | LCMS 2 | 701.1 | 0.63 |
| I-6 | 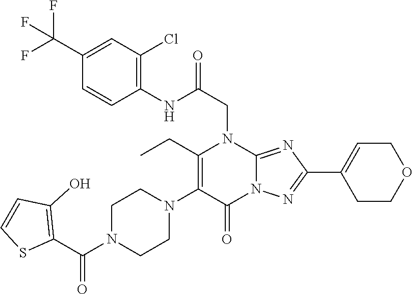 [SMILES: CCc1c(N2CCN(C(=O)c3sccc3O)CC2)c(=O)n2nc(C3=CCOCC3)nc2n1CC(=O)Nc1ccc(C(F)(F)F)cc1Cl] | LCMS 1 | 692.3 | 0.66 |
| I-7 | 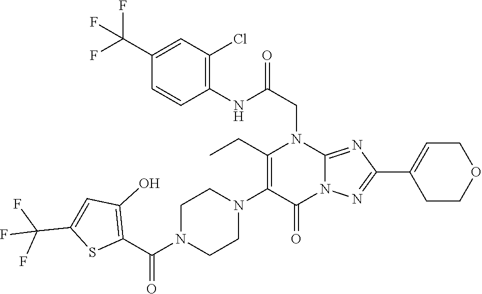 [SMILES: CCc1c(N2CCN(C(=O)c3sc(C(F)(F)F)cc3O)CC2)c(=O)n2nc(C3=CCOCC3)nc2n1CC(=O)Nc1ccc(C(F)(F)F)cc1Cl] | LCMS 2 | 760 | 0.95 |
| I-8 | 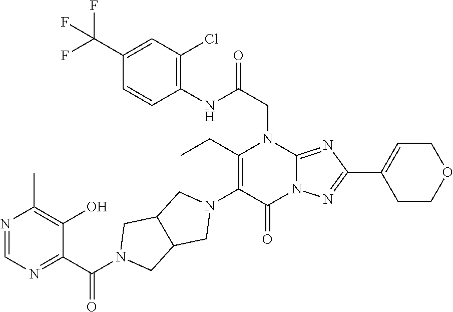 [SMILES: CCc1c(N2CC3CN(C(=O)c4ncnc(C)c4O)CC3C2)c(=O)n2nc(C3=CCOCC3)nc2n1CC(=O)Nc1ccc(C(F)(F)F)cc1Cl] | LCMS 1 | 728.5 | 0.66 |
| I-9 | 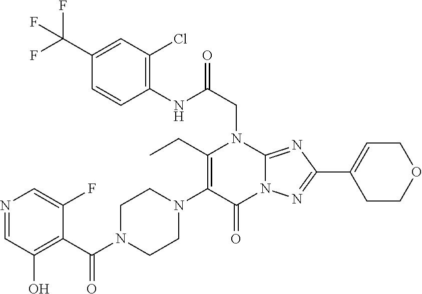 [SMILES: CCc1c(N2CCN(C(=O)c3c(O)cncc3F)CC2)c(=O)n2nc(C3=CCOCC3)nc2n1CC(=O)Nc1ccc(C(F)(F)F)cc1Cl] | LCMS 1 | 705.2 | 0.56 |
| I-10 | 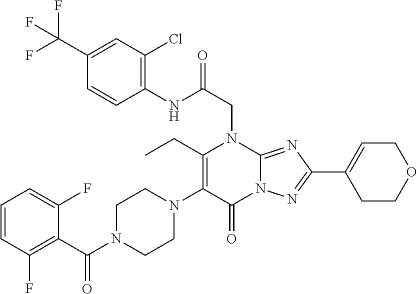 [SMILES: CCc1c(N2CCN(C(=O)c3c(F)cccc3F)CC2)c(=O)n2nc(C3=CCOCC3)nc2n1CC(=O)Nc1ccc(C(F)(F)F)cc1Cl] | LCMS 1 | 706.3 | 0.58 |
| I-11 | 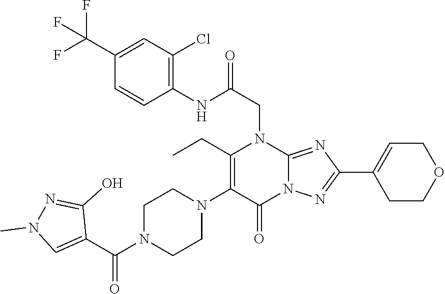 [SMILES: CCc1c(N2CCN(C(=O)c3cn(C)nc3O)CC2)c(=O)n2nc(C3=CCOCC3)nc2n1CC(=O)Nc1ccc(C(F)(F)F)cc1Cl] | LCMS 1 | 690.3 | 0.53 |
| I-12 | 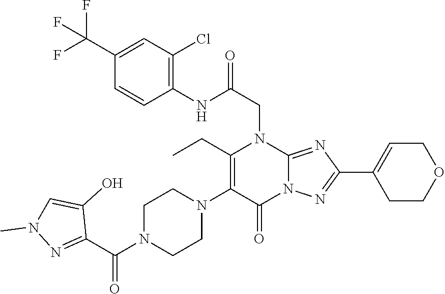 [SMILES: CCc1c(N2CCN(C(=O)c3nn(C)cc3O)CC2)c(=O)n2nc(C3=CCOCC3)nc2n1CC(=O)Nc1ccc(C(F)(F)F)cc1Cl] | LCMS 1 | 690.3 | 0.62 |
| I-13 | 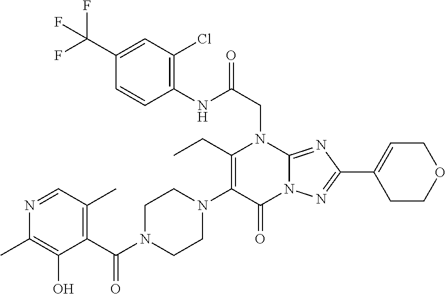 [SMILES: CCc1c(N2CCN(C(=O)c3c(C)cnc(C)c3O)CC2)c(=O)n2nc(C3=CCOCC3)nc2n1CC(=O)Nc1ccc(C(F)(F)F)cc1Cl] | LCMS 1 | 715.3 | 0.55 |
| I-14 | 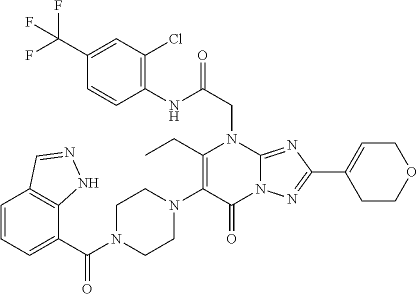 [SMILES: CCc1c(N2CCN(C(=O)c3cccc4cn[nH]c34)CC2)c(=O)n2nc(C3=CCOCC3)nc2n1CC(=O)Nc1ccc(C(F)(F)F)cc1Cl] | LCMS 1 | 710.3 | 0.62 |
| I-15 | 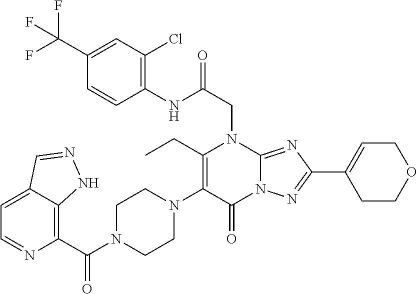 [SMILES: CCc1c(N2CCN(C(=O)c3nccc4cn[nH]c34)CC2)c(=O)n2nc(C3=CCOCC3)nc2n1CC(=O)Nc1ccc(C(F)(F)F)cc1Cl] | LCMS 1 | 711.1 | 0.57 |
| I-16 | 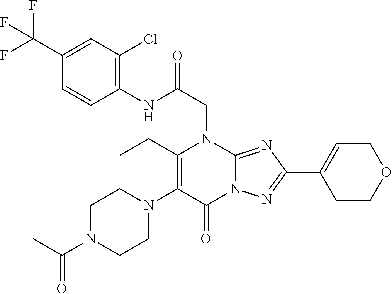 [SMILES: CCc1c(N2CCN(C(C)=O)CC2)c(=O)n2nc(C3=CCOCC3)nc2n1CC(=O)Nc1ccc(C(F)(F)F)cc1Cl] | LCMS 1 | 608.2 | 0.55 |
| I-17 | 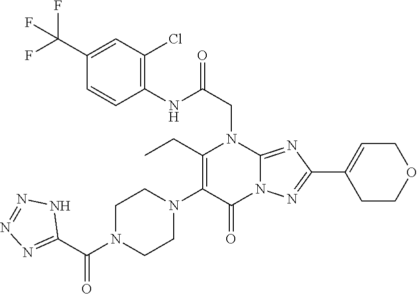 [SMILES: CCc1c(N2CCN(C(=O)c3nnn[nH]3)CC2)c(=O)n2nc(C3=CCOCC3)nc2n1CC(=O)Nc1ccc(C(F)(F)F)cc1Cl] | LCMS 1 | 662.2 | 0.56 |
| I-18 | 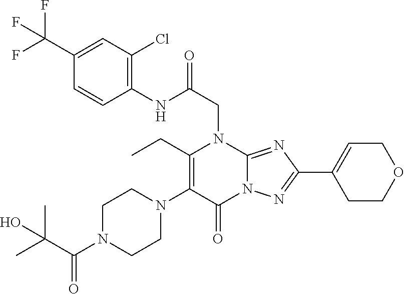 [SMILES: CCc1c(N2CCN(C(=O)C(C)(C)O)CC2)c(=O)n2nc(C3=CCOCC3)nc2n1CC(=O)Nc1ccc(C(F)(F)F)cc1Cl] | LCMS 1 | 652.4 | 0.6 |
| I-19 | 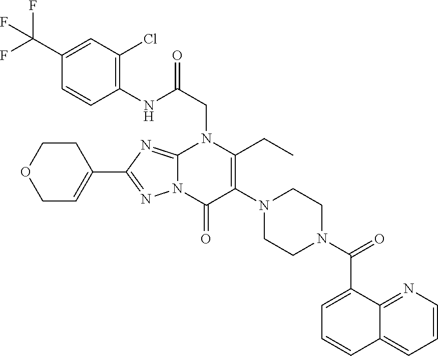 [SMILES: CCc1c(N2CCN(C(=O)c3cccc4cccnc34)CC2)c(=O)n2nc(C3=CCOCC3)nc2n1CC(=O)Nc1ccc(C(F)(F)F)cc1Cl] | LCMS 1 | 721.4 | 0.61 |
| I-20 | 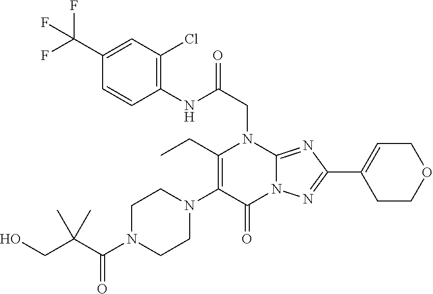 [SMILES: CCc1c(N2CCN(C(=O)C(C)(C)CO)CC2)c(=O)n2nc(C3=CCOCC3)nc2n1CC(=O)Nc1ccc(C(F)(F)F)cc1Cl] | LCMS 2 | 666.4 | 0.75 |
| I-21 | 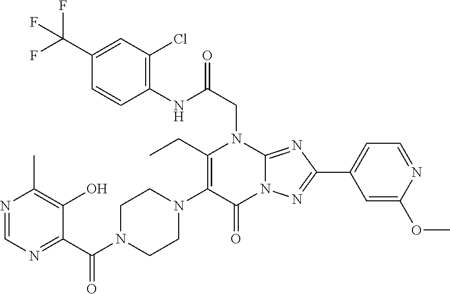 [SMILES: CCc1c(N2CCN(C(=O)c3ncnc(C)c3O)CC2)c(=O)n2nc(-c3ccnc(OC)c3)nc2n1CC(=O)Nc1ccc(C(F)(F)F)cc1Cl] | LCMS 1 | 727.1 | 0.59 |
| I-22 | 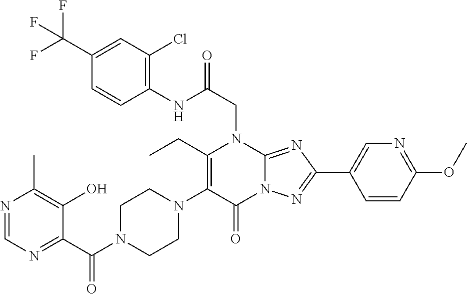 [SMILES: CCc1c(N2CCN(C(=O)c3ncnc(C)c3O)CC2)c(=O)n2nc(-c3ccc(OC)nc3)nc2n1CC(=O)Nc1ccc(C(F)(F)F)cc1Cl] | LCMS 2 | 727.4 | 0.8 |
| I-23 | 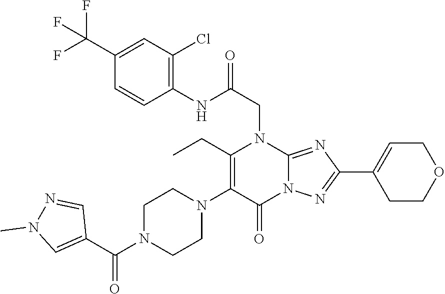 [SMILES: CCc1c(N2CCN(C(=O)c3cnn(C)c3)CC2)c(=O)n2nc(C3=CCOCC3)nc2n1CC(=O)Nc1ccc(C(F)(F)F)cc1Cl] | LCMS 2 | 674.4 | 0.73 |
| I-24 | 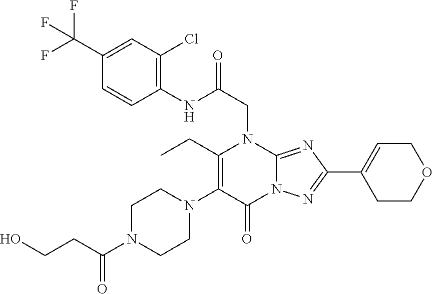 [SMILES: CCc1c(N2CCN(C(=O)CCO)CC2)c(=O)n2nc(C3=CCOCC3)nc2n1CC(=O)Nc1ccc(C(F)(F)F)cc1Cl] | LCMS 2 | 638.3 | 0.72 |
| I-25 | 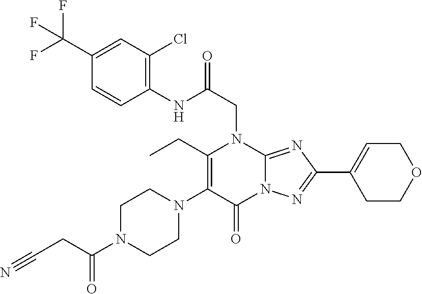 [SMILES: CCc1c(N2CCN(C(=O)CC#N)CC2)c(=O)n2nc(C3=CCOCC3)nc2n1CC(=O)Nc1ccc(C(F)(F)F)cc1Cl] | LCMS 2 | 633.4 | 0.73 |
| I-26 | 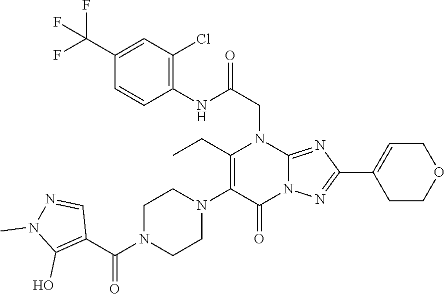 [SMILES: CCc1c(N2CCN(C(=O)c3cnn(C)c3O)CC2)c(=O)n2nc(C3=CCOCC3)nc2n1CC(=O)Nc1ccc(C(F)(F)F)cc1Cl] | LCMS 3 | 690.4 | 1.3 |
| I-27 | 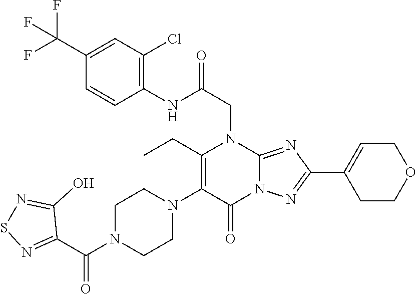 [SMILES: CCc1c(N2CCN(C(=O)c3nsnc3O)CC2)c(=O)n2nc(C3=CCOCC3)nc2n1CC(=O)Nc1ccc(C(F)(F)F)cc1Cl] | LCMS 3 | 694.2 | 1.43 |
| I-28 | 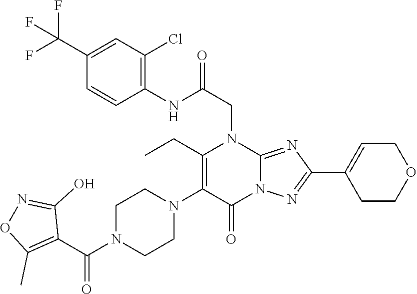 [SMILES: CCc1c(N2CCN(C(=O)c3c(O)noc3C)CC2)c(=O)n2nc(C3=CCOCC3)nc2n1CC(=O)Nc1ccc(C(F)(F)F)cc1Cl] | LCMS 3 | 691.2 | 1.31 |
| I-29 | 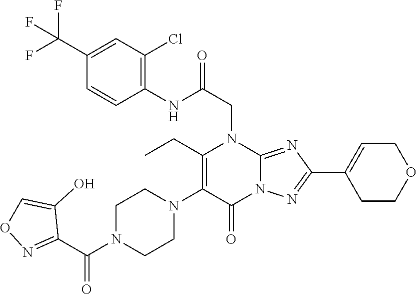 [SMILES: CCc1c(N2CCN(C(=O)c3nocc3O)CC2)c(=O)n2nc(C3=CCOCC3)nc2n1CC(=O)Nc1ccc(C(F)(F)F)cc1Cl] | LCMS 3 | 677.2 | 1.41 |
| I-30 | 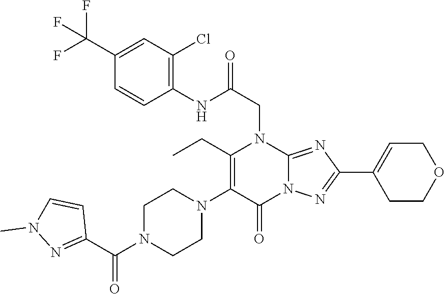 [SMILES: CCc1c(N2CCN(C(=O)c3ccn(C)n3)CC2)c(=O)n2nc(C3=CCOCC3)nc2n1CC(=O)Nc1ccc(C(F)(F)F)cc1Cl] | LCMS 2 | 674.4 | 0.74 |
| I-31 | 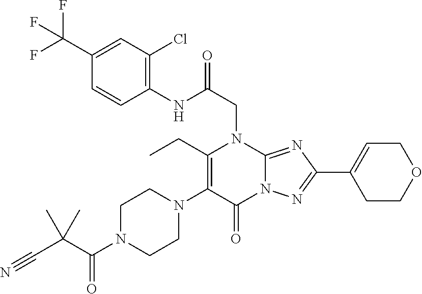 [SMILES: CCc1c(N2CCN(C(=O)C(C)(C)C#N)CC2)c(=O)n2nc(C3=CCOCC3)nc2n1CC(=O)Nc1ccc(C(F)(F)F)cc1Cl] | LCMS 2 | 661.4 | 0.81 |
| I-32 | 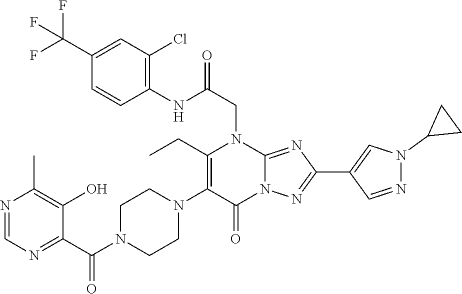 [SMILES: CCc1c(N2CCN(C(=O)c3ncnc(C)c3O)CC2)c(=O)n2nc(-c3cnn(C4CC4)c3)nc2n1CC(=O)Nc1ccc(C(F)(F)F)cc1Cl] | LCMS 1 | 726.2 | 0.57 |
| I-33 | 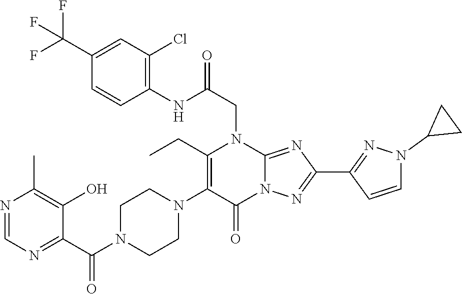 [SMILES: CCc1c(N2CCN(C(=O)c3ncnc(C)c3O)CC2)c(=O)n2nc(-c3ccn(C4CC4)n3)nc2n1CC(=O)Nc1ccc(C(F)(F)F)cc1Cl] | LCMS 1 | 726.2 | 0.58 |
| I-34 | 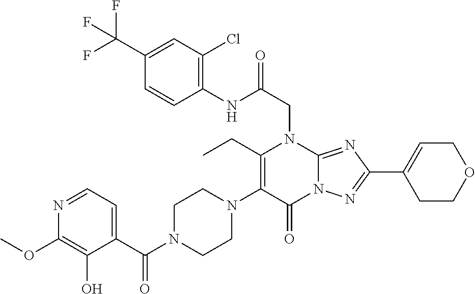 [SMILES: CCc1c(N2CCN(C(=O)c3ccnc(OC)c3O)CC2)c(=O)n2nc(C3=CCOCC3)nc2n1CC(=O)Nc1ccc(C(F)(F)F)cc1Cl] | LCMS 1 | 717.4 | 0.59 |
| I-35 | 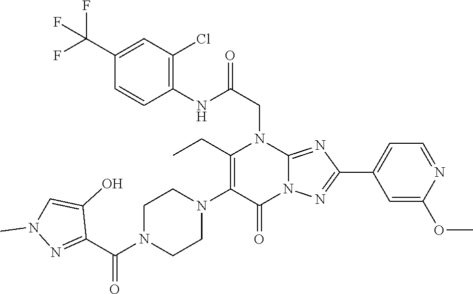 | LCMS 1 | 715.1 | 0.66 |
| I-36 | 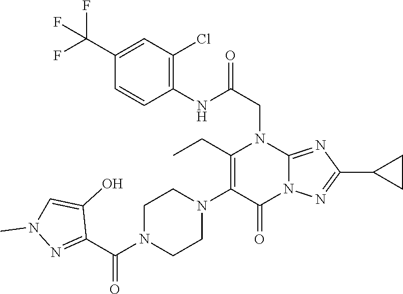 [SMILES: CCc1c(N2CCN(C(=O)c3nn(C)cc3O)CC2)c(=O)n2nc(C3CC3)nc2n1CC(=O)Nc1ccc(C(F)(F)F)cc1Cl] | LCMS 1 | 648.4 | 0.62 |
| I-37 | 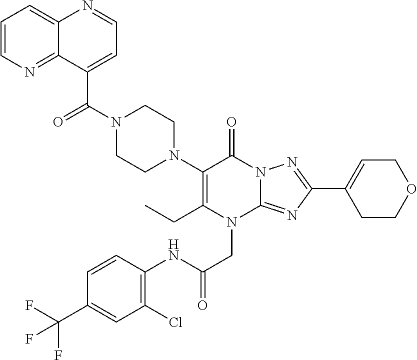 [SMILES: CCc1c(N2CCN(C(=O)c3ccnc4cccnc34)CC2)c(=O)n2nc(C3=CCOCC3)nc2n1CC(=O)Nc1ccc(C(F)(F)F)cc1Cl] | LCMS 1 | 722.2 | 0.57 |
| I-38 | 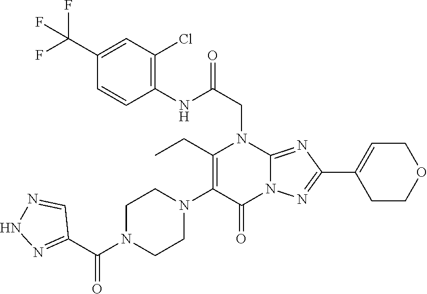 [SMILES: CCc1c(N2CCN(C(=O)c3cn[nH]n3)CC2)c(=O)n2nc(C3=CCOCC3)nc2n1CC(=O)Nc1ccc(C(F)(F)F)cc1Cl] | LCMS 3 | 661.2 | 1.14 |
| I-39 | 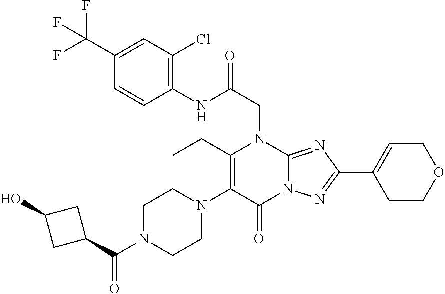 [SMILES: CCc1c(N2CCN(C(=O)[C@H]3C[C@@H](O)C3)CC2)c(=O)n2nc(C3=CCOCC3)nc2n1CC(=O)Nc1ccc(C(F)(F)F)cc1Cl] | LCMS 3 | 664.2 | 1.14 |
| I-40 | 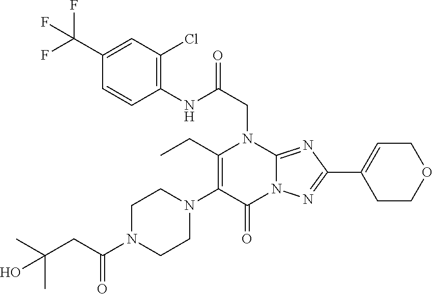 [SMILES: CCc1c(N2CCN(C(=O)CC(C)(C)O)CC2)c(=O)n2nc(C3=CCOCC3)nc2n1CC(=O)Nc1ccc(C(F)(F)F)cc1Cl] | LCMS 3 | 664.4 | 1.09 |
| I-41 | 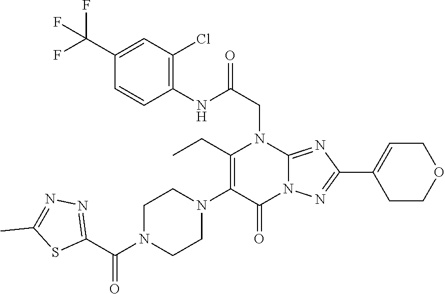 [SMILES: CCc1c(N2CCN(C(=O)c3nnc(C)s3)CC2)c(=O)n2nc(C3=CCOCC3)nc2n1CC(=O)Nc1ccc(C(F)(F)F)cc1Cl] | LCMS 3 | 692.2 | 1.38 |
| I-42 | 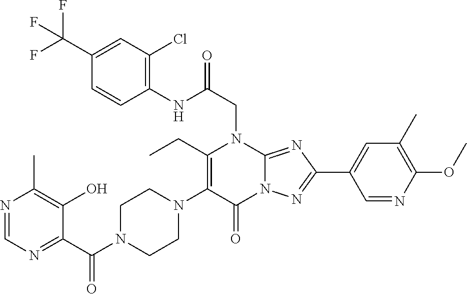 [SMILES: CCc1c(N2CCN(C(=O)c3ncnc(C)c3O)CC2)c(=O)n2nc(-c3cnc(OC)c(C)c3)nc2n1CC(=O)Nc1ccc(C(F)(F)F)cc1Cl] | LCMS 1 | 741.5 | 0.66 |
| I-43 | 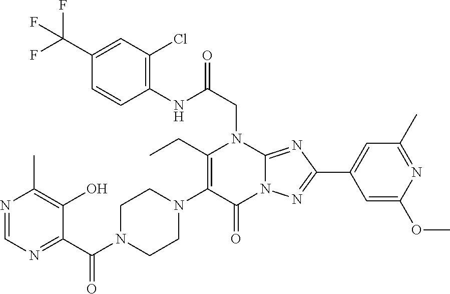 [SMILES: CCc1c(N2CCN(C(=O)c3ncnc(C)c3O)CC2)c(=O)n2nc(-c3cc(C)nc(OC)c3)nc2n1CC(=O)Nc1ccc(C(F)(F)F)cc1Cl] | LCMS 1 | 741.2 | 0.6 |
| I-44 | 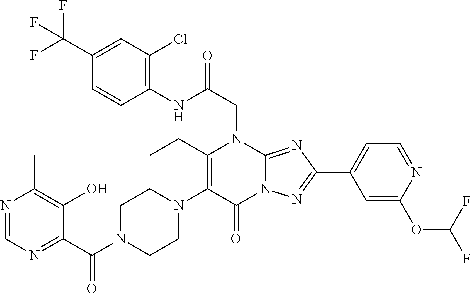 [SMILES: CCc1c(N2CCN(C(=O)c3ncnc(C)c3O)CC2)c(=O)n2nc(-c3ccnc(OC(F)F)c3)nc2n1CC(=O)Nc1ccc(C(F)(F)F)cc1Cl] | LCMS 1 | 763.1 | 0.62 |
| I-45 | 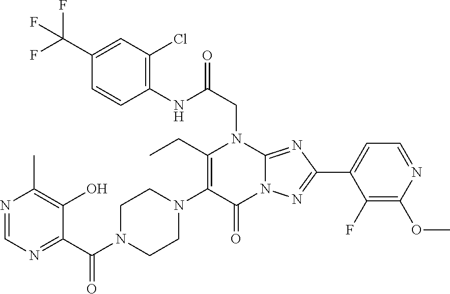 [SMILES: CCc1c(N2CCN(C(=O)c3ncnc(C)c3O)CC2)c(=O)n2nc(-c3ccnc(OC)c3F)nc2n1CC(=O)Nc1ccc(C(F)(F)F)cc1Cl] | LCMS 1 | 745.1 | 0.59 |
| I-46 | 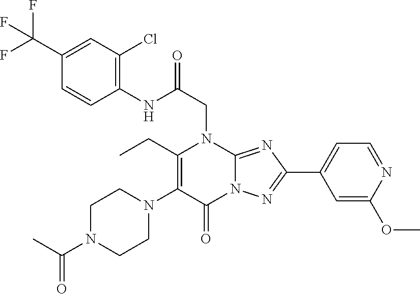 [SMILES: CCc1c(N2CCN(C(C)=O)CC2)c(=O)n2nc(-c3ccnc(OC)c3)nc2n1CC(=O)Nc1ccc(C(F)(F)F)cc1Cl] | LCMS 1 | 633.2 | 0.58 |
| I-47 | 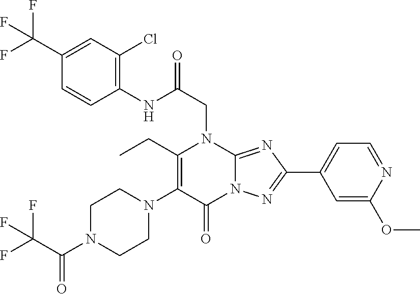 [SMILES: CCc1c(N2CCN(C(=O)C(F)(F)F)CC2)c(=O)n2nc(-c3ccnc(OC)c3)nc2n1CC(=O)Nc1ccc(C(F)(F)F)cc1Cl] | LCMS 1 | 687.1 | 0.63 |
| I-48 | 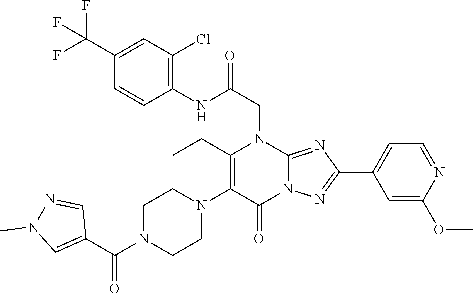 | LCMS 1 | 699.1 | 0.58 |
| I-49 | 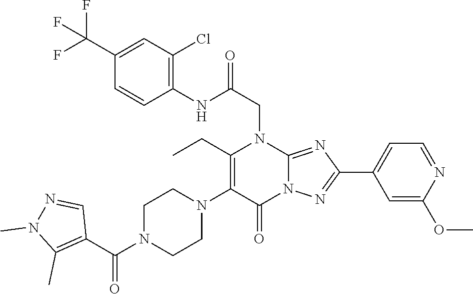 | LCMS 1 | 713.1 | 0.58 |
| I-50 | 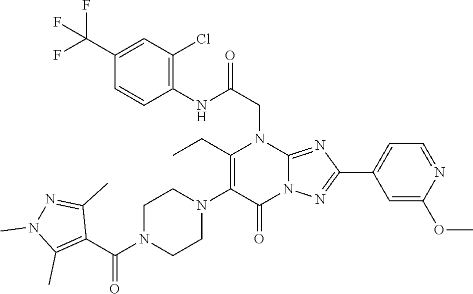 | LCMS 1 | 727.2 | 0.57 |
| I-51 | 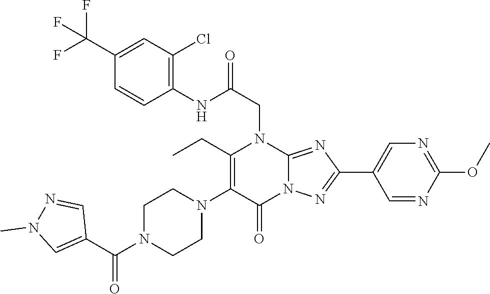 | LCMS 1 | 700.4 | 0.58 |
| I-52 | 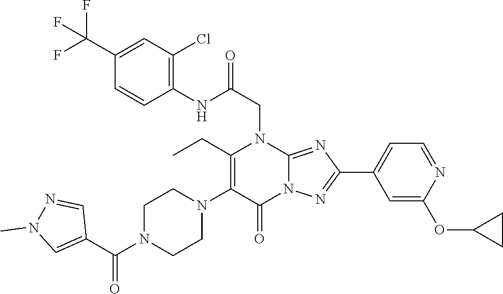 [SMILES: CCc1c(N2CCN(C(=O)c3cnn(C)c3)CC2)c(=O)n2nc(-c3ccnc(OC4CC4)c3)nc2n1CC(=O)Nc1ccc(C(F)(F)F)cc1Cl] | LCMS 1 | 725.4 | 0.61 |
| I-53 | 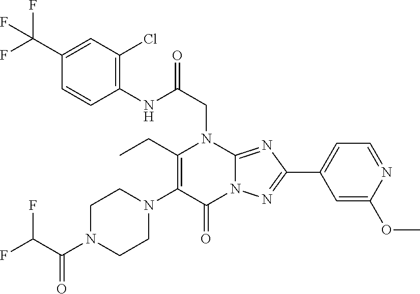 [SMILES: CCc1c(N2CCN(C(=O)C(F)F)CC2)c(=O)n2nc(-c3ccnc(OC)c3)nc2n1CC(=O)Nc1ccc(C(F)(F)F)cc1Cl] | LCMS 1 | 669.4 | 0.63 |
| I-54 | 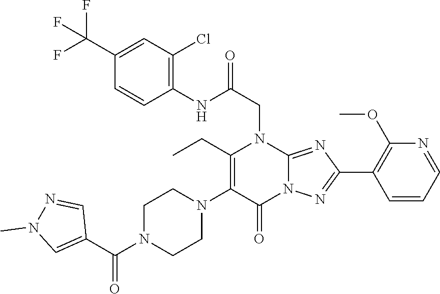 [SMILES: CCc1c(N2CCN(C(=O)c3cnn(C)c3)CC2)c(=O)n2nc(-c3cccnc3OC)nc2n1CC(=O)Nc1ccc(C(F)(F)F)cc1Cl] | LCMS 1 | 699.2 | 0.57 |
| I-55 | 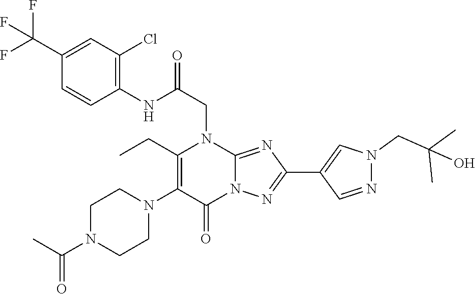 [SMILES: CCc1c(N2CCN(C(C)=O)CC2)c(=O)n2nc(-c3cnn(CC(C)(C)O)c3)nc2n1CC(=O)Nc1ccc(C(F)(F)F)cc1Cl] | LCMS 1 | 664.2 | 0.53 |
| I-56 | 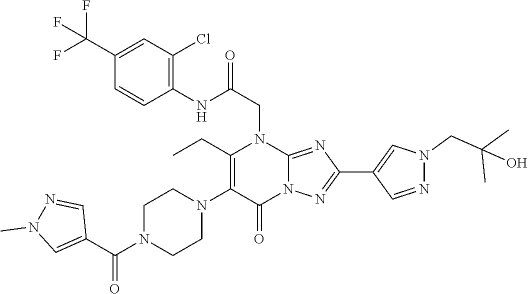 [SMILES: CCc1c(N2CCN(C(=O)c3cnn(C)c3)CC2)c(=O)n2nc(-c3cnn(CC(C)(C)O)c3)nc2n1CC(=O)Nc1ccc(C(F)(F)F)cc1Cl] | LCMS 1 | 730.2 | 0.53 |
| I-57 | 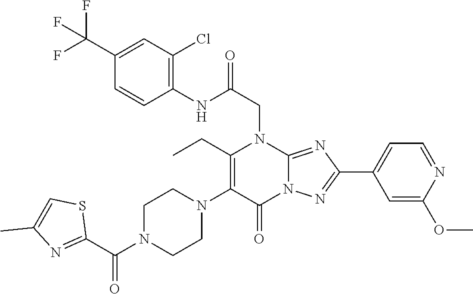 | LCMS 1 | 716.1 | 0.63 |
| I-58 | 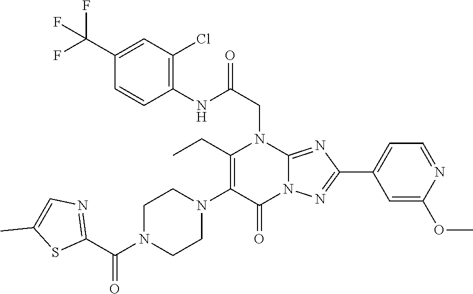 | LCMS 1 | 716.1 | 0.63 |
| I-59 | 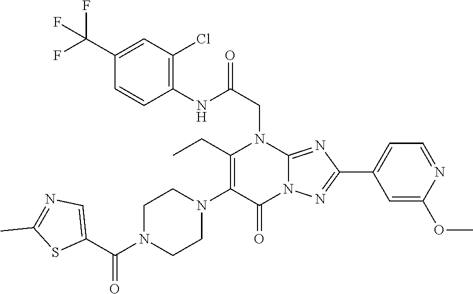 | LCMS 1 | 716.1 | 0.59 |
| I-60 | 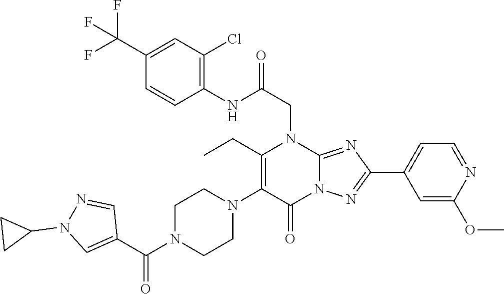 [SMILES: CCc1c(N2CCN(C(=O)c3cnn(C4CC4)c3)CC2)c(=O)n2nc(-c3ccnc(OC)c3)nc2n1CC(=O)Nc1ccc(C(F)(F)F)cc1Cl] | LCMS 1 | 725.4 | 0.63 |
| I-61 | 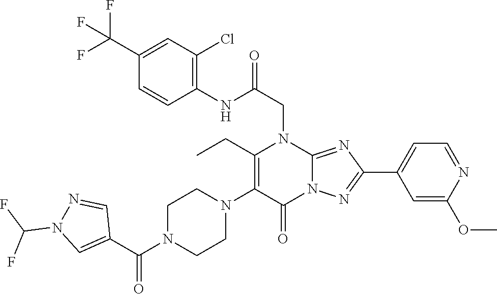 [SMILES: CCc1c(N2CCN(C(=O)c3cnn(C(F)F)c3)CC2)c(=O)n2nc(-c3ccnc(OC)c3)nc2n1CC(=O)Nc1ccc(C(F)(F)F)cc1Cl] | LCMS 1 | 735.4 | 0.63 |
| I-62 | 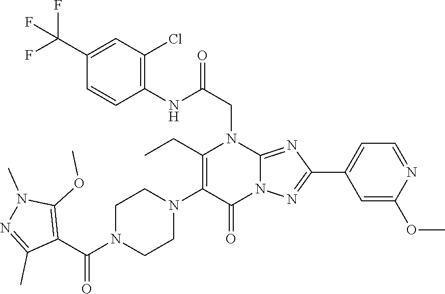 [SMILES: CCc1c(N2CCN(C(=O)c3c(C)nn(C)c3OC)CC2)c(=O)n2nc(-c3ccnc(OC)c3)nc2n1CC(=O)Nc1ccc(C(F)(F)F)cc1Cl] | LCMS 1 | 743.4 | 0.61 |
| I-63 | 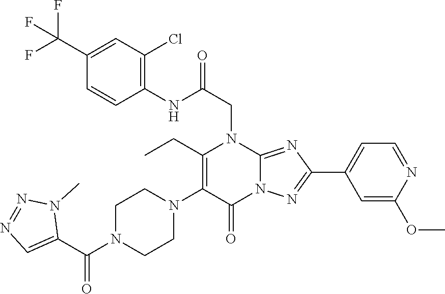 [SMILES: CCc1c(N2CCN(C(=O)c3cnnn3C)CC2)c(=O)n2nc(-c3ccnc(OC)c3)nc2n1CC(=O)Nc1ccc(C(F)(F)F)cc1Cl] | LCMS 1 | 700.4 | 0.61 |
| I-64 | 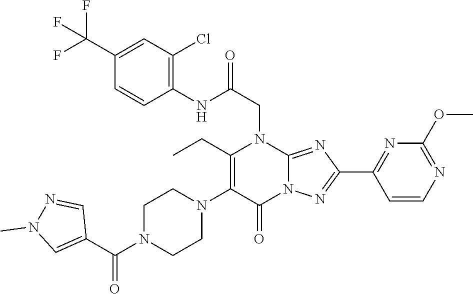 | LCMS 1 | 700.3 | 0.58 |
| I-65 | 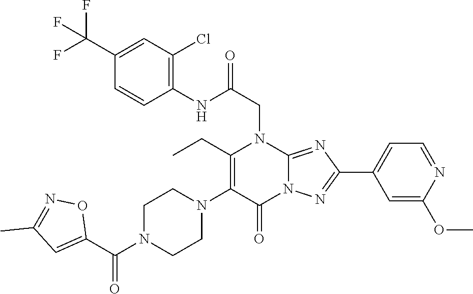 | LCMS 1 | 700.3 | 0.64 |
| I-66 | 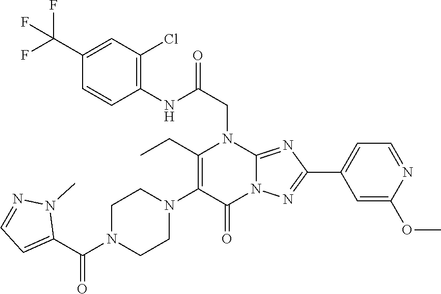 [SMILES: CCc1c(N2CCN(C(=O)c3ccnn3C)CC2)c(=O)n2nc(-c3ccnc(OC)c3)nc2n1CC(=O)Nc1ccc(C(F)(F)F)cc1Cl] | LCMS 1 | 699.4 | 0.63 |
| I-67 | 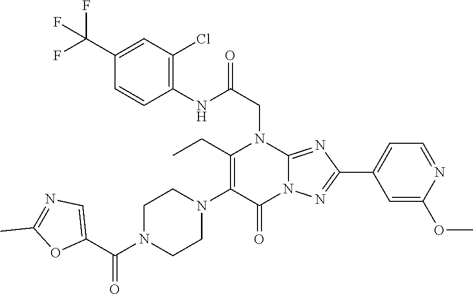 | LCMS 1 | 700.3 | 0.62 |
| I-68 | 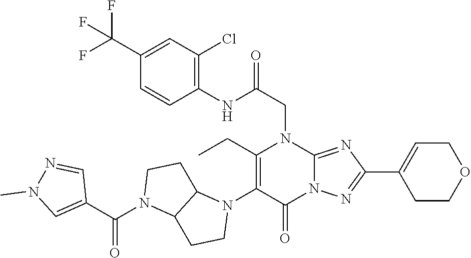 [SMILES: CCc1c(N2CCC3C2CCN3C(=O)c2cnn(C)c2)c(=O)n2nc(C3=CCOCC3)nc2n1CC(=O)Nc1ccc(C(F)(F)F)cc1Cl] | LCMS 1 | 700.2 | 0.56 |
| I-69 | 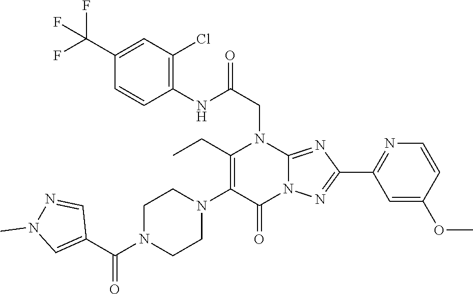 | LCMS 1 | 699.1 | 0.5 |
| I-70 | 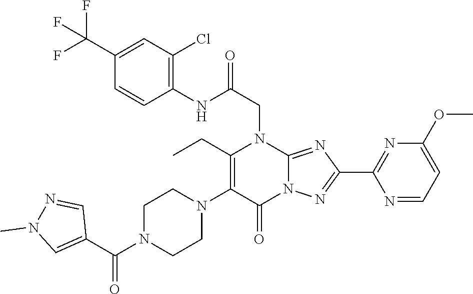 | LCMS 1 | 700.2 | 0.55 |
| I-71 | 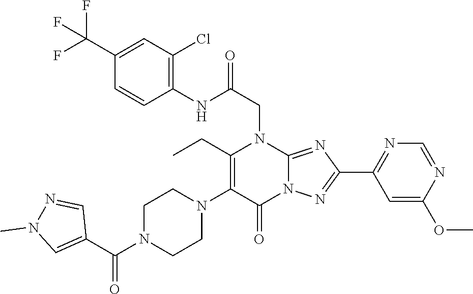 | LCMS 1 | 700.2 | 0.55 |
| I-72 | 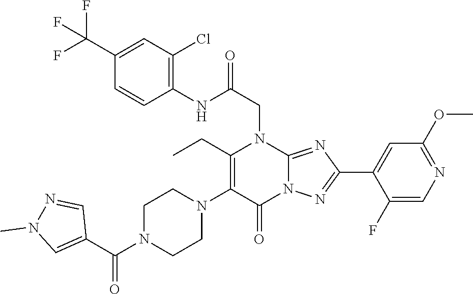 | LCMS 1 | 717.1 | 0.59 |
| I-73 | 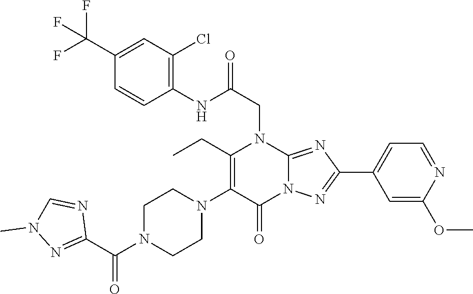 | LCMS 1 | 700.1 | 0.57 |
| I-74 | 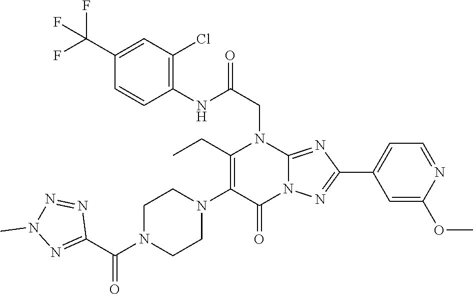 | LCMS 1 | 701.1 | 0.59 |
| I-75 | 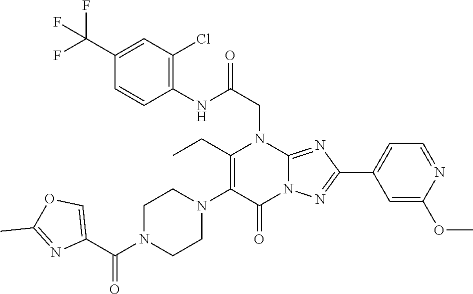 | LCMS 1 | 700.1 | 0.6 |
| I-76 | 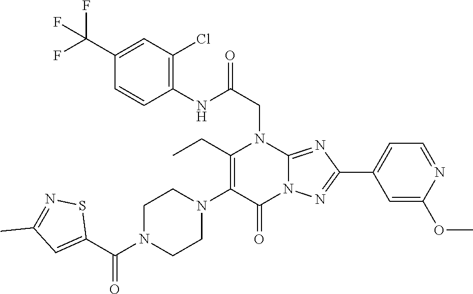 | LCMS 1 | 716.3 | 0.65 |
| I-77 | 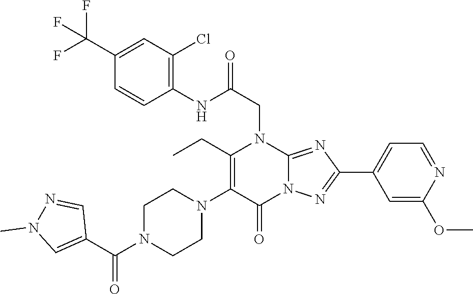 | LCMS 1 | 713.4 | 0.61 |
| I-78 | 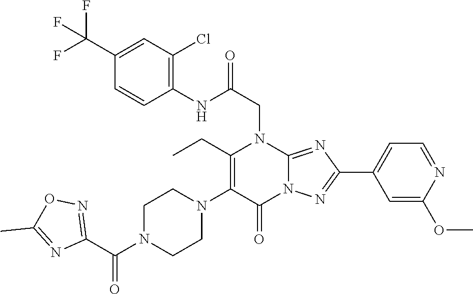 | LCMS 1 | 701.2 | 0.63 |
| I-79 | 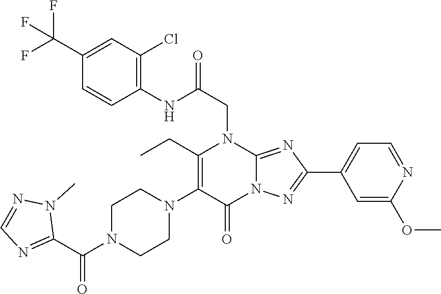 [SMILES: CCc1c(N2CCN(C(=O)c3ncnn3C)CC2)c(=O)n2nc(-c3ccnc(OC)c3)nc2n1CC(=O)Nc1ccc(C(F)(F)F)cc1Cl] | LCMS 1 | 700.2 | 0.62 |
| I-80 | 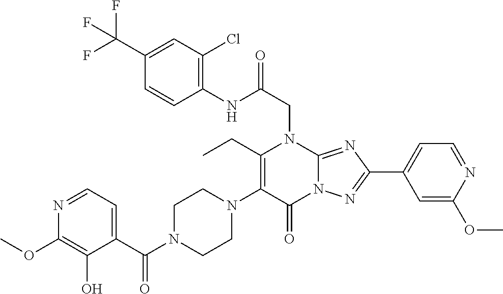 | LCMS 1 | 742.4 | 0.62 |
| I-81 | 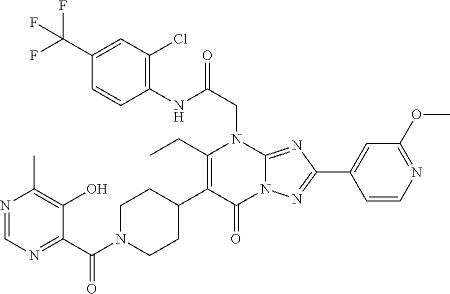 | LCMS 1 | 698.2 | 0.54 |
| I-82 | 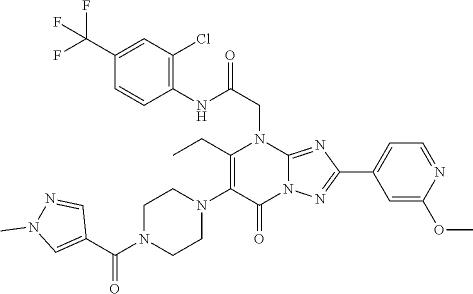 | LCMS 1 | 757.3 | 0.59 |
| I-83 | 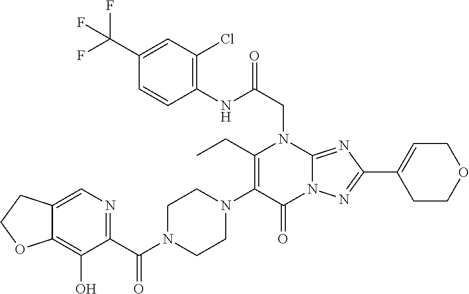 [SMILES: CCc1c(N2CCN(C(=O)c3ncc4c(c3O)OCC4)CC2)c(=O)n2nc(C3=CCOCC3)nc2n1CC(=O)Nc1ccc(C(F)(F)F)cc1Cl] | LCMS 5 | 729.2 | 0.95 |
| I-84 | 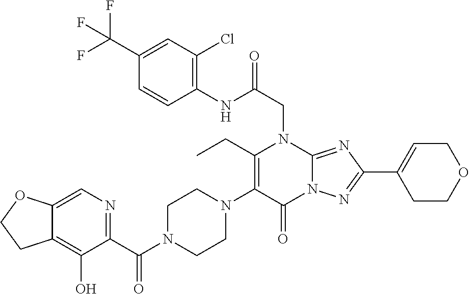 [SMILES: CCc1c(N2CCN(C(=O)c3ncc4c(c3O)CCO4)CC2)c(=O)n2nc(C3=CCOCC3)nc2n1CC(=O)Nc1ccc(C(F)(F)F)cc1Cl] | LCMS 6 | 729.4 | 0.95 |
| I-85 | 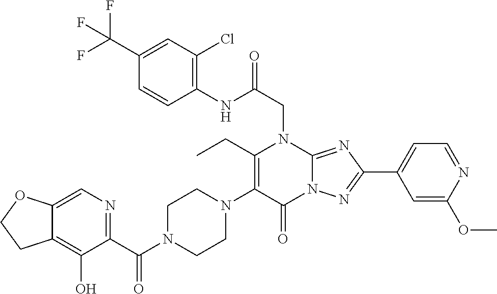 [SMILES: CCc1c(N2CCN(C(=O)c3ncc4c(c3O)CCO4)CC2)c(=O)n2nc(-c3ccnc(OC)c3)nc2n1CC(=O)Nc1ccc(C(F)(F)F)cc1Cl] | LCMS 7 | 754.3 | 1.48 |
| I-86 | 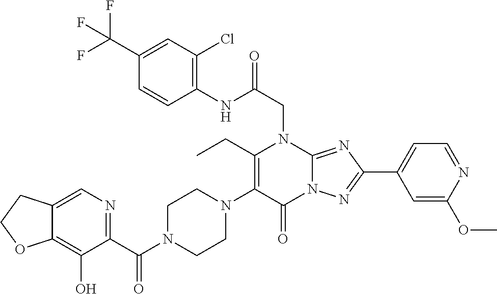 [SMILES: CCc1c(N2CCN(C(=O)c3ncc4c(c3O)OCC4)CC2)c(=O)n2nc(-c3ccnc(OC)c3)nc2n1CC(=O)Nc1ccc(C(F)(F)F)cc1Cl] | LCMS 4 | 754.3 | 0.82 |
| I-87 | 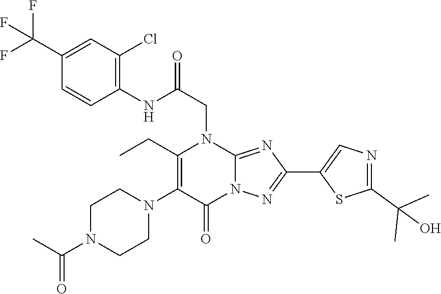 [SMILES: CCc1c(N2CCN(C(C)=O)CC2)c(=O)n2nc(-c3cnc(C(C)(C)O)s3)nc2n1CC(=O)Nc1ccc(C(F)(F)F)cc1Cl] | LCMS 1 | 667.4 | 0.58 |
| I-88 | 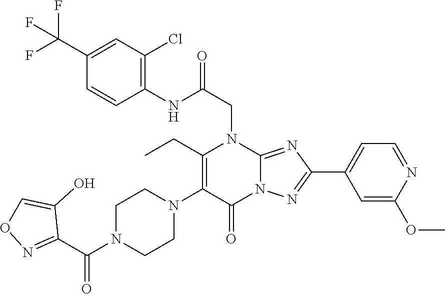 [SMILES: CCc1c(N2CCN(C(=O)c3nocc3O)CC2)c(=O)n2nc(-c3ccnc(OC)c3)nc2n1CC(=O)Nc1ccc(C(F)(F)F)cc1Cl] | LCMS 1 | 702.4 | 0.62 |

### LCMS Methods

### LCMS 1

| | |
|---|---|
| Instrument | Shimadzu LCMS-2020 |
| Stationary Phase | HALO C18 3.0 × 30 mm, 5.0 μm |
| Mode | Binary Gradient |
| Mobile Phase A | 0.0375% TFA in water (v/v) |
| Mobile Phase B | 0.01875% TFA in Acetonitrile (v/v) |
| Gradient | 5 to 95% B in 0.5 min, 95% B for 0.3 min, |
| | 95 to 5% B in 0.25 min |
| Flow Rate | 1.5 mL/min |
| Column Temperature | 50 °C |
| Column | 3.0 × 30 mm, 5.0 μm |

### LCMS 2

| | |
|---|---|
| Instrument | Shimadzu LCMS-2020 |
| Stationary Phase | HALO C18 3.0 × 30 mm, 5.0 μm |
| Mode | Binary Gradient |
| Mobile Phase A | 0.0375% TFA in water (v/v) |
| Mobile Phase B | 0.01875% TFA in Acetonitrile (v/v) |
| Gradient | 0 to 60% B in 0.5min, 60% B for 0.3 min, |
| | 60 to 0% B in 0.25 min |
| Flow Rate | 1.5 mL/min |
| Column Temperature | 50 °C |
| Column | 3.0 × 30 mm, 5.0 μm |

### LCMS 3

| | |
|---|---|
| Instrument | Agilent 1100/1200/1290/6100 |
| | Series/Alltech 2000ES/3300 |
| Stationary Phase | Agilent Poroshell 120 SB-C18 |
| | 4.6 × 30 mm 2.7 μm |
| Mode | Binary Gradient |
| Mobile Phase A | water (0.1% formic acid) |
| Mobile Phase B | acetonitrile (0.1% formic acid) |
| Gradient | 0.01 min -1% B, 1.5 min - 100% B, |
| | 1.73 min - 100% B |
| Flow Rate | 3 mL/min |
| Column Temperature | 60 °C |
| Column | 4.6 × 30 mm, 2.7 μm |

### Example 2: WRN (BV08) ADP-Glo Assay Protocol

Bovine skin gelatin (BSG), dimethyl sulfoxide (DMSO), Pluronic F-127 and tris(2-carboxyethyl)phosphine hydrochloride solution (TCEP) were purchased from Sigma-Aldrich (St. Louis, MO) at the highest level of purity possible. Bicine buffer solution was purchased from Alfa Aesar (Tewksbury, MA) and compound NSC-617145 was purchased from Tocris (Minneapolis, MN). DNA duplex was synthesized at BGI (Shenzhen, China) and was composed of strand 1 with the sequence 5′-GCACTGGCCGTCGTTTTACGGTCG-3′ (SEQ ID NO.: 1) and strand 2 with the sequence 5′-TCCAAGTAAAACGACGGCCAGTGC-3′ (SEQ ID NO.: 2). DNA strands were annealed by heating to 95 °C for 5 minutes followed by slow cooling to room temperature. Compounds in 100% DMSO (0.1 μl) were spotted into a 384-well white polystyrene Optiplate-384 (Perkin Elmer; Waltham, MA) assay plate using a LabCyte Echo 550 (Agilent; Santa Clara, CA). DMSO (0.1 μl) was added to columns 12, rows A-H and column 24, rows I-P for the maximum signal control. Compound NSC-617145 (0.1 μl) was added to columns 12, rows I-P and 24, rows A-H for the minimum signal control (100% inhibition). Compounds/DMSO were preincubated for 15 minutes at 25 °C with 5 μl 2× WRN (BV08), prepared as described below, in assay buffer containing 20 mM Bicine (pH=7.5), 1 mM MgCl₂, 10 mM KCl, 0.1% Pluronic F-127, 0.005% BSG, 1 mM TCEP. The reaction was initiated by the addition of 5 μl 2× substrate mixture in assay buffer and incubated for 60 minutes at 25 °C. The final concentrations of the assay components were 0.15 nM WRN, 5 M ATP, and 0.1 nM DNA duplex. The final DMSO concentration was 1% and the reference compound concentration (NSC-617145) used for the minimal signal control was 20 μM. The reaction was stopped by the addition of the ADP-Glo Kit components (Promega; Madison, WI) as directed and the relative luminescence units (RLU) were read on an Envision 2104 (Perkin Elmer; Waltham, MA).
% inhibition calculation:
% INH=(RLU MAX-RLU sample)/(RLU MAX-RLU MIN))×100

Where RLU=relative luminescence units, sample=signal in sample well, and MIN and MAX are the respective minimum and maximum signal controls.
Four-parameter IC₅₀ fit equation:

Y=Bottom+(Top−Bottom)/(1+(IC50/X)\hat{ }Hill Slope)

Where top and bottom are normally allowed to float but may be fixed at 100 or 0 respectively in a 3-parameter fit. Y is the % inhibition and X is the compound concentration.

### WRN Protein Production

Molecular Biology and virus production. The DNA encoding human Werner helicase (Uniprot Q14191, amino acids 517-1235 with L1074F point mutation) was generated with codon-optimization for E. coli expression and subcloned into the pFastBac vector with a TEV cleavable 8×His tag (WRN-BV08). The baculovirus from the expression plasmid WRN-BV08 was generated from transfection and amplification following the manufacturer's instructions.

Gene sequence of WRN-BV08
[pFastBac1-WRN-(517-1235 L1074F)-TEV-8His]
(SEQ ID NO.: 3)
ATGAACGAGGGCGAAGAAGACGACGACAAGGACTTCCTGTGGCCTGCCCC
TAACGAAGAACAAGTGACATGCCTGAAGATGTACTTCGGACACAGTAGCT
TCAAGCCTGTGCAATGGAAGGTCATCCACTCCGTGCTGGAAGAAAGAAGG
GACAACGTGGCTGTGATGGCTACCGGATACGGTAAGTCCCTGTGCTTCCA
GTACCCTCCCGTGTACGTGGGCAAGATCGGTCTGGTGATCTCCCCTCTGA
TCTCTCTGATGGAGGACCAGGTGCTGCAATTGAAGATGTCCAACATCCCC
GCTTGCTTCCTGGGTTCCGCTCAAAGTGAGAACGTGCTGACAGACATCAA
GCTGGGCAAGTACCGCATCGTGTACGTGACCCCTGAGTACTGCTCCGGTA
ACATGGGTCTGCTGCAACAGCTGGAGGCTGACATCGGAATCACCCTGATC
GCTGTGGACGAGGCTCACTGCATCTCCGAGTGGGGACACGACTTCCGCGA
CTCCTTCCGTAAGCTGGGATCCTTGAAGACCGCTCTCCCTATGGTGCCTA
TCGTGGCCCTGACCGCCACTGCTTCCTCCTCCATCCGCGAGGACATCGTG
CGTTGCCTGAACCTGCGCAACCCTCAGATCACTTGCACCGGTTTCGACCG
CCCTAACTTGTACCTCGAGGTGCGTCGCAAGACCGGTAACATCCTCCAGG
ACCTGCAGCCTTTCCTGGTCAAGACCTCCTCCCACTGGGAATTTGAGGGC
CCTACCATCATCTACTGCCCTTCCCGCAAGATGACCCAGCAAGTCACCGG
CGAGCTGCGCAAGCTCAACCTCTCCTGCGGTACCTACCACGCTGGTATGT
CCTTCTCCACCCGCAAGGACATCCACCACCGCTTCGTCCGTGACGAAATC
CAATGCGTCATCGCTACCATCGCTTTCGGAATGGGCATCAACAAGGCTGA
CATCCGCCAGGTGATCCACTACGGCGCCCCCAAGGACATGGAATCCTACT
ACCAGGAAATCGGTCGCGCCGGTCGCGACGGTCTGCAGTCTTCCTGTCAC
GTGCTGTGGGCCCCCGCTGACATCAACCTGAACCGCCACCTGCTGACCGA
AATCCGCAACGAGAAGTTCCGCCTGTACAAGCTCAAGATGATGGCTAAGA
TGGAGAAGTACCTGCACTCCTCCCGCTGTCGCCGTCAGATCATCCTCTCC
CACTTCGAGGACAAGCAAGTGCAAAAGGCTAGCCTGGGTATCATGGGCAC
CGAAAAGTGTTGTGACAACTGCCGCTCCCGCCTCGACCACTGCTACTCCA
TGGACGACAGCGAGGACACCTCCTGGGACTTCGGTCCTCAAGCTTTCAAG
CTCTTGTCCGCTGTGGACATCCTGGGCGAGAAGTTCGGTATCGGTCTCCC
CATCCTCTTCCTGCGTGGTAGCAACTCCCAACGCCTGGCTGACCAGTACC
GCCGCCACTCCCTCTTCGGTACCGGTAAGGACCAGACCGAGTCCTGGTGG
AAGGCTTTCTCTCGCCAACTGATCACCGAAGGTTTCCTGGTGGAGGTGTC
CCGCTACAACAAGTTCATGAAGATCTGCGCTCTCACTAAGAAGGGAAGGA
ACTGGCTGCACAAGGCTAACACTGAGTCCCAATCCCTCATCCTGCAGGCT
AACGAGGAGCTGTGCCCTAAGAAGTTCCTGCTGCCTTCCTCCAAGACCGT
GTCCTCCGGAACAAAGGAACACTGCTACAACCAAGTCCCTGTGGAGCTCT
CCACCGAGAAGAAGTCCAACCTGGAGAAGCTGTACAGCTACAAGCCTTGC
GACAAGATCAGCTCCGGTTCCAACATCAGCAAGAAGTCCATCATGGTGCA
ATCCCCTGAAAAGGCCTACTCCAGCTCCCAACCTGTCATCTCCGCTCAAG
AGCAAGAGACCCAGATCGTGCTGTACGGTAAGCTGGTCGAAGCCCGCCAA
AAGCACGCTAACAAGATGGACGTCCCTCCCGCTATCCTCGCCACCAACAA
GATCCTCGTGGATATGGCTAAGATGCGCCCCACCACCGTCGAGAACGTGA
AGCGCATCGACGGTGTCTCCGAGGGTAAGGCCGCTATGCTGGCTCCTCTG
CTGGAAGTGATCAAGCACTTCTGCCAGACCAACTCCGTGCAGACCGACCT
GTTCAGTAGTGAGAACCTGTACTTCCAAGGCCACCATCATCATCATCATC
ACCACTAA
Protein sequence of WRN-BV08
[pFastBac1-WRN-(517-1235 L1074F)-TEV-8His]
(SEQ ID NO.: 4)
MNEGEEDDDKDFLWPAPNEEQVTCLKMYFGHSSFKPVQWKVIHSVLEERR
DNVAVMATGYGKSLCFQYPPVYVGKIGLVISPLISLMEDQVLQLKMSNIP
ACFLGSAQSENVLTDIKLGKYRIVYVTPEYCSGNMGLLQQLEADIGITLI
AVDEAHCISEWGHDFRDSFRKLGSLKTALPMVPIVALTATASSSIREDIV
RCLNLRNPQITCTGFDRPNLYLEVRRKTGNILQDLQPFLVKTSSHWEFEG
PTIIYCPSRKMTQQVTGELRKLNLSCGTYHAGMSFSTRKDIHHRFVRDEI
QCVIATIAFGMGINKADIRQVIHYGAPKDMESYYQEIGRAGRDGLQSSCH
VLWAPADINLNRHLLTEIRNEKFRLYKLKMMAKMEKYLHSSRCRRQIILS
HFEDKQVQKASLGIMGTEKCCDNCRSRLDHCYSMDDSEDTSWDFGPQAFK
LLSAVDILGEKFGIGLPILFLRGSNSQRLADQYRRHSLFGTGKDQTESWW
KAFSRQLITEGFLVEVSRYNKFMKICALTKKGRNWLHKANTESQSLILQA
NEELCPKKFLLPSSKTVSSGTKEHCYNQVPVELSTEKKSNLEKLYSYKPC
DKISSGSNISKKSIMVQSPEKAYSSSQPVISAQEQETQIVLYGKLVEARQ
KHANKMDVPPAILATNKILVDMAKMRPTTVENVKRIDGVSEGKAAMLAPL
LEVIKHFCQTNSVQTDLFSSENLYFQGHHHHHHHH

Sf9 cells grown in SF₉₀₀II media were infected with 1:200 WRN-BV08 P2 virus and incubated for protein expression for 72 h at 27 °C. The WRN protein was purified using the following protocol. The cell pellets were thawed and resuspended in buffer A (50 mM Tris, pH 7.5, 500 mM NaCl, 1 mM TCEP, 10% Glycerol) supplemented with 0.5% CHAPS, 1 mM PMSF, 1 μg/ml Leupeptin, 1 μg/ml Pepstatin, and the Pierce Universal Nuclease and cocktail tablet. Cleared lysates were loaded onto a Ni Sepharose™ excel column and washed with buffer A and bound protein was eluted with buffer A supplemented with 300 mM imidazole. The eluted protein was dialyzed against buffer A and digested by His-tagged TEV (1:5 ratio) overnight at 4 °C. ZnCl₂ was added into the sample at final 15 μM before loading onto a second Ni Sepharose™ excel column. Untagged WRN protein was eluted from the column with buffer A supplemented with 20 mM imidazole, dialyzed overnight into buffer B (50 mM Tris, pH 7.5, 1 mM TCEP, 10% Glycerol) supplemented with 150 mM NaCl and loaded onto a Heparin column. Proteins were eluted with a step gradient of buffer B supplemented with 150 mM, 200 mM, 300 mM and 500 mM NaCl. WRN containing fractions were pooled and concentrated prior to loading on to size exclusion chromatography using a HiLoad 16/600 Superdex™ 200 μg column (GE Healthcare) in buffer C (20 mM HEPES, pH 7.5, 250 mM NaCl, 0.25 mM TCEP, 2.5% Glycerol).

The resultant IC₅₀ results obtained for the tested compounds are shown below in Table 3. Compounds with an IC₅₀ less than or equal to 0.005 μM are designated as “A.” Compounds with an IC₅₀ greater than 0.005 μM and less than or equal to 0.05 μM are designated as “B.” Compounds with an IC₅₀ greater than 0.05 μM and less than or equal to 0.1 μM are designated as “C.” Compounds with an IC₅₀ greater than 0.1 μM or equal to 0.5 μM are designated as “D.”

TABLE 3

| Cmpd. No. | ADP-Glo_hWRN_IC50 [μM] |
|---|---|
| I-1 | B |
| I-2 | B |
| I-3 | B |
| I-4 | B |
| I-5 | B |
| I-6 | B |
| I-7 | B |
| I-8 | B |
| I-9 | B |
| I-10 | B |
| I-11 | A |
| I-12 | B |
| I-13 | B |
| I-14 | A |
| I-15 | A |
| I-16 | B |
| I-17 | B |
| I-18 | C |
| I-19 | B |
| I-20 | B |
| I-21 | B |
| I-22 | B |
| I-23 | B |
| I-24 | B |
| I-25 | B |
| I-26 | B |
| I-27 | B |
| I-28 | A |
| I-29 | A |
| I-30 | B |
| I-31 | C |
| I-32 | B |
| I-33 | B |
| I-34 | B |
| I-35 | B |
| I-36 | D |
| I-37 | B |
| I-38 | A |
| I-39 | B |
| I-40 | C |
| I-41 | B |
| I-42 | B |
| I-43 | B |
| I-44 | B |
| I-45 | B |
| I-46 | B |
| I-47 | C |
| I-48 | A |
| I-49 | B |
| I-50 | B |
| I-51 | B |
| I-52 | B |
| I-53 | B |
| I-54 | B |
| I-55 | B |
| I-56 | B |
| I-57 | D |
| I-58 | C |
| I-59 | B |
| I-60 | B |
| I-61 | B |
| I-62 | B |
| I-63 | B |
| I-64 | B |
| I-65 | B |
| I-66 | B |
| I-67 | B |
| I-68 | C |
| I-69 | B |
| I-70 | D |
| I-71 | B |
| I-72 | B |
| I-73 | B |
| I-74 | B |
| I-75 | B |
| I-76 | B |
| I-77 | B |
| I-78 | B |
| I-79 | B |
| I-80 | B |
| I-81 | D |
| I-82 | B |
| I-83 | B |
| I-84 | A |
| I-85 | A |
| I-86 | A |
| I-87 | B |
| I-88 | B |

We claim:

1. A compound of Formula I, or a pharmaceutically acceptable salt thereof: 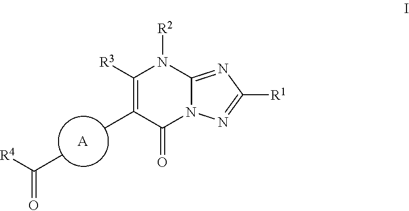
wherein Ring A represents:
a) a 4-7 membered saturated or partially unsaturated bivalent monocyclic ring system selected from carbocyclylene or heterocyclylene (having 1-4 heteroatoms independently selected from nitrogen, oxygen, and sulfur, and 0 or 1 nitrogen atoms in addition to the 1-4 heteroatoms); or
b) a 4-12 membered saturated or partially unsaturated bivalent bicyclic ring system that is fused, bridged, or spirocyclic selected from carbocyclylene or heterocyclylene (having 1-4 heteroatoms independently selected from nitrogen, oxygen, and sulfur);
wherein Ring A is substituted with 0-4 independently selected R^{A} substituents;
R¹ is:
a) a 5- or 6-membered heteroaryl (having 1-4 heteroatoms independently selected from nitrogen, oxygen, and sulfur) substituted with 1 or 2 groups independently selected from C₁-C₆alkoxy, C₃-C₆cycloalkyl, and C₃-C₆cycloalkoxy, wherein said 5- or 6-membered heteroaryl is further substituted with 0-3 independently selected R^{A};
b) a 5- or 6-membered saturated or partially unsaturated heterocyclyl (having 1-2 heteroatoms independently selected from nitrogen, oxygen, and sulfur), said heterocyclyl substituted with 0-2 groups independently selected from halogen, oxo, —NR₂, optionally substituted C₁₋₄ aliphatic, —OR, azetidinyl optionally substituted with 1 or 2 independently selected halogen, and pyrrolidinyl optionally substituted with 1 or 2 independently selected halogen;
c) a 6-8 membered saturated or partially unsaturated bridged bicyclic heterocyclyl (having 1-4 heteroatoms independently selected from nitrogen, oxygen, and sulfur), said heterocyclyl substituted with 0-2 groups independently selected from halogen, oxo, —NR₂, optionally substituted C₁₋₄ aliphatic, —OR, azetidinyl optionally substituted with 1 or 2 independently selected halogen, and pyrrolidinyl optionally substituted with 1 or 2 independently selected halogen; or
d) a 3-7 membered optionally substituted carbocyclyl or an optionally substituted C₂-C₄alkenyl;
R^{A} is independently selected at each occurrence from the group consisting of halogen, —CN, —NO₂, —OR, —SR, —NR₂, —S(O)₂R, —S(O)₂NR₂, —S(O)R, —S(O)NR₂, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)C(NR)NR₂, —N(R)S(O)₂NR₂, —N(R)S(O)₂R, and an optionally substituted C₁₋₆ aliphatic group;
R² is C(R^{B})₂C(O)N(R)R^{2A};
R^{B} is independently selected at each occurrence from hydrogen, —CH₃, or —CH₂CH₃, or two R^{B} taken together with the carbon to which they are attached form a cyclopropyl ring;
R^{2A} is phenyl or pyridyl, each of which is optionally substituted with 1, 2, or 3 substituents independently selected from halogen, C₁-C₄alkyl, haloC₁-C₄alkyl, C₃-C₆cycloalkyl, haloC₃-C₆-cycloalkyl, —OH, —CN, C₁-C₄alkoxy, haloC₁-C₄alkoxy, —SF₅, two substituents on adjacent atoms of the phenyl or pyridyl together with their intervening atoms form a 4- to 7-membered carbocyclyl fused to the phenyl or pyridyl, and two substituents on adjacent atoms of the phenyl or pyridyl together with their intervening atoms form a 4- to 7-membered heterocyclyl (having 1-4 heteroatoms independently selected from nitrogen, oxygen, and sulfur) fused to the phenyl or pyridyl; or
R^{2A} is 2-benzimidazolyl or 3-quinolinyl, each of which is optionally substituted with 1, 2 or 3 substituents independently selected from halogen, C₁₋₄alkyl and —OH;
R³ is hydrogen, C₁-C₄alkyl, C₃-C₅cycloalkyl, C₁-C₄alkoxy, —NHR^{3A}, —N(R^{3A})₂ or C₁-C₄alkylthio each of which, besides hydrogen, is optionally substituted with —OH, 1-5 independently selected halogen, or C₁-C₄alkoxy;
each R^{3A} is independently selected at each occurrence from C₁-C₄alkyl;
R⁴ is selected from one of a), b), and c):
a) R⁴ is a Ring B that is selected from the group consisting of 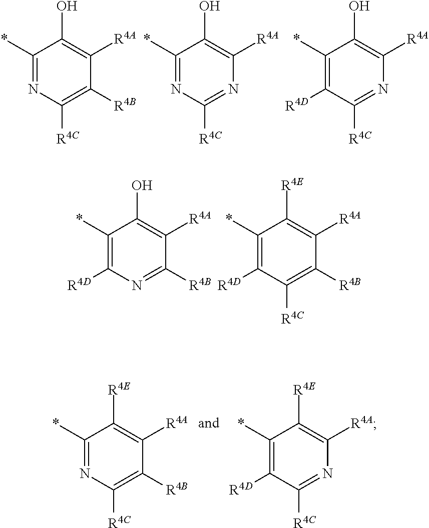
wherein * is a point of attachment to the carbonyl group that is bonded to Ring A in Formula I;
and wherein:
any substituents that are present on Ring B selected from R^{4A}, R^{4B}, R^{4C}, R^{4D}, R^{4E}, and R^{F} are each independently selected from hydrogen; halogen; —CN; C₁-C₄alkyl; C₂-C₄alkenyl; C₂-C₄alkynyl; C₁-C₄alkoxy; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; or NR^{C}₂; or
R^{4A} and R^{4B}, along with their intervening atoms, join to form 4- to 7-membered optionally substituted carbocyclyl or optionally substituted heterocyclyl (having 1-4 heteroatoms independently selected from nitrogen, oxygen, and sulfur) that is fused to Ring B; and any substituents that are present on Ring B selected from R^{4C}, R^{4D}, R^{4E}, and R^{4F} are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; or NR^{C}₂; or
R^{4B} and R^{4C}, along with their intervening atoms, join to form 4- to 7-membered optionally substituted carbocyclyl or optionally substituted heterocyclyl (having 1-4 heteroatoms independently selected from nitrogen, oxygen, and sulfur) that is fused to Ring B; and any substituents that are present on Ring B selected from R^{4A}, R^{4D}, R^{4E}, and R^{4F} are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; or NR^{C}₂; or
R^{4C} and R^{4D}, along with their intervening atoms, join to form 4- to 7-membered optionally substituted carbocyclyl or optionally substituted heterocyclyl (having 1-4 heteroatoms independently selected from nitrogen, oxygen, and sulfur) that is fused to Ring B; and any substituents that are present on Ring B selected from R^{4A}, R^{4B}, and R^{4E} are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; or NR^{C}₂; or
R^{4E} is halogen or —OH, and R^{4A}, R^{4B}, R^{4C}, and R^{4D} are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; or NR^{C}₂; or
R^{4E} and R^{4A}, along with their intervening atoms, join to form 5- to 6-membered optionally substituted heteroaryl (having 1-4 heteroatoms independently selected from nitrogen, oxygen, and sulfur) that is fused to Ring B; and R^{4B}, R^{4C}, and R^{4D} are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; or NR^{C}₂; or
R^{4F} and R^{4A}, along with their intervening atoms, join to form 5- to 6-membered optionally substituted heteroaryl (having 1-4 heteroatoms independently selected from nitrogen, oxygen, and sulfur) that is fused to Ring B; and any substituents that are present on Ring B selected from R^{4B}, R^{4C}, and R^{4D} are each independently selected from hydrogen; halogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; or NR^{C}₂; and
R^{C} is independently selected at each occurrence from hydrogen or C₁-C₄alkyl optionally substituted with —OH, —OCH₃, or —OCH₂CH₃; or NR^{C}₂, taken in combination form a heterocyclic ring selected from azetidinyl, pyrrolidinyl, or piperidinyl, said heterocyclic ring optionally substituted with —CH₃; or
b) R⁴ is a 5-membered heteroaryl (having 1 heteroatom independently selected from nitrogen, oxygen, and sulfur and 0, 1, 2, or 3 additional ring nitrogen atoms), wherein said heteroaryl is optionally substituted with 0-4 groups independently selected from halogen, —OH, —CN, C₁-C₄alkyl, haloC₁-C₄alkyl, cycloC₃-C₆alkyl, and C₁-C₄alkoxy; or
c) R⁴ is a C₁-C₄alkyl, C₁-C₄alkoxy, or cycloC₃-C₆alkyl, each of which is substituted with 0-3 groups independently selected from halogen, —CN, —OH, C₁-C₄alkyl, C₁-C₄alkoxy, optionally substituted 5- to 6-membered heterocyclyl, and optionally substituted 5- to 6-membered heterocyclyloxy;
each R is independently hydrogen, or an optionally substituted C₁₋₆ aliphatic group, an optionally substituted phenyl, an optionally substituted 3-7 membered saturated or partially unsaturated carbocyclic ring, an optionally substituted 3-7 membered saturated or partially unsaturated heterocyclic ring (having 1-2 heteroatoms independently selected from nitrogen, oxygen, and sulfur), or an optionally substituted 5-6 membered heteroaryl ring (having 1-4 heteroatoms independently selected from nitrogen, oxygen, and sulfur); or
two R groups on the same atom are taken together with the same atom to form an optionally substituted 4-7 membered saturated ring, partially unsaturated ring, or heteroaryl ring (wherein said 4-7 membered saturated ring, partially unsaturated ring, or heteroaryl ring, has 0-3 heteroatoms independently selected from nitrogen, oxygen, and sulfur);
with the proviso that one or two of the following provisions 1)-7) is satisfied, further provided that two of 1), 2), and 3) are not concurrently satisfied, and further provided that two of 4), 5), 6) and 7) are not concurrently satisfied:
1) R¹ is a) a 5- or 6-membered heteroaryl (having 1-4 heteroatoms independently selected from nitrogen, oxygen, and sulfur) substituted with 1 or 2 groups independently selected from C₁-C₆alkoxy, C₃-C₆cycloalkyl, or C₃-C₆cycloalkoxy, wherein said 5- or 6-membered heteroaryl is further substituted with 0-3 independently selected R^{A};
2) R¹ is c) 6-8 membered saturated or partially unsaturated bridged bicyclic heterocyclyl (having 1-4 heteroatoms independently selected from nitrogen, oxygen, and sulfur), said heterocyclyl substituted with 0-2 groups independently selected from halogen, oxo, —NR₂, optionally substituted C₁₋₄ aliphatic, —OR, azetidinyl optionally substituted with 1 or 2 independently selected halogen, and pyrrolidinyl optionally substituted with 1 or 2 independently selected halogen;
3) R¹ is d) a 3-7 membered optionally substituted carbocyclyl or an optionally substituted C₂-C₄alkenyl;
4) R⁴ is a 5-membered heteroaryl (having 1 heteroatom independently selected from nitrogen, oxygen, and sulfur and 0, 1, 2, or 3 additional ring nitrogen atoms), wherein said heteroaryl is optionally substituted with 0-4 groups independently selected from halogen, —OH, —CN, C₁-C₄alkyl, haloC₁-C₄alkyl, cycloC₃-C₆alkyl, and C₁-C₄alkoxy;
5) R⁴ is selected from: 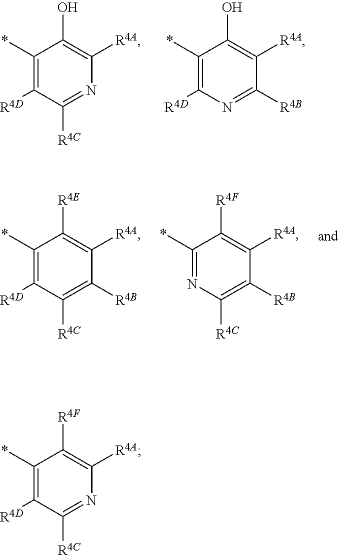
6) R⁴ is: 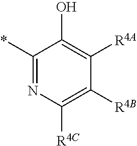
wherein
R^{4A} and R^{4B}, along with their intervening atoms, join to form 4- to 7-membered optionally substituted carbocyclyl or optionally substituted heterocyclyl (having 1-4 heteroatoms independently selected from nitrogen, oxygen, and sulfur), or wherein R^{4B} and R^{4C} along with their intervening atoms, join to form 4- to 7-membered optionally substituted carbocyclyl or optionally substituted heterocyclyl (having 1-4 heteroatoms independently selected from nitrogen, oxygen, and sulfur);
7) R⁴ is a C₁-C₄alkyl, C₁-C₄alkoxy, or cycloC₃-C₆alkyl, each of which is substituted with 0-3 groups independently selected from halogen, —CN, —OH, C₁-C₄alkyl, optionally substituted 5- to 6-membered heterocyclyl, and C₁-C₄alkoxy.

2. The compound of claim 1, wherein R¹ is a 5- or 6-membered heteroaryl (having 1-4 heteroatoms independently selected from nitrogen, oxygen, and sulfur) substituted with 1 group selected from C₁-C₆alkoxy and C₃-C₆cycloalkyl, wherein said 5- or 6-membered heteroaryl is further substituted with 0-3 independently selected R^{A}.

3. The compound of claim 1 or claim 2, wherein R¹ is pyridyl substituted with C₁-C₄alkoxy and further substituted with 0-2 R^{A}.

4. The compound of claim 1 or claim 2, wherein R¹ is 5-membered heteroaryl (having 1 heteroatom independently selected from nitrogen, oxygen, and sulfur, and 0 or 1 additional ring nitrogen atoms), wherein said 5-membered heteroaryl is substituted with a C₃-C₅cycloalkyl and further substituted with 0-2 independently selected R⁴.

5. The compound of claim 1 or claim 2, wherein R¹ is
b) a 5- or 6-membered saturated or partially unsaturated heterocyclyl (having 1-2 heteroatoms independently selected from nitrogen, oxygen, and sulfur), said heterocyclyl substituted with 0-2 groups independently selected from halogen, oxo, —NR₂, optionally substituted C₁₋₄ aliphatic, —OR, azetidinyl optionally substituted with 1 or 2 independently selected halogen, and pyrrolidinyl optionally substituted with 1 or 2 independently selected halogen; or
c) a 6-8 membered saturated or partially unsaturated bridged bicyclic heterocyclyl (having 1-4 heteroatoms independently selected from nitrogen, oxygen, and sulfur), said heterocyclyl substituted with 0-2 groups independently selected from halogen, oxo, —NR₂, optionally substituted C₁₋₄ aliphatic, —OR, azetidinyl optionally substituted with 1 or 2 independently selected halogen, and pyrrolidinyl optionally substituted with 1 or 2 independently selected halogen;
provided that each optionally substituted C₁₋₄ aliphatic or —OR substituent of b) or c), is independently unsubstituted or has substitutions limited to —OH; C₁-C₂alkoxy; or 1, 2 or 3 independently selected halogens.

6. The compound of claim 1 or claim 2, wherein R¹ is b) a 5- or 6-membered saturated or partially unsaturated heterocyclyl (having 1-2 heteroatoms independently selected from nitrogen, oxygen, and sulfur), said heterocyclyl substituted with 0-2 groups independently selected from halogen, oxo, —NR₂, optionally substituted C₁₋₄ aliphatic, —OR, azetidinyl optionally substituted with 1 or 2 independently selected halogen, and pyrrolidinyl optionally substituted with 1 or 2 independently selected halogen.

7. The compound of claim 1 or claim 2, wherein R¹ is selected from the group consisting of:

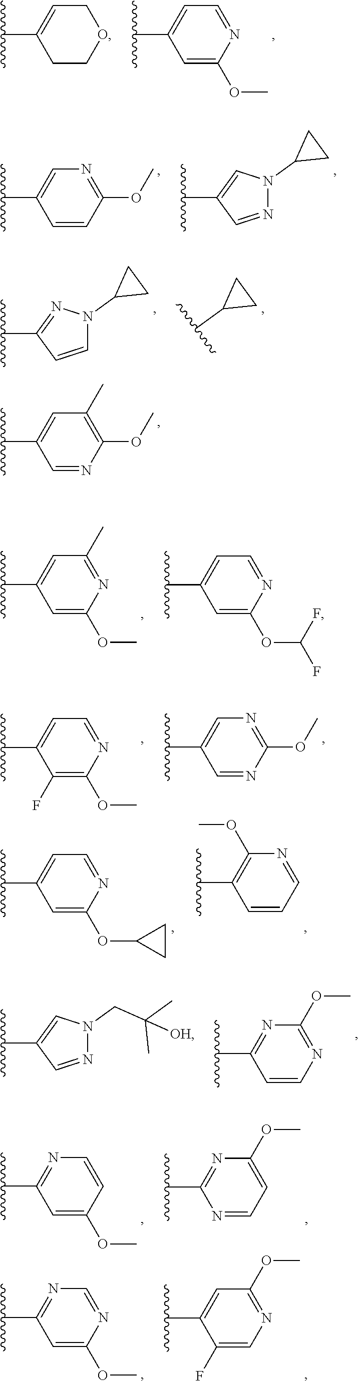

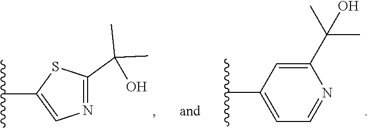

8. The compound of any one of claims 1-7, wherein R⁴ is Ring B of the following structure: 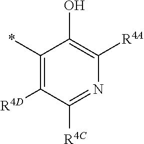
wherein * is a point of attachment to the carbonyl group that is bonded to Ring A in Formula I;
R^{4A} is —OCH₃, —OCH₂CH₃, or —OCHF₂;
R^{4C} and R^{4D} are each independently selected from hydrogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR^{C}₂; and
R^{C} is independently selected at each occurrence from hydrogen or C₁-C₄alkyl optionally substituted with —OH, —OCH₃, or —OCH₂CH₃; or NR^{C}₂, taken in combination form a heterocyclic ring selected from azetidinyl, pyrrolidinyl, or piperidinyl, said heterocyclic ring optionally substituted with —CH₃; or
R⁴ is a 5-membered heteroaryl (having 1 heteroatom independently selected from nitrogen, oxygen, and sulfur and 0, 1, 2, or 3 additional ring nitrogen atoms), wherein said heteroaryl is optionally substituted with 0-4 groups independently selected from halogen, —OH, —CN, C₁-C₄alkyl, haloC₁-C₄alkyl, cycloC₃-C₆alkyl, and C₁-C₄alkoxy.

9. The compound of any one of claims 1-8, wherein R⁴ is: 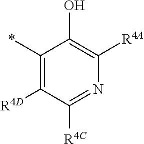
wherein * is a point of attachment to the carbonyl group that is bonded to Ring A in Formula I;
wherein:
R^{4A} is —OCH₃, —OCH₂CH₃, or —OCHF₂;
R^{4C} and R^{4D} are each independently selected from hydrogen; —CN; C₁-C₄alkyl; C₂-C₄alkenyl; C₂-C₄alkynyl; haloC₁-C₄alkyl; C₁-C₃alkyl substituted with —OH, —OCH₃, or —OCH₂CH₃; haloC₁-C₄alkoxy; C₃-C₆cycloalkyl; C₃-C₆cycloalkoxy; and NR^{C}₂; and
R^{C} is independently selected at each occurrence from hydrogen or C₁-C₄alkyl optionally substituted with —OH, —OCH₃, or —OCH₂CH₃; or NR^{C}₂, taken in combination form a heterocyclic ring selected from azetidinyl, pyrrolidinyl, or piperidinyl, said heterocyclic ring optionally substituted with —CH₃.

10. The compound of any one of claims 1-8, wherein R⁴ is a 5-membered heteroaryl (having 1 heteroatom independently selected from nitrogen, oxygen, and sulfur and 0, 1, 2, or 3 additional ring nitrogen atoms), wherein said heteroaryl is optionally substituted with 0-4 groups independently selected from halogen, —OH, —CN, C₁-C₄alkyl, haloC₁-C₄alkyl, cycloC₃-C₆alkyl, and C₁-C₄alkoxy.

11. The compound of claim 10, wherein R⁴ is an isoxazolyl substituted with —OH or C₁-C₄alkoxy.

12. The compound of any one of claims 1-8, wherein R⁴ is a 5-membered heteroaryl (having 1 heteroatom independently selected from nitrogen, oxygen, and sulfur and 0, 1, 2, or 3 additional ring nitrogen atoms) selected from the group consisting of thiophenyl, imidazolyl, pyrazolyl, tetrazolyl, thiazolyl, isothiazolyl, 1,2,5-thiadiazolyl, 1,3,4-thiadiazolyl, oxazolyl, isoxazolyl, 1,2,4-oxadiazolyl, 1,2,3-triazolyl, and 1,2,4-triazolyl, wherein said heteroaryl is optionally substituted with 0-4 groups independently selected from halogen, —OH, —CN, C₁-C₄alkyl, haloC₁-C₄alkyl, cycloC₃-C₆alkyl, and C₁-C₄alkoxy.

13. The compound of any one of claims 1-8, wherein R⁴ is

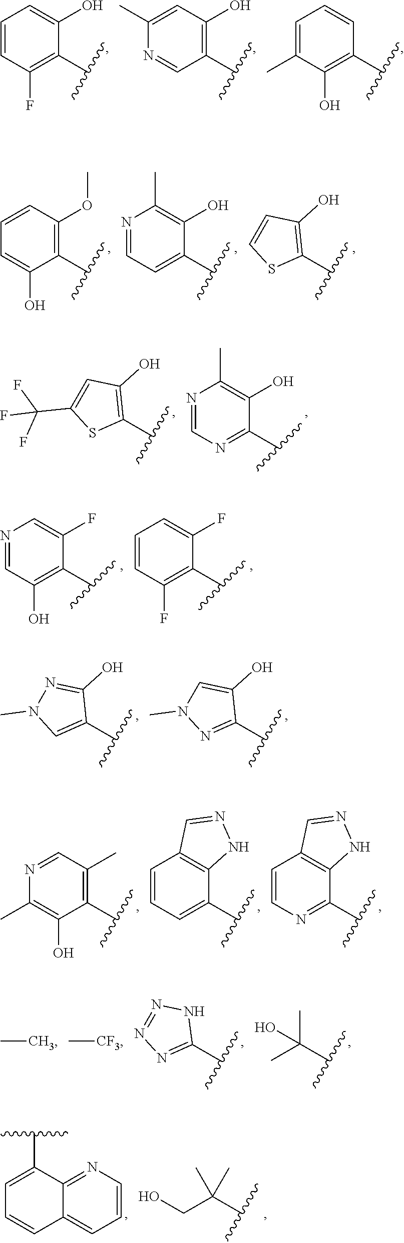

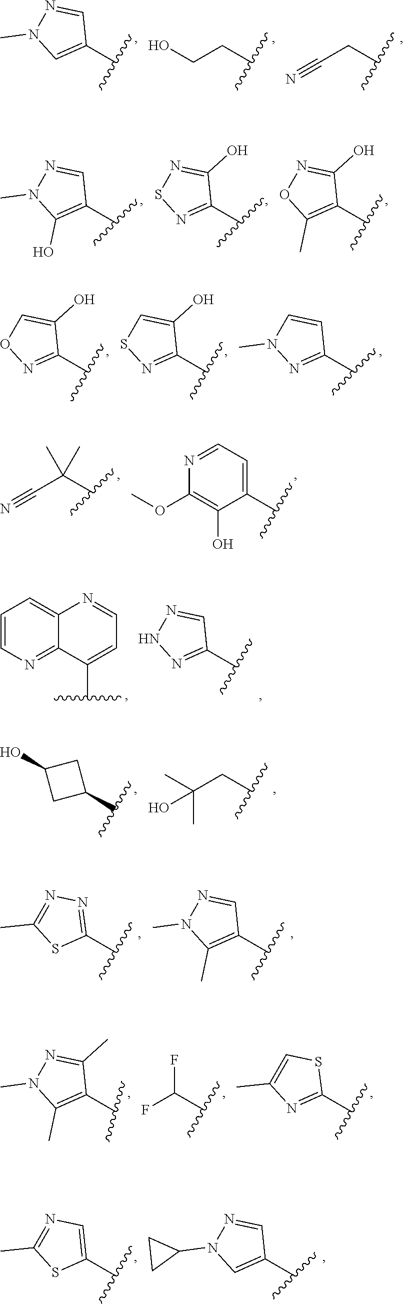

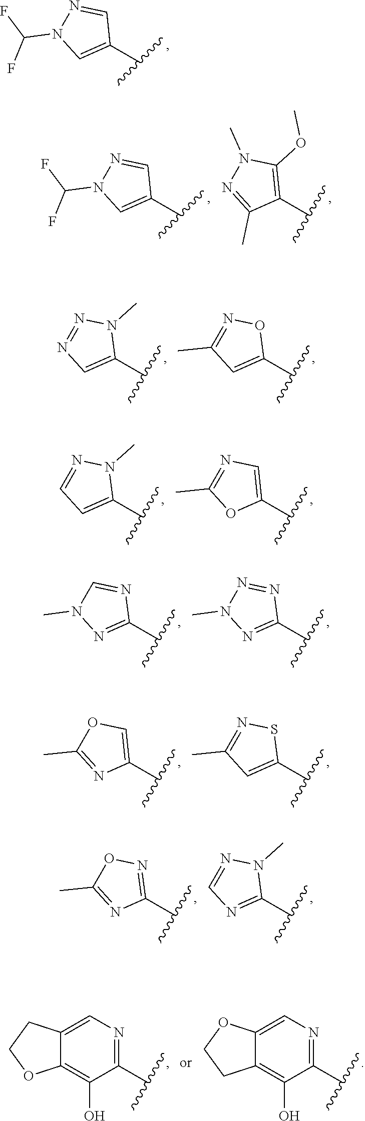

14. The compound of any one of claims 1-13, wherein R^{2A} is phenyl comprising a —CF₃ substituent or pyridyl comprising a —CF₃ substituent.

15. The compound of any one of claims 1-14, wherein R² is

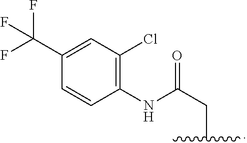 [SMILES: *CC(=O)Nc1ccc(C(F)(F)F)cc1Cl]

16. The compound of any one of claims 1-15, wherein R³ is C₁-C₄alkyl or C₃-C₅cycloalkyl.

17. The compound of any one of claims 1-16, wherein Ring A and the 0-4 independently selected R^{A} substituents with which Ring A is substituted is selected from

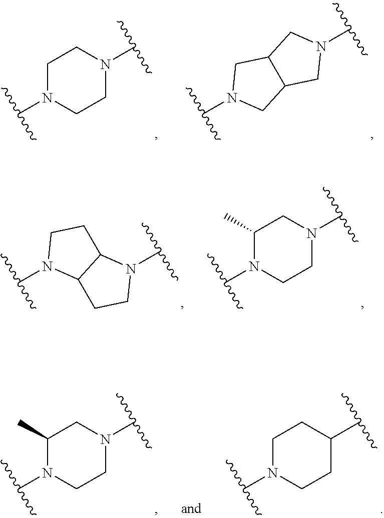

18. The compound of any one of claims 1-16, wherein Ring A and the 0-4 independently selected R^{A} substituents with which Ring A is substituted, is:

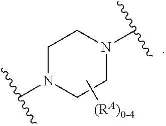

19. A pharmaceutical composition comprising a compound or pharmaceutically acceptable salt thereof according to any one of claims 1-18, and one or more pharmaceutically acceptable carriers.

20. A method of treating cancer in a subject, wherein the cancer is characterized as microsatellite instability-high (MSI-H) or mismatch repair deficient (dMMR), comprising administering to the subject a therapeutically effective amount of a compound according to any one of claims 1-18, or a pharmaceutically acceptable salt thereof.

21. A method of modulating WRN activity in a subject, wherein the method comprises administering to the subject a therapeutically effective amount of the compound according to any one of claims 1-18, or a pharmaceutically acceptable salt thereof.

22. A method of treating a disorder or disease which can be treated by WRN inhibition in a subject, wherein the method comprises administering to the subject a therapeutically effective amount of the compound according to any one of claims 1-18, or a pharmaceutically acceptable salt thereof.

23. A method of inhibiting WRN in a subject, wherein the method comprises administering to the subject a therapeutically effective amount of the compound according to any one of claims 1-18, or a pharmaceutically acceptable salt thereof.

24. The method of claim 22, wherein the disorder or disease is a cancer characterized as microsatellite instability-high (MSI-H) or mismatch repair deficient (dMMR).

25. The method of claim 24, wherein the cancer characterized as microsatellite instability-high (MSI-H) or mismatch repair deficient (dMMR) is selected from colorectal, gastric, prostate, endometrial, adrenocortical, uterine, cervical, esophageal, breast, kidney and ovarian cancer.
